SEQUENCE LISTING (837 sequences)

SEQ ID NO: 1-237 - skipped (237 entries)

SEQ ID NO: 238 - protein, 447 aa, Artificial Sequence
  source: /note=“Description of Artificial Sequence: Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 239 - protein, 215 aa, Artificial Sequence
  source: /note=“Description of Artificial Sequence: Synthetic polypeptide”
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 121 aa, Artificial Sequence
  source: /note=“Description of Artificial Sequence: Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLWEVRALPSVYWGQGTLVTVSS

SEQ ID NO: 241 - protein, 109 aa, Artificial Sequence
  source: /note=“Description of Artificial Sequence: Synthetic polypeptide”
SYELTQPPSVSVAPGQTARITCGANDIGSKSVHWYQQKAGQAPVLVVSEDIIRPSGIPERISGSNSGNTATLTISRVEAGDEADYYCQVWDRDSDQYVFGTGTKVTVLG

SEQ ID NO: 242-500 - skipped (259 entries)

SEQ ID NO: 501 - protein, 5 aa, Artificial Sequence
  source: /note=“Description of Artificial Sequence: Synthetic peptide”
TYWMH

SEQ ID NO: 502 - protein, 17 aa, Artificial Sequence
  source: /note=“Description of Artificial Sequence: Synthetic peptide”
NIYPGTGGSNFDEKFKN

SEQ ID NO: 503 - protein, 8 aa, Artificial Sequence
  source: /note=“Description of Artificial Sequence: Synthetic peptide”
WTTGTGAY

SEQ ID NO: 504 - protein, 7 aa, Artificial Sequence
  source: /note=“Description of Artificial Sequence: Synthetic peptide”
GYTFTTY

SEQ ID NO: 505 - protein, 6 aa, Artificial Sequence
  source: /note=“Description of Artificial Sequence: Synthetic peptide”
YPGTGG

SEQ ID NO: 506 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507 - DNA, 351 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 508 - protein, 443 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 509 - DNA, 1329 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 510 - protein, 17 aa, Artificial Sequence
  source: /note=“Description of Artificial Sequence: Synthetic peptide”
KSSQSLLDSGNQKNFLT

SEQ ID NO: 511 - protein, 7 aa, Artificial Sequence
  source: /note=“Description of Artificial Sequence: Synthetic peptide”
WASTRES

SEQ ID NO: 512 - protein, 9 aa, Artificial Sequence
  source: /note=“Description of Artificial Sequence: Synthetic peptide”
QNDYSYPYT

SEQ ID NO: 513 - protein, 13 aa, Artificial Sequence
  source: /note=“Description of Artificial Sequence: Synthetic peptide”
SQSLLDSGNQKNF

SEQ ID NO: 514 - protein, 3 aa, Artificial Sequence
  source: /note=“Description of Artificial Sequence: Synthetic peptide”
WAS

SEQ ID NO: 515 - protein, 6 aa, Artificial Sequence
  source: /note=“Description of Artificial Sequence: Synthetic peptide”
DYSYPY

SEQ ID NO: 516 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 518 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 520 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 522 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 524 - DNA, 15 bp, Artificial Sequence
  source: /note=“Description of Artificial Sequence: Synthetic oligonucleotide”
acctactggatgcac

SEQ ID NO: 525 - DNA, 51 bp, Artificial Sequence
  source: /note=“Description of Artificial Sequence: Synthetic oligonucleotide”
aatatctaccccggcaccggcggctctaacttcgacgagaagtttaagaat

SEQ ID NO: 526 - DNA, 24 bp, Artificial Sequence
  source: /note=“Description of Artificial Sequence: Synthetic oligonucleotide”
tggactaccggcacaggcgcctac

SEQ ID NO: 527 - DNA, 21 bp, Artificial Sequence
  source: /note=“Description of Artificial Sequence: Synthetic oligonucleotide”
ggctacaccttcactacctac

SEQ ID NO: 528 - DNA, 18 bp, Artificial Sequence
  source: /note=“Description of Artificial Sequence: Synthetic oligonucleotide”
taccccggcaccggcggc

SEQ ID NO: 529 - DNA, 51 bp, Artificial Sequence
  source: /note=“Description of Artificial Sequence: Synthetic oligonucleotide”
aaatctagtcagtcactgctggatagcggtaatcagaagaacttcctgacc

SEQ ID NO: 530 - DNA, 21 bp, Artificial Sequence
  source: /note=“Description of Artificial Sequence: Synthetic oligonucleotide”
tgggcctctactagagaatca

SEQ ID NO: 531 - DNA, 27 bp, Artificial Sequence
  source: /note=“Description of Artificial Sequence: Synthetic oligonucleotide”
cagaacgactatagctacccctacacc

SEQ ID NO: 532 - DNA, 39 bp, Artificial Sequence
  source: /note=“Description of Artificial Sequence: Synthetic oligonucleotide”
agtcagtcactgctggatagcggtaatcagaagaacttc

SEQ ID NO: 533 - DNA, 9 bp, Artificial Sequence
  source: /note=“Description of Artificial Sequence: Synthetic oligonucleotide”
tgggcctct

SEQ ID NO: 534 - DNA, 18 bp, Artificial Sequence
  source: /note=“Description of Artificial Sequence: Synthetic oligonucleotide”
gactatagctacccctac

SEQ ID NO: 535 - protein, 440 aa, Artificial Sequence
  source: /note=“Description of Artificial Sequence: Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 536 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 537 - protein, 447 aa, Artificial Sequence
  source: /note=“Description of Artificial Sequence: Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 538 - protein, 218 aa, Artificial Sequence
  source: /note=“Description of Artificial Sequence: Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 539 - protein, 447 aa, Artificial Sequence
  source: /note=“Description of Artificial Sequence: Synthetic polypeptide”
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 213 aa, Artificial Sequence
  source: /note=“Description of Artificial Sequence: Synthetic polypeptide”
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 541 - protein, 10 aa, Artificial Sequence
  source: /note=“Description of Artificial Sequence: Synthetic peptide”
GYTFTTYWMH

SEQ ID NO: 542-800 - skipped (259 entries)

SEQ ID NO: 801 - protein, 5 aa, Artificial Sequence
  source: /note=“Description of Artificial Sequence: Synthetic peptide”
SYNMH

SEQ ID NO: 802 - protein, 17 aa, Artificial Sequence
  source: /note=“Description of Artificial Sequence: Synthetic peptide”
DIYPGNGDTSYNQKFKG

SEQ ID NO: 803 - protein, 9 aa, Artificial Sequence
  source: /note=“Description of Artificial Sequence: Synthetic peptide”
VGGAFPMDY

SEQ ID NO: 804 - protein, 7 aa, Artificial Sequence
  source: /note=“Description of Artificial Sequence: Synthetic peptide”
GYTFTSY

SEQ ID NO: 805 - protein, 6 aa, Artificial Sequence
  source: /note=“Description of Artificial Sequence: Synthetic peptide”
YPGNGD

SEQ ID NO: 806 - protein, 118 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 807 - DNA, 354 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 808 - protein, 444 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 809 - DNA, 1332 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 810 - protein, 15 aa, Artificial Sequence
  source: /note=“Description of Artificial Sequence: Synthetic peptide”
RASESVEYYGTSLMQ

SEQ ID NO: 811 - protein, 7 aa, Artificial Sequence
  source: /note=“Description of Artificial Sequence: Synthetic peptide”
AASNVES

SEQ ID NO: 812 - protein, 9 aa, Artificial Sequence
  source: /note=“Description of Artificial Sequence: Synthetic peptide”
QQSRKDPST

SEQ ID NO: 813 - protein, 11 aa, Artificial Sequence
  source: /note=“Description of Artificial Sequence: Synthetic peptide”
SESVEYYGTSL

SEQ ID NO: 814 - protein, 3 aa, Artificial Sequence
  source: /note=“Description of Artificial Sequence: Synthetic peptide”
AAS

SEQ ID NO: 815 - protein, 6 aa, Artificial Sequence
  source: /note=“Description of Artificial Sequence: Synthetic peptide”
SRKDPS

SEQ ID NO: 816 - protein, 111 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 817 - DNA, 333 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 818 - protein, 218 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 819 - DNA, 654 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 820 - protein, 17 aa, Artificial Sequence
  source: /note=“Description of Artificial Sequence: Synthetic peptide”
DIYPGQGDTSYNQKFKG

SEQ ID NO: 821 - protein, 6 aa, Artificial Sequence
  source: /note=“Description of Artificial Sequence: Synthetic peptide”
YPGQGD

SEQ ID NO: 822 - protein, 118 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 823 - DNA, 354 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 824 - protein, 444 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 825 - DNA, 1332 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 826 - protein, 111 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 827 - DNA, 333 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 828 - protein, 218 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 829 - DNA, 654 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 830 - protein, 114 aa, Artificial Sequence
  source: /note=“Description of Artificial Sequence: Synthetic polypeptide”
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, Artificial Sequence
  source: /note=“Description of Artificial Sequence: Synthetic polypeptide”
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, Artificial Sequence
  source: /note=“Description of Artificial Sequence: Synthetic polypeptide”
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, Artificial Sequence
  source: /note=“Description of Artificial Sequence: Synthetic polypeptide”
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 834 - protein, 137 aa, Artificial Sequence
  source: /note=“Description of Artificial Sequence: Synthetic polypeptide”
MEFGLSWVFLVALLRGVQCQVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSS

SEQ ID NO: 835 - protein, 126 aa, Artificial Sequence
  source: /note=“Description of Artificial Sequence: Synthetic polypeptide”
MLPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIK

SEQ ID NO: 836 - protein, 444 aa, Artificial Sequence
  source: /note=“Description of Artificial Sequence: Synthetic polypeptide”
QVQLQESGPGLVKPSQTLSLTCSFSGFSLSTSGMGVGWIRQPSGKGLEWLAHIWWDGDESYNPSLKSRLTISKDTSKNQVSLKITSVTAADTAVYFCARNRYDPPWFVDWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 837 - protein, 214 aa, Artificial Sequence
  source: /note=“Description of Artificial Sequence: Synthetic polypeptide”
DIQMTQSTSSLSASVGDRVTITCRASQDISNYLSWYQQKPGKAVKLLIYYTSKLHSGVPSRFSGSGSGTDYTLTISSLQQEDFATYFCLQGKMLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0057071 A1
(43) Pub. Date: Feb. 23, 2023

(54) COMBINATION OF ANTI TIM-3 ANTIBODY MBG453 AND ANTI TGF-BETA ANTIBODY NIS793, WITH OR WITHOUT DECITABINE OR THE ANTI PD-1 ANTIBODY SPARTALIZUMAB, FOR TREATING MYELOFIBROSIS AND MYELODYSPLASTIC SYNDROME

(71) Applicant: NOVARTIS AG, Basel (CH)

(72) Inventors: K. Gary J. VANASSE, Wellesley, MA (US); Haiying SUN, East Hanover, NJ (US); Mirek DOSTALEK, Binningen (CH); Mikael RINNE, Cambridge, MA (US); Anshu MARATHE, Basking Ridge, NJ (US); Luigi MANENTI, Cenate Sopra (IT); Claire FABRE, Basel (CH); Fariba KHANSHAN, Brookline, MA (US)

(21) Appl. No.: 17/786,717

(22) PCT Filed: Dec. 3, 2020
(86) PCT No.: PCT/IB2020/000987

§ 371 Date: Jun. 17, 2022

Related U.S. Application Data
(60) Provisional application No. 63/117,206, filed on Nov. 23, 2020.
(60) Provisional application No. 63/090,259, filed on Oct. 11, 2020.
(60) Provisional application No. 63/090,264, filed on Oct. 11, 2020.
(60) Provisional application No. 63/055,230, filed on Jul. 22, 2020.
(60) Provisional application No. 62/951,632, filed on Dec. 20, 2019.
(60) Provisional application No. 62/978,267, filed on Feb. 18, 2020.

(51) Int. Cl. C07K 16/22; C07K 16/28; C07K 16/24; A61K 31/706; A61P 35/00

(52) U.S. Cl. CPC C07K 16/22; C07K 16/2803; C07K 16/2818; C07K 16/245; A61K 31/706; A61P 35/00; A61K 2039/545

(57) ABSTRACT

Combination therapies comprising TIM-3 inhibitors and TGF-β inhibitors are disclosed. The combinations can be used to treat or prevent cancerous conditions and disorders, including myelofibrosis or myelodysplastic syndrome.

### CROSS REFERENCE TO RELATED APPLICATIONS

This application claims the benefit of U.S. Provisional Application No. 62/951,632, filed Dec. 20, 2019, U.S. Provisional Application No. 62/978,267 filed on Feb. 18, 2020, U.S. Provisional Application No. 63/055,230, filed on Jul. 22, 2020, U.S. Provisional Application No. 63/090,259 filed on Oct. 11, 2020, U.S. Provisional Application No. 63/090,264 filed on Oct. 11, 2020, and U.S. Provisional Application No. 63/117,206, filed on Nov. 23, 2020. The contents of the aforementioned applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Dec. 1, 2020, is named C2160-7031WO_SL.txt and is 116,296 bytes in size.

### BACKGROUND

Myelofibrosis (MF) is a Philadelphia chromosome-negative myeloproliferative neoplasm (MPN) characterized by the presence of megakaryocyte proliferation and atypia, usually accompanied by either reticulin and/or collagen fibrosis (Tefferi and Vardiman (2008) Leukemia 22(1):14-22), splenomegaly (due to extramedullary hematopoiesis), anemia (due to bone marrow failure and splenic sequestration), and debilitating constitutional symptoms (due to overexpression of inflammatory cytokines) that include fatigue, weight loss, pruritus, night sweats, fever, and bone, muscle, or abdominal (Mesa et al. (2007) Cancer 109(1):68-76; Abdel Wahab and Levine (2009) Annu Rev Med. 60:233-45; Naymagon et al. (2017) HemaSphere 1(1): p e1).

MF is defined by the National Institutes of Health (NIH) as a “rare disease” with a prevalence of 0.3 to 1.5 cases per 100 000 with median age at diagnosis of 65 years (Mehta et al. (2014) Leuk Lymphoma 55(3):595-600; Rollison et al. (2008) Blood 112(1):45-52).

MF can develop de novo, as a primary hematologic malignancy, primary myelofibrosis (PMF) or arise from the progression of preexisting myeloproliferative neoplasms, namely: polycythemia vera (PV), post-PV MF (PPV-MF) and essential thrombocythemia (ET), post-ET MF (PET-MF)) (Mesa et al. (2007) Leuk Res. 31(6)737-40; Naymagon et al. (2017) HemaSphere 1(1): p e1).

The only potential curative treatment for MF is allogeneic hematopoietic stem cell transplantation (ASCT), for which the great majority of patients is ineligible. Therefore, treatment options remain primarily palliative, and aimed at controlling disease symptoms, complications, and improving the patients' quality of life (QoL). The therapeutic landscape of MF has changed with the discovery of the V617F mutation of the Janus kinase JAK2 gene present in 60% of patients with PMF or PET-MF and in 95% of patients with PPV-MF, triggering the development of molecular targeted therapy for MF (Cervantes (2014) Blood 124(17):2635-2642). JAKs play an important role in signal transduction following cytokine and growth factor binding to their receptors. Aberrant activation of JAKs has been associated with increased malignant cell proliferation and survival (Valentino and Pierre (2006) Biochem Pharmacol. 71(6):713-721). JAKs activate a number of downstream signaling pathways implicated in the proliferation and survival of malignant cells including members of the Signal Transducer and Activator of Transcriptions (STAT) family of transcription factors. JAK inhibitors were developed to target JAK2 thereby inhibiting JAK signaling.

Current treatment options post JAK inhibitors are limited in their efficacy, durability and tolerability. Multiple efforts are currently ongoing to improve the outcome of patients with MF post JAK inhibitors identifying new agents or combinations, such as those targeting cellular metabolic and apoptotic pathways, cell cycle and immune therapy. There is a need for improved treatments for MF.

Myelodysplastic syndromes (MDS) correspond to a heterogeneous group of hematological malignancies associated with impaired bone marrow function, ineffective hematopoiesis, elevated bone marrow blasts, and persistent peripheral blood cytopenias. Anemia is one of the most common symptoms of MDS and as a result, most patients with MDS undergo at least one red blood cell transfusion. MDS can also progress to acute myeloid leukemia (AML) (Heaney and Golde (1999) N. Engl, J. Med. 340(21):1649-60). Although progression to AML can lead to death in patients with MDS, MDS-related deaths can also result from cytopenias and marrow failure in the absence of leukemic transformation. Prognosis of MDS is typically determined using the revised International Prognostic Scoring System (IPSS-R), which considers the percentage of bone marrow blasts, the number of cytopenias, and bone marrow cytogenetics. Patients with untreated MDS are classified into five IPSS-R prognostic risk categories: very low, low, intermediate, high and very high, (Greenberg et al. (2012) Blood 108(11):2623). Very low, low, and intermediate risk MDS constitute lower risk MDS. High and very high risk MDS are referred to as higher risk MDS.

Patients with very low and low risk MDS are treated with supportive care to control symptoms resulting from cytopenia. Lower risk MDS can progress to bone marrow failure. Prognosis is poor and life expectancy is short in intermediate, high, or very high risk MDS. The current standard of care is the use of a hypomethylating agent, chemotherapy, and/or hematopoietic stem cell transplant (HSCT). HSCT is the only curative option. However, only a minority of MDS patients are candidates for HSCT and intensive chemotherapy (Steensma (2018) Blood Cancer J 8(5): 47; Platzbecker (2019) Blood 133(10): 1096-1107; Itzykson et al. (2018) HemaSphere 2(6):150). Complete remission is only reported in a minority of patients treated by azacitidine alone, and clinical benefits of this drug are frequently transient. When treatment fails, additional treatment options are limited. Despite the fact that single-agent hypomethylating agents are available for the treatment of patients with MDS, alternative treatment strategies are needed.

### SUMMARY

Disclosed herein, at least in part, are combinations comprising inhibitors of T-cell immunoglobulin domain and mucin domain 3 (TIM-3). In some embodiments, the combination comprises an antibody molecule (e.g., a humanized antibody molecule) that binds to TIM-3 with high affinity and specificity. In some embodiments, the combination further comprises an inhibitor of TGF-β. In some embodiments, the combination further comprises a hypomethylating agent, and/or an inhibitor of PD-1 or an inhibitor of IL-1β. Pharmaceutical compositions and dose formulations relating to the combinations described herein are also provided. The combinations described herein can be used to treat or prevent disorders, such as myelofibrosis (e.g., a primary myelofibrosis (PMF), post-essential thrombocythemia myelofibrosis (PET-MF), post-polycythemia vera myelofibrosis (PPV-MF)), or a myelodysplastic syndrome (MDS) (e.g., a lower risk MDS (e.g., a very low risk MDS, a low risk MDS, or an intermediate risk MDS) or a higher risk MDS (e.g., a high risk MDS or a very high risk MDS)). Thus, methods, including dosage regimens, for treating various disorders using the combinations are disclosed herein.

Accordingly, in one aspect, the disclosure features a method of treating a myelofibrosis or a myelodysplastic syndrome (MDS) in a subject, comprising administering to the subject a combination of a TIM-3 inhibitor and a TGF-β inhibitor.

In some embodiments, the TIM-3 inhibitor comprises an anti-TIM-3 antibody molecule. In some embodiments, the TIM-3 inhibitor comprises MBG453, TSR-022, LY3321367, Sym023, BGB-A425, INCAGN-2390, MBS-986258, RO-7121661, BC-3402, SHR-1702, or LY-3415244. In some embodiments, the TIM-3 inhibitor comprises MBG453. In some embodiments, the TIM-3 inhibitor is administered at a dose of about 400 mg to about 1200 mg once every two weeks, once every three weeks, once every four weeks, once every six weeks, or once every eight weeks. In some embodiments, the TIM-3 inhibitor is administered at a dose of about 700 mg to about 900 mg. In some embodiments, the TIM-3 inhibitor is administered at a dose of about 800 mg. In some embodiments, the TIM-3 inhibitor is administered at a dose of about 300 mg to about 500 mg. In some embodiments, the TIM-3 inhibitor is administered at a dose of about 400 mg. In some embodiments, the TIM-3 inhibitor is administered once every eight weeks. In some embodiments, the TIM-3 inhibitor is administered once every four weeks. In some embodiments, the TIM-3 inhibitor is administered at a dose of about 700 mg to about 900 mg (e.g., about 800 mg) once every eight weeks. In some embodiments, the TIM-3 inhibitor is administered at a dose of about 700 mg to about 900 mg (e.g., about 800 mg) once every four weeks. In some embodiments, the TIM-3 inhibitor is administered at a dose of about 300 mg to about 500 mg (e.g., about 400 mg) once every eight weeks. In some embodiments, the TIM-3 inhibitor is administered at a dose of about 300 mg to about 500 mg (e.g., about 400 mg) once every four weeks. In some embodiments, the TIM-3 inhibitor is administered at a dose of about 500 mg to about 700 mg. In some embodiments, the TIM-3 inhibitor is administered at a dose of about 600 mg. In some embodiments, the TIM-3 inhibitor is administered once every three weeks. In some embodiments, the TIM-3 inhibitor is administered once every six weeks. In some embodiments, the TIM-3 inhibitor is administered once every four weeks. In some embodiments, the TIM-3 inhibitor is administered at a dose of about 500 mg to about 700 mg (e.g., about 600 mg) once every three weeks. In some embodiments, the TIM-3 inhibitor is administered once every four weeks. In some embodiments, the TIM-3 inhibitor is administered at a dose of about 500 mg to about 700 mg (e.g., about 600 mg) once every six weeks. In some embodiments, the TIM-3 inhibitor is administered intravenously. In some embodiments, the TIM-3 inhibitor is administered intravenously over a period of about 20 minutes to about 40 minutes. In some embodiments, the TIM-3 inhibitor is administered intravenously over a period of about 30 minutes.

In some embodiments, the TGF-β inhibitor is an anti-TGF-β antibody molecule. In some embodiments the TGF-β inhibitor comprises NIS793, fresolimumab, PF-06952229 or AVID200. In some embodiments the TGF-β inhibitor comprises NIS793. In some embodiments, the TGF-β inhibitor is administered at a dose of about 1200 mg to about 2200 mg. In some embodiments, the TGF-β inhibitor is administered at a dose of about 600 mg to about 2200 mg. In some embodiments, the TGF-β inhibitor is administered at a dose of about 1400 mg to about 2100 mg. In some embodiments, the TGF-β inhibitor is administered at a dose of about 600 mg to about 800 mg. In some embodiments, the TGF-β inhibitor is administered at a dose of about 700 mg. In some embodiments, the TGF-β inhibitor is administered once every three weeks. In some embodiments, the TGF-β inhibitor is administered at a dose of about 600 mg to about 2200 mg (e.g., about 1200 mg to about 2200 mg, about 1400 mg to about 2100 mg, or about 600 mg to about 800 mg (e.g., about 700 mg)) once every three weeks. In some embodiments, the TGF-β inhibitor is administered at a dose of about 1300 mg to about 1500 mg. In some embodiments, the TGF-β inhibitor is administered at a dose of about 1400 mg. In some embodiments, the TGF-β inhibitor is administered once every two weeks. In some embodiments, the TGF-β inhibitor is administered once every three weeks. In some embodiments, the TGF-β inhibitor is administered once every six weeks. In some embodiments, the TGF-β inhibitor is administered at a dose of about 1300 mg to about 1500 mg (e.g., about 1400 mg) once every two weeks, once every three weeks, or once every six weeks. In some embodiments, the TGF-β inhibitor is administered at a dose of about 2000 mg to about 2200 mg. In some embodiments, the TGF-β inhibitor is administered at a dose of about 2100 mg. In some embodiments, the TGF-β inhibitor is administered once every three weeks. In some embodiments, the TGF-β inhibitor is administered at a dose of about 2000 mg to about 2200 mg (e.g., about 2100 mg) once every three weeks. In some embodiments, the TGF-β inhibitor is administered at a flat dose. In some embodiments, the TGF-β inhibitor is administered according to a dose escalation regimen. In some embodiments, the TGF-β inhibitor is administered over a period of about 20 to about 40 minutes. In some embodiments, the TGF-β inhibitor is administered over a period of about 30 minutes. In some embodiments, the TGF-β inhibitor is administered on the same day as the TIM-3 inhibitor. In some embodiments, the TGF-β inhibitor is administered after administration of the TIM-3 inhibitor is started.

In some embodiments, the combination further comprises a PD-1 inhibitor. In some embodiments, the PD-1 inhibitor comprises spartalizumab, nivolumab, pembrolizumab, pidilizumab, MEDI0680, REGN2810, TSR-042, PF-06801591, BGB-A317, BGB-108, INCSHR1210, or AMP-224. In some embodiments, the PD-1 inhibitor comprises spartalizumab. In some embodiments, the PD-1 inhibitor is administered at a dose of about 200 mg to about 400 mg every three or four weeks. In some embodiments, the PD-1 inhibitor is administered at a dose of about 300 mg to about 500 mg. In some embodiments, the PD-1 inhibitor is administered at a dose of about 400 mg. In some embodiments, the PD-1 inhibitor is administered once every four weeks. In some embodiments, the PD-1 inhibitor is administered at a dose of about 300 mg to about 500 mg (e.g., about 400 mg) once every four weeks. In some embodiments, the PD-1 inhibitor is administered at a dose of about 200 mg to about 400 mg. In some embodiments, the PD-1 inhibitor is administered at a dose of about 300 mg. In some embodiments, the PD-1 inhibitor is administered once every three weeks. In some embodiments, the PD-1 inhibitor is administered at a dose of about 200 mg to about 400 mg (e.g., about 300 mg) once every three weeks. In some embodiments, the PD-1 inhibitor is administered intravenously. In some embodiments, the PD-1 inhibitor is administered over a period of about 20 to about 40 minutes. In some embodiments, the PD-1 inhibitor is administered over a period of about 30 minutes.

In some embodiments, the combination further comprises an interleukin-1 beta (IL-1β) inhibitor. In some embodiments, the IL-1β inhibitor is canakinumab or gevokizumab. In some embodiments, the IL-1β inhibitor is canakinumab. In some embodiments, the IL-1β inhibitor is administered at a dose of about 300 mg to about 500 mg. In some embodiments, the IL-1β inhibitor is administered at a dose of about 200 mg. In some embodiments, the IL-1β inhibitor is administered at a dose of about 250 mg. In some embodiments, the IL-1β inhibitor is administered once every three weeks, once every four weeks, or once every eight weeks. In some embodiments, the IL-1β inhibitor is administered once every three weeks. In some embodiments, the IL-1β inhibitor is administered once every four weeks. In some embodiments, the IL-1β inhibitor is administered once every eight weeks. In some embodiments, the IL-1β inhibitor is administered at a dose of about 300 mg to about 500 mg (e.g., about 200 mg or about 250 mg) once every three weeks, once every four weeks, or once every eight weeks. In some embodiments, the IL-1β inhibitor is administered intravenously. In some embodiments, the IL-1β inhibitor is administered subcutaneously.

In some embodiments, the combination further comprises a hypomethylating agent. In some embodiments, the hypomethylating agent comprises decitabine, azacitidine, CC-486, or ASTX727. In some embodiments, the hypomethylating agent comprises decitabine or azacitidine. In some embodiments, the hypomethylating agent comprises decitabine. In some embodiments, the hypomethylating agent is administered at a dose of about 2 mg/m² to about 25 mg/m². In some embodiments, the hypomethylating agent is administered at a dose of about 2.5 mg/m², about 5 mg/m², about 10 mg/m², or about 20 mg/m². In some embodiments, the hypomethylating agent is administered at a starting dose of about 5 mg/m² and escalated up to 20 mg/m². In some embodiments, the hypomethylating agent is administered once a day. In some embodiments, the hypomethylating agent is administered at a dose of about 2 mg/m² to about 25 mg/m² (e.g., about 2.5 mg/m², about 5 mg/m², about 10 mg/m², or about 20 mg/m²) once a day. In some embodiments, the hypomethylating agent is administered for 2-7 consecutive days, e.g., 3 or 5 consecutive days. In some embodiments, the hypomethylating agent is administered for 5 consecutive days. In some embodiments, the hypomethylating agent is administered according to a 3 day regimen, every 6 weeks. In some embodiments, the hypomethylating agent is administered according to a 5 day regimen, every 6 weeks. In some embodiments, the hypomethylating agent is administered according to a 3 day regimen, every 4 weeks. In some embodiments, the hypomethylating agent is administered on days 1, 2, 3, 4, and 5 of a 28 days cycle. In some embodiments, the hypomethylating agent is administered over a period of about 0.5 hour to about 1.5 hour. In some embodiments, the hypomethylating agent is administered over a period of about 1 hour. In some embodiments, the hypomethylating agent is administered at a dose of about 2 mg/m² to about 20 mg/m². In some embodiments, the hypomethylating agent is administered at a dose of about 2.5 mg/m², about 5 mg/m², about 7.5 mg/m², about 15 mg/m², or about 20 mg/m². In some embodiments, the hypomethylating agent is administered once every eight hours. In some embodiments, the hypomethylating agent is administered at a dose of about 2 mg/m² to about 20 mg/m² (e.g., about 2.5 mg/m², about 5 mg/m², about 7.5 mg/m², about 15 mg/m², or about 20 mg/m²) once every eight hours. In some embodiments, the hypomethylating agent is administered for 3 consecutive days. In some embodiments, the hypomethylating agent is administered for 5 consecutive days. In some embodiments, the hypomethylating agent is administered over a period of about 2 hours to about 4 hours. In some embodiments, the hypomethylating agent is administered over a period of about 3 hours. In some embodiments, the hypomethylating agent is administered subcutaneously or intravenously.

In some embodiments, the combination further comprise a CD47 inhibitor, a CD70 inhibitor, a NEDD8 inhibitor, a CDK9 inhibitor, an FLT3 inhibitor, a KIT inhibitor, or a p53 activator, or any combination thereof, e.g., a CD47 inhibitor, a CD70 inhibitor, a NEDD8 inhibitor, a CDK9 inhibitor, an FLT3 inhibitor, a KIT inhibitor, or a p53 activator, all as described herein.

In some embodiments, the myelofibrosis is a primary myelofibrosis (PMF), a post-essential thrombocythemia myelofibrosis (PET-MF), or a post-polycythemia vera myelofibrosis (PPV-MF). In some embodiments, the myelofibrosis is a primary myelofibrosis (PMF).

In some embodiments, the myelodysplastic syndrome (MDS) is a lower risk MDS (e.g., a very low risk MDS, a low risk MDS, or an intermediate MDS), or a higher risk MDS (e.g., a high risk MDS or a very high risk MDS). In some embodiments, the MDS is a lower risk MDS.

In another aspect, the disclosure features a method of treating a myelofibrosis in a subject, comprising administering to the subject a combination of a TIM-3 inhibitor and a TGF-β inhibitor.

In another aspect, the disclosure features a method of treating myelofibrosis in a subject, comprising administering to the subject a combination of a TIM-3 inhibitor, TGF-β inhibitor, and a hypomethylating agent.

In another aspect, the disclosure features a method of treating myelofibrosis in a subject, comprising administering to the subject a combination of a TIM-3 inhibitor, TGF-β inhibitor, and a PD-1 inhibitor, and optionally a hypomethylating agent.

In another aspect, the disclosure features a method of treating myelofibrosis in a subject, comprising administering to the subject a combination of a TIM-3 inhibitor, TGF-β inhibitor, and an IL-1β inhibitor, and optionally a hypomethylating agent.

In another aspect, the disclosure features a combination comprising MBG453 and NIS793 for use in treating a myelofibrosis in a subject, optionally wherein the combination further comprises decitabine, and optionally wherein the combination further comprises PDR001, and optionally wherein MGB453 is administered at a dose of 500 mg to 700 mg (e.g., 600 mg) once every three weeks, NIS793 is administered at a dose of 2000 mg to 2200 mg (e.g., 2100 mg) once every three weeks, PDR001 is administered at a dose of 200 mg to 400 mg (e.g., 300 mg) once every three weeks and/or decitabine is administered at a dose of about 5 mg/m² to about 20 mg/m² on days 1, 2, and 3 of a 42 day cycle.

In another aspect, the disclosure features a method of treating a myelofibrosis in a subject, comprising administering to the subject a combination of a MBG453 and NIS793, optionally wherein the combination further comprises decitabine, and optionally wherein the combination further comprises PDR001, and optionally wherein MGB453 is administered at a dose of 500 mg to 700 mg (e.g., 600 mg) once every three weeks, NIS793 is administered at a dose of 2000 mg to 2200 mg (e.g., 2100 mg) once every three weeks, PDR001 is administered at a dose of 200 mg to 400 mg (e.g., 300 mg) once every three weeks and/or decitabine is administered at a dose of about 5 mg/m² to about 20 mg/m² on days 1, 2, and 3 of a 42 day cycle.

In another aspect, the disclosure features a method of treating myelofibrosis in a subject, comprising administering to the subject a combination of a MBG453 and NIS793, optionally wherein the combination further comprises decitabine, and optionally wherein the combination further comprises canakinumab, and optionally wherein MGB453 is administered at a dose of 500 mg to 700 mg (e.g., 600 mg once every three weeks, NIS793 is administered at a dose of 2000 mg to 2200 mg (e.g., 2100 mg once every three weeks, and canakinumab is administered at a dose of 150 mg to 250 mg (e.g., 200 mg) once every three weeks, and decitabine is administered at a dose of about 5 mg/m² to about 20 mg/m² on days 1, 2, and 3 of a 42 day cycle.

In another aspect, the disclosure features a combination comprising MBG453 and NIS793 for use in treating a myelofibrosis in a subject, optionally wherein the combination further comprises decitabine, and optionally wherein the combination further comprises canakinumab, and optionally wherein MGB453 is administered at a dose of 500 mg to 700 mg (e.g., 600 mg) once every three weeks, NIS793 is administered at a dose of 2000 mg to 2200 mg (e.g., 2100 mg) once every three weeks, and canakinumab is administered at a dose of 150 mg to 250 mg (e.g., 200 mg) once every three weeks, and decitabine is administered at a dose of about 5 mg/m² to about 20 mg/m² on days 1, 2, and 3 of a 42 day cycle.

In another aspect, the disclosure features a method of treating myelofibrosis in a subject, comprising administering to the subject a combination of a MBG453 and NIS793, optionally wherein the combination further comprises decitabine, and optionally wherein the combination further comprises canakinumab, and optionally wherein MGB453 is administered at a dose of 700 mg to 900 mg (e.g., 800 mg) once every four weeks, NIS793 is administered at a dose of 1300 mg to 1500 mg (e.g., 1400 mg) once every two weeks, and canakinumab is administered at a dose of 200 mg to 300 mg (e.g., 250 mg) once every four weeks, and decitabine is administered at a dose of about 5 mg/m² to about 20 mg/m² on days 1, 2, and 3 of a 42 day cycle.

In another aspect, the disclosure features a combination comprising MBG453 and NIS793 for use in treating a myelofibrosis in a subject, optionally wherein the combination further comprises decitabine, and optionally wherein the combination further comprises canakinumab, and optionally wherein MGB453 is administered at a dose of 700 mg to 900 mg (e.g., 800 mg) once every four weeks, NIS793 is administered at a dose of 1300 mg to 1500 mg (e.g., 1400 mg) once every two weeks, and canakinumab is administered at a dose of 200 mg to 300 mg (e.g., 250 mg) once every four weeks, and decitabine is administered at a dose of about 5 mg/m² to about 20 mg/m² on days 1, 2, and 3 of a 42 day cycle.

In another aspect, the disclosure features a method of reducing an activity (e.g., growth, survival, or viability, or all), of a cancer cell, e.g., hematological cancer cell. The method includes contacting the cell with a combination described herein. The method can be performed in a subject, e.g., as part of a therapeutic protocol. The hematological cancer cell can be, e.g., a cell from a hematological cancer described herein, such as a myeloproliferative neoplasm (MPN), e.g., myelofibrosis (e.g., a primary myelofibrosis (PMF), post-essential thrombocythemia myelofibrosis (PET-MF), post-polycythemia vera myelofibrosis (PPV-MF)), or a myelodysplastic syndrome (MDS) (e.g., a lower risk MDS (e.g., a very low risk MDS, a low risk MDS, or an intermediate MDS) or a higher risk MDS (e.g., a high risk MDS or a very high risk MDS)).

In certain embodiments of the methods disclosed herein, the method further includes determining the level of TIM-3 expression in tumor infiltrating lymphocytes (TILs) in the subject. In other embodiments, the level of TIM-3 expression is determined in a sample (e.g., a liquid biopsy) acquired from the subject (e.g., using immunohistochemistry). In certain embodiments, responsive to a detectable level, or an elevated level, of TIM-3 in the subject, the combination is administered. The detection steps can also be used, e.g., to monitor the effectiveness of a therapeutic agent described herein. For example, the detection step can be used to monitor the effectiveness of the combination.

In another aspect, the disclosure features a composition (e.g., one or more compositions or dosage forms), that includes a TIM-3 inhibitor, TGF-β inhibitor, optionally further comprising a hypomethylating agent, and optionally further comprising a PD-1 inhibitor or an IL-1β inhibitor, as described herein. Formulations, e.g., dosage formulations, and kits, e.g., therapeutic kits, that include a TIM-3 inhibitor, TGF-β inhibitor, optionally further comprising a hypomethylating agent, and optionally further comprising a PD-1 inhibitor or an IL-1β inhibitor, are also described herein. In certain embodiments, the composition or formulation is used to treat myelofibrosis (e.g., a primary myelofibrosis (PMF), post-essential thrombocythemia myelofibrosis (PET-MF), post-polycythemia vera myelofibrosis (PPV-MF)).

In another aspect, the disclosure features a composition (e.g., one or more compositions or dosage forms), that includes a TIM-3 inhibitor, and a TGF-β inhibitor. Formulations, e.g., dosage formulations, and kits, e.g., therapeutic kits, that include a TIM-3 inhibitor and a TGF-β inhibitor, are also described herein. In certain embodiments, the composition or formulation is used to treat a myelodysplastic syndrome (MDS) (e.g., a lower risk MDS (e.g., a very low risk MDS, a low risk MDS, or an intermediate MDS) or a higher risk MDS (e.g., a high risk MDS or a very high risk MDS)).

In another aspect, the disclosure features a method of treating a myelodysplastic syndrome (MDS) (e.g., lower risk MDS) in a subject, comprising administering to the subject a combination of a TIM-3 inhibitor and a TGF-β inhibitor.

In another aspect, the disclosure features a method of treating a myelodysplastic syndrome (MDS) (e.g., lower risk MDS) in a subject, comprising administering to the subject a combination of a TIM-3 inhibitor, a TGF-β inhibitor, and an IL-1β inhibitor.

In another aspect, the disclosure features a combination comprising MBG453 and NIS793 for use in treating a myelodysplastic syndrome (MDS) (e.g., lower risk MDS) in a subject, optionally wherein MGB453 is administered at a dose of 700 mg to 900 mg (e.g., 800 mg) once every four weeks, and NIS793 is administered at a dose of 2000 mg to 2200 mg (e.g., 2100 mg) once every three weeks.

In another aspect, the disclosure features a combination comprising MBG453 and NIS793 for use in treating a myelodysplastic syndrome (MDS) (e.g., lower risk MDS) in a subject, optionally wherein MGB453 is administered at a dose of 500 mg to 700 mg (e.g., 600 mg) once every three weeks, and NIS793 is administered at a dose of 2000 mg to 2200 mg (e.g., 2100 mg) once every three weeks.

In another aspect, the disclosure features a combination comprising MBG453, NIS793, and canakinumab for use in treating a myelodysplastic syndrome (MDS) (e.g., lower risk MDS) in a subject, optionally wherein MGB453 is administered at a dose of 500 mg to 700 mg (e.g., 600 mg) once every three weeks, NIS793 is administered at a dose of 2000 mg to 2200 mg (e.g., 2100 mg) once every three weeks, and canakinumab is administered at a dose of 200 mg to 300 mg (e.g., 250 mg) once every four weeks.

In another aspect, the disclosure features a combination comprising MBG453, NIS793, and canakinumab for use in treating a myelodysplastic syndrome (MDS) (e.g., lower risk MDS) in a subject, optionally wherein MGB453 is administered at a dose of 700 mg to 900 mg (e.g., 800 mg) once every four weeks, NIS793 is administered at a dose of 2000 mg to 2200 mg (e.g., 2100 mg) once every three weeks, and canakinumab is administered at a dose of 200 mg to 300 mg (e.g., 250 mg) once every four weeks.

In another aspect, the disclosure features a combination comprising MBG453, NIS793, and canakinumab for use in treating a myelodysplastic syndrome (MDS) (e.g., lower risk MDS) in a subject, optionally wherein MGB453 is administered at a dose of 700 mg to 900 mg (e.g., 800 mg) once every four weeks, NIS793 is administered at a dose of 1300 mg to 1500 mg (e.g., 1400 mg) once every three weeks, and canakinumab is administered at a dose of 200 mg to 300 mg (e.g., 250 mg) once every four weeks.

In another aspect, the disclosure features a method of treating a myelodysplastic syndrome (MDS) (e.g., lower risk MDS) in a subject, comprising administering to the subject a combination of a MBG453 and NIS793, optionally wherein MGB453 is administered at a dose of 500 mg to 700 mg (e.g., 600 mg) once every three weeks, and NIS793 is administered at a dose of 2000 mg to 2200 mg (e.g., 2100 mg) once every three weeks.

In another aspect, the disclosure features a method of treating a myelodysplastic syndrome (MDS) (e.g., lower risk MDS) in a subject, comprising administering to the subject a combination of a MBG453, NIS793 and canakinumab, optionally wherein MGB453 is administered at a dose of 500 mg to 700 mg (e.g., 600 mg) once every three weeks, NIS793 is administered at a dose of 2000 mg to 2200 mg (e.g., 2100 mg) once every three weeks, and canakinumab is administered at a dose of 200 mg to 300 mg (e.g., 250 mg) once every four weeks.

In another aspect, the disclosure features a combination comprising MBG453 and NIS793 for use in treating a myelodysplastic syndrome (MDS) (e.g., lower risk MDS) in a subject, optionally wherein MGB453 is administered at a dose of 700 mg to 900 mg (e.g., 800 mg) once every four weeks, and NIS793 is administered at a dose of 2000 mg to 2200 mg (e.g., 2100 mg) once every three weeks.

In another aspect, the disclosure features a combination comprising MBG453, NIS793, canakinumab for use in treating a myelodysplastic syndrome (MDS) (e.g., lower risk MDS) in a subject, optionally wherein MGB453 is administered at a dose of 700 mg to 900 mg (e.g., 800 mg) once every four weeks, NIS793 is administered at a dose of 2000 mg to 2200 mg (e.g., 2100 mg) once every three weeks, and canakinumab is administered at a dose of 200 mg to 300 mg (e.g., 250 mg) once every four weeks.

In another aspect, the disclosure features a combination comprising MBG453, NIS793, canakinumab for use in treating a myelodysplastic syndrome (MDS) (e.g., lower risk MDS) in a subject, optionally wherein MGB453 is administered at a dose of 700 mg to 900 mg (e.g., 800 mg) once every four weeks, NIS793 is administered at a dose of 1300 mg to 1500 mg (e.g., 1400 mg) once every three weeks, and canakinumab is administered at a dose of 200 mg to 300 mg (e.g., 250 mg) once every four weeks.

In another aspect, the disclosure features a method of treating a myelodysplastic syndrome (MDS) (e.g., lower risk MDS) in a subject, comprising administering to the subject a combination of MBG453 and NIS793, optionally wherein MGB453 is administered at a dose of 700 mg to 900 mg (e.g., 800 mg) once every four weeks, and NIS793 is administered at a dose of 2000 mg to 2200 mg (e.g., 2100 mg) once every three weeks.

In another aspect, the disclosure features a method of treating a myelodysplastic syndrome (MDS) (e.g., lower risk MDS) in a subject, comprising administering to the subject a combination of MBG453 and NIS793, optionally wherein MGB453 is administered at a dose of 500 mg to 700 mg (e.g., 600 mg) once every three weeks, and NIS793 is administered at a dose of 2000 mg to 2200 mg (e.g., 2100 mg) once every three weeks.

In another aspect, the disclosure features a method of treating a myelodysplastic syndrome (MDS) (e.g., lower risk MDS) in a subject, comprising administering to the subject a combination of MBG453, NIS793, and canakinumab, optionally wherein MGB453 is administered at a dose of 500 mg to 700 mg (e.g., 600 mg) once every three weeks, NIS793 is administered at a dose of 2000 mg to 2200 mg (e.g., 2100 mg) once every three weeks, and canakinumab is administered at a dose of 200 mg to 300 mg (e.g., 250 mg) once every four weeks.

In another aspect, the disclosure features a method of treating a myelodysplastic syndrome (MDS) (e.g., lower risk MDS) in a subject, comprising administering to the subject a combination of MBG453, NIS793, and canakinumab, optionally wherein MGB453 is administered at a dose of 700 mg to 900 mg (e.g., 800 mg) once every four weeks, NIS793 is administered at a dose of 2000 mg to 2200 mg (e.g., 2100 mg) once every three weeks, and canakinumab is administered at a dose of 200 mg to 300 mg (e.g., 250 mg) once every four weeks.

In another aspect, the disclosure features a method of treating a myelodysplastic syndrome (MDS) (e.g., lower risk MDS) in a subject, comprising administering to the subject a combination of MBG453, NIS793, and canakinumab, optionally wherein MGB453 is administered at a dose of 700 mg to 900 mg (e.g., 800 mg) once every four weeks, NIS793 is administered at a dose of 1300 mg to 1500 mg (e.g., 1400 mg) once every three weeks, and canakinumab is administered at a dose of 200 mg to 300 mg (e.g., 250 mg) once every four weeks.

Additional features or embodiments of the methods, uses, compositions, dosage formulations, and kits described herein include one or more of the following.

### TIM-3 Inhibitors

In some embodiments, the combination described herein comprises a TIM-3 inhibitor, e.g., an anti-TIM-3 antibody. In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 1 (e.g., from the heavy and light chain variable region sequences of ABTIM3-hum11 or ABTIM3-hum03 disclosed in Table 1), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, or encoded by a nucleotide sequence shown in Table 1.

In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 802, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 1. In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 820, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 1.

In one embodiment, the anti-TIM-3 antibody molecule comprises a VH comprising the amino acid sequence of SEQ ID NO: 806, or an amino acid sequence at least 85%, 90%, 95%, or 99% identical or higher to SEQ ID NO: 806. In one embodiment, the anti-TIM-3 antibody molecule comprises a VL comprising the amino acid sequence of SEQ ID NO: 816, or an amino acid sequence at least 85%, 90%, 95%, or 99% identical or higher to SEQ ID NO: 816. In one embodiment, the anti-TIM-3 antibody molecule comprises a VH comprising the amino acid sequence of SEQ ID NO: 822, or an amino acid sequence at least 85%, 90%, 95%, or 99% identical or higher to SEQ ID NO: 822. In one embodiment, the anti-TIM-3 antibody molecule comprises a VL comprising the amino acid sequence of SEQ ID NO: 826, or an amino acid sequence at least 85%, 90%, 95%, or 99% identical or higher to SEQ ID NO: 826. In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment, the antibody molecule comprises a VH encoded by the nucleotide sequence of SEQ ID NO: 807, or a nucleotide sequence at least 85%, 90%, 95%, or 99% identical or higher to SEQ ID NO: 807. In one embodiment, the antibody molecule comprises a VL encoded by the nucleotide sequence of SEQ ID NO: 817, or a nucleotide sequence at least 85%, 90%, 95%, or 99% identical or higher to SEQ ID NO: 817. In one embodiment, the antibody molecule comprises a VH encoded by the nucleotide sequence of SEQ ID NO: 823, or a nucleotide sequence at least 85%, 90%, 95%, or 99% identical or higher to SEQ ID NO: 823. In one embodiment, the antibody molecule comprises a VL encoded by the nucleotide sequence of SEQ ID NO: 827, or a nucleotide sequence at least 85%, 90%, 95%, or 99% identical or higher to SEQ ID NO: 827. In one embodiment, the antibody molecule comprises a VH encoded by the nucleotide sequence of SEQ ID NO: 807 and a VL encoded by the nucleotide sequence of SEQ ID NO: 817. In one embodiment, the antibody molecule comprises a VH encoded by the nucleotide sequence of SEQ ID NO: 823 and a VL encoded by the nucleotide sequence of SEQ ID NO: 827.

In one embodiment, the anti-TIM-3 antibody molecule comprises a heavy chain comprising the amino acid sequence of SEQ ID NO: 808, or an amino acid sequence at least 85%, 90%, 95%, or 99% identical or higher to SEQ ID NO: 808. In one embodiment, the anti-TIM-3 antibody molecule comprises a light chain comprising the amino acid sequence of SEQ ID NO: 818, or an amino acid sequence at least 85%, 90%, 95%, or 99% identical or higher to SEQ ID NO: 818. In one embodiment, the anti-TIM-3 antibody molecule comprises a heavy chain comprising the amino acid sequence of SEQ ID NO: 824, or an amino acid sequence at least 85%, 90%, 95%, or 99% identical or higher to SEQ ID NO: 824. In one embodiment, the anti-TIM-3 antibody molecule comprises a light chain comprising the amino acid sequence of SEQ ID NO: 828, or an amino acid sequence at least 85%, 90%, 95%, or 99% identical or higher to SEQ ID NO: 828. In one embodiment, the anti-TIM-3 antibody molecule comprises a heavy chain comprising the amino acid sequence of SEQ ID NO: 808 and a light chain comprising the amino acid sequence of SEQ ID NO: 818. In one embodiment, the anti-TIM-3 antibody molecule comprises a heavy chain comprising the amino acid sequence of SEQ ID NO: 824 and a light chain comprising the amino acid sequence of SEQ ID NO: 828.

In one embodiment, the antibody molecule comprises a heavy chain encoded by the nucleotide sequence of SEQ ID NO: 809, or a nucleotide sequence at least 85%, 90%, 95%, or 99% identical or higher to SEQ ID NO: 809. In one embodiment, the antibody molecule comprises a light chain encoded by the nucleotide sequence of SEQ ID NO: 819, or a nucleotide sequence at least 85%, 90%, 95%, or 99% identical or higher to SEQ ID NO: 819. In one embodiment, the antibody molecule comprises a heavy chain encoded by the nucleotide sequence of SEQ ID NO: 825, or a nucleotide sequence at least 85%, 90%, 95%, or 99% identical or higher to SEQ ID NO: 825. In one embodiment, the antibody molecule comprises a light chain encoded by the nucleotide sequence of SEQ ID NO: 829, or a nucleotide sequence at least 85%, 90%, 95%, or 99% identical or higher to SEQ ID NO: 829. In one embodiment, the antibody molecule comprises a heavy chain encoded by the nucleotide sequence of SEQ ID NO: 809 and a light chain encoded by the nucleotide sequence of SEQ ID NO: 819. In one embodiment, the antibody molecule comprises a heavy chain encoded by the nucleotide sequence of SEQ ID NO: 825 and a light chain encoded by the nucleotide sequence of SEQ ID NO: 829.

In some embodiments, the anti-TIM-3 antibody is MBG453.

Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 2. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of F38-2E2.

In one embodiment, the anti-TIM-3 antibody molecule is LY3321367 (Eli Lilly). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of LY3321367.

In one embodiment, the anti-TIM-3 antibody molecule is Sym023 (Symphogen). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of Sym023.

In one embodiment, the anti-TIM-3 antibody molecule is BGB-A425 (Beigene). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of BGB-A425.

In one embodiment, the anti-TIM-3 antibody molecule is INCAGN-2390 (Agenus/Incyte). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of INCAGN-2390.

In one embodiment, the anti-TIM-3 antibody molecule is MBS-986258 (BMS/Five Prime). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of MBS-986258.

In one embodiment, the anti-TIM-3 antibody molecule is RO-7121661 (Roche). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of RO-7121661.

In one embodiment, the anti-TIM-3 antibody molecule is LY-3415244 (Eli Lilly). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of LY-3415244.

In one embodiment, the anti-TIM-3 antibody molecule is BC-3402 (Wuxi Zhikanghongyi Biotechnology). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of BC-3402.

In one embodiment, the anti-TIM-3 antibody molecule is SHR-1702 (Medicine Co Ltd.). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of SHR-1702. SHR-1702 is disclosed, e.g., in WO 2020/038355, the content of which is incorporated by reference in its entirety.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

### TGF-β Inhibitors

In some embodiments, the combination described herein comprises a transforming growth factor beta (also known as TGF-β, TGFβ, TGFb, or TGF-beta, used interchangeably herein) inhibitor (e.g., an anti-TGF-β antibody molecule). In some embodiments described herein the TGF-β inhibitor (e.g., an anti-TGF-β antibody molecule) is used in combination with a TIM-3 inhibitor (e.g., an anti-TIM-3 antibody molecule). In some embodiments, the TGF-β inhibitor (e.g., an anti-TGF-β antibody molecule) is used in combination with a TIM-3 inhibitor (e.g., an anti-TIM-3 antibody) and a PD-1 inhibitor (e.g., an anti-PD-1 antibody). In some embodiments, the TGF-β inhibitor (e.g., an anti-TGF-β antibody molecule) is used in combination with a TIM-3 inhibitor (e.g., an anti-TIM-3 antibody) and a hypomethylating agent. In some embodiments, the TGF-β inhibitor (e.g., an anti-TGF-β antibody molecule) is used in combination with a TIM-3 inhibitor (e.g., an anti-TIM-3 antibody), a PD-1 inhibitor (e.g., an anti-PD-1 antibody), and a hypomethylating agent. In some embodiments, the TGF-β inhibitor (e.g., an anti-TGF-β antibody molecule) is used in combination with a TIM-3 inhibitor (e.g., an anti-TIM-3 antibody), optionally further in combination with a hypomethylating agent, and optionally further in combination with a PD-1 inhibitor (e.g. an anti-PD-1 antibody) or an IL-1β inhibitor (e.g., an anti-IL-1β antibody molecule) to treat myelofibrosis. In some embodiments, the myelofibrosis is a primary myelofibrosis (PMF), a post-essential thrombocythemia myelofibrosis (PET-MF), or a post-polycythemia vera myelofibrosis (PPV-MF). In some embodiments, the TGF-β inhibitor is NIS793, fresolimumab, PF-06952229, or AVID200. In some embodiments, the TGF-β inhibitor is NIS793. In certain embodiments, the TGF-β inhibitor (e.g., NIS793) is used in combination with an anti-TIM-3 antibody molecule (e.g., MBG453), optionally further in combination with a hypomethylating agent (e.g., decitabine), and optionally further in combination with a PD-1 inhibitor (e.g., spartalizumab) or an IL-1β inhibitor (e.g., canakinumab) to treat myelofibrosis (e.g., a primary myelofibrosis (PMF), post-essential thrombocythemia myelofibrosis (PET-MF), post-polycythemia vera myelofibrosis (PPV-MF)). In some embodiments, the TGF-β inhibitor (e.g., an anti-TGF-β antibody molecule) is used in combination with a TIM-3 inhibitor (e.g., an anti-TIM-3 antibody) to treat a myelodysplastic syndrome (MDS) (e.g., a lower risk MDS (e.g., a very low risk MDS, a low risk MDS, or an intermediate risk MDS) or a higher risk MDS (e.g., a high risk MDS or a very high risk MDS). In certain embodiments, the TGF-β inhibitor (e.g., NIS793) is used in combination with an anti-TIM-3 antibody (e.g., MBG453) to treat a myelodysplastic syndrome (MDS) (e.g., a lower risk MDS (e.g., a very low risk MDS, a low risk MDS, or an intermediate risk MDS) or a higher risk MDS (e.g., a high risk MDS or a very high risk MDS)). In certain embodiments, the TGF-β inhibitor (e.g., NIS793) is administered on the same day as the anti-TIM-3 antibody molecule (e.g., MBG453). In certain embodiments, the TGF-β inhibitor (e.g., NIS793) is administered after administration of the anti-TIM-3 antibody (e.g., MBG453) has started. In some embodiments, the TGF-β inhibitor (e.g., NIS793) is administered after administration of the anti-TIM-3 antibody (e.g., MBG453) has completed. In some embodiments, the TGF-β inhibitor (e.g., NIS793) is administered about 30 minutes to about four hours (e.g., about one hour) after administration of the anti-TIM-3 antibody (e.g., MBG453) has completed.

### Hypomethylating Agents

In some embodiments, the combination described herein comprises a hypomethylating agent. In some embodiments, the hypomethylating agent is used in combination with a TIM-3 inhibitor (e.g., an anti-TIM-3 antibody molecule) and a TGF-β inhibitor. In some embodiments, the hypomethylating agent is used in combination with a TIM-3 inhibitor (e.g., an anti-TIM-3 antibody molecule) and a TGF-β inhibitor (e.g., an anti-TGF-β antibody molecule) to treat myelofibrosis. In some embodiments, the hypomethylating agent is used in combination with a TIM-3 inhibitor (e.g., an anti-TIM-3 antibody molecule) and a TGF-β inhibitor, optionally further in combination with a PD-1 inhibitor (e.g. an anti-PD-1 antibody) or an IL-1β inhibitor (e.g., an anti-IL-1β antibody molecule) to treat myelofibrosis. In certain embodiments, myelofibrosis is a primary myelofibrosis (PMF), post-essential thrombocythemia myelofibrosis (PET-MF), post-polycythemia vera myelofibrosis (PPV-MF). In some embodiments, the hypomethylating agent is decitabine, azacitidine, CC-486, or ASTX727. In some embodiments, the hypomethylating agent is decitabine. In certain embodiments, the hypomethylating agent (e.g., decitabine) is used in combination with an anti-TIM-3 antibody molecule (e.g., MBG453) and a TGF-β inhibitor (e.g., NIS793) to treat myelofibrosis (e.g., primary myelofibrosis (PMF), post-ET (PET-MF) myelofibrosis, or post-PV myelofibrosis (PPV-MF)). In certain embodiments, the hypomethylating agent (e.g., decitabine) is used in combination with an anti-TIM-3 antibody molecule (e.g., MBG453), a TGF-β inhibitor (e.g., NIS793), optionally further in combination with a PD-1 inhibitor (e.g., spartalizumab) or an IL-1β inhibitor (e.g., canakinumab) to treat myelofibrosis (e.g., primary myelofibrosis (PMF), post-ET (PET-MF) myelofibrosis, or post-PV myelofibrosis (PPV-MF)).

### PD-1 Inhibitors

In some embodiments, the combination described herein comprises a PD-1 inhibitor. In some embodiments the PD-1 inhibitor is used in combination with a TIM-3 inhibitor (e.g., an anti-TIM-3 antibody molecule) and a TGF-β inhibitor. In some embodiments the PD-1 inhibitor is used in combination with a TIM-3 inhibitor (e.g., an anti-TIM-3 antibody molecule) and a TGF-β inhibitor (e.g., an anti-TGF-β antibody molecule) to treat myelofibrosis. In some embodiments, the PD-1 inhibitor is used in combination with a TIM-3 inhibitor (e.g., an anti-TIM-3 antibody molecule), a TGF-β inhibitor, and a hypomethylating agent to treat myelofibrosis. In certain embodiments, myelofibrosis is a primary myelofibrosis (PMF), post-essential thrombocythemia myelofibrosis (PET-MF), post-polycythemia vera myelofibrosis (PPV-MF). In some embodiments, the PD-1 inhibitor is spartalizumab (also known as PDR001), nivolumab, pembrolizumab, pidilizumab, MEDI0680, REGN2810, TSR-042, PF-06801591, BGB-A317, BGB-108, INCSHR1210, or AMP-224. In some embodiments, the PD-1 inhibitor is spartalizumab. In certain embodiments, the anti-PD-1 inhibitor (e.g., spartalizumab) is used in combination with an anti-TIM-3 antibody molecule (e.g., MBG453), and a TGF-β inhibitor (e.g., NIS793) to treat myelofibrosis (e.g., primary myelofibrosis (PMF), post-ET (PET-MF) myelofibrosis, or post-PV myelofibrosis (PPV-MF)). In certain embodiments, the anti-PD-1 inhibitor (e.g., spartalizumab) is used in combination with an anti-TIM-3 antibody molecule (e.g., MBG453), a TGF-β inhibitor (e.g., NIS793), and a hypomethylating agent (e.g., decitabine) to treat myelofibrosis (e.g., primary myelofibrosis (PMF), post-ET (PET-MF) myelofibrosis, or post-PV myelofibrosis (PPV-MF)).

### IL-1β Inhibitors

In some embodiments, the combination described herein comprises an IL-1β inhibitor. In some embodiments the IL-1β inhibitor is used in combination with a TIM-3 inhibitor (e.g., an anti-TIM-3 antibody molecule) and a TGF-β inhibitor. In some embodiments the IL-1β inhibitor is used in combination with a TIM-3 inhibitor (e.g., an anti-TIM-3 antibody molecule) and a TGF-β inhibitor (e.g., an anti-TGF-β antibody molecule) to treat myelofibrosis. In some embodiments, the IL-1β inhibitor is used in combination with a TIM-3 inhibitor (e.g., an anti-TIM-3 antibody molecule), a TGF-β inhibitor, and a hypomethylating agent to treat myelofibrosis. In certain embodiments, myelofibrosis is a primary myelofibrosis (PMF), post-essential thrombocythemia myelofibrosis (PET-MF), post-polycythemia vera myelofibrosis (PPV-MF). In some embodiments, the IL-1β inhibitor is canakinumab (also known as ACZ885 or ILARIS®), gevokizumab, Anakinra, diacerein, IL-1 Affibody (SOBI 006, Z-FC (Swedish Orphan Biovitrum/Affibody)), Rilonacept, Lutikizumab (ABT-981), CDP-484, LY-2189102 and PBF509 (NIR178). In some embodiments, the IL-1β inhibitor is canakinumab. In certain embodiments, the IL-1β inhibitor (e.g., canakinumab) is used in combination with an anti-TIM-3 antibody molecule (e.g., MBG453), and a TGF-β inhibitor (e.g., NIS793) to treat myelofibrosis (e.g., primary myelofibrosis (PMF), post-ET (PET-MF) myelofibrosis, or post-PV myelofibrosis (PPV-MF)). In certain embodiments, the IL-1β inhibitor (e.g., canakinumab) is used in combination with an anti-TIM-3 antibody molecule (e.g., MBG453), a TGF-β inhibitor (e.g., NIS793), and a hypomethylating agent (e.g., decitabine) to treat myelofibrosis (e.g., primary myelofibrosis (PMF), post-ET (PET-MF) myelofibrosis, or post-PV myelofibrosis (PPV-MF)). In certain embodiments, the IL-1β inhibitor (e.g., canakinumab) is used in combination with an anti-TIM-3 antibody molecule (e.g., MBG453), and a TGF-β inhibitor (e.g., NIS793) to treat a myelodysplastic syndrome (MDS) (e.g., a lower risk MDS (e.g., a very low risk MDS, a low risk MDS, or an intermediate risk MDS) or a higher risk MDS (e.g., a high risk MDS or a very high risk MDS)).

### Therapeutic Use

Without wishing to be bound by theory, it is believed that in some embodiments, the combinations described herein can inhibit, reduce, or neutralize one or more activities of TIM-3, TGF-β, PD-1, IL-1β, or DNA methyltransferase, resulting in, e.g., one or more of immune checkpoint inhibition, programmed cell death, hypomethylation, or cytotoxicity. Thus, the combinations described herein can be used to treat or prevent disorders (e.g., cancer), where enhancing an immune response in a subject is desired.

Accordingly, in another aspect, a method of modulating an immune response in a subject is provided. The method comprises administering to the subject a therapeutically effective amount of a combination described herein, e.g., in accordance with a dosage regimen described herein, such that the immune response in the subject is modulated. In one embodiment, the combination enhances, stimulates or increases the immune response in the subject. The subject can be a mammal, e.g., a primate, preferably a higher primate, e.g., a human (e.g., a patient having, or at risk of having, a disorder described herein). In one embodiment, the subject is in need of enhancing an immune response. In one embodiment, the subject has, or is at risk of, having a disorder described herein, e.g., a cancer as described herein. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection. In certain embodiments, the subject is unfit for a chemotherapy, e.g., an intensive induction chemotherapy.

In one aspect, a method of treating (e.g., one or more of reducing, inhibiting, or delaying progression) a cancer in a subject is provided. The method comprises administering to the subject a therapeutically effective amount of a combination disclosed herein, e.g., in accordance with a dosage regimen described herein, thereby treating the cancer in the subject. In certain embodiments, the cancer treated with the combination includes, but is not limited to, a hematological cancer (e.g., myeloproliferative neoplasm, leukemia, lymphoma, or myeloma), a solid tumor, and a metastatic lesion. In one embodiment, the cancer a hematological cancer. Examples of hematological cancers include, e.g., a myeloproliferative neoplasm (e.g., a myelofibrosis, a polycythemia vera (PV), or an essential thrombocythemia (ET)), a myelodysplastic syndrome (e.g., a lower risk MDS (e.g., a very low risk MDS, a low risk MDS, or an intermediate risk MDS) or a higher risk MDS (e.g., a high risk MDS or a very high risk MDS)), a leukemia (e.g., an acute myeloid leukemia (AML) or A chronic lymphocytic leukemia (CLL), a lymphoma (e.g., small lymphocytic lymphoma (SLL)), and a myeloma (e.g., a multiple myeloma (MM)). The cancer may be at an early, intermediate, late stage or metastatic cancer.

In certain embodiments, the cancer treated with the combination includes, but is not limited to, myelofibrosis (e.g., a primary myelofibrosis (PMF), post-essential thrombocythemia myelofibrosis (PET-MF), post-polycythemia vera myelofibrosis (PPV-MF)). In certain embodiments, the cancer treated with the combination is a primary myelofibrosis (MF).

In certain embodiments, the cancer treated with the combination includes, but is not limited to, myelodysplastic syndrome (e.g., a lower risk MDS (e.g., a very low risk MDS, a low risk MDS, or an intermediate risk MDS) or a higher risk MDS (e.g., a high risk MDS or a very high risk MDS)). In certain embodiments, the cancer treated with the combination is a lower risk MDS.

In certain embodiments, the cancer is an MSI-high cancer. In some embodiments, the cancer is a metastatic cancer. In other embodiments, the cancer is an advanced cancer. In other embodiments, the cancer is a relapsed or refractory cancer.

In other embodiments, the subject has, or is identified as having, TIM-3 expression in tumor-infiltrating lymphocytes (TILs). In one embodiment, the cancer microenvironment has an elevated level of TIM-3 expression. In one embodiment, the cancer microenvironment has an elevated level of PD-L1 expression. Alternatively, or in combination, the cancer microenvironment can have increased IFNγ and/or CD8 expression.

In some embodiments, the subject has, or is identified as having, a tumor that has one or more of high PD-L1 level or expression, or as being tumor infiltrating lymphocyte (TIL)+ (e.g., as having an increased number of TILs), or both. In certain embodiments, the subject has, or is identified as having, a tumor that has high PD-L1 level or expression and that is TIL+. In some embodiments, the methods described herein further include identifying a subject based on having a tumor that has one or more of high PD-L1 level or expression, or as being TIL+, or both. In certain embodiments, the methods described herein further include identifying a subject based on having a tumor that has high PD-L1 level or expression and as being TIL+. In some embodiments, tumors that are TIL+ are positive for CD8 and IFNγ. In some embodiments, the subject has, or is identified as having, a high percentage of cells that are positive for one, two or more of PD-L1, CD8, and/or IFNγ. In certain embodiments, the subject has or is identified as having a high percentage of cells that are positive for all of PD-L1, CD8, and IFNγ.

In some embodiments, the methods described herein further include identifying a subject based on having a high percentage of cells that are positive for one, two or more of PD-L1, CD8, and/or IFNγ. In certain embodiments, the methods described herein further include identifying a subject based on having a high percentage of cells that are positive for all of PD-L1, CD8, and IFNγ. In some embodiments, the subject has, or is identified as having, one, two or more of PD-L1, CD8, and/or IFNγ, and one or more of a hematological cancer, e.g., a leukemia (e.g., an AML or CLL), a lymphoma, (e.g., an SLL), and/or a myeloma (e.g., an MM). In certain embodiments, the methods described herein further describe identifying a subject based on having one, two or more of PD-L1, CD8, and/or IFNγ, and one or more of a leukemia (e.g., an AML or CLL), a lymphoma, (e.g., an SLL), and/or a myeloma (e.g., an MM).

Methods, compositions, and formulations disclosed herein are useful for treating metastatic lesions associated with the aforementioned cancers.

Still further, the invention provides a method of enhancing an immune response to an antigen in a subject, comprising administering to the subject: (i) the antigen; and (ii) a combination described herein, in accordance with a dosage regimen described herein, such that an immune response to the antigen in the subject is enhanced. The antigen can be, for example, a tumor antigen, a viral antigen, a bacterial antigen or an antigen from a pathogen.

The combination described herein can be administered to the subject systemically (e.g., orally, parenterally, subcutaneously, intravenously, rectally, intramuscularly, intraperitoneally, intranasally, transdermally, or by inhalation or intracavitary installation), topically, or by application to mucous membranes, such as the nose, throat and bronchial tubes. In certain embodiments, the anti-TIM-3 antibody molecule, anti-TGF-β antibody molecule, anti-IL-1β antibody molecule, or anti-PD-1 antibody molecule is administered intravenously at a flat dose described herein.

### Immunomodulators

The combinations described herein (e.g., a combination comprising a therapeutically effective amount of an anti-TIM-3 antibody molecule described herein and an anti-TGF-beta antibody molecule described herein) can be used further in combination with one or more immunomodulators.

In certain embodiments, the immunomodulator is an inhibitor of an immune checkpoint molecule. In one embodiment, the immunomodulator is an inhibitor of PD-1, PD-L1, PD-L2, CTLA-4, LAG-3, CEACAM (e.g., CEACAM-1, -3 and/or -5), VISTA, BTLA, TIGIT, LAIR1, or CD160, or 2B4. In one embodiment, the inhibitor of an immune checkpoint molecule inhibits PD-1, PD-L1, LAG-3, CEACAM (e.g., CEACAM-1, -3 and/or -5), CTLA-4, or any combination thereof.

Inhibition of an inhibitory molecule can be performed at the DNA, RNA or protein level. In embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand (e.g., PD-1-Ig or CTLA-4 Ig), or an antibody molecule that binds to the inhibitory molecule; e.g., an antibody molecule that binds to PD-1, PD-L1, PD-L2, CEACAM (e.g., CEACAM-1, -3 and/or -5), CTLA-4, LAG-3, VISTA, BTLA, TIGIT, LAIR1, CD160, or 2B4, or a combination thereof.

In certain embodiments, the combination comprises an anti-TIM-3 antibody molecule that is in the form of a bispecific or multispecific antibody molecule. In one embodiment, the bispecific antibody molecule has a first binding specificity to TIM-3 and a second binding specificity, e.g., a second binding specificity to, PD-1, PD-L1, CEACAM (e.g., CEACAM-1, -3 and/or -5), LAG-3, or PD-L2. In one embodiment, the bispecific antibody molecule binds to (i) PD-1 or PD-L1 (ii) and TIM-3. In another embodiment, the bispecific antibody molecule binds to TIM-3 and LAG-3. In another embodiment, the bispecific antibody molecule binds to TIM-3 and CEACAM (e.g., CEACAM-1, -3 and/or -5). In another embodiment, the bispecific antibody molecule binds to TIM-3 and CEACAM-1. In still another embodiment, the bispecific antibody molecule binds to TIM-3 and CEACAM-3. In yet another embodiment, the bispecific antibody molecule binds to TIM-3 and CEACAM-5.

In other embodiments, the combination further comprises a bispecific or multispecific antibody molecule. In another embodiment, the bispecific antibody molecule binds to PD-1 or PD-L1. In yet another embodiment, the bispecific antibody molecule binds to PD-1 and PD-L2. In another embodiment, the bispecific antibody molecule binds to CEACAM (e.g., CEACAM-1, -3 and/or -5) and LAG-3.

Any combination of the aforesaid molecules can be made in a multispecific antibody molecule, e.g., a trispecific antibody that includes a first binding specificity to TIM-3, and a second and third binding specificities to two or more of: PD-1, PD-L1, CEACAM (e.g., CEACAM-1, -3 and/or -5), LAG-3, or PD-L2.

In certain embodiments, the immunomodulator is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1 (e.g., an anti-PD-1 or anti-PD-L1 antibody molecule as described herein).

The combination of the PD-1 or PD-L1 inhibitor with the anti-TIM-3 antibody molecule can further include one or more additional immunomodulators, e.g., in combination with an inhibitor of LAG-3, CEACAM (e.g., CEACAM-1, -3 and/or -5) or CTLA-4. In one embodiment, the inhibitor of PD-1 or PD-L1 (e.g., the anti-PD-1 or PD-L1 antibody molecule) is administered in combination with the anti-TIM-3 antibody molecule and a LAG-3 inhibitor (e.g., an anti-LAG-3 antibody molecule). In another embodiment, the inhibitor of PD-1 or PD-L1 (e.g., the anti-PD-1 or PD-L1 antibody molecule) is administered in combination with the anti-TIM-3 antibody molecule and a CEACAM inhibitor (e.g., CEACAM-1, -3 and/or -5 inhibitor), e.g., an anti-CEACAM antibody molecule. In another embodiment, the inhibitor of PD-1 or PD-L1 (e.g., the anti-PD-1 or PD-L1 antibody molecule) is administered in combination with the anti-TIM-3 antibody molecule and a CEACAM-1 inhibitor (e.g., an anti-CEACAM-1 antibody molecule). In another embodiment, the inhibitor of PD-1 or PD-L1 (e.g., the anti-PD-1 or PD-L1 antibody molecule) is administered in combination with the anti-TIM-3 antibody molecule and a CEACAM-5 inhibitor (e.g., an anti-CEACAM-5 antibody molecule). In yet other embodiments, the inhibitor of PD-1 or PD-L1 (e.g., the anti-PD-1 or PD-L1 antibody molecule) is administered in combination with the anti-TIM-3 antibody molecule, a LAG-3 inhibitor (e.g., an anti-LAG-3 antibody molecule), and a TIM-3 inhibitor (e.g., an anti-TIM-3 antibody molecule). Other combinations of immunomodulators with the anti-TIM-3 antibody molecule and a PD-1 inhibitor (e.g., one or more of PD-L2, CTLA-4, LAG-3, CEACAM (e.g., CEACAM-1, -3 and/or -5), VISTA, BTLA, TIGIT, LAIR1, CD160, 2B4 and/or TGF beta) are also within the present invention. Any of the antibody molecules known in the art or disclosed herein can be used in the aforesaid combinations of inhibitors of checkpoint molecule.

In other embodiments, the immunomodulator is an inhibitor of CEACAM (e.g., CEACAM-1, -3 and/or -5), e.g., human CEACAM (e.g., CEACAM-1, -3 and/or -5). In one embodiment, the immunomodulator is an inhibitor of CEACAM-1, e.g., human CEACAM-1. In another embodiment, the immunomodulator is an inhibitor of CEACAM-3, e.g., human CEACAM-3. In another embodiment, the immunomodulator is an inhibitor of CEACAM-5, e.g., human CEACAM-5. In one embodiment, the inhibitor of CEACAM (e.g., CEACAM-1, -3 and/or -5) is an antibody molecule to CEACAM (e.g., CEACAM-1, -3 and/or -5). The combination of the CEACAM (e.g., CEACAM-1, -3 and/or -5) inhibitor and the anti-TIM-3 antibody molecule can further include one or more additional immunomodulators, e.g., in combination with an inhibitor of LAG-3, PD-1, PD-L1 or CTLA-4.

In other embodiments, the immunomodulator is an inhibitor of LAG-3, e.g., human LAG-3. In one embodiment, the inhibitor of LAG-3 is an antibody molecule to LAG-3. The combination of the LAG-3 inhibitor and the anti-TIM-3 antibody molecule can further include one or more additional immunomodulators, e.g., in combination with an inhibitor of CEACAM (e.g., CEACAM-1, -3 and/or -5), PD-1, PD-L1 or CTLA-4.

In certain embodiments, the immunomodulator used in the combinations disclosed herein (e.g., in combination with a therapeutic agent chosen from an antigen-presentation combination) is an activator or agonist of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CD28, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3, or CD83 ligand.

In other embodiments, the immunomodulator is a GITR agonist. In one embodiment, the GITR agonist is an antibody molecule to GITR. The anti-GITR antibody molecule and the anti-TIM-3 antibody molecule may be in the form of separate antibody composition, or as a bispecific antibody molecule. The combination of the GITR agonist with the anti-TIM-3 antibody molecule can further include one or more additional immunomodulators, e.g., in combination with an inhibitor of PD-1, PD-L1, CTLA-4, CEACAM (e.g., CEACAM-1, -3 and/or -5), or LAG-3. In some embodiments, the anti-GITR antibody molecule is a bispecific antibody that binds to GITR and PD-1, PD-L1, CTLA-4, CEACAM (e.g., CEACAM-1, -3 and/or -5), or LAG-3. In other embodiments, a GITR agonist can be administered in combination with one or more additional activators of costimulatory molecules, e.g., an agonist of OX40, CD2, CD27, CD28, CDS, ICAM-1, LFA-1 (CD11a/CD18), ICOS (CD278), 4-1BB (CD137), CD30, CD40, BAFFR, HVEM, CD7, LIGHT, NKG2C, SLAMF7, NKp80, CD160, B7-H3, or CD83 ligand.

In other embodiments, the immunomodulator is an OX40 agonist. In one embodiment, the OX40 agonist is an antibody molecule to OX40. The OX40 antibody molecule and the anti-TIM-3 antibody molecule may be in the form of separate antibody composition, or as a bispecific antibody molecule. The combination of the OX40 agonist with the anti-TIM-3 antibody molecule can further include one or more additional immunomodulators, e.g., in combination with an inhibitor of PD-1, PD-L1, CTLA-4, CEACAM (e.g., CEACAM-1, -3 and/or -5), or LAG-3. In some embodiments, the anti-OX40 antibody molecule is a bispecific antibody that binds to OX40 and PD-1, PD-L1, CTLA-4, CEACAM (e.g., CEACAM-1, -3 and/or -5), or LAG-3. In other embodiments, the OX40 agonist can be administered in combination with other costimulatory molecule, e.g., an agonist of GITR, CD2, CD27, CD28, CDS, ICAM-1, LFA-1 (CD11a/CD18), ICOS (CD278), 4-1BB (CD137), CD30, CD40, BAFFR, HVEM, CD7, LIGHT, NKG2C, SLAMF7, NKp80, CD160, B7-H3, or CD83 ligand.

In other embodiments, the immunomodulator is an inhibitor of IL-1β. In some embodiments, the inhibitor of IL-1β is an antibody molecule to IL-1β. The combination of the IL-1β inhibitor and the anti-TIM-3 antibody molecule and anti-TGF-β antibody molecule can further include one or more additional immunomodulators.

It is noted that only exemplary combinations of inhibitors of checkpoint inhibitors or agonists of costimulatory molecules are provided herein. Additional combinations of these agents are within the scope of the present invention.

### Biomarkers

In certain embodiments, any of the methods or use disclosed herein further includes evaluating or monitoring the effectiveness of a therapy (e.g., a combination therapy) described herein, in a subject (e.g., a subject having a cancer, e.g., a cancer described herein). The method includes acquiring a value of effectiveness to the therapy, wherein said value is indicative of the effectiveness of the therapy.

In embodiments, the value of effectiveness to the therapy comprises a measure of one, two, three, four, five, six, seven, eight, nine or more (e.g., all) of the following:

(i) a parameter of a tumor infiltrating lymphocyte (TIL) phenotype;

(ii) a parameter of a myeloid cell population;

(iii) a parameter of a surface expression marker;

(iv) a parameter of a biomarker of an immunologic response;

(v) a parameter of a systemic cytokine modulation;

(vi) a parameter of circulating free DNA (cfDNA);

(vii) a parameter of systemic immune-modulation;

(viii) a parameter of microbiome;

(ix) a parameter of a marker of activation in a circulating immune cell;

(x) a parameter of a circulating cytokine; or

(xi) a parameter of RNA expression.

In some embodiments, the parameter of a TIL phenotype comprises the level or activity of one, two, three, four or more (e.g., all) of Hematoxylin and eosin (H&E) staining for TIL counts, CD8, FOXP3, CD4, or CD3, in the subject, e.g., in a sample from the subject (e.g., a tumor sample, blood sample, or a bone marrow sample).

In some embodiments, the parameter of a myeloid cell population comprises the level or activity of one or both of CD68 or CD163, in the subject, e.g., in a sample from the subject (e.g., a tumor sample).

In some embodiments, the parameter of a surface expression marker comprises the level or activity of one, two, three or more (e.g., all) of TIM-3, PD-1, PD-L1, or LAG-3, in the subject, e.g., in a sample from the subject (e.g., a tumor sample or a bone marrow sample). In certain embodiments, the level of TIM-3, PD-1, PD-L1, or LAG-3 is determined by immunohistochemistry (IHC). In certain embodiments, the level of TIM-3 is determined.

In some embodiments, the parameter of a biomarker of an immunologic response comprises the level or sequence of one or more nucleic acid-based markers, in the subject, e.g., in a sample from the subject (e.g., a tumor sample or a bone marrow sample).

In some embodiments, the parameter of systemic cytokine modulation comprises the level or activity of one, two, three, four, five, six, seven, eight, or more (e.g., all) of IL-2, IL-8, IL-18, IFN-γ, ITAC (CXCL11), IL-6, IL-10, IL-4, IL-17, IL-15, MIP1α, MCP1, TNF-α, IP-10, or TGF-beta, in the subject, e.g., in a sample from the subject (e.g., a blood sample, e.g., a plasma sample).

In some embodiments, the parameter of cfDNA comprises the sequence or level of one or more circulating tumor DNA (cfDNA) molecules, in the subject, e.g., in a sample from the subject (e.g., a blood sample, e.g., a plasma sample).

In some embodiments, the parameter of systemic immune-modulation comprises phenotypic characterization of an activated immune cell, e.g., a CD3-expressing cell, a CD8-expressing cell, or both, in the subject, e.g., in a sample from the subject (e.g., a blood sample, e.g., a PBMC sample).

In some embodiments, the parameter of microbiome comprises the sequence or expression level of one or more genes in the microbiome, in the subject, e.g., in a sample from the subject (e.g., a stool sample).

In some embodiments, the parameter of a marker of activation in a circulating immune cell comprises the level or activity of one, two, three, four, five or more (e.g., all) of circulating CD8+, HLA-DR+Ki67+, T cells, IFN-γ, IL-18, or CXCL11 (IFN-γ induced CCK) expressing cells, in a sample (e.g., a blood sample, e.g., a plasma sample).

In some embodiments, the parameter of a circulating cytokine comprises the level or activity of IL-6, in the subject, e.g., in a sample from the subject (e.g., a blood sample, e.g., a plasma sample).

In some embodiments, the parameter of RNA expression comprises the level or sequence of an immune and/or a cancer related gene, e.g., a MF-related gene or an MDS-related gene, in the subject, e.g., in a sample from the subject (e.g., a tumor sample, a bone marrow sample, or a blood sample, e.g., a plasma sample). In some embodiments, the MDS-related gene comprises DNMT3, ASXL1, TET2, RUNX1, TP53, or any combination thereof.

In some embodiments of any of the methods disclosed herein, the therapy comprises a combination of an anti-TIM-3 antibody molecule described herein and a second inhibitor of an immune checkpoint molecule, e.g., an inhibitor of PD-1 (e.g., an anti-PD-1 antibody molecule) or an inhibitor of PD-L1 (e.g., an anti-PD-L1 antibody molecule).

In some embodiments of any of the methods disclosed herein, the measure of one or more of (i)-(xi) is obtained from a sample acquired from the subject. In some embodiments, the sample is chosen from a tumor sample, a blood sample (e.g., a plasma sample or a PBMC sample), or a stool sample.

In some embodiments of any of the methods disclosed herein, the subject is evaluated prior to receiving, during, or after receiving, the therapy.

In some embodiments of any of the methods disclosed herein, the measure of one or more of (i)-(xi) evaluates a profile for one or more of gene expression, flow cytometry or protein expression.

In some embodiments of any of the methods disclosed herein, the presence of an increased level or activity of one, two, three, four, five, or more (e.g., all) of circulating CD8+, HLA-DR+Ki67+, T cells, IFN-γ, IL-18, or CXCL11 (IFN-γ induced CCK) expressing cells, and/or the presence of an decreased level or activity of IL-6, in the subject or sample, is a positive predictor of the effectiveness of the therapy.

Alternatively, or in combination with the methods disclosed herein, responsive to said value, performing one, two, three, four or more (e.g., all) of:

(i) administering to the subject the therapy;

(ii) administered an altered dosing of the therapy;

(iii) altering the schedule or time course of the therapy;

(iv) administering to the subject an additional agent (e.g., a therapeutic agent described herein) in combination with the therapy; or

(v) administering to the subject an alternative therapy.

### Additional Embodiments

In certain embodiments, any of the methods disclosed herein further includes identifying in a subject or a sample (e.g., a subject's sample comprising cancer cells and/or immune cells such as TILs) the presence of TIM-3, thereby providing a value for TIM-3. The method can further include comparing the TIM-3 value to a reference value, e.g., a control value. If the TIM-3 value is greater than the reference value, e.g., the control value, administering a therapeutically effective amount of the combination described herein that comprises an anti-TIM-3 antibody molecule described herein to the subject, and optionally, in combination with a second therapeutic agent (e.g., a TGF-β inhibitor, e.g., NIS793) and/or additional therapeutic agents (e.g., a PD-1 inhibitor (e.g., spartalizumab) and/or a hypomethylating agent (e.g., decitabine), and/or an IL-1β inhibitor (e.g., canakinumab), or a procedure, or modality described herein, thereby treating a cancer.

In other embodiments, any of the methods disclosed herein further includes identifying in a subject or a sample (e.g., a subject's sample comprising cancer cells and/or immune cells such as TILs) the presence of PD-L1, thereby providing a value for PD-L1. The method can further include comparing the PD-L1 value to a reference value, e.g., a control value. If the PD-L1 value is greater than the reference value, e.g., the control value, administering a therapeutically effective amount of an anti-TIM-3 antibody molecule described herein to the subject, and optionally, in combination with a second therapeutic agent, procedure, or modality described herein, thereby treating a cancer.

In other embodiments, any of the methods disclosed herein further includes identifying in a subject or a sample (e.g., a subject's sample comprising cancer cells and optionally immune cells such as TILs) the presence of one, two or all of PD-L1, CD8, or IFN-γ, thereby providing a value for one, two or all of PD-L1, CD8, and IFN-γ. The method can further include comparing the PD-L1, CD8, and/or IFN-γ values to a reference value, e.g., a control value. If the PD-L1, CD8, and/or IFN-γ values are greater than the reference value, e.g., the control values, administering a therapeutically effective amount of an anti-TIM-3 antibody molecule described herein to the subject, and optionally, in combination with a second therapeutic agent, procedure, or modality described herein, thereby treating a cancer.

The subject may have a cancer described herein, such as a hematological cancer or a solid tumor, e.g., a myeloproliferative neoplasm (e.g., a myelofibrosis, a primary myelofibrosis (PMF), post-essential thrombocythemia myelofibrosis (PET-MF), post-polycythemia vera myelofibrosis (PPV-MF)), a leukemia (e.g., an acute myeloid leukemia (AML), e.g., a relapsed or refractory AML or a de novo AML), a lymphoma, a myeloma, an ovarian cancer, a lung cancer (e.g., a small cell lung cancer (SCLC) or a non-small cell lung cancer (NSCLC)), a mesothelioma, a skin cancer (e.g., a Merkel cell carcinoma (MCC) or a melanoma), a kidney cancer (e.g., a renal cell carcinoma), a bladder cancer, a soft tissue sarcoma (e.g., a hemangiopericytoma (HPC)), a bone cancer (e.g., a bone sarcoma), a colorectal cancer, a pancreatic cancer, a nasopharyngeal cancer, a breast cancer, a duodenal cancer, an endometrial cancer, an adenocarcinoma (an unknown adenocarcinoma), a liver cancer (e.g., a hepatocellular carcinoma), a cholangiocarcinoma, a sarcoma, a myelodysplastic syndrome (MDS) (e.g., a high risk MDS). The subject may have a myelofibrosis, e.g., a primary myelofibrosis (PMF), post-ET (PET-MF) myelofibrosis, or post-PV myelofibrosis (PPV-MF).

All publications, patent applications, patents, and other references mentioned herein are incorporated by reference in their entirety.

Other features, objects, and advantages of the invention will be apparent from the description and drawings, and from the claims.

### BRIEF DESCRIPTION OF THE DRAWINGS

FIG. 1 is a graph depicting the impact of MBG453 on the interaction between TIM3 and galectin-9. Competition was assessed as a measure of the ability of the antibody to block Gal9-SULFOTag signal to TIM-3 receptor, which is shown on the Y-axis. Concentration of the antibody is shown on the X-axis.

FIG. 2 is graph showing that MBG453 mediates modest antibody-dependent cellular phagocytosis (ADCP). The percentage of phagocytosis was quantified at various concentrations tested of MBG453, Rituximab, and a control hIgG4 monoclonal antibody (mAB).

FIG. 3 is a graph demonstrating MBG453 engagement of FcγR1a as measured by luciferase activity. The activation of the NFAT dependent reporter gene expression induced by the binding of MBG453 or the anti-CD20 MabThera reference control to FcγRIa was quantified by luciferase activity at various concentrations of the antibody tested.

FIG. 4 shows that MBG453 enhances immune-mediated killing of decitabine pre-treated AML cells.

FIG. 5 is a graph depicting the anti-leukemic activity of MBG453 with and without decitabine in the AML patient-derived xenograft (PDX) model, HAMLX21432. MBG453 was administered i.p. at 10 mg/kg, once weekly (starting at day 6 of dosing) either as a single agent or in combination with decitabine i.p. at 1 mg/kg, once daily for a total of 5 doses (from initiation of dosing). Initial group size: 4 animals. Body weights were recorded weekly during a 21-day dosing period that commenced on day 27 post implantation (AML PDX model #21432 2×10⁶ cells/animal). All final data were recorded on day 56. Leukemic burden was measured as a percentage of human CD45+ cells in peripheral blood by FACS analysis.

FIG. 6 is a graph depicting the anti-leukemic activity of MBG453 with and without decitabine in the AML patient-derived xenograft (PDX) model, HAMLX5343. Treatments started on day 32 post implantation (2 million cells/animal). MBG453 was administered i.p. at 10 mg/kg, once weekly (starting on day 6 of dosing), either as a single agent or in combination with decitabine i.p. at 1 mg/kg, once daily for a total of 5 doses (from initiation of dosing). Initial group size: 4 animals. Body weights were recorded weekly during a 21 day dosing period. All final data were recorded on day 56. Leukemic burden was measured as a percentage of CD45+ cells in peripheral blood by FACS analysis.

FIG. 7 is a graph depicting MBG453 enhanced killing of THP-1 AML cells that were engineered to overexpress TIM-3 relative to parental control THP-1 cells. The ratio between TIM-3-expressing THP-1 cells and parental THP-1 cells (“fold” in y-axis of graph) was calculated and normalized to conditions without anti-CD3/anti-CD28 bead stimulation. The x-axis of the graph denotes the stimulation amount as number of beads per cell. Data represents one of two independent experiments.

FIG. 8 depicts the baseline levels of IL-1β produced by wild-type control cells or TIM-3 overexpressing cells.

FIG. 9 depicts IL-1β mRNA expression levels in baseline (Screening) in bone marrow samples of AML/MDS patients in the Decitabine+MBG453 combination arm of PDR001X2105. Expression is plotted as log₂ counts per million (CPM). Patients were grouped by indicated best overall response. IL-1β mRNA expression at baseline tended to be higher in AML/MDS patients with progressive disease.

FIGS. 10A-10C depicts the log₂ fold change of IL-1β mRNA expression levels, calculated as C3D1 divided by Screening for paired patient bone marrow samples, are shown. FIG. 10A depicts a Volcano plot showing differential gene expression upon treatment comparing responders (CR/PR) to non-responders (SD/PD) in Decitabine+MBG453 combination cohort. Log₂ fold change (C3D1/Screening) of gene expression (x-axis) and unadjusted p-values (y-axis) that were calculated using the Limma package are shown. IL-1β is highlighted as it is one of the top differentially expressed genes upon treatment between two response groups. FIG. 10B depicts expression levels of IL-1β mRNA at Screening and C3D1 for patients with paired timepoints are shown for each response group (CR, PR, SD and PD). FIG. 10C depicts the Log₂ fold change in IL-1β mRNA (C3D1/Screening) plotted against best percent change in blasts.

### DETAILED DESCRIPTION

T-cell immunoglobulin and mucin domain-containing 3 (TIM-3; also known as hepatitis A virus cellular receptor 2) has a widespread and complex role in immune system regulation, with published roles both in both the adaptive immune response (CD4+ and CD8+ T effector cells, regulatory T cells) and innate immune responses (macrophages, dendritic cells, NK cells). TIM-3 has an important role in tumor-induced immune suppression as it marks the most suppressed or dysfunctional populations of CD8+ T cells in animal models of solid and hematologic malignancies (Sakuishi et al. (2010) J Exp Med. 207(10):2187-94; Zhou et al. (2011) Blood 117(17):4501-10; Yang et al. (2012) J Clin Invest. 122(4):1271-82) and is expressed on FoxP3+ regulatory T cells (Tregs), which correlate with disease severity in many cancer indications (Gao et al. (2012) PLoS One 7(2):e30676; Yan et al. (2013) PLoS One 8(3):e58006). TIM-3 is expressed on exhausted or dysfunctional T cells in cancer, and ex vivo TIM-3 blockade of TIM-3+ NY-ESO-1+ T cells from melanoma patients restored IFN-γ and TNF-α production as well as the proliferation in response to antigenic stimulation (Fourcade et al. (2010) J Exp Med. 207(10):2175-86). Blockade of TIM-3 on macrophages and antigen cross-presenting dendritic cells enhances activation and inflammatory cytokine/chemokine production (Zhang et al. (2011) J. Immunol 186(5):3096-103; Zhang et al. (2012) J. Leukoc Biol 91(2):189-96; Chiba et al. (2012) Nat Immunol. 13(9):832-42; de Mingo Pulido et al. (2018) Cancer Cell 33(1):60-74), ultimately leading to enhanced effector T cells responses. Further, increased expression of TIM-3 was also observed on myelofibrosis progenitor cells (unpublished, data on file).

In myelofibrosis patients, constitutive JAK2/STAT3/STAT5 activation, mainly in monocytes, megakaryocytes, and platelets, likely causes TIM-3-mediated immune escape by reducing T cell activation, metabolic activity, and cell cycle progression (potentially similarly to the PD-L1-mediated immune escape described by Prestipino et al (2018) Sci Tranl Med. 10(429): eaam7729). Therefore, an anti-TIM-3 antibody holds promise in myelofibrosis patients to help mounting an immune response against myelofibrosis progenitor in order to reduce disease burden and progressive disease.

NIS793 is a fully human IgG2, human/mouse cross-reactive, TGF-β-neutralizing antibody. In patients with primary myelofibrosis (PMF), increased levels of TGFβ1 in serum and bone marrow have been shown to correlate with the extent of both bone marrow fibrosis and leukemic cell infiltration, and data from preclinical models have established an important role for TGF-β in disease progression. In particular, TGF-β1 is associated with increased synthesis of types I, III and IV collagens as well as other extracellular matrix proteins such as fibronectin and tenascin, all elements that are actively deposited and accumulate in the bone marrow of patients affected with PMF, thereby implicating TGF-β in pathogenesis of bone marrow fibrosis (Tefferi (2005) J Clin Oncol. 23(33):8520-30). Accordingly, in thrombopoietin-high mice, absence of TGF-β1 was shown to prevent the occurrence of bone marrow fibrosis, despite the development of myeloproliferative syndrome (Chagraoui et al. (2002) Blood 100(10):3495-503). A similar correlation was reported in another murine model of PMF, Gata1-low mice, in which pharmacologic inhibition of TGF-β receptor kinase activity was shown to reduce fibrosis and osteogenesis in the bone marrow (Zingariello et al. (2013) Blood 121(17):3345-3363). Furthermore, TGF-β inhibition significantly reduced fibrosis in JAK2 V617F+ and MF mouse models (Agarwal et al. (2016) Stem Cell Investig. 3:5; Zingariello et al. (2013) Blood 121(17):3345-3363). Given the potent immunomodulatory and pro-fibrotic properties of TGF-β, NIS793 might prove useful in the reversal of bone marrow fibrosis in patients with PMF, and could provide significant therapeutic benefit in conjunction with therapies directed at limiting disease burden, including TIM-3 blockade.

Hypomethylating agents induce broad epigenetic effects, e.g., downregulating genes involved in cell cycle, cell division and mitosis, and upregulating genes involved in cell differentiation. These anti-leukemic effects are accompanied by increased expression of TIM-3 as well as PD-1, PD-L1, PD-L2 and CTLA4, potentially downregulating immune-mediated anti-leukemic effects (Yang et al., (2014) Leukemia, 28(6):1280-8; Ørskov et al. (2015) Oncotarget, 6(11): 9612-9626). Without wishing to be bound by theory, it is believed that in some embodiments, a combination described herein (e.g., a combination comprising an anti-TIM-3 antibody molecule described herein) can be used to decrease an immunosuppressive tumor microenvironment.

IL-1β secreted by MPN clones has been shown to remodel the stem cell niche in a murine disease model and to support the growth of the malignant clone. In a mouse model of disease it was shown that blockade of IL-1 signaling using the recombinant IL-1 receptor antagonist (IL-1Ra) anakinra reduced platelet counts and increased BM MSC frequency (Arranz et al Nature. 2014 Aug. 7; 512(7512):78-81).

In MF patients, the level of IL-1β and mean number of circulating CD34+ cells were shown to be increased regardless of mutational status and behavior of the MF-derived HSPCs in vitro can be upregulated by cooperation between various pro-inflammatory factors in the inflammatory microenvironment, which appears to select for the malignant clone (Sollazzo et al Oncotarget. 2016; 7:43974-43988). Without wishing to be bound by theory, it is believed that in some embodiments, a combination comprising a TIM-3 inhibitor and a TGF-β inhibitor, optionally further comprising a hypomethylating agent, and optionally further comprising a PD-1 inhibitor or an IL-1β inhibitor, can be administered safely with little overlapping toxicity contributed by the TIM-3 inhibitor, and that the TIM-3 inhibitor can improve the efficacy of the TGF-β inhibitor, the PD-1 inhibitor, the hypomethylating agent, and/or the IL-1β inhibitor in treating MF. Patients with myelodysplastic syndrome (MDS) overexpress TIM-3, which inhibits immune recognition by cytotoxic T cells (Kikushige et al. Cell Stem Cell. 2010; 7(6): 708-717), and TIM-3 expression levels on MDS blasts increases as MDS progresses to the advanced stage. It has been observed that the proliferation of TIM-3 and MDS blasts is inhibited by the blockade of TIM-3 using an anti-TIM-3 antibody (Asayama et al. Oncotarget 2017; 8(51):88904-17).

Elevated levels of TGF-β signaling can contribute to the pathogenesis of MDS, as demonstrated by elevated plasma levels of TGFβ (Zorat et al. Br J Haematol 2001; 115(4):881-94; Allampallam et al. Int J Hematol 2002; 75(3):289-97), and the fact that SMAD2/3 are constitutively activated in bone marrow samples collected from MDS. Similarly, RNAseq analysis of bone marrow stroma from MDS patients demonstrated upregulation of TGF-β as the dominant cytokine signal (Geyh et al. Haematologica 2018; 103:1462-1471). TGF-β1 can also cause function deficits. For instance, elevated TGF-β1 is sufficient to block erythroid maturation (Gao et al. Blood 2016; 128(23):2637-2641) and induce functional deficits in the bone marrow stroma (Geyh et al. Haematologica 2018; 103:1462-1471). Further, a subset of anemic, lower risk MDS patients that were administered a TGF-β superfamily ligand trap demonstrated hematologic improvements and a reduced need for red blood cell transfusions (Fenaux et al. Presented at: 2019 European Hematology Association Congress 2018; Abstract S837). Without wishing to be bound by theory, it is believed that in some embodiments, a combination comprising a TIM-3 inhibitor and a TGF-β inhibitor, can be used to suppress aberrant immune activation implicated in the pathogenesis of MDS, e.g., lower risk MDS.

Accordingly, disclosed herein are, at least in part, are combination therapies that can be used to treat or prevent disorders, such as cancerous disorders (e.g., myelofibrosis, or myelodysplastic syndrome (MDS)). In certain embodiments, the combination comprises a TIM-3 inhibitor and a TGF-β inhibitor, and optionally a hypomethylating agent. In some embodiments, the TIM-3 inhibitor comprises an antibody molecule (e.g., humanized antibody molecule) that binds to TIM-3 with high affinity and specificity. In some embodiments, the TGF-β inhibitor comprises an antibody molecule (e.g., humanized antibody molecule) that binds to TGF-β with high affinity and specificity. In some embodiments, the combination further comprises a hypomethylating agent. In some embodiments, the combination further comprises a PD-1 inhibitor or an IL-1β inhibitor. In some embodiments, the PD-1 inhibitor comprises an antibody molecule (e.g., humanized antibody molecule) that binds to PD-1 with high affinity and specificity. In some embodiments, the IL-1β inhibitor comprises an antibody molecule (e.g., humanized antibody molecule) that binds IL-1β with high affinity and specificity. The combinations described herein can be used according to a dosage regimen described herein. Pharmaceutical compositions and dose formulations relating to the combinations described herein are also provided.

### Definitions

Additional terms are defined below and throughout the application.

As used herein, the articles “a” and “an” refer to one or to more than one (e.g., to at least one) of the grammatical object of the article.

The term “or” is used herein to mean, and is used interchangeably with, the term “and/or,” unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

By “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In embodiments, the additional therapeutic agent is administered at a therapeutic or lower-than therapeutic dose. In certain embodiments, the concentration of the second therapeutic agent that is required to achieve inhibition, e.g., growth inhibition, is lower when the second therapeutic agent is administered in combination with the first therapeutic agent, e.g., the anti-TIM-3 antibody molecule, than when the second therapeutic agent is administered individually. In certain embodiments, the concentration of the first therapeutic agent that is required to achieve inhibition, e.g., growth inhibition, is lower when the first therapeutic agent is administered in combination with the second therapeutic agent than when the first therapeutic agent is administered individually. In certain embodiments, in a combination therapy, the concentration of the second therapeutic agent that is required to achieve inhibition, e.g., growth inhibition, is lower than the therapeutic dose of the second therapeutic agent as a monotherapy, e.g., 10-20%, 20-30%, 30-40%, 40-50%, 50-60%, 60-70%, 70-80%, or 80-90% lower. In certain embodiments, in a combination therapy, the concentration of the first therapeutic agent that is required to achieve inhibition, e.g., growth inhibition, is lower than the therapeutic dose of the first therapeutic agent as a monotherapy, e.g., 10-20%, 20-30%, 30-40%, 40-50%, 50-60%, 60-70%, 70-80%, or 80-90% lower.

The term “inhibition,” “inhibitor,” or “antagonist” includes a reduction in a certain parameter, e.g., an activity, of a given molecule, e.g., an immune checkpoint inhibitor. For example, inhibition of an activity, e.g., a TIM-3 activity, of at least 5%, 10%, 20%, 30%, 40% or more is included by this term. Thus, inhibition need not be 100%.

The term “activation,” “activator,” or “agonist” includes an increase in a certain parameter, e.g., an activity, of a given molecule, e.g., a costimulatory molecule. For example, increase of an activity, e.g., a costimulatory activity, of at least 5%, 10%, 25%, 50%, 75% or more is included by this term.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, solid tumors, e.g., lung cancer, breast cancer, prostate cancer, ovarian cancer, cervical cancer, skin cancer, pancreatic cancer, colorectal cancer, renal cancer, liver cancer, and brain cancer, and hematologic malignancies, e.g., lymphoma and leukemia, and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to, an MHC class I molecule, TNF receptor proteins, Immunoglobulin-like proteins, cytokine receptors, integrins, signalling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

“Immune effector cell,” or “effector cell” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector” or “effector” “function” or “response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

As used herein, the terms “treat,” “treatment” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat,” “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat,” “treatment” and “treating” refer to the reduction or stabilization of tumor size or cancerous cell count.

The compositions, formulations, and methods of the present invention encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases, for example, to identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### Myeloproliferative Neoplasms

The combinations described herein can be used to treat a myeloproliferative neoplasm. Myeloproliferative neoplasms (MPNs) are typically considered as a group of hematological cancers that result from clonal and abnormal growth and proliferation of one or more hematopoietic cell lineages in the bone marrow of an individual. Common myeloproliferative neoplasms include, but are not limited to, myelofibrosis (MF) (e.g., a primary myelofibrosis (PMF), post-essential thrombocythemia myelofibrosis (PET-MF), post-polycythemia vera myelofibrosis (PPV-MF)), essential thrombocytosis (ET), polycythemia vera (PV). In some embodiments, myelofibrosis is characterized by an excessive build-up of scar tissue (fibrosis) in the bone marrow, preventing the ability of the bone marrow to generate new blood cells. Presently, the only potential curative treatment for myelofibrosis is allogeneic hematopoietic stem cell transplantation (ASCT), for which the great majority of patients is ineligible. Treatment options remain primarily palliative, and aimed at controlling disease symptoms, complications, and improving the patients' quality of life. Therefore, there is a need for the development of novel treatment compositions and combinations for myelofibrosis.

Myelofibrosis is typically considered as a Philadelphia chromosome-negative myeloproliferative neoplasm characterized by the presence of megakaryocyte proliferation and atypia, usually accompanied by either reticulin and/or collagen fibrosis, splenomegaly (e.g., due to extramedullary hematopoiesis), anemia (e.g., due to bone marrow failure and splenic sequestration), and debilitating constitutional symptoms (e.g., due to overexpression of inflammatory cytokines) that include fatigue, weight loss, pruritus, night sweats, fever, and bone, muscle, or abdominal pain. TGF-β, an important regulator of pathological fibrosis, is generally overexpressed in all fibrotic tissues and it induces collagen production in cultured fibroblasts, regardless of their origin (Lafyatis, Nat Rev Rheumatol. 2014; 10(12):706-719). An increasing number of niche components have been identified revealing a complex network of cell and matrix interactions and signaling pathways, which together create a unique microenvironment with TGF-β being an integral part of this environment. Cell-cell and cell-matrix interactions with the bone marrow are important components of the orchestrated process of activation of latent TGF-β (Arranz et al., Nature. 2014; 512(7512): 78-81). TGF-β production correlates with the progression of fibrotic diseases and TGF-β inhibition has been shown to reduce fibrotic processes in many experimental models (Massagué, FEBS Lett. 2012; 586(14): 1833). Bone marrow microenvironment and its interactions with TGF-β can contribute to myelofibrosis (Blank and Karlsson, Blood. 2015; 125(23): 3542-50). In the bone marrow of MPNs patients, TGF-β is believed to be produced by hematopoietic cells and necrotic and viable megakaryocytes are an important source of latent TGF-β stored within the alpha-granules of these bone marrow cells (Lataillade et al., Blood. 2008; 112(8):3026-35). Taken together, these data suggest that TGF-β plays a role in the physiopathology of myelofibrosis and can be beneficial to block TGF-β an inhibitor or with combination therapies.

In some embodiments, the compounds and combinations described herein, (e.g., a TIM-3 inhibitor and a TGF-β inhibitor; a TIM-3 inhibitor, TGF-β inhibitor, and a hypomethylating agent; a TIM-3 inhibitor, TGF-β inhibitor, and a PD-1 inhibitor, and optionally a hypomethylating agent or a TIM-3 inhibitor, TGF-β inhibitor, and an IL-1β inhibitor, and optionally a hypomethylating agent), are administered to a subject having or diagnosed with having myelofibrosis (MF) (e.g., a primary myelofibrosis (PMF), post-essential thrombocythemia myelofibrosis (PET-MF), post-polycythemia vera myelofibrosis (PPV-MF)), wherein the subject has previously received or is receiving a Janus kinase (JAK) inhibitor. In some embodiments, the JAK inhibitor is administered, for at least 1 month to at least 4 months (e.g., at least 1 month to at least 4 months, at least 1 month to at least 3 months, at least 1 month to at least 2 months, at least 2 month to at least 4 months, at least 2 month to at least 3 months, at least 3 months to at least 4 months) before the subject receive the combination therapy, e.g., a combination therapy described herein. In some embodiments, the JAK inhibitor is administered, for at least 1 month, at least 2 months, at least 3 months or at least 4 months, before the subject receive the combination therapy, e.g., a combination therapy described herein. In some embodiments, the JAK inhibitor is administered, for at least 3 months, before the subject receive the combination therapy, e.g., a combination therapy described herein. In some embodiments, the JAK inhibitor is administered, for at least 28 days, before the subject receive the combination therapy, e.g., a combination therapy described herein.

Without wishing to be bound by theory, it is believed that in some embodiments, treatment with the compounds and combinations described herein may result in anemia improvement of hemoglobin (Hb) ≥2.0 g/dL for transfusion independent subjects or improvement of hemoglobin (Hb) ≥1.5 g/dL for transfusion dependent subjects. In some embodiments, spleen volume and response is measured following treatment.

### Myelodysplastic Syndromes (MDS)

The combinations described herein can be used to treat a myelodysplastic syndrome (MDS). Myelodysplastic Syndromes (MDS) are typically regarded as a group of heterogeneous hematologic malignancies characterized by dysplastic and ineffective hematopoiesis, with a clinical presentation marked by bone marrow failure, peripheral blood cytopenias. MDS is categorized into subgroups, including but not limited to, very low risk MDS, low risk MDS, intermediate risk MDS, high risk MDS, or very high risk MDS. In some embodiments, MDS is characterized by cytogenic abnormalities, marrow blasts, and cytopenias.

In some embodiments, the combination described herein, e.g., a combination comprising a TIM-3 inhibitor and a TGFβ inhibitor, is used to treat a myelodysplastic syndrome (MDS), e.g., a very low risk MDS, low risk MDS, an intermediate risk MDS, a high risk MDS, or a very high risk MDS. In some embodiments, MDS is lower risk MDS, e.g., a very low risk MDS, a low risk MDS, or an intermediate risk MDS. In some embodiments, the combination described herein, e.g., a combination comprising a TIM-3 inhibitor, a TGFβ inhibitor, and an IL-1β inhibitor, is used to treat a myelodysplastic syndrome (MDS), e.g., a very low risk MDS, low risk MDS, an intermediate risk MDS, a high risk MDS, or a very high risk MDS. In some embodiments, the MDS is lower risk MDS, e.g., a very low risk MDS, a low risk MDS, or an intermediate risk MDS. In some embodiments, the MDS is a higher risk MDS, e.g., a high risk MDS or a very high risk MDS. In some embodiments, a score of less than or equal to 1.5 points on the International Prognostic Scoring System (IPSS-R) is classified as very low risk MDS. In some embodiments, a score of greater than 2 but less than or equal to 3 points on the International Prognostic Scoring System (IPSS-R) is classified as low risk MDS. In some embodiments, a score of greater than 3 but less than or equal to 4.5 points on the International Prognostic Scoring System (IPSS-R) is classified as intermediate risk MDS. In some embodiments, a score of greater than 4.5 but less than or equal to 6 points on the International Prognostic Scoring System (IPSS-R) is classified as high risk MDS. In some embodiments, a score of greater 6 points on the International Prognostic Scoring System (IPSS-R) is classified as very high risk MDS.

In some embodiments, the combinations and compounds described herein can be used in combination with as blood transfusions, iron chelation therapy or antibiotic or antifungal treatment to treat an MDS, e.g., a very low risk MDS, low risk MDS, an intermediate risk MDS, a high risk MDS, or a very high risk MDS. In some embodiments, the combinations and compounds described herein can be used in combination with an erythroid stimulating agent (ESA) to treat an MDS, e.g., a very low risk MDS, low risk MDS, an intermediate risk MDS, a high risk MDS, or a very high risk MDS. In some embodiments, a subject having or diagnosed with having an MDS (e.g., a very low risk MDS, low risk MDS, an intermediate risk MDS, a high risk MDS, or a very high risk MDS) with a serum erythropoietin (EPO) level <500 μ/L, is treated with an erythroid stimulating agent (ESA). In some embodiments, the combinations and compounds described herein can be used in combination with an erythroid lenalidomide to treat an MDS, e.g., a very low risk MDS, low risk MDS, an intermediate risk MDS, a high risk MDS, or a very high risk MDS. In some embodiments, a subject having or diagnosed with having an MDS (e.g., a very low risk MDS, low risk MDS, an intermediate risk MDS, a high risk MDS, or a very high risk MDS) comprising a deletion of the long arm of chromosome 5 [del(5q)] is treated with lenalidomide.

### TIM-3 Inhibitors

TIM-3 is expressed on the majority of CD34+CD38− leukemic stem cells (LSCs) and CD34+CD38+ leukemic progenitors in AML, not but in CD34+CD38 normal hematopoietic stem cells (HSCs) (Kikushige et al. Cell Stem Cell. 2010; 7(6):708-717; Jan et al. Proc Natl Acad Sci USA. 2011; 108(12):5009-5014). Functional evidence for a key role for TIM-3 in AML was established by use of an anti-TIM-3 antibody which inhibited engraftment and development of human AML in immunodeficient murine hosts (Kikushige et al. Cell Stem Cell. 2010; 7(6):708-717). Upregulation of TIM-3 is also associated with leukemic transformation of pre-leukemic disease, include myelodysplastic syndromes (MDSs) and myeloproliferative neoplasms (MPNs), such as chronic myelogenous leukemia (CML) (Kikushige et al., Cell Stem Cell. 2015; 17(3):341-352). TIM-3 expression on MDS blasts was also found to correlate with disease progression (Asayama et al. Oncotarget. 2017; 8(51):88904-88917).

In addition to its cell-autonomous role on pre-leukemic and leukemic stem cells, TIM-3 has a widespread and complex role in immune system regulation, with roles in both the adaptive immune response (CD4+ and CD8+ T effector cells, regulatory T cells) and innate immune responses (macrophages, dendritic cells, NK cells). TIM-3 has an important role in tumor-induced immune suppression as it marks the most suppressed or dysfunctional populations of CD8+ T cells in animal models of solid and hematologic malignancies, and is expressed on FoxP3+ regulatory T cells (Tregs), which correlate with disease severity in many cancer indications (Sakuishi et al. J Exp Med. 2010; 207(10): 2187-2194; Zhou et al., Blood. 2011; 117(17): 4501-10; Gao et al. PLoS One. 2012; 7(2):e30676; Yan et al. PLoS One. 2013; 8(3): e58006). TIM-3 is expressed on exhausted or dysfunctional T cells in cancer, and ex vivo TIM-3 blockade of TIM-3+ NY-ESO-1+ T cells from melanoma patients restored IFN-γ and TNF-α production as well as the proliferation in response to antigenic stimulation (Fourcade et al. J Exp Med. 2010; 207(10): 2175-2186). Blockade of TIM-3 on macrophages and antigen cross-presenting dendritic cells enhances activation and inflammatory cytokine/chemokine production (Zhang et al., PLoS One. 2011; 6(5): e19664; Zhang et al. J Leukoc Biol. 2012; 91(2): 189-96, Chiba et al., Nat Immunol. 2012; 13(9):832-842; de Mingo Pulido et al., Cancer Cell. 2018; 33(1): 60-74. e6), ultimately leading to enhanced effector T cells responses.

Without wishing to be bound by theory, it is believed that in some embodiments, constitutive JAK2/STAT3/STAT5 activation in MF patients, mainly in monocytes, megakaryocytes, and platelets, can cause TIM-3-mediated immune escape by reducing T cell activation, metabolic activity, and cell cycle progression (potentially similarly to the PD-L1-mediated immune escape). A TIM-3 inhibitor, e.g., an anti-TIM-3 antibody molecule described herein, can be used to mount an immune response against MF progenitor in order to reduce disease burden and progressive disease in MF patients.

In addition to its immunomodulatory role, TIM-3 is expressed on leukemic stem cells in MDS. Without wishing to be bound by theory, it is believed in some embodiments, use of TIM-3 inhibitor, e.g., an anti-TIM-3 antibody described herein, can restore an anti-tumor immune response and target MDS stem cells in a subject with MDS. A TIM-3 inhibitor, e.g., an anti-TIM-3 antibody molecule described herein, can be used to mount an immune response against an MDS progenitor in order to reduce disease burden and progressive disease in MDS patients.

In certain embodiments, the combination described herein includes a TIM-3 inhibitor, e.g., an anti-TIM-3 antibody molecule. In some embodiments, the anti-TIM-3 antibody molecule binds to a mammalian, e.g., human, TIM-3. For example, the antibody molecule binds specifically to an epitope, e.g., linear or conformational epitope on TIM-3.

As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full-length antibody which has an immunoglobulin Fc region). In an embodiment, an antibody molecule comprises a full-length antibody, or a full-length immunoglobulin chain. In an embodiment, an antibody molecule comprises an antigen binding or functional fragment of a full-length antibody, or a full-length immunoglobulin chain. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule.

In an embodiment, an antibody molecule is a monospecific antibody molecule and binds a single epitope. For example, a monospecific antibody molecule can have a plurality of immunoglobulin variable domain sequences, each of which binds the same epitope.

In an embodiment, an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule,

In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap. In an embodiment the first and second epitopes do not overlap. In an embodiment, the first and second epitopes are on different antigens, e.g., the different proteins (or different subunits of a multimeric protein). In an embodiment, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or fragment thereof, having binding specificity for a first epitope and a half antibody, or fragment thereof, having binding specificity for a second epitope. In an embodiment, a bispecific antibody molecule comprises a scFv, or fragment thereof, have binding specificity for a first epitope and a scFv, or fragment thereof, have binding specificity for a second epitope. In an embodiment, the first epitope is located on TIM-3 and the second epitope is located on a PD-1, LAG-3, CEACAM (e.g., CEACAM-1 and/or CEACAM-5), PD-L1, or PD-L2.

Protocols for generating multi-specific (e.g., bispecific or trispecific) or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US 2002/004587A1, US 2002/076406A1, US 2002/103345A1, US 2003/207346A1, US 2003/211078A1, US 2004/219643A1, US 2004/220388A1, US 2004/242847A1, US 2005/003403A1, US 2005/004352A1, US 2005/069552A1, US 2005/079170A1, US 2005/100543A1, US 2005/136049A1, US 2005/136051A1, US 2005/163782A1, US 2005/266425A1, US 2006/083747A1, US 2006/120960A1, US 2006/204493A1, US 2006/263367A1, US 2007/004909A1, US 2007/087381A1, US 2007/128150A1, US 2007/141049A1, US 2007/154901A1, US 2007/274985A1, US 2008/050370A1, US 2008/069820A1, US 2008/152645A1, US 2008/171855A1, US 2008/241884A1, US 2008/254512A1, US 2008/260738A1, US 2009/130106A1, US 2009/148905A1, US 2009/155275A1, US 2009/162359A1, US 2009/162360A1, US 2009/175851A1, US 2009/175867A1, US 2009/232811A1, US 2009/234105A1, US 2009/263392A1, US 2009/274649A1, EP 346087A2, WO 00/06605A2, WO 02/072635A2, WO 04/081051A1, WO 06/020258A2, WO 2007/044887A2, WO 2007/095338A2, WO 2007/137760A2, WO 2008/119353A1, WO 2009/021754A2, WO 2009/068630A1, WO 91/03493A1, WO 93/23537A1, WO 94/09131A1, WO 94/12625A2, WO 95/09917A1, WO 96/37621A2, WO 99/64460A1. The contents of the above-referenced applications are incorporated herein by reference in their entireties.

In other embodiments, the anti-TIM-3 antibody molecule (e.g., a monospecific, bispecific, or multispecific antibody molecule) is covalently linked, e.g., fused, to another partner e.g., a protein e.g., one, two or more cytokines, e.g., as a fusion molecule for example a fusion protein. In other embodiments, the fusion molecule comprises one or more proteins, e.g., one, two or more cytokines. In one embodiment, the cytokine is an interleukin (IL) chosen from one, two, three or more of IL-1, IL-2, IL-12, IL-15 or IL-21. In one embodiment, a bispecific antibody molecule has a first binding specificity to a first target (e.g., to PD-1), a second binding specificity to a second target (e.g., LAG-3 or TIM-3), and is optionally linked to an interleukin (e.g., IL-12) domain e.g., full length IL-12 or a portion thereof.

A “fusion protein” and a “fusion polypeptide” refer to a polypeptide having at least two portions covalently linked together, where each of the portions is a polypeptide having a different property. The property may be a biological property, such as activity in vitro or in vivo. The property can also be simple chemical or physical property, such as binding to a target molecule, catalysis of a reaction, etc. The two portions can be linked directly by a single peptide bond or through a peptide linker, but are in reading frame with each other.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, and HCDR3) and three CDRs in each light chain variable region (LCDR1, LCDR2, and LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Generally, unless specifically indicated, the anti-TIM-3 antibody molecules can include any combination of one or more Kabat CDRs and/or Chothia hypervariable loops, e.g., described in Table 1. In one embodiment, the following definitions are used for the anti-TIM-3 antibody molecules described in Table 1: HCDR1 according to the combined CDR definitions of both Kabat and Chothia, and HCCDRs 2-3 and LCCDRs 1-3 according the CDR definition of Kabat. Under all definitions, each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to the TIM-3 polypeptide, or an epitope thereof. With respect to proteins (or protein mimetics), the antigen-binding site typically includes one or more loops (of at least four amino acids or amino acid mimics) that form an interface that binds to the TIM-3 polypeptide. Typically, the antigen-binding site of an antibody molecule includes at least one or two CDRs and/or hypervariable loops, or more typically at least three, four, five or six CDRs and/or hypervariable loops.

The terms “compete” or “cross-compete” are used interchangeably herein to refer to the ability of an antibody molecule to interfere with binding of an anti-TIM-3 antibody molecule, e.g., an anti-TIM-3 antibody molecule provided herein, to a target, e.g., human TIM-3. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a FACS assay, an ELISA or BIACORE assay. In some embodiments, a competition binding assay is a quantitative competition assay. In some embodiments, a first anti-TIM-3 antibody molecule is said to compete for binding to the target with a second anti-TIM-3 antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see e.g., Saleh et al., Cancer Immunol. Immunother. 32:180-190 (1990)) and also because of potential allergic reactions (see e.g., LoBuglio et al., Hybridoma, 5:5117-5123 (1986)).

The antibody molecule can be a polyclonal or a monoclonal antibody. In other embodiments, the antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02 809; Fuchs et al. (1991) Bio/Technology 9:1370-1372; Hay et al. (1992) Hum Antibody Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to PD-1. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (see e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody molecule of the invention may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecule can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule may be conjugated to another molecular entity, typically a label or a therapeutic (e.g., a cytotoxic or cytostatic) agent or moiety. Radioactive isotopes can be used in diagnostic or therapeutic applications.

The invention provides radiolabeled antibody molecules and methods of labeling the same. In one embodiment, a method of labeling an antibody molecule is disclosed. The method includes contacting an antibody molecule, with a chelating agent, to thereby produce a conjugated antibody.

As is discussed above, the antibody molecule can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see, e.g., U.S. Pat. No. 5,208,020), CC-1065 (see, e.g., U.S. Pat. Nos. 5,475,092, 5,585,499, 5,846, 545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In one aspect, the disclosure provides a method of providing a target binding molecule that specifically binds to a target disclosed herein, e.g., TIM-3. For example, the target binding molecule is an antibody molecule. The method includes: providing a target protein that comprises at least a portion of non-human protein, the portion being homologous to (at least 70, 75, 80, 85, 87, 90, 92, 94, 95, 96, 97, 98% identical to) a corresponding portion of a human target protein, but differing by at least one amino acid (e.g., at least one, two, three, four, five, six, seven, eight, or nine amino acids); obtaining an antibody molecule that specifically binds to the antigen; and evaluating efficacy of the binding agent in modulating activity of the target protein. The method can further include administering the binding agent (e.g., antibody molecule) or a derivative (e.g., a humanized antibody molecule) to a human subject.

This disclosure provides an isolated nucleic acid molecule encoding the above antibody molecule, vectors and host cells thereof. The nucleic acid molecule includes but is not limited to RNA, genomic DNA and cDNA.

Exemplary TIM-3 Inhibitors

In certain embodiments, the combination described herein comprises an anti-TIM3 antibody molecule. In one embodiment, the anti-TIM-3 antibody molecule is disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 1 (e.g., from the heavy and light chain variable region sequences of ABTIM3-hum11 or ABTIM3-hum03 disclosed in Table 1), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, or encoded by a nucleotide sequence shown in Table 1.

In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 802, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 1. In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 820, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 1.

In one embodiment, the anti-TIM-3 antibody molecule comprises a VH comprising the amino acid sequence of SEQ ID NO: 806, or an amino acid sequence at least 85%, 90%, 95%, or 99% identical or higher to SEQ ID NO: 806. In one embodiment, the anti-TIM-3 antibody molecule comprises a VL comprising the amino acid sequence of SEQ ID NO: 816, or an amino acid sequence at least 85%, 90%, 95%, or 99% identical or higher to SEQ ID NO: 816. In one embodiment, the anti-TIM-3 antibody molecule comprises a VH comprising the amino acid sequence of SEQ ID NO: 822, or an amino acid sequence at least 85%, 90%, 95%, or 99% identical or higher to SEQ ID NO: 822. In one embodiment, the anti-TIM-3 antibody molecule comprises a VL comprising the amino acid sequence of SEQ ID NO: 826, or an amino acid sequence at least 85%, 90%, 95%, or 99% identical or higher to SEQ ID NO: 826. In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment, the antibody molecule comprises a VH encoded by the nucleotide sequence of SEQ ID NO: 807, or a nucleotide sequence at least 85%, 90%, 95%, or 99% identical or higher to SEQ ID NO: 807. In one embodiment, the antibody molecule comprises a VL encoded by the nucleotide sequence of SEQ ID NO: 817, or a nucleotide sequence at least 85%, 90%, 95%, or 99% identical or higher to SEQ ID NO: 817. In one embodiment, the antibody molecule comprises a VH encoded by the nucleotide sequence of SEQ ID NO: 823, or a nucleotide sequence at least 85%, 90%, 95%, or 99% identical or higher to SEQ ID NO: 823. In one embodiment, the antibody molecule comprises a VL encoded by the nucleotide sequence of SEQ ID NO: 827, or a nucleotide sequence at least 85%, 90%, 95%, or 99% identical or higher to SEQ ID NO: 827. In one embodiment, the antibody molecule comprises a VH encoded by the nucleotide sequence of SEQ ID NO: 807 and a VL encoded by the nucleotide sequence of SEQ ID NO: 817. In one embodiment, the antibody molecule comprises a VH encoded by the nucleotide sequence of SEQ ID NO: 823 and a VL encoded by the nucleotide sequence of SEQ ID NO: 827.

In one embodiment, the anti-TIM-3 antibody molecule comprises a heavy chain comprising the amino acid sequence of SEQ ID NO: 808, or an amino acid sequence at least 85%, 90%, 95%, or 99% identical or higher to SEQ ID NO: 808. In one embodiment, the anti-TIM-3 antibody molecule comprises a light chain comprising the amino acid sequence of SEQ ID NO: 818, or an amino acid sequence at least 85%, 90%, 95%, or 99% identical or higher to SEQ ID NO: 818. In one embodiment, the anti-TIM-3 antibody molecule comprises a heavy chain comprising the amino acid sequence of SEQ ID NO: 824, or an amino acid sequence at least 85%, 90%, 95%, or 99% identical or higher to SEQ ID NO: 824. In one embodiment, the anti-TIM-3 antibody molecule comprises a light chain comprising the amino acid sequence of SEQ ID NO: 828, or an amino acid sequence at least 85%, 90%, 95%, or 99% identical or higher to SEQ ID NO: 828. In one embodiment, the anti-TIM-3 antibody molecule comprises a heavy chain comprising the amino acid sequence of SEQ ID NO: 808 and a light chain comprising the amino acid sequence of SEQ ID NO: 818. In one embodiment, the anti-TIM-3 antibody molecule comprises a heavy chain comprising the amino acid sequence of SEQ ID NO: 824 and a light chain comprising the amino acid sequence of SEQ ID NO: 828.

In one embodiment, the antibody molecule comprises a heavy chain encoded by the nucleotide sequence of SEQ ID NO: 809, or a nucleotide sequence at least 85%, 90%, 95%, or 99% identical or higher to SEQ ID NO: 809. In one embodiment, the antibody molecule comprises a light chain encoded by the nucleotide sequence of SEQ ID NO: 819, or a nucleotide sequence at least 85%, 90%, 95%, or 99% identical or higher to SEQ ID NO: 819. In one embodiment, the antibody molecule comprises a heavy chain encoded by the nucleotide sequence of SEQ ID NO: 825, or a nucleotide sequence at least 85%, 90%, 95%, or 99% identical or higher to SEQ ID NO: 825. In one embodiment, the antibody molecule comprises a light chain encoded by the nucleotide sequence of SEQ ID NO: 829, or a nucleotide sequence at least 85%, 90%, 95%, or 99% identical or higher to SEQ ID NO: 829. In one embodiment, the antibody molecule comprises a heavy chain encoded by the nucleotide sequence of SEQ ID NO: 809 and a light chain encoded by the nucleotide sequence of SEQ ID NO: 819. In one embodiment, the antibody molecule comprises a heavy chain encoded by the nucleotide sequence of SEQ ID NO: 825 and a light chain encoded by the nucleotide sequence of SEQ ID NO: 829.

The antibody molecules described herein can be made by vectors, host cells, and methods described in US 2015/0218274, incorporated by reference in its entirety.

TABLE 1
Amino acid and nucleotide sequences of exemplary anti-TIM-3 antibody molecules
ABTIM3-hum11

| | | |
|---|---|---|
| SEQ ID NO: 801 | HCDR1 | SYNMH |
| (Kabat) | | |
| SEQ ID NO: 802 | HCDR2 | DIYPGNGDTSYNQKFKG |
| (Kabat) | | |
| SEQ ID NO: 803 | HCDR3 | VGGAFPMDY |
| (Kabat) | | |
| SEQ ID NO: 804 | HCDR1 | GYTFTSY |
| (Chothia) | | |
| SEQ ID NO: 805 | HCDR2 | YPGNGD |
| (Chothia) | | |
| SEQ ID NO: 803 | HCDR3 | VGGAFPMDY |
| (Chothia) | | |
| SEQ ID NO: 806 | VH | QVQLVQSGAEVKKPGSSVKVSCKASGYTFTSYNMHWVR |
| | | QAPGQGLEWMGDIYPGNGDTSYNQKFKGRVTITADKSTS |
| | | TVYMELSSLRSEDTAVYYCARVGGAFPMDYWGQGTTVT |
| | | VSS |
| SEQ ID NO: 807 | DNA VH | CAGGTGCAGCTGGTGCAGTCAGGCGCCGAAGTGAAGA |
| | | AACCCGGCTCTAGCGTGAAAGTTTCTTGTAAAGCTAGT |
| | | GGCTACACCTTCACTAGCTATAATATGCACTGGGTTCGC |
| | | CAGGCCCCAGGGCAAGGCCTCGAGTGGATGGGCGATAT |
| | | CTACCCCGGGAACGGCGACACTAGTTATAATCAGAAGT |
| | | TTAAGGGTAGAGTCACTATCACCGCCGATAAGTCTACT |
| | | AGCACCGTCTATATGGAACTGAGTTCCCTGAGGTCTGA |
| | | GGACACCGCCGTCTACTACTGCGCTAGAGTGGGCGGAG |
| | | CCTTCCCTATGGACTACTGGGGTCAAGGCACTACCGTG |
| | | ACCGTGTCTAGC |
| SEQ ID NO: 808 | Heavy | QVQLVQSGAEVKKPGSSVKVSCKASGYTFTSYNMHWVR |
| | chain | QAPGQGLEWMGDIYPGNGDTSYNQKFKGRVTITADKSTS |
| | | TVYMELSSLRSEDTAVYYCARVGGAFPMDYWGQGTTVT |
| | | VSSASTKGPSVFPLAPCSRSTSESTAALGCLVKDYFPEPVT |
| | | VSWNSGALTSGVHTFPAVLQSSGLYSLSSVVTVPSSSLGT |
| | | KTYTCNVDHKPSNTKVDKRVESKYGPPCPPCPAPEFLGGP |
| | | SVFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPP |
| | | VLDSDGSFFLYSRLTVDKSRWQEGNVFSCSVMHEALHNH |
| | | YTQKSLSLSLG |
| SEQ ID NO: 809 | DNA | CAGGTGCAGCTGGTGCAGTCAGGCGCCGAAGTGAAGA |
| | heavy | AACCCGGCTCTAGCGTGAAAGTTTCTTGTAAAGCTAGT |
| | chain | GGCTACACCTTCACTAGCTATAATATGCACTGGGTTCGC |
| | | CAGGCCCCAGGGCAAGGCCTCGAGTGGATGGGCGATAT |
| | | CTACCCCGGGAACGGCGACACTAGTTATAATCAGAAGT |
| | | TTAAGGGTAGAGTCACTATCACCGCCGATAAGTCTACT |
| | | AGCACCGTCTATATGGAACTGAGTTCCCTGAGGTCTGA |
| | | GGACACCGCCGTCTACTACTGCGCTAGAGTGGGCGGAG |
| | | CCTTCCCTATGGACTACTGGGGTCAAGGCACTACCGTG |
| | | ACCGTGTCTAGCGCTAGCACTAAGGGCCCGTCCGTGTT |
| | | CCCCCTGGCACCTTGTAGCCGGAGCACTAGCGAATCCA |
| | | CCGCTGCCCTCGGCTGCCTGGTCAAGGATTACTTCCCGG |
| | | AGCCCGTGACCGTGTCCTGGAACAGCGGAGCCCTGACC |
| | | TCCGGAGTGCACACCTTCCCCGCTGTGCTGCAGAGCTCC |
| | | GGGCTGTACTCGCTGTCGTCGGTGGTCACGGTGCCTTCA |
| | | TCTAGCCTGGGTACCAAGACCTACACTTGCAACGTGGA |
| | | CCACAAGCCTTCCAACACTAAGGTGGACAAGCGCGTCG |
| | | AATCGAAGTACGGCCCACCGTGCCCGCCTTGTCCCGCG |
| | | CCGGAGTTCCTCGGCGGTCCCTCGGTCTTTCTGTTCCCA |
| | | CCGAAGCCCAAGGACACTTTGATGATTTCCCGCACCCC |
| | | TGAAGTGACATGCGTGGTCGTGGACGTGTCACAGGAAG |
| | | ATCCGGAGGTGCAGTTCAATTGGTACGTGGATGGCGTC |
| | | GAGGTGCACAACGCCAAAACCAAGCCGAGGGAGGAGC |
| | | AGTTCAACTCCACTTACCGCGTCGTGTCCGTGCTGACGG |
| | | TGCTGCATCAGGACTGGCTGAACGGGAAGGAGTACAAG |
| | | TGCAAAGTGTCCAACAAGGGACTTCCTAGCTCAATCGA |
| | | AAAGACCATCTCGAAAGCCAAGGGACAGCCCCGGGAA |
| | | CCCCAAGTGTATACCCTGCCACCGAGCCAGGAAGAAAT |
| | | GACTAAGAACCAAGTCTCATTGACTTGCCTTGTGAAGG |
| | | GCTTCTACCCATCGGATATCGCCGTGGAATGGGAGTCC |
| | | AACGGCCAGCCGGAAAACAACTACAAGACCACCCCTCC |
| | | GGTGCTGGACTCAGACGGATCCTTCTTCCTCTACTCGCG |
| | | GCTGACCGTGGATAAGAGCAGATGGCAGGAGGGAAAT |
| | | GTGTTCAGCTGTTCTGTGATGCATGAAGCCCTGCACAAC |
| | | CACTACACTCAGAAGTCCCTGTCCCTCTCCCTGGGA |
| SEQ ID NO: 810 | LCDR1 | RASESVEYYGTSLMQ |
| (Kabat) | | |
| SEQ ID NO: 811 | LCDR2 | AASNVES |
| (Kabat) | | |
| SEQ ID NO: 812 | LCDR3 | QQSRKDPST |
| (Kabat) | | |
| SEQ ID NO: 813 | LCDR1 | SESVEYYGTSL |
| (Chothia) | | |
| SEQ ID NO: 814 | LCDR2 | AAS |
| (Chothia) | | |
| SEQ ID NO: 815 | LCDR3 | SRKDPS |
| (Chothia) | | |
| SEQ ID NO: 816 | VL | AIQLTQSPSSLSASVGDRVTITCRASESVEYYGTSLMQWY |
| | | QQKPGKAPKLLIYAASNVESGVPSRFSGSGSGTDFTLTISS |
| | | LQPEDFATYFCQQSRKDPSTFGGGTKVEIK |
| SEQ ID NO: 817 | DNA VL | GCTATTCAGCTGACTCAGTCACCTAGTAGCCTGAGCGCT |
| | | AGTGTGGGCGATAGAGTGACTATCACCTGTAGAGCTAG |
| | | TGAATCAGTCGAGTACTACGGCACTAGCCTGATGCAGT |
| | | GGTATCAGCAGAAGCCCGGGAAAGCCCCTAAGCTGCTG |
| | | ATCTACGCCGCCTCTAACGTGGAATCAGGCGTGCCCTCT |
| | | AGGTTTAGCGGTAGCGGTAGTGGCACCGACTTCACCCT |
| | | GACTATCTCTAGCCTGCAGCCCGAGGACTTCGCTACCTA |
| | | CTTCTGTCAGCAGTCTAGGAAGGACCCTAGCACCTTCG |
| | | GCGGAGGCACTAAGGTCGAGATTAAG |
| SEQ ID NO: 818 | Light | AIQLTQSPSSLSASVGDRVTITCRASESVEYYGTSLMQWY |
| | chain | QQKPGKAPKLLIYAASNVESGVPSRFSGSGSGTDFTLTISS |
| | | LQPEDFATYFCQQSRKDPSTFGGGTKVEIKRTVAAPSVFIF |
| | | PPSDEQLKSGTASVVCLLNNFYPREAKVQWKVDNALQSG |
| | | NSQESVTEQDSKDSTYSLSSTLTLSKADYEKHKVYACEVT |
| | | HQGLSSPVTKSFNRGEC |
| SEQ ID NO: 819 | DNA | GCTATTCAGCTGACTCAGTCACCTAGTAGCCTGAGCGCT |
| | light | AGTGTGGGCGATAGAGTGACTATCACCTGTAGAGCTAG |
| | chain | TGAATCAGTCGAGTACTACGGCACTAGCCTGATGCAGT |
| | | GGTATCAGCAGAAGCCCGGGAAAGCCCCTAAGCTGCTG |
| | | ATCTACGCCGCCTCTAACGTGGAATCAGGCGTGCCCTCT |
| | | AGGTTTAGCGGTAGCGGTAGTGGCACCGACTTCACCCT |
| | | GACTATCTCTAGCCTGCAGCCCGAGGACTTCGCTACCTA |
| | | CTTCTGTCAGCAGTCTAGGAAGGACCCTAGCACCTTCG |
| | | GCGGAGGCACTAAGGTCGAGATTAAGCGTACGGTGGCC |
| | | GCTCCCAGCGTGTTCATCTTCCCCCCCAGCGACGAGCA |
| | | GCTGAAGAGCGGCACCGCCAGCGTGGTGTGCCTGCTGA |
| | | ACAACTTCTACCCCCGGGAGGCCAAGGTGCAGTGGAAG |
| | | GTGGACAACGCCCTGCAGAGCGGCAACAGCCAGGAGA |
| | | GCGTCACCGAGCAGGACAGCAAGGACTCCACCTACAGC |
| | | CTGAGCAGCACCCTGACCCTGAGCAAGGCCGACTACGA |
| | | GAAGCATAAGGTGTACGCCTGCGAGGTGACCCACCAGG |
| | | GCCTGTCCAGCCCCGTGACCAAGAGCTTCAACAGGGGC |
| | | GAGTGC |

ABTIM-hum03

| | | |
|---|---|---|
| SEQ ID NO: 801 | HCDR1 | SYNMH |
| (Kabat) | | |
| SEQ ID NO: 820 | HCDR2 | DIYPGQGDTSYNQKFKG |
| (Kabat) | | |
| SEQ ID NO: 803 | HCDR3 | VGGAFPMDY |
| (Kabat) | | |
| SEQ ID NO: 804 | HCDR1 | GYTFTSY |
| (Chothia) | | |
| SEQ ID NO: 821 | HCDR2 | YPGQGD |
| (Chothia) | | |
| SEQ ID NO: 803 | HCDR3 | VGGAFPMDY |
| (Chothia) | | |
| SEQ ID NO: 822 | VH | QVQLVQSGAEVKKPGASVKVSCKASGYTFTSYNMHWVR |
| | | QAPGQGLEWIGDIYPGQGDTSYNQKFKGRATMTADKSTS |
| | | TVYMELSSLRSEDTAVYYCARVGGAFPMDYWGQGTLVT |
| | | VSS |
| SEQ ID NO: 823 | DNA VH | CAGGTGCAGCTGGTGCAGTCAGGCGCCGAAGTGAAGA |
| | | AACCCGGCGCTAGTGTGAAAGTTAGCTGTAAAGCTAGT |
| | | GGCTATACTTTCACTTCTTATAATATGCACTGGGTCCGC |
| | | CAGGCCCCAGGTCAAGGCCTCGAGTGGATCGGCGATAT |
| | | CTACCCCGGTCAAGGCGACACTTCCTATAATCAGAAGT |
| | | TTAAGGGTAGAGCTACTATGACCGCCGATAAGTCTACT |
| | | TCTACCGTCTATATGGAACTGAGTTCCCTGAGGTCTGAG |
| | | GACACCGCCGTCTACTACTGCGCTAGAGTGGGCGGAGC |
| | | CTTCCCAATGGACTACTGGGGTCAAGGCACCCTGGTCA |
| | | CCGTGTCTAGC |
| SEQ ID NO: 824 | Heavy | QVQLVQSGAEVKKPGASVKVSCKASGYTFTSYNMHWVR |
| | chain | QAPGQGLEWIGDIYPGQGDTSYNQKFKGRATMTADKSTS |
| | | TVYMELSSLRSEDTAVYYCARVGGAFPMDYWGQGTLVT |
| | | VSSASTKGPSVFPLAPCSRSTSESTAALGCLVKDYFPEPVT |
| | | VSWNSGALTSGVHTFPAVLQSSGLYSLSSVVTVPSSSLGT |
| | | KTYTCNVDHKPSNTKVDKRVESKYGPPCPPCPAPEFLGGP |
| | | SVFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPP |
| | | VLDSDGSFFLYSRLTVDKSRWQEGNVFSCSVMHEALHNH |
| | | YTQKSLSLSLG |
| SEQ ID NO: 825 | DNA | CAGGTGCAGCTGGTGCAGTCAGGCGCCGAAGTGAAGA |
| | heavy | AACCCGGCGCTAGTGTGAAAGTTAGCTGTAAAGCTAGT |
| | chain | GGCTATACTTTCACTTCTTATAATATGCACTGGGTCCGC |
| | | CAGGCCCCAGGTCAAGGCCTCGAGTGGATCGGCGATAT |
| | | CTACCCCGGTCAAGGCGACACTTCCTATAATCAGAAGT |
| | | TTAAGGGTAGAGCTACTATGACCGCCGATAAGTCTACT |
| | | TCTACCGTCTATATGGAACTGAGTTCCCTGAGGTCTGAG |
| | | GACACCGCCGTCTACTACTGCGCTAGAGTGGGCGGAGC |
| | | CTTCCCAATGGACTACTGGGGTCAAGGCACCCTGGTCA |
| | | CCGTGTCTAGCGCTAGCACTAAGGGCCCGTCCGTGTTCC |
| | | CCCTGGCACCTTGTAGCCGGAGCACTAGCGAATCCACC |
| | | GCTGCCCTCGGCTGCCTGGTCAAGGATTACTTCCCGGA |
| | | GCCCGTGACCGTGTCCTGGAACAGCGGAGCCCTGACCT |
| | | CCGGAGTGCACACCTTCCCCGCTGTGCTGCAGAGCTCC |
| | | GGGCTGTACTCGCTGTCGTCGGTGGTCACGGTGCCTTCA |
| | | TCTAGCCTGGGTACCAAGACCTACACTTGCAACGTGGA |
| | | CCACAAGCCTTCCAACACTAAGGTGGACAAGCGCGTCG |
| | | AATCGAAGTACGGCCCACCGTGCCCGCCTTGTCCCGCG |
| | | CCGGAGTTCCTCGGCGGTCCCTCGGTCTTTCTGTTCCCA |
| | | CCGAAGCCCAAGGACACTTTGATGATTTCCCGCACCCC |
| | | TGAAGTGACATGCGTGGTCGTGGACGTGTCACAGGAAG |
| | | ATCCGGAGGTGCAGTTCAATTGGTACGTGGATGGCGTC |
| | | GAGGTGCACAACGCCAAAACCAAGCCGAGGGAGGAGC |
| | | AGTTCAACTCCACTTACCGCGTCGTGTCCGTGCTGACGG |
| | | TGCTGCATCAGGACTGGCTGAACGGGAAGGAGTACAAG |
| | | TGCAAAGTGTCCAACAAGGGACTTCCTAGCTCAATCGA |
| | | AAAGACCATCTCGAAAGCCAAGGGACAGCCCCGGGAA |
| | | CCCCAAGTGTATACCCTGCCACCGAGCCAGGAAGAAAT |
| | | GACTAAGAACCAAGTCTCATTGACTTGCCTTGTGAAGG |
| | | GCTTCTACCCATCGGATATCGCCGTGGAATGGGAGTCC |
| | | AACGGCCAGCCGGAAAACAACTACAAGACCACCCCTCC |
| | | GGTGCTGGACTCAGACGGATCCTTCTTCCTCTACTCGCG |
| | | GCTGACCGTGGATAAGAGCAGATGGCAGGAGGGAAAT |
| | | GTGTTCAGCTGTTCTGTGATGCATGAAGCCCTGCACAAC |
| | | CACTACACTCAGAAGTCCCTGTCCCTCTCCCTGGGA |
| SEQ ID NO: 810 | LCDR1 | RASESVEYYGTSLMQ |
| (Kabat) | | |
| SEQ ID NO: 811 | LCDR2 | AASNVES |
| (Kabat) | | |
| SEQ ID NO: 812 | LCDR3 | QQSRKDPST |
| (Kabat) | | |
| SEQ ID NO: 813 | LCDR1 | SESVEYYGTSL |
| (Chothia) | | |
| SEQ ID NO: 814 | LCDR2 | AAS |
| (Chothia) | | |
| SEQ ID NO: 815 | LCDR3 | SRKDPS |
| (Chothia) | | |
| SEQ ID NO: 826 | VL | DIVLTQSPDSLAVSLGERATINCRASESVEYYGTSLMQWY |
| | | QQKPGQPPKLLIYAASNVESGVPDRFSGSGSGTDFTLTISS |
| | | LQAEDVAVYYCQQSRKDPSTFGGGTKVEIK |
| SEQ ID NO: 827 | DNA VL | GATATCGTCCTGACTCAGTCACCCGATAGCCTGGCCGTC |
| | | AGCCTGGGCGAGCGGGCTACTATTAACTGTAGAGCTAG |
| | | TGAATCAGTCGAGTACTACGGCACTAGCCTGATGCAGT |
| | | GGTATCAGCAGAAGCCCGGTCAACCCCCTAAGCTGCTG |
| | | ATCTACGCCGCCTCTAACGTGGAATCAGGCGTGCCCGA |
| | | TAGGTTTAGCGGTAGCGGTAGTGGCACCGACTTCACCC |
| | | TGACTATTAGTAGCCTGCAGGCCGAGGACGTGGCCGTC |
| | | TACTACTGTCAGCAGTCTAGGAAGGACCCTAGCACCTT |
| | | CGGCGGAGGCACTAAGGTCGAGATTAAG |
| SEQ ID NO: 828 | Light | DIVLTQSPDSLAVSLGERATINCRASESVEYYGTSLMQWY |
| | chain | QQKPGQPPKLLIYAASNVESGVPDRFSGSGSGTDFTLTISS |
| | | LQAEDVAVYYCQQSRKDPSTFGGGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACEV |
| | | THQGLSSPVTKSFNRGEC |
| SEQ ID NO: 829 | DNA | GATATCGTCCTGACTCAGTCACCCGATAGCCTGGCCGTC |
| | light | AGCCTGGGCGAGCGGGCTACTATTAACTGTAGAGCTAG |
| | chain | TGAATCAGTCGAGTACTACGGCACTAGCCTGATGCAGT |
| | | GGTATCAGCAGAAGCCCGGTCAACCCCCTAAGCTGCTG |
| | | ATCTACGCCGCCTCTAACGTGGAATCAGGCGTGCCCGA |
| | | TAGGTTTAGCGGTAGCGGTAGTGGCACCGACTTCACCC |
| | | TGACTATTAGTAGCCTGCAGGCCGAGGACGTGGCCGTC |
| | | TACTACTGTCAGCAGTCTAGGAAGGACCCTAGCACCTT |
| | | CGGCGGAGGCACTAAGGTCGAGATTAAGCGTACGGTGG |
| | | CCGCTCCCAGCGTGTTCATCTTCCCCCCCAGCGACGAGC |
| | | AGCTGAAGAGCGGCACCGCCAGCGTGGTGTGCCTGCTG |
| | | AACAACTTCTACCCCCGGGAGGCCAAGGTGCAGTGGAA |
| | | GGTGGACAACGCCCTGCAGAGCGGCAACAGCCAGGAG |
| | | AGCGTCACCGAGCAGGACAGCAAGGACTCCACCTACAG |
| | | CCTGAGCAGCACCCTGACCCTGAGCAAGGCCGACTACG |
| | | AGAAGCATAAGGTGTACGCCTGCGAGGTGACCCACCAG |
| | | GGCCTGTCCAGCCCCGTGACCAAGAGCTTCAACAGGGG |
| | | CGAGTGC |

In one embodiment, the anti-TIM-3 antibody molecule includes at least one or two heavy chain variable domain (optionally including a constant region), at least one or two light chain variable domain (optionally including a constant region), or both, comprising the amino acid sequence of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences. The anti-TIM-3 antibody molecule, optionally, comprises a leader sequence from a heavy chain, a light chain, or both, as shown in US 2015/0218274; or a sequence substantially identical thereto.

In yet another embodiment, the anti-TIM-3 antibody molecule includes at least one, two, or three complementarity determining regions (CDRs) from a heavy chain variable region and/or a light chain variable region of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In yet another embodiment, the anti-TIM-3 antibody molecule includes at least one, two, or three CDRs (or collectively all of the CDRs) from a heavy chain variable region comprising an amino acid sequence shown in Tables 1-4 of US 2015/0218274, or encoded by a nucleotide sequence shown in Tables 1-4. In one embodiment, one or more of the CDRs (or collectively all of the CDRs) have one, two, three, four, five, six or more changes, e.g., amino acid substitutions or deletions, relative to the amino acid sequence shown in Tables 1-4, or encoded by a nucleotide sequence shown in Tables 1-4.

In yet another embodiment, the anti-TIM-3 antibody molecule includes at least one, two, or three CDRs (or collectively all of the CDRs) from a light chain variable region comprising an amino acid sequence shown in Tables 1-4 of US 2015/0218274, or encoded by a nucleotide sequence shown in Tables 1-4. In one embodiment, one or more of the CDRs (or collectively all of the CDRs) have one, two, three, four, five, six or more changes, e.g., amino acid substitutions or deletions, relative to the amino acid sequence shown in Tables 1-4, or encoded by a nucleotide sequence shown in Tables 1-4. In certain embodiments, the anti-TIM-3 antibody molecule includes a substitution in a light chain CDR, e.g., one or more substitutions in a CDR1, CDR2 and/or CDR3 of the light chain.

In another embodiment, the anti-TIM-3 antibody molecule includes at least one, two, three, four, five or six CDRs (or collectively all of the CDRs) from a heavy and light chain variable region comprising an amino acid sequence shown in Tables 1-4 of US 2015/0218274, or encoded by a nucleotide sequence shown in Tables 1-4. In one embodiment, one or more of the CDRs (or collectively all of the CDRs) have one, two, three, four, five, six or more changes, e.g., amino acid substitutions or deletions, relative to the amino acid sequence shown in Tables 1-4, or encoded by a nucleotide sequence shown in Tables 1-4.

In another embodiment, the anti-TIM3 antibody molecule is MBG453, which is a high-affinity, ligand-blocking, humanized anti-TIM-3 IgG4 antibody that can block the binding of TIM-3 to phosphatidyserine (PtdSer). MBG453 is also known as sabatolimab.

In some embodiments, the TIM-3 inhibitor (e.g., MBG453) is administered at a dose of about 300 mg to about 900 mg, e.g., 300 mg to about 800 mg, about 300 mg to about 700 mg, about 300 mg to about 600 mg, about 300 mg to about 500 mg, about 300 mg to about 400 mg, about 400 mg to about 900 mg, about 400 mg to about 800 mg, about 400 mg to about 700 mg, about 400 mg to about 600 mg, about 400 mg to about 500 mg, about 500 mg to about 900 mg, about 500 mg to about 800 mg, about 500 mg to about 700 mg, about 500 mg to about 600 mg, about 600 mg to about 900 mg, about 600 mg to about 800 mg, about 600 mg to about 700 mg, about 700 mg to about 900 mg, about 700 mg to about 800 mg, about or 800 mg to about 900 mg. In some embodiments, the TIM-3 inhibitor (e.g., MBG453) is administered at a dose of about 300 mg, about 400 mg, about 500 mg, about 600 mg, about 700 mg, about 800 mg, or about 900 mg. In some embodiments, the TIM-3 inhibitor (e.g., MBG453) is administered once every three weeks. In some embodiments, the TIM-3 inhibitor (e.g., MBG453) is administered once every four weeks. In some embodiments, the TIM-3 inhibitor (e.g., MBG453) is administered once every six weeks. In some embodiments, the TIM-3 inhibitor (e.g., MBG453) is administered once every eight weeks. In some embodiments, the TIM-3 inhibitor (e.g., MBG453) is administered at a dose of 800 mg once every four weeks. In some embodiments, the TIM-3 inhibitor (e.g., MBG453) is administered at a dose of 800 mg once every eight weeks. In some embodiments, the TIM-3 inhibitor (e.g., MBG453) is administered at a dose of 600 mg once every three weeks. In some embodiments, the TIM-3 inhibitor (e.g., MBG453) is administered at a dose of 600 mg once every six weeks. In some embodiments, the TIM-3 inhibitor (e.g., MBG453) is administered at a dose of 400 mg once every three weeks. In some embodiments, the TIM-3 inhibitor (e.g., MBG453) is administered at a dose of 400 mg once every four weeks.

Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 2. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

In one embodiment, the anti-TIM-3 antibody molecule is LY3321367 (Eli Lilly). In one embodiment, the anti-TIM-3 antibody molecule comprises one or more of the CDR sequences (or collectively all of the CDR sequences), the heavy chain or light chain variable region sequence, or the heavy chain or light chain sequence of LY3321367.

In one embodiment, the anti-TIM-3 antibody molecule is Sym023 (Symphogen). In one embodiment, the anti-TIM-3 antibody molecule comprises one or more of the CDR sequences (or collectively all of the CDR sequences), the heavy chain or light chain variable region sequence, or the heavy chain or light chain sequence of Sym023.

In one embodiment, the anti-TIM-3 antibody molecule is BGB-A425 (Beigene). In one embodiment, the anti-TIM-3 antibody molecule comprises one or more of the CDR sequences (or collectively all of the CDR sequences), the heavy chain or light chain variable region sequence, or the heavy chain or light chain sequence of BGB-A425.

In one embodiment, the anti-TIM-3 antibody molecule is INCAGN-2390 (Agenus/Incyte). In one embodiment, the anti-TIM-3 antibody molecule comprises one or more of the CDR sequences (or collectively all of the CDR sequences), the heavy chain or light chain variable region sequence, or the heavy chain or light chain sequence of INCAGN-2390.

In one embodiment, the anti-TIM-3 antibody molecule is MBS-986258 (BMS/Five Prime). In one embodiment, the anti-TIM-3 antibody molecule comprises one or more of the CDR sequences (or collectively all of the CDR sequences), the heavy chain or light chain variable region sequence, or the heavy chain or light chain sequence of MBS-986258.

In one embodiment, the anti-TIM-3 antibody molecule is LY-3415244 (Eli Lilly). In one embodiment, the anti-TIM-3 antibody molecule comprises one or more of the CDR sequences (or collectively all of the CDR sequences), the heavy chain or light chain variable region sequence, or the heavy chain or light chain sequence of LY-3415244.

In one embodiment, the anti-TIM-3 antibody molecule is RO-7121661 (Roche). In one embodiment, the anti-TIM-3 antibody molecule comprises one or more of the CDR sequences (or collectively all of the CDR sequences), the heavy chain or light chain variable region sequence, or the heavy chain or light chain sequence of RO-7121661.

In one embodiment, the anti-TIM-3 antibody molecule is BC-3402 (Wuxi Zhikanghongyi Biotechnology). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of BC-3402.

In one embodiment, the anti-TIM-3 antibody molecule is SHR-1702 (Medicine Co Ltd.). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of SHR-1702. SHR-1702 is disclosed, e.g., in WO 2020/038355, the content of which is incorporated by reference in its entirety.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 2
Amino acid sequences of other exemplary anti-TIM-
3 antibody molecules
APE5137

| | | |
|---|---|---|
| SEQ ID | VH | EVQLLESGGGLVQPGGSLRLSCAAASGFTFSSYDMSWVRQ |
| NO: 830 | | APGKGLDWVSTISGGGTYTYYQDSVKGRFTISRDNSKNTL |
| | | YLQMNSLRAEDTAVYYCASMDYWGQGTTVTVSSA |
| SEQ ID | VL | DIQMTQSPSSLSASVGDRVTITCRASQSIRRYLNWYHQKP |
| NO: 831 | | GKAPKLLIYGASTLQSGVPSRFSGSGSGTDFTLTISSLQP |
| | | EDFAVYYCQQSHSAPLTFGGGTKVEIKR |

APE5121

| | | |
|---|---|---|
| SEQ ID | VH | EVQVLESGGGLVQPGGSLRLYCVASGFTFSGSYAMSWVRQ |
| NO: 832 | | APGKGLEWVSAISGSGGSTYYADSVKGRFTISRDNSKNTL |
| | | YLQMNSLRAEDTAVYYCAKKYYVGPADYWGQGTLVTVSSG |
| SEQ ID | VL | DIVMTQSPDSLAVSLGERATINCKSSQSVLYSSNNKNYLA |
| NO: 833 | | WYQHKPGQPPKLLIYWASTRESGVPDRFSGSGSGTDFTLT |
| | | ISSLQAEDVAVYYCQQYYSSPLTFGGGTKIEVK |

Formulations

The anti-TIM-3 antibody molecules described herein can be formulated into a formulation (e.g., a dose formulation or dosage form) suitable for administration (e.g., intravenous administration) to a subject as described herein. The formulation described herein can be a liquid formulation, a lyophilized formulation, or a reconstituted formulation.

In certain embodiments, the formulation is a liquid formulation. In some embodiments, the formulation (e.g., liquid formulation) comprises an anti-TIM-3 antibody molecule (e.g., an anti-TIM-3 antibody molecule described herein) and a buffering agent.

In some embodiments, the formulation (e.g., liquid formulation) comprises an anti-TIM-3 antibody molecule present at a concentration of 25 mg/mL to 250 mg/mL, e.g., 50 mg/mL to 200 mg/mL, 60 mg/mL to 180 mg/mL, 70 mg/mL to 150 mg/mL, 80 mg/mL to 120 mg/mL, 90 mg/mL to 110 mg/mL, 50 mg/mL to 150 mg/mL, 50 mg/mL to 100 mg/mL, 150 mg/mL to 200 mg/mL, or 100 mg/mL to 200 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL. In certain embodiments, the anti-TIM-3 antibody molecule is present at a concentration of 80 mg/mL to 120 mg/mL, e.g., 100 mg/mL.

In some embodiments, the formulation (e.g., liquid formulation) comprises a buffering agent comprising histidine (e.g., a histidine buffer). In certain embodiments, the buffering agent (e.g., histidine buffer) is present at a concentration of 1 mM to 100 mM, e.g., 2 mM to 50 mM, 5 mM to 40 mM, 10 mM to 30 mM, 15 to 25 mM, 5 mM to 40 mM, 5 mM to 30 mM, 5 mM to 20 mM, 5 mM to 10 mM, 40 mM to 50 mM, 30 mM to 50 mM, 20 mM to 50 mM, 10 mM to 50 mM, or 5 mM to 50 mM, e.g., 2 mM, 5 mM, 10 mM, 15 mM, 20 mM, 25 mM, 30 mM, 35 mM, 40 mM, 45 mM, or 50 mM. In some embodiments, the buffering agent (e.g., histidine buffer) is present at a concentration of 15 mM to 25 mM, e.g., 20 mM. In other embodiments, the buffering agent (e.g., a histidine buffer) or the formulation has a pH of 4 to 7, e.g., 5 to 6, e.g., 5, 5.5, or 6. In some embodiments, the buffering agent (e.g., histidine buffer) or the formulation has a pH of 5 to 6, e.g., 5.5. In certain embodiments, the buffering agent comprises a histidine buffer at a concentration of 15 mM to 25 mM (e.g., 20 mM) and has a pH of 5 to 6 (e.g., 5.5). In certain embodiments, the buffering agent comprises histidine and histidine-HCl.

In some embodiments, the formulation (e.g., liquid formulation) comprises an anti-TIM-3 antibody molecule present at a concentration of 80 to 120 mg/mL, e.g., 100 mg/mL; and a buffering agent that comprises a histidine buffer at a concentration of 15 mM to 25 mM (e.g., 20 mM), at a pH of 5 to 6 (e.g., 5.5).

In some embodiments, the formulation (e.g., liquid formulation) further comprises a carbohydrate. In certain embodiments, the carbohydrate is sucrose. In some embodiments, the carbohydrate (e.g., sucrose) is present at a concentration of 50 mM to 500 mM, e.g., 100 mM to 400 mM, 150 mM to 300 mM, 180 mM to 250 mM, 200 mM to 240 mM, 210 mM to 230 mM, 100 mM to 300 mM, 100 mM to 250 mM, 100 mM to 200 mM, 100 mM to 150 mM, 300 mM to 400 mM, 200 mM to 400 mM, or 100 mM to 400 mM, e.g., 100 mM, 150 mM, 180 mM, 200 mM, 220 mM, 250 mM, 300 mM, 350 mM, or 400 mM. In some embodiments, the formulation comprises a carbohydrate or sucrose present at a concentration of 200 mM to 250 mM, e.g., 220 mM.

In some embodiments, the formulation (e.g., liquid formulation) comprises an anti-TIM-3 antibody molecule present at a concentration of 80 to 120 mg/mL, e.g., 100 mg/mL; a buffering agent that comprises a histidine buffer at a concentration of 15 mM to 25 mM (e.g., 20 mM); and a carbohydrate or sucrose present at a concentration of 200 mM to 250 mM, e.g., 220 mM, at a pH of 5 to 6 (e.g., 5.5).

In some embodiments, the formulation (e.g., liquid formulation) further comprises a surfactant. In certain embodiments, the surfactant is polysorbate 20. In some embodiments, the surfactant or polysorbate 20) is present at a concentration of 0.005% to 0.1% (w/w), e.g., 0.01% to 0.08%, 0.02% to 0.06%, 0.03% to 0.05%, 0.01% to 0.06%, 0.01% to 0.05%, 0.01% to 0.03%, 0.06% to 0.08%, 0.04% to 0.08%, or 0.02% to 0.08% (w/w), e.g., 0.01%, 0.02%, 0.03%, 0.04%, 0.05%, 0.06%, 0.07%, 0.08%, 0.09%, or 0.1% (w/w). In some embodiments, the formulation comprises a surfactant or polysorbate 20 present at a concentration of 0.03% to 0.05%, e.g., 0.04% (w/w).

In some embodiments, the formulation (e.g., liquid formulation) comprises an anti-TIM-3 antibody molecule present at a concentration of 80 to 120 mg/mL, e.g., 100 mg/mL; a buffering agent that comprises a histidine buffer at a concentration of 15 mM to 25 mM (e.g., 20 mM); a carbohydrate or sucrose present at a concentration of 200 mM to 250 mM, e.g., 220 mM; and a surfactant or polysorbate 20 present at a concentration of 0.03% to 0.05%, e.g., 0.04% (w/w), at a pH of 5 to 6 (e.g., 5.5).

In some embodiments, the formulation (e.g., liquid formulation) comprises an anti-TIM-3 antibody molecule present at a concentration of 100 mg/mL; a buffering agent that comprises a histidine buffer (e.g., histidine/histidine-HCL) at a concentration of 20 mM); a carbohydrate or sucrose present at a concentration of 220 mM; and a surfactant or polysorbate 20 present at a concentration of 0.04% (w/w), at a pH of 5 to 6 (e.g., 5.5).

A formulation described herein can be stored in a container. The container used for any of the formulations described herein can include, e.g., a vial, and optionally, a stopper, a cap, or both. In certain embodiments, the vial is a glass vial, e.g., a 6R white glass vial. In other embodiments, the stopper is a rubber stopper, e.g., a grey rubber stopper. In other embodiments, the cap is a flip-off cap, e.g., an aluminum flip-off cap. In some embodiments, the container comprises a 6R white glass vial, a grey rubber stopper, and an aluminum flip-off cap. In some embodiments, the container (e.g., vial) is for a single-use container. In certain embodiments, 25 mg/mL to 250 mg/mL, e.g., 50 mg/mL to 200 mg/mL, 60 mg/mL to 180 mg/mL, 70 mg/mL to 150 mg/mL, 80 mg/mL to 120 mg/mL, 90 mg/mL to 110 mg/mL, 50 mg/mL to 150 mg/mL, 50 mg/mL to 100 mg/mL, 150 mg/mL to 200 mg/mL, or 100 mg/mL to 200 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL, of the anti-TIM-3 antibody molecule, is present in the container (e.g., vial).

In another aspect, the disclosure features therapeutic kits that include the anti-TIM-3 antibody molecules, compositions, or formulations described herein, and instructions for use, e.g., in accordance with dosage regimens described herein.

### TGF-β Inhibitors

In patients with primary myelofibrosis (PMF), increased levels of TGF-β1 in serum and bone marrow have been shown to correlate with the extent of both bone marrow fibrosis and leukemic cell infiltration, and data from preclinical models have established an important role for TGF-β in disease progression. In particular, TGF-β1 is associated with increased synthesis of types I, III and IV collagens as well as other extracellular matrix proteins such as fibronectin and tenascin, all elements that are actively deposited and accumulate in the bone marrow of patients affected with PMF, thereby implicating TGF-β in pathogenesis of bone marrow fibrosis (Tefferi, J Clin Oncol. 2005; 23(33): 8520-8530). Accordingly, in thrombopoietin-high mice, absence of TGF-β1 was shown to prevent the occurrence of bone marrow fibrosis, despite the development of myeloproliferative syndrome (Chagraoui et al., Blood. 2002; 100(10): 3495-3503). A similar correlation was reported in another murine model of PMF, Gata1-low mice, in which pharmacologic inhibition of TGF-β receptor kinase activity was shown to reduce fibrosis and osteogenesis in the bone marrow (Zingariello et al. Blood. 2013; 121(17): 3345-3363). Furthermore, TGF-β inhibition significantly reduced fibrosis in JAK2 V617F+ and MF mouse models (Agarwal et al., Stem Cell Investig. 2016; 3:5; Zingariello et al. Blood. 2013; 121(17): 3345-3363). Based on these observations, a TGF-β trap against TGF-β1 and -β3 is currently being assessed for patients with high grade PMF (ClinicalTrials.gov Identifier: NCT03895112).

Given the potent immunomodulatory and pro-fibrotic properties of TGF-β, a TGF-β inhibitor (e.g., an TGF-β inhibitor described herein) can be useful in the reversal of bone marrow fibrosis in patients with MF, and can provide significant therapeutic benefit in conjunction with therapies directed at limiting disease burden, including TIM-3 blockade by an TIM-3 inhibitor described herein (e.g., an anti-TIM-3 antibody molecule described herein).

In patients with a myelodysplastic syndrome (MDS), elevated levels of TGF-β have been implicated in the pathogenesis of MDS (Zorat et al. Br J Haematol 2001; 115(4):881-94; Allampallam et al. Int J Hematol 2002; 75(3):289-97). Further, elevated levels of TGF-β have been shown to result in bone marrow deficits (Geyh et al. Haematologica 2018; 103:1462-1471).

Given the potent immunomodulatory properties of TGF-β, a TGF-β inhibitor (e.g., an TGF-β inhibitor described herein), can be useful in the reversal of the aberrant immune activation implicated in the pathogenesis of MDS, e.g., a lower risk MDS (e.g., a very low risk MDS, a low risk MDS, or an intermediate risk MDS), and can provide significant therapeutic benefit in conjunction with therapies directed at limiting disease burden, including TIM-3 blockade by a TIM-3 inhibitor described herein (e.g., an anti-TIM-3 antibody molecule described herein).

In certain embodiments, a combination described herein comprises a transforming growth factor beta (also known as TGF-β TGFβ, TGFb, or TGF-beta, used interchangeably herein) inhibitor.

TGF-β belongs to a large family of structurally-related cytokines including, e.g., bone morphogenetic proteins (BMPs), growth and differentiation factors, activins and inhibins. In some embodiments, the TGF-β inhibitors described herein can bind and/or inhibit one or more isoforms of TGF-β (e.g., one, two, or all of TGF-β1, TGF-β2, or TGF-β3).

In some embodiments, the TGF-β inhibitor is used in combination with a TIM-3 inhibitor. In some embodiments, the TGF-β inhibitor is used in combination with a TIM-3 inhibitor, and optionally, a hypomethylating agent, and optionally further in combination with a PD-1 inhibitor or an IL-1β inhibitor. In some embodiments, the combination is used to treat a cancer (e.g., a myelofibrosis or a myelodysplastic syndrome (MDS) (e.g., a lower risk MDS (e.g., a very low risk MDS, a low risk MDS, or an intermediate risk MDS) or a higher risk MDS (e.g., a high risk MDS or a very high risk MDS))). In some embodiments, the TGF-β inhibitor is chosen from NIS793, fresolimumab, PF-06952229, or AVID200.

Exemplary TGF-β Inhibitors

In some embodiments, the TGF-β inhibitor comprises NIS973, or a compound disclosed in International Application Publication No. WO 2012/167143, which is incorporated by reference in its entirety.

NIS793 is also known as XOMA 089 or XPA.42.089. NIS793 is a fully human monoclonal antibody that specifically binds and neutralizes TGF-beta 1 and 2 ligands.

The heavy chain variable region of NIS793 has the amino acid sequence of: QVQLVQSGAEVKKPGSSVKVSCKASGGTFSSYAISWVRQAPGQGLEWMGGIIPIFGTANYAQ KFQGRVTITADESTSTAYMELSSLRSEDTAVYYCARGLWEVRALPSVYWGQGTLVTVSS (SEQ ID NO: 240) (disclosed as SEQ ID NO: 6 in WO 2012/167143). The light chain variable region of NIS793 has the amino acid sequence of:

(SEQ ID NO: 241)
SYELTQPPSVSVAPGQTARITCGANDIGSKSVHWYQQKAGQAPVLVVSED
IIRPSGIPERISGSNSGNTATLTISRVEAGDEADYYCQVWDRDSDQYVFG
TGTKVTVLG

(disclosed as SEQ ID NO: 8 in WO 2012/167143).

NIS793 binds with high affinity to the human TGF-β isoforms. Generally, NIS793 binds with high affinity to TGF-β1 and TGF-β2, and to a lesser extent to TGF-β3. In Biacore assays, the K_{D} of NIS793 on human TGF-β is 14.6 pM for TGF-β1, 67.3 pM for TGF-β2, and 948 pM for TGF-β3. Given the high affinity binding to all three TGF-β isoforms, in certain embodiments, NIS793 is expected to bind to TGF-β1, 2 and 3 at a dose of NIS793 as described herein. NIS793 cross-reacts with rodent and cynomolgus monkey TGF-β and shows functional activity in vitro and in vivo, making rodent and cynomolgus monkey relevant species for toxicology studies.

In certain embodiments, a combined inhibition of TGF-β with a checkpoint inhibitor (e.g., an inhibitor of TIM-3 described herein) is used to treat a cancer (e.g., a myelofibrosis or a myelodysplastic syndrome (MDS) (e.g., a lower risk MDS (e.g., a very low risk MDS, a low risk MDS, or an intermediate risk MDS) or a higher risk MDS (e.g., a high risk MDS or a very high risk MDS))).

In some embodiments, the TGF-β inhibitor (e.g., NIS793) is administered at a dose between about 500 mg to about 1000 mg, e.g., about 500 mg to about 900 mg, about 500 mg to about 800 mg, about 500 mg to about 700 mg, about 500 mg to about 600 mg, about 600 mg to about 1000 mg, about 600 mg to about 900 mg, about 600 mg to about 800 mg, about 600 mg to about 700 mg, about 700 mg to about 1000 mg, about 700 mg to about 900 mg, about 700 mg to about 800 mg, about 800 mg to about 1000 mg, about 800 mg to about 900 mg, about 900 mg to about 1000 mg. In some embodiments, the TGF-β inhibitor (e.g., NIS793) is administered at a dose of about 500 mg, about 600 mg, about 700 mg, about 800 mg, about 900 mg, or about 1000 mg. In some embodiments, the TGF-β inhibitor (e.g., NIS793) is administered at a dose of 700 mg. In some embodiments, the TGF-β inhibitor (e.g., NIS793) is administered once every three weeks. In some embodiments, the TGF-β inhibitor (e.g., NIS793) is administered once every six weeks. In some embodiments, the TGF-β inhibitor (e.g., NIS793) is administered at a dose of about 700 mg once every three weeks. In some embodiments, the TGF-β inhibitor (e.g., NIS793) is administered intravenously. In some embodiments, the TGF-β inhibitor (e.g., NIS793) is administered over a period of about 20 minutes to about 40 minutes (e.g., about 30 minutes).

In some embodiments, the TGF-β inhibitor (e.g., NIS793) is administered at a dose between about 1000 mg to about 1600 mg, e.g., about 1100 mg to about 1500 mg, about 1200 to about 1400 mg, about 1300 mg to about 1400 mg, about 1300 mg to about 1500 mg, about 1300 mg to about 1600 mg, about 1200 mg to about 1500 mg, about 1200 mg to about 1600 mg, about 1400 mg to about 1500 mg, about 1400 mg to about 1600 mg, about 1100 mg to about 1600 mg, 1100 mg to about 1400 mg, about 1100 mg to about 1300 mg, about 1100 mg to about 1200 mg, about 1000 mg to about 1500 mg, about 1000 mg to about 1400 mg, about 1000 mg to about 1300 mg, about 1000 mg to about 1200 mg, or about 1000 mg to about 1100 mg. In some embodiments, the TGF-β inhibitor (e.g., NIS793) is administered at a dose of about 1000 mg, about 1100 mg, about 1200 mg, about 1300 mg, about 1400 mg, about 1500 mg, about 1600 mg. In some embodiments, the TGF-β inhibitor (e.g., NIS793) is administered once every two weeks. In some embodiments, the TGF-β inhibitor (e.g., NIS793) is administered once every three weeks. In some embodiments, the TGF-β inhibitor (e.g., NIS793) is administered once every six weeks. In some embodiments, the TGF-β inhibitor (e.g., NIS793) is administered at a dose of about 1400 mg once every three weeks. In some embodiments, the TGF-β inhibitor (e.g., NIS793) is administered at a dose of about 1400 mg once every six weeks. In some embodiments, the TGF-β inhibitor (e.g., NIS793) is administered intravenously. In some embodiments, the TGF-β inhibitor (e.g., NIS793) is administered over a period of about 20 minutes to about 40 minutes (e.g., about 30 minutes).

In some embodiments, the TGF-β inhibitor (e.g., NIS793) is administered at a dose between 2000 mg to 2500 mg, e.g., about 2000 mg to about 2400 mg, about 2000 mg to about 2300 mg, about 2000 mg to about 2200, about 2000 mg to about 2100 mg, about 2100 mg to about 2500 mg, about 2100 mg to about 2400 mg, about 2100 mg to about 2300 mg, about 2100 mg to about 2200 mg, about 2200 mg to about 2500 mg, about 2200 to about 2400 mg, about 2200 to about 2300 mg, about 2300 mg to about 2500 mg, about 2300 mg to about 2400 mg, or about 2400 mg to about 2500 mg. In some embodiments, the TGF-β inhibitor (e.g., NIS793) is administered at a dose of about 2000 mg, about 2100 mg, about 2200 mg, about 2300 mg, about 2400 mg, or about 2500 mg. In some embodiments, the TGF-β inhibitor (e.g., NIS793) is administered once every two weeks. In some embodiments, the TGF-β inhibitor (e.g., NIS793) is administered once every three weeks. In some embodiments, the TGF-β inhibitor (e.g., NIS793) is administered once every six weeks. In some embodiments, the TGF-β inhibitor (e.g., NIS793) is administered at a dose of 2100 mg once every two weeks. In some embodiments, the TGF-β inhibitor (e.g., NIS793) is administered at a dose of 2100 mg once every three weeks. In some embodiments, the TGF-β inhibitor (e.g., NIS793) is administered at a dose of 2100 mg once every six weeks. In some embodiments, the TGF-β inhibitor (e.g., NIS793) is administered intravenously. In some embodiments, the TGF-β inhibitor (e.g., NIS793) is administered over a period of about 20 minutes to about 40 minutes (e.g., about 30 minutes).

In some embodiments, the TGF-β inhibitor (e.g., NIS793) is administered at a dose between about 600 mg to about 800 mg (e.g., about 700 mg), intravenously, over a period of about 20 minutes to about 40 minutes (e.g., about 30 minutes), once every three weeks. In some embodiments, the TGF-β inhibitor (e.g., NIS793) is administered at a dose between about 1300 mg to about 1500 mg (e.g., about 1400 mg), intravenously, over a period of about 20 minutes to about 40 minutes (e.g., about 30 minutes), once every two weeks. In some embodiments, the TGF-β inhibitor (e.g., NIS793) is administered at a dose between about 1300 mg to about 1500 mg (e.g., about 1400 mg), intravenously, over a period of about 20 minutes to about 40 minutes (e.g., about 30 minutes), once every three weeks. In some embodiments, the TGF-β inhibitor (e.g., NIS793) is administered at a dose between about 1300 mg to about 1500 mg (e.g., about 1400 mg), intravenously, over a period of about 20 minutes to about 40 minutes (e.g., about 30 minutes), once every six weeks. In some embodiments, the TGF-β inhibitor (e.g., NIS793) is administered at a dose between about 1400 mg to about 2100 mg, intravenously, over a period of about 20 minutes to about 40 minutes (e.g., about 30 minutes), once every three weeks. In some embodiments, the TGF-β inhibitor (e.g., NIS793) is administered at a dose between about 2000 mg to about 2200 mg (e.g., about 2100 mg), intravenously, over a period of about 20 minutes to about 40 minutes (e.g., about 30 minutes), once every three weeks. In some embodiments, the TGF-β inhibitor (e.g., NIS793) is administered at a dose between about 2000 mg to about 2200 mg (e.g., about 2100 mg), intravenously, over a period of about 20 minutes to about 40 minutes (e.g., about 30 minutes), once every six weeks.

In some embodiments, the TGF-β inhibitor (e.g., NIS793) is administered in combination with a TIM-3 inhibitor (e.g., an anti-TIM-3 antibody molecule). In some embodiments, the TGF-β inhibitor (e.g., NIS793) is administered on the same day as the TIM-3 inhibitor (e.g., an anti-TIM-3 antibody). In some embodiments, the TGF-β inhibitor (e.g., NIS793) is administered after the administration of the TIM-3 inhibitor (e.g., an anti-TIM-3 antibody) is started. In some embodiments, the TGF-β inhibitor (e.g., NIS793) is administered one hour after the administration of the TIM-3 inhibitor (e.g., an anti-TIM-3 antibody) is finished.

In some embodiments, the TGF-β inhibitor (e.g., NIS793) is administered at a dose between 600 mg and 800 mg (e.g., about 700 mg), e.g., once every three weeks, e.g., by intravenous infusion, and the TIM-3 inhibitor (e.g., the anti-TIM-3 antibody molecule) is administered at a dose between 700 mg to 900 mg (e.g., about 800 mg), e.g., once every four weeks, e.g., by intravenous infusion.

In some embodiments, the TGF-β inhibitor (e.g., NIS793) is administered at a dose between 600 mg and 800 mg (e.g., about 700 mg), e.g., once every three weeks, e.g., by intravenous infusion, and the TIM-3 inhibitor (e.g., the anti-TIM-3 antibody molecule) is administered at a dose between 700 mg to 900 mg (e.g., about 800 mg), e.g., once every eight weeks, e.g., by intravenous infusion.

In some embodiments, the TGF-β inhibitor (e.g., NIS793) is administered at a dose between 600 mg and 800 mg (e.g., about 700 mg), e.g., once every three weeks, e.g., by intravenous infusion, and the TIM-3 inhibitor (e.g., the anti-TIM-3 antibody molecule) is administered at a dose between 500 mg to 700 mg (e.g., about 600 mg), e.g., once every three weeks, e.g., by intravenous infusion.

In some embodiments, the TGF-β inhibitor (e.g., NIS793) is administered at a dose between 600 mg and 800 mg (e.g., about 700 mg), e.g., once every three weeks, e.g., by intravenous infusion, and the TIM-3 inhibitor (e.g., the anti-TIM-3 antibody molecule) is administered at a dose between 500 mg to 700 mg (e.g., about 600 mg), e.g., once every six weeks, e.g., by intravenous infusion.

In some embodiments, the TGF-β inhibitor (e.g., NIS793) is administered at a dose between 600 mg and 800 mg (e.g., about 700 mg), e.g., once every three weeks, e.g., by intravenous infusion, and the TIM-3 inhibitor (e.g., the anti-TIM-3 antibody molecule) is administered at a dose between 300 mg to 500 mg (e.g., about 400 mg), e.g., once every three weeks, e.g., by intravenous infusion.

In some embodiments, the TGF-β inhibitor (e.g., NIS793) is administered at a dose between 600 mg and 800 mg (e.g., about 700 mg), e.g., once every three weeks, e.g., by intravenous infusion, and the TIM-3 inhibitor (e.g., the anti-TIM-3 antibody molecule) is administered at a dose between 300 mg to 500 mg (e.g., about 400 mg), e.g., once every four weeks, e.g., by intravenous infusion.

In some embodiments, the TGF-β inhibitor (e.g., NIS793) is administered at a dose between 1300 mg and 1500 mg (e.g., about 1400 mg), e.g., once every two weeks, e.g., by intravenous infusion, and the TIM-3 inhibitor (e.g., the anti-TIM-3 antibody molecule) is administered at a dose between 700 mg to 900 mg (e.g., 800 mg), e.g., once every four weeks, e.g., by intravenous infusion.

In some embodiments, the TGF-β inhibitor (e.g., NIS793) is administered at a dose between 1300 mg and 1500 mg (e.g., about 1400 mg), e.g., once every three weeks, e.g., by intravenous infusion, and the TIM-3 inhibitor (e.g., the anti-TIM-3 antibody molecule) is administered at a dose between 700 mg to 900 mg (e.g., about 800 mg), e.g., once every four weeks, e.g., by intravenous infusion.

In some embodiments, the TGF-β inhibitor (e.g., NIS793) is administered at a dose between 1300 mg and 1500 mg (e.g., about 1400 mg), e.g., once every three weeks, e.g., by intravenous infusion, and the TIM-3 inhibitor (e.g., the anti-TIM-3 antibody molecule) is administered at a dose between 700 mg to 900 mg (e.g., about 800 mg), e.g., once every eight weeks, e.g., by intravenous infusion.

In some embodiments, the TGF-β inhibitor (e.g., NIS793) is administered at a dose between 1300 mg and 1500 mg (e.g., about 1400 mg), e.g., once every six weeks, e.g., by intravenous infusion, and the TIM-3 inhibitor (e.g., the anti-TIM-3 antibody molecule) is administered at a dose between 700 mg to 900 mg (e.g., about 800 mg), e.g., once every eight weeks, e.g., by intravenous infusion.

In some embodiments, the TGF-β inhibitor (e.g., NIS793) is administered at a dose between 1300 mg and 1500 mg (e.g., about 1400 mg), e.g., once every six weeks, e.g., by intravenous infusion, and the TIM-3 inhibitor (e.g., the anti-TIM-3 antibody molecule) is administered at a dose between 700 mg to 900 mg (e.g., about 800 mg), e.g., once every four weeks, e.g., by intravenous infusion.

In some embodiments, the TGF-β inhibitor (e.g., NIS793) is administered at a dose between 1300 mg and 1500 mg (e.g., about 1400 mg), e.g., once every two weeks, e.g., by intravenous infusion, and the TIM-3 inhibitor (e.g., the anti-TIM-3 antibody molecule) is administered at a dose between 500 mg to 700 mg (e.g., 600 mg), e.g., once every three weeks, e.g., by intravenous infusion.

In some embodiments, the TGF-β inhibitor (e.g., NIS793) is administered at a dose between 1300 mg and 1500 mg (e.g., about 1400 mg), e.g., once every three weeks, e.g., by intravenous infusion, and the TIM-3 inhibitor (e.g., the anti-TIM-3 antibody molecule) is administered at a dose between 500 mg to 700 mg (e.g., about 600 mg), e.g., once every three weeks, e.g., by intravenous infusion.

In some embodiments, the TGF-β inhibitor (e.g., NIS793) is administered at a dose between 1300 mg and 1500 mg (e.g., about 1400 mg), e.g., once every three weeks, e.g., by intravenous infusion, and the TIM-3 inhibitor (e.g., the anti-TIM-3 antibody molecule) is administered at a dose between 500 mg to 700 mg (e.g., about 600 mg), e.g., once every six weeks, e.g., by intravenous infusion.

In some embodiments, the TGF-β inhibitor (e.g., NIS793) is administered at a dose between 1300 mg and 1500 mg (e.g., about 1400 mg), e.g., once every six weeks, e.g., by intravenous infusion, and the TIM-3 inhibitor (e.g., the anti-TIM-3 antibody molecule) is administered at a dose between 500 mg to 700 mg (e.g., about 600 mg), e.g., once every three weeks, e.g., by intravenous infusion.

In some embodiments, the TGF-β inhibitor (e.g., NIS793) is administered at a dose between 1300 mg and 1500 mg (e.g., about 1400 mg), e.g., once every six weeks, e.g., by intravenous infusion, and the TIM-3 inhibitor (e.g., the anti-TIM-3 antibody molecule) is administered at a dose between 500 mg to 700 mg (e.g., about 600 mg), e.g., once every six weeks, e.g., by intravenous infusion.

In some embodiments, the TGF-β inhibitor (e.g., NIS793) is administered at a dose between 1300 mg and 1500 mg (e.g., about 1400 mg), e.g., once every three weeks, e.g., by intravenous infusion, and the TIM-3 inhibitor (e.g., the anti-TIM-3 antibody molecule) is administered at a dose between 300 mg to 500 mg (e.g., about 400 mg), e.g., once every three weeks, e.g., by intravenous infusion.

In some embodiments, the TGF-β inhibitor (e.g., NIS793) is administered at a dose between 1300 mg and 1500 mg (e.g., about 1400 mg), e.g., once every three weeks, e.g., by intravenous infusion, and the TIM-3 inhibitor (e.g., the anti-TIM-3 antibody molecule) is administered at a dose between 300 mg to 500 mg (e.g., about 400 mg), e.g., once every four weeks, e.g., by intravenous infusion.

In some embodiments, the TGF-β inhibitor (e.g., NIS793) is administered at a dose between 1300 mg and 1500 mg (e.g., about 1400 mg), e.g., once every six weeks, e.g., by intravenous infusion, and the TIM-3 inhibitor (e.g., the anti-TIM-3 antibody molecule) is administered at a dose between 300 mg to 500 mg (e.g., about 400 mg), e.g., once every three weeks, e.g., by intravenous infusion.

In some embodiments, the TGF-β inhibitor (e.g., NIS793) is administered at a dose between 1300 mg and 1500 mg (e.g., about 1400 mg), e.g., once every six weeks, e.g., by intravenous infusion, and the TIM-3 inhibitor (e.g., the anti-TIM-3 antibody molecule) is administered at a dose between 300 mg to 500 mg (e.g., about 400 mg), e.g., once every four weeks, e.g., by intravenous infusion.

In some embodiments, the TGF-β inhibitor (e.g., NIS793) is administered at a dose between 2000 mg and 2200 mg (e.g., about 2100 mg), e.g., once every three weeks, e.g., by intravenous infusion, and the TIM-3 inhibitor (e.g., the anti-TIM-3 antibody molecule) is administered at a dose between 700 mg to 900 mg (e.g., 800 mg), e.g., once every four weeks, e.g., by intravenous infusion.

In some embodiments, the TGF-β inhibitor (e.g., NIS793) is administered at a dose between 2000 mg and 2200 mg (e.g., about 2100 mg), e.g., once every six weeks, e.g., by intravenous infusion, and the TIM-3 inhibitor (e.g., the anti-TIM-3 antibody molecule) is administered at a dose between 700 mg to 900 mg (e.g., about 800 mg), e.g., once every four weeks, e.g., by intravenous infusion.

In some embodiments, the TGF-β inhibitor (e.g., NIS793) is administered at a dose between 2000 mg and 2200 mg (e.g., about 2100 mg), e.g., once every three weeks, e.g., by intravenous infusion, and the TIM-3 inhibitor (e.g., the anti-TIM-3 antibody molecule) is administered at a dose between 700 mg to 900 mg (e.g., about 800 mg), e.g., once every eight weeks, e.g., by intravenous infusion.

In some embodiments, the TGF-β inhibitor (e.g., NIS793) is administered at a dose between 2000 mg and 2200 mg (e.g., about 2100 mg), e.g., once every six weeks, e.g., by intravenous infusion, and the TIM-3 inhibitor (e.g., the anti-TIM-3 antibody molecule) is administered at a dose between 700 mg to 900 mg (e.g., about 800 mg), e.g., once every eight weeks, e.g., by intravenous infusion.

In some embodiments, the TGF-β inhibitor (e.g., NIS793) is administered at a dose between 2000 mg and 2200 mg (e.g., about 2100 mg), e.g., once every three weeks, e.g., by intravenous infusion, and the TIM-3 inhibitor (e.g., the anti-TIM-3 antibody molecule) is administered at a dose between 500 mg to 700 mg (e.g., 600 mg), e.g., once every three weeks, e.g., by intravenous infusion.

In some embodiments, the TGF-β inhibitor (e.g., NIS793) is administered at a dose between 2000 mg and 2200 mg (e.g., about 2100 mg), e.g., once every three weeks, e.g., by intravenous infusion, and the TIM-3 inhibitor (e.g., the anti-TIM-3 antibody molecule) is administered at a dose between 500 mg to 700 mg (e.g., about 600 mg), e.g., once every six weeks, e.g., by intravenous infusion.

In some embodiments, the TGF-β inhibitor (e.g., NIS793) is administered at a dose between 2000 mg and 2200 mg (e.g., about 2100 mg), e.g., once every six weeks, e.g., by intravenous infusion, and the TIM-3 inhibitor (e.g., the anti-TIM-3 antibody molecule) is administered at a dose between 500 mg to 700 mg (e.g., about 600 mg), e.g., once every three weeks, e.g., by intravenous infusion.

In some embodiments, the TGF-β inhibitor (e.g., NIS793) is administered at a dose between 2000 mg and 2200 mg (e.g., about 2100 mg), e.g., once every six weeks, e.g., by intravenous infusion, and the TIM-3 inhibitor (e.g., the anti-TIM-3 antibody molecule) is administered at a dose between 500 mg to 700 mg (e.g., about 600 mg), e.g., once every six weeks, e.g., by intravenous infusion.

In some embodiments, the TGF-β inhibitor (e.g., NIS793) is administered at a dose between 2000 mg and 2200 mg (e.g., about 2100 mg), e.g., once every three weeks, e.g., by intravenous infusion, and the TIM-3 inhibitor (e.g., the anti-TIM-3 antibody molecule) is administered at a dose between 300 mg to 500 mg (e.g., about 400 mg), e.g., once every four weeks, e.g., by intravenous infusion.

In some embodiments, the TGF-β inhibitor (e.g., NIS793) is administered at a dose between 2000 mg and 2200 mg (e.g., about 2100 mg), e.g., once every three weeks, e.g., by intravenous infusion, and the TIM-3 inhibitor (e.g., the anti-TIM-3 antibody molecule) is administered at a dose between 300 mg to 500 mg (e.g., about 400 mg), e.g., once every three weeks, e.g., by intravenous infusion.

In some embodiments, the TGF-β inhibitor (e.g., NIS793) is administered at a dose between 2000 mg and 2200 mg (e.g., about 2100 mg), e.g., once every six weeks, e.g., by intravenous infusion, and the TIM-3 inhibitor (e.g., the anti-TIM-3 antibody molecule) is administered at a dose between 300 mg to 500 mg (e.g., about 400 mg), e.g., once every four weeks, e.g., by intravenous infusion.

In some embodiments, the TGF-β inhibitor (e.g., NIS793) is administered at a dose between 2000 mg and 2200 mg (e.g., about 2100 mg), e.g., once every six weeks, e.g., by intravenous infusion, and the TIM-3 inhibitor (e.g., the anti-TIM-3 antibody molecule) is administered at a dose between 300 mg to 500 mg (e.g., about 400 mg), e.g., once every three weeks, e.g., by intravenous infusion.

Other Exemplary TGF-β Inhibitors

In some embodiments, the TGF-β inhibitor comprises fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab has the amino acid sequence of:

(SEQ ID NO: 238)
QVQLVQSGAEVKKPGSSVKVSCKASGYTFSSNVISWVRQAPGQGLEWMGG
VIPIVDIANYAQRFKGRVTITADESTSTTYMELSSLRSEDTAVYYCASTL
GLVLDAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK.

The light chain of fresolimumab has the amino acid sequence of:

(SEQ ID NO: 239)
ETVLTQSPGTLSLSPGERATLSCRASQSLGSSYLAWYQQKPGQAPRLLIY
GASSRAPGIPDRFSGSGSGTDFTLTISRLEPEDFAVYYCQQYADSPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

Fresolimumab is disclosed, e.g., in International Application Publication No. WO 2006/086469, and U.S. Pat. Nos. 8,383,780 and 8,591,901, which are incorporated by reference in their entirety.

In some embodiments, the TGF-β inhibitor is PF-06952229. PF-06952229 is an inhibitor of TGF-βR1, preventing signaling through the receptor and TGF-βR1-mediated immunosuppression thereby enhancing the anti-tumor immune response. PF-06952229 is disclosed, e.g., in Yano et al. Immunology 2019; 157(3) 232-47.

In some embodiments, the TGF-β inhibitor is AVID200. AVID200 is a TGF-β receptor ectodomain-IgG Fc fusion protein, which selectively targets and neutralizes TGF-β isoforms 1 and 3. AVID200 is disclosed, e.g., in O'Connor-McCourt, M D et al. Can. Res. 2018; 78(13).

### Hypomethylating Agents

Hypomethylating agents (HMA) including decitabine (and azacitidine, CC-486, and ASTX727) have been shown to alter the immune microenvironment in both solid tumors and hematological malignancies. HMAs have been shown to: (1) increase the expression of killer-cell immunoglobulin-like receptors (KIR) and in some instances, the activity of NK cells, which may play a role in anti-tumor immunity; (2) increase the expression of major histocompatibility complex (MHC) class I on tumor cells; (3) increase expression of endogenous retroviral elements (ERVs); and (4) increase the expression of checkpoint proteins, including PD-1, PD-L1 and Cytotoxic T-Lymphocyte-Associated protein 4 (CTLA-4) (reviewed in Lindblad et al., Expert Rev Hematol. 2017; 10(8): 745-752).

In vitro studies demonstrate that hypomethylating agents can reduce the number of circulating malignant progenitor cells in idiopathic myelofibrosis (Shi et al., Cancer Res. 2007; 67(13): 6417-6424. 2007). A phase 2 trial with 34 patients given 5-azacytidine showed hypomethylation in all patients, but clinical improvement was recorded in only 8 patients and myelosuppression was commonly observed (Quintas-Cardama et al. Leukemia. 2008; 22(5): 965-970). Similarly, in 21 patients with myelofibrosis treated with decitabine, a response was seen in 7 of 19 evaluable patients; reduction in spleen size was not reported. Grade 3/4 neutropenia and thrombocytopenia was seen in 95% and 52% of patients in this cohort (Odenike et al. 2008).

These data support for combining immunodulatory agents that stimulate a cytotoxic immune response and reduce an immunosuppressive bone marrow phenotype (e.g., a hypomethylating agent described herein, e.g., decitabine) with an immune-based therapy (e.g., a TIM-3 inhibitor described, e.g., an anti-TIM-3 antibody molecule described herein) in myelofibrosis.

In certain embodiments, the combination described herein further includes a hypomethylating agent. Hypomethylating agents are also known as HMAs or demethylating agents, which inhibits DNA methylation. In certain embodiments, the hypomethylating agent blocks the activity of DNA methyltransferase. In certain embodiments, the hypomethylating agent comprises decitabine, azacitidine, CC-486 (Bristol Meyers Squibb), or ASTX727 (Astex).

In some embodiments, the combination described herein to treat myelofibrosis (e.g., a primary myelofibrosis (PMF), post-essential thrombocythemia myelofibrosis (PET-MF), post-polycythemia vera myelofibrosis (PPV-MF)), comprises a TIM3 inhibitor described herein (e.g., MBG453), administered intravenously at a dose of 600 mg over 30 minutes on day 8 and day 29 of a 42 day cycle; a TGF-β inhibitor described herein (e.g., NIS793), administered intravenously at a dose of 2100 mg over 30 minutes on day 8 and day 29 of a 42 day cycle; and a hypomethylating agent described herein (e.g., decitabine), administered intravenously at a dose of at least 5 mg/m² (e.g., a dose of about 5 mg/m² to about 20 mg/m²) over one hour on days 1, 2, and 3 of a 42 day cycle, or on days 1, 2, 3, 4, and 5 of a 42 day cycle. In other embodiments, the combination described herein to treat myelofibrosis (e.g., a primary myelofibrosis (PMF), post-essential thrombocythemia myelofibrosis (PET-MF), post-polycythemia vera myelofibrosis (PPV-MF)), comprises a TIM3 inhibitor described herein (e.g., MBG453), administered intravenously at a dose of 800 mg over 30 minutes on day 8 of each 28 day cycle; a TGF-β inhibitor described herein (e.g., NIS793), administered intravenously at a dose of 1400 mg over 30 minutes on day 8 and day 22 of each 28 day cycle; and a hypomethylating agent described herein (e.g., decitabine), administered intravenously at a dose of at least 5 mg/m² (e.g., a dose of about 5 mg/m² to about 20 mg/m²) over one hour on days 1, 2, and 3 of a 42 day cycle, or on days 1, 2, 3, 4, and 5 of a 42 day cycle. In some embodiments, the hypomethylating agent (e.g., decitabine) will be administered first, followed by the TIM-3 inhibitor (e.g., MBG453), and the TGF-β inhibitor (e.g., NIS793). In some embodiments, the TIM-3 inhibitor (e.g., MBG453), and the TGF-β inhibitor (e.g., NIS793), are administered on the same day. In some embodiments, the TGF-β inhibitor (e.g., NIS793) is administered after administration of the TIM-3 inhibitor (e.g., MBG453) has completed. In some embodiments, the TGF-β inhibitor (e.g., NIS793) is administered about 30 minutes to about four hours (e.g., about one hour) after administration of the TIM-3 inhibitor (e.g., MBG453) has completed.

Exemplary Hypomethylating Agents

In some embodiments, the hypomethylating agent comprises decitabine. Decitabine is also known as 5-aza-dCyd, deoxyazacytidine, dezocitidine, 5AZA, DAC, 2′-deoxy-5-azacytidine, 4-amino-1-(2-deoxy-beta-D-erythro-pentofuranosyl)-1,3,5-triazin-2(1H)-one, 5-aza-2′-deoxycytidine, 5-aza-2-deoxycytidine, 5-azadeoxycytidine, or DACOGEN®. Decitabine has the following structural formula:

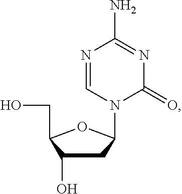

or a pharmaceutically acceptable salt thereof.

Decitabine is a cytidine antimetabolite analogue with potential antineoplastic activity. Decitabine incorporates into DNA and inhibits DNA methyltransferase, resulting in hypomethylation of DNA and intra-S-phase arrest of DNA replication.

In some embodiments, decitabine is administered at a dose of about 2 mg/m² to about 50 mg/m², e.g., about 10 mg/m² to about 40 mg/m², about 20 mg/m² to about 30 mg/m², about 2 mg/m² to about 40 mg/m², about 2 mg/m² to about 30 mg/m², about 2 mg/m² to about 20 mg/m², about 2 mg/m² to about 10 mg/m², about 10 mg/m² to about 50 mg/m², about 20 mg/m² to about 50 mg/m², about 30 mg/m² to about 50 mg/m², about 40 mg/m² to about 50 mg/m², about 10 mg/m² to about 20 mg/m², about 15 mg/m² to about 25 mg/m², about 5 mg/m², about 10 mg/m², about 15 mg/m², about 20 mg/m², about 25 mg/m², about 30 mg/m², about 35 mg/m², about 40 mg/m², about 45 mg/m², or about 50 mg/m². In some embodiments, decitabine is administered at a dose of about 2.5 mg/m2, about 5 mg/m², about 7.5 mg/m² about 10 mg/m², about 15 mg/m², or about 20 mg/m². In some embodiments, decitabine is administered at a starting dose of 5 mg/m² and escalated to a dose up to 20 mg/m². In some embodiments, decitabine is administered intravenously. In some embodiments, the hypomethylating agent is administered subcutaneously. In some embodiments, decitabine is administered according to a three-day regimen, e.g., administered at a dose of about 2 mg/m² to about 20 mg/m² (e.g., 5 mg/m²) by continuous intravenous infusion (e.g., over about 1 hour) daily for three days (in a 42 day cycle, e.g., every six weeks). In some embodiments, decitabine is administered according to a three-day regimen, e.g., administered at a dose of about 2 mg/m² to about 20 mg/m² (e.g., 5 mg/m²) by continuous intravenous infusion (e.g., over about 1 hour) daily for three days (in a 28 day cycle, e.g., every four weeks). In some embodiments, decitabine is administered according to a three-day regimen, e.g., administered at a dose of about 2 mg/m² to about 20 mg/m² (e.g., 5 mg/m²) by continuous intravenous infusion (e.g., over about 1 hour) daily for five days (in a 42 day cycle, e.g., every six weeks). In certain embodiments, decitabine is administered according a three-day regimen, e.g., administered at a dose of about 2 mg/m² to about 20 mg/m² (e.g., 5 mg/m²) by continuous intravenous infusion over about 3 hours repeated every 8 hours for 3 days (in a 42 day cycle). In other embodiments, decitabine is administered according to a five-day regimen, e.g., administered at a dose of about 2 mg/m² to about 20 mg/m² (e.g., 5 mg/m²) by continuous intravenous infusion over about 1 hour daily for 5 days (in a 28 day cycle). In some embodiments, decitabine is administered at a fixed dose. In other embodiments, the dose of decitabine is ramped-up over a period of three days in each cycle, e.g., a 42 day cycle, to achieve the dose of 20 mg/m². In other embodiments, the dose of decitabine is ramped-up over a period of three days in each cycle, e.g., a 28 day cycle, to achieve the dose of 20 mg/m². In other embodiments, the dose of decitabine is ramped-up over a period of five days in each cycle, e.g., a 42 day cycle, to achieve the dose of 20 mg/m². In other embodiments, the dose of decitabine is ramped-up over a period of about three to about five days in each cycle, e.g., a 42 day cycle, to achieve the dose of 20 mg/m². For example, the doses for Cycle 1 Day 1, Day 2, and Day 3 and beyond are about 5 mg/m², about 10 mg/m², and about 20 mg/m², respectively.

Other Exemplary Hypomethylating Agents

In some embodiments, the hypomethylating agent comprises, azacitidine, CC-486, and ASTX727. In some embodiments, the hypomethylating agent comprises azacitidine. Azacitidine is also known as 5-AC, 5-azacytidine, azacytidine, ladakamycin, 5-AZC, AZA-CR, U-18496, 4-amino-1-beta-D-ribofuranosyl-1,3,5-triazin-2(1H)-one, 4-amino-1-[(2R,3R,4S,5R)-3,4-dihydroxy-5-(hydroxymethyl)oxolan-2-yl]-1,3,5-triazin-2-one, or VIDAZA®. Azacitidine has the following structural formula:

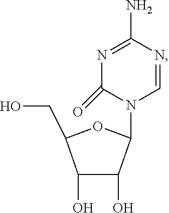 [SMILES: Nc1ncn(C2OC(CO)C(O)C2O)c(=O)n1]

or a pharmaceutically acceptable salt thereof.

Azacitidine is a pyrimidine nucleoside analogue of cytidine with antineoplastic activity. Azacitidine is incorporated into DNA, where it reversibly inhibits DNA methyltransferase, thereby blocking DNA methylation. Hypomethylation of DNA by azacitidine can activate tumor suppressor genes silenced by hypermethylation, resulting in an antitumor effect. Azacitidine can also be incorporated into RNA, thereby disrupting normal RNA function and impairing tRNA cytosine-5-methyltransferase activity.

In some embodiments, azacitidine is administered at a dose of about 25 mg/m² to about 150 mg/m², e.g., about 50 mg/m² to about 100 mg/m², about 70 mg/m² to about 80 mg/m², about 50 mg/m² to about 75 mg/m², about 75 mg/m² to about 125 mg/m², about 50 mg/m², about 75 mg/m², about 100 mg/m², about 125 mg/m², or about 150 mg/m². In some embodiments, azacitidine is administered once a day. In some embodiments, azacitidine is administered intravenously. In other embodiments, azacitidine is administered subcutaneously. In some embodiments, azacitidine is administered at a dose of about 50 mg/m² to about 100 mg/m² (e.g., about 75 mg/m²), e.g., for about 5-7 consecutive days, e.g., in a 28-day cycle. For example, azacitidine can be administered at a dose of about 75 mg/m² for seven consecutive days on days 1-7 of a 28-day cycle. As another example, azacitidine can be administered at a dose of about 75 mg/m² for five consecutive days on days 1-5 of a 28-day cycle, followed by a two-day break, then two consecutive days on days 8-9. As yet another example, azacitidine can be administered at a dose of about 75 mg/m² for six consecutive days on days 1-6 of a 28-day cycle, followed by a one-day break, then one administration on day 8 will be permitted.

In some embodiments, the hypomethylating agent comprises an oral azacitidine (e.g., CC-486). In some embodiments, the hypomethylating agent comprises CC-486. CC-486 is an orally bioavailable formulation of azacitidine, a pyrimidine nucleoside analogue of cytidine, with antineoplastic activity. Upon oral administration, azacitidine is taken up by cells and metabolized to 5-azadeoxycitidine triphosphate. The incorporation of 5-azadeoxycitidine triphosphate into DNA reversibly inhibits DNA methyltransferase, and blocks DNA methylation. Hypomethylation of DNA by azacitidine can re-activate tumor suppressor genes previously silenced by hypermethylation, resulting in an antitumor effect. The incorporation of 5-azacitidine triphosphate into RNA can disrupt normal RNA function and impairs tRNA (cytosine-5)-methyltransferase activity, resulting in an inhibition of RNA and protein synthesis. CC-486 is described, e.g., in Laille et al. J Clin Pharmacol. 2014; 54(6):630-639; Mesia et al. European Journal of Cancer 2019 123:138-154. Oral formulations of cytidine analogs are also described, e.g., in PCT Publication No. WO 2009/139888 and U.S. Pat. No. 8,846,628. In some embodiments, CC-486 is administered orally. In some embodiments, CC-486 is administered on once daily. In some embodiments, CC-486 is administered at a dose of about 200 mg to about 500 mg (e.g., 300 mg). In some embodiments, CC-486 is administered on 5-15 consecutive days (e.g., days 1-14) of, e.g., a 21 day or 28 day cycle. In some embodiments, CC-486 is administered once a day.

In some embodiments, the hypomethylating agent comprises a CDA inhibitor (e.g., cedazuridine)/decitabine combination agent (e.g., ASTX727). In some embodiments, the hypomethylating agent comprises ASTX727. ASTX727 is an orally available combination agent comprising the cytidine deaminase (CDA) inhibitor cedazuridine (also known as E7727) and the cytidine antimetabolite decitabine, with antineoplastic activity. Upon oral administration of ASTX727, the CDA inhibitor E7727 binds to and inhibits CDA, an enzyme primarily found in the gastrointestinal (GI) tract and liver that catalyzes the deamination of cytidine and cytidine analogs. This can prevent the breakdown of decitabine, increasing its bioavailability and efficacy while decreasing GI toxicity due to the administration of lower doses of decitabine. Decitabine exerts its antineoplastic activity through the incorporation of its triphosphate form into DNA, which inhibits DNA methyltransferase and results in hypomethylation of DNA. This can interfere with DNA replication and decreases tumor cell growth. ASTX727 is disclosed in, e.g., Montalaban-Bravo et al. Current Opinions in Hematology 2018 25(2):146-153. In some embodiments, ASTX727 comprises cedazuridine, e.g., about 50-150 mg (e.g., about 100 mg), and decitabine, e.g., about 300-400 mg (e.g., 345 mg). In some embodiments, ASTX727 is administered orally. In some embodiments, ASTX727 is administered on 5-15 consecutive days (e.g., days 1-5) of, e.g., a 28 day cycle. In some embodiments, ASTX727 is administered once a day.

### Cytarabine

In some embodiments, the combination described herein includes cytarabine. Cytarabine is also known as cytosine arabinoside or 4-amino-1-[(2R,3S,4S,5R)-3,4-dihydroxy-5-(hydroxymethyl)oxolan-2-yl]pyrimidin-2-one. Cytarabine has the following structural formula:

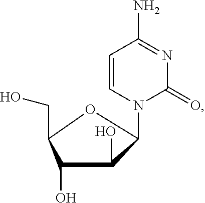

or a pharmaceutically acceptable salt thereof.

Cytarabine is a cytidine antimetabolite analogue with a modified sugar moiety (arabinose in place of ribose). Cytarabine is converted to a triphosphate form which competes with cytidine for incorporation into DNA. Due to the arabinose sugar, the rotation of the DNA molecule is sterically hindered and DNA replication ceases. Cytarabine also interferes with DNA polymerase.

In some embodiments, cytarabine is administered at about 5 mg/m² to about 75 mg/m², e.g., 30 mg/m². In some embodiments, cytarabine is administered about 100 mg/m² to about 400 mg/m², e.g., 100 mg/m². In some embodiments, cytarabine is administered by intravenous infusion or injection, subcutaneously, or intrathecally. In some embodiments, cytarabine is administered at a dose of 100 mg/m²/day by continuous IV infusion or 100 mg/m² intravenously every 12 hours. In some embodiments, cytarabine is administered for 7 days (e.g., on days 1 to 7). In some embodiments, cytarabine is administered intrathecally at a dose ranging from 5 to 75 mg/m² of body surface area. In some embodiments, cytarabine is intrathecally administered from once every 4 days to once a day for 4 days. In some embodiments, cytarabine is administered at a dose of 30 mg/m² every 4 days.

### PD-1 Inhibitors

The co-blockade of TIM-3 (e.g., by a TIM-3 inhibitor described herein (e.g., an anti-TIM-3 antibody molecule described herein) and PD-1 (e.g., by a PD-1 inhibitor described herein (e.g., an anti-PD-1 antibody molecule described herein) in MF is supported, at least in part, by the combined ability for greater anti-tumor activity in PD-1 and TIM-3 co-blockade, coupled with evidence for activity with PD-1 pathway blockade in MF.

For example, preclinical evidence indicates that the concurrent blockade of TIM-3 and PD-1 promotes greater activation of T-cells than either therapy alone, and synergistically inhibits tumor growth in experimental cancer models (Sakuishi et al. Exp Med. 2010; 207(10): 2187-2194, Ngiow et al. Cancer Res. 2011; 71(21):6567-6571; Anderson, Cancer Immunol Res. 2014; 2(5): 393-398).

Recent evidence suggests for MF patients that oncogenic mutations may confer immune escape (Prestipino et al., Sci Transl Med. 2018; 10(429). pii: eaam7729) have shown that oncogenic JAK2 activity caused STAT3 and STAT5 phosphorylation, which enhanced PD-L1 promoter activity and PD-L1 protein expression in JAK2V617F-mutant cells, whereas blockade of JAK2 reduced PD-L1 expression in myeloid JAK2V617F-mutant cells. PD-L1 expression was higher on primary cells isolated from patients with JAK2V617F MPNs compared to healthy individuals and declined upon JAK2 inhibition. JAK2V617F mutational burden, pSTAT3, and PD-L1 expression were highest in primary MPN patient-derived monocytes, megakaryocytes, and platelets. PD-1 inhibition prolonged survival in human MPN xenograft and primary murine MPN models. This effect was dependent on T cells. Mechanistically, PD-L1 surface expression in JAK2V617F-mutant cells affected metabolism and cell cycle progression of T cells (Prestipino et al., Sci Transl Med. 2018; 10(429). pii: eaam7729).

In certain embodiments, the combination described herein is further administered in combination with a PD-1 inhibitor. In some embodiments, the PD-1 inhibitor is chosen from spartalizumab (PDR001, Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

In some embodiments, the combination described herein to treat myelofibrosis (e.g., a primary myelofibrosis (PMF), post-essential thrombocythemia myelofibrosis (PET-MF), post-polycythemia vera myelofibrosis (PPV-MF)), comprises a TIM3 inhibitor described herein (e.g., MBG453), administered intravenously at a dose of 600 mg over 30 minutes on day 1 of each 21 day cycle; a TGF-β inhibitor described herein (e.g., NIS793), administered intravenously at a dose of 2100 mg over 30 minutes on day 1 of each 21 day cycle; and a PD-1 inhibitor described herein (e.g., spartalizumab), administered intravenously at a dose of 300 mg over 30 minutes on day 1 of each 21 day cycle. In other embodiments, the combination described herein to treat myelofibrosis (e.g., a primary myelofibrosis (PMF), post-essential thrombocythemia myelofibrosis (PET-MF), post-polycythemia vera myelofibrosis (PPV-MF)), comprises a TIM3 inhibitor described herein (e.g., MBG453), administered intravenously at a dose of 800 mg over 30 minutes on day 1 of each 28 day cycle; a TGF-β inhibitor described herein (e.g., NIS793), administered intravenously at a dose of 1400 mg over 30 minutes on day 1 and day 15 of each 28 day cycle; and a PD-1 inhibitor described herein (e.g., spartalizumab), administered intravenously at a dose of 400 mg over 30 minutes on day 1 of each 28 day cycle. In some embodiments, the TIM-3 inhibitor (e.g., MBG453), the TGF-β inhibitor (e.g., NIS793), and the PD-1 inhibitor (e.g., spartalizumab) are administered on the same day. In some embodiments, the TGF-β inhibitor (e.g., NIS793) is administered after administration of the TIM3 inhibitor (e.g., MBG453) has completed. In some embodiments, the TGF-β inhibitor (e.g., NIS793) is administered about 30 minutes to about four hours (e.g., about one hour) after administration of the anti-TIM-3 antibody (e.g., MBG453) has completed. In some embodiments, the PD-1 inhibitor (e.g., spartalizumab), is administered after administration of the TGF-β inhibitor (e.g., NIS793) has completed. In some embodiments, the PD-1 inhibitor (e.g., spartalizumab) is administered about 30 minutes to about four hours (e.g., about one hour) after administration of the TGF-β inhibitor (e.g., NIS793) has completed.

In some embodiments, the combination described herein to treat myelofibrosis (e.g., a primary myelofibrosis (PMF), post-essential thrombocythemia myelofibrosis (PET-MF), post-polycythemia vera myelofibrosis (PPV-MF)), comprises a TIM3 inhibitor described herein (e.g., MBG453), administered intravenously at a dose of 600 mg over 30 minutes on day 8 and day 29 of a 42 day cycle; a TGF-β inhibitor described herein (e.g., NIS793), administered intravenously at a dose of 2100 mg over 30 minutes on day 8 and day 29 of a 42 day cycle; a PD-1 inhibitor (e.g. spartalizumab) administered intravenously at a dose of 300 mg over 30 minutes on day 8 and day 29 of a 42 day cycle; and a hypomethylating agent described herein (e.g., decitabine), administered intravenously at a dose of at least 5 mg/m² (e.g., starting at 5 mg/m² and escalating up to 20 mg/m²) over one hour on days 1, 2, and 3 of a 42 day cycle, or on days 1, 2, 3, 4, and 5 of a 42 day cycle. In other embodiments, the combination described herein to treat myelofibrosis (e.g., a primary myelofibrosis (PMF), post-essential thrombocythemia myelofibrosis (PET-MF), post-polycythemia vera myelofibrosis (PPV-MF)), comprises a TIM3 inhibitor described herein (e.g., MBG453), administered intravenously at a dose of 800 mg over 30 minutes on day 8 of each 28 day cycle; a TGF-β inhibitor described herein (e.g., NIS793), administered intravenously at a dose of 1400 mg over 30 minutes on day 8 and day 22 of each 28 day cycle; a PD-1 inhibitor described herein (e.g., spartalizumab), administered intravenously at a dose of 400 mg over 30 minutes on day 8 of each 28 day cycle and a hypomethylating agent described herein (e.g., decitabine), administered intravenously at a dose of at least 5 mg/m² over one hour on days 1, 2, and 3 of a 42 day cycle, or on days 1, 2, 3, 4, and 5 of a 42 day cycle. In some embodiments, the hypomethylating agent (e.g., decitabine) will be administered first, followed by the TIM-3 inhibitor (e.g., MBG453), and the TGF-β inhibitor (e.g., NIS793). In some embodiments, the TIM-3 inhibitor (e.g., MBG453), and the TGF-β inhibitor (e.g., NIS793), are administered on the same day. In some embodiments, the TGF-β inhibitor (e.g., NIS793) is administered after administration of the TIM-3 inhibitor (e.g., MBG453) has completed. In some embodiments, the TGF-β inhibitor (e.g., NIS793) is administered about 30 minutes to about four hours (e.g., about one hour) after administration of the TIM-3 inhibitor (e.g., MBG453) has completed. In some embodiments, the PD-1 inhibitor (e.g., spartalizumab), is administered after administration of the TGF-β inhibitor (e.g., NIS793) has completed. In some embodiments, the PD-1 inhibitor (e.g., spartalizumab) is administered about 30 minutes to about four hours (e.g., about one hour) after administration of the TGF-β inhibitor (e.g., NIS793) has completed.

Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety. In one embodiment, the anti-PD-1 inhibitor is spartalizumab, also known as PDR001.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 3 (e.g., from the heavy and light chain variable region sequences of BAP049-Clone-E or BAP049-Clone-B disclosed in Table 3), or encoded by a nucleotide sequence shown in Table 3. In some embodiments, the CDRs are according to the Kabat definition (e.g., as set out in Table 3). In some embodiments, the CDRs are according to the Chothia definition (e.g., as set out in Table 3). In some embodiments, the CDRs are according to the combined CDR definitions of both Kabat and Chothia (e.g., as set out in Table 3). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 3, or encoded by a nucleotide sequence shown in Table 3.

In one embodiment, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 3.

In one embodiment, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 3.

In one embodiment, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In one embodiment, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In one embodiment, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

In one embodiment, the antibody molecule comprises a VH encoded by the nucleotide sequence of SEQ ID NO: 507, or a nucleotide sequence at least 85%, 90%, 95%, or 99% identical or higher to SEQ ID NO: 507. In one embodiment, the antibody molecule comprises a VL encoded by the nucleotide sequence of SEQ ID NO: 521 or 517, or a nucleotide sequence at least 85%, 90%, 95%, or 99% identical or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In one embodiment, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In one embodiment, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In one embodiment, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In one embodiment, the antibody molecule comprises a heavy chain encoded by the nucleotide sequence of SEQ ID NO: 509, or a nucleotide sequence at least 85%, 90%, 95%, or 99% identical or higher to SEQ ID NO: 509. In one embodiment, the antibody molecule comprises a light chain encoded by the nucleotide sequence of SEQ ID NO: 523 or 519, or a nucleotide sequence at least 85%, 90%, 95%, or 99% identical or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

In certain embodiments, a combined inhibition of a checkpoint inhibitor (e.g., an inhibitor of TIM-3 described herein) with a TGF-β inhibitor is further combined with a PD-1 inhibitor and used to treat a cancer (e.g., a myelofibrosis).

In some embodiments, the PD-1 inhibitor (e.g., spartalizumab) is administered at a dose between about 100 mg to about 600 mg. e.g., about 100 mg to about 500 mg, about 100 mg to about 400 mg, about 100 mg to about 300 mg, about 100 mg to about 200 mg, about 200 mg to about 600 mg, about 200 mg to about 500 mg, about 200 mg to about 400 mg, about 200 mg to about 300 mg, about 300 mg to about 600 mg, about 300 mg to about 500 mg, about 300 mg to about 400 mg, about 400 mg to about 600 mg, about 400 mg to about 500 mg, or about 500 mg to about 600 mg. In some embodiments, the PD-1 inhibitor (e.g., spartalizumab) is administered at a dose of about 100 mg, about 200 mg, about 300 mg, about 400 mg, about 500 mg, or about 600 mg. In some embodiments, the PD-1 inhibitor (e.g., spartalizumab) is administered once every four weeks. In some embodiments, (e.g., spartalizumab) is administered once every three weeks. In some embodiments, (e.g., spartalizumab) is administered intravenously. In some embodiments, (e.g., spartalizumab) is administered over a period of about 20 minutes to 40 minutes (e.g., about 30 minutes).

In some embodiments, the PD-1 inhibitor (e.g., spartalizumab) is administered at a dose between about 300 mg to about 500 mg (e.g., about 400 mg), intravenously, over a period of about 20 minutes to about 40 minutes (e.g., about 30 minutes), once every two weeks. In some embodiments, the PD-1 inhibitor (e.g., spartalizumab) is administered at a dose between about 200 mg to about 400 mg (e.g., about 300 mg), intravenously, over a period of about 20 minutes to about 40 minutes (e.g., about 30 minutes), once every three weeks.

In some embodiments, the PD-1 inhibitor (e.g., spartalizumab) is administered in combination with a TIM-3 inhibitor (e.g., an anti-TIM3 antibody) and a TGF-β inhibitor (e.g., NIS793).

TABLE 3
Amino acid and nucleotide sequences of exemplary anti-PD-1 antibody molecules
BAP049-Clone-B HC

| | | |
|---|---|---|
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQA |
| | | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 507 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 508 | Heavy | EVQLVQSGAEvkKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 509 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |

BAP049-Clone-B LC

| | | |
|---|---|---|
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 516 | VL | EIVLTQSPATLSLSPGERATLSCKSSQSLLDSGNQKNFLTWY |
| | | QQKPGKAPKLLIYWASTRESGVPSRFSGSGSGTDFTFTISSLQ |
| | | PEDIATYYCQNDYSYPYTFGQGTKVEIK |
| SEQ ID NO: 517 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAG |
| SEQ ID NO: 518 | Light | EIVLTQSPATLSLSPGERATLSCKSSQSLLDSGNQKNFLTWY |
| | chain | QQKPGKAPKLLIYWASTRESGVPSRFSGSGSGTDFTFTISSLQ |
| | | PEDIATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 519 | DNA | GAGATCGTCCTGACTCAGTCACCCGCTACCCTGAGCCTGA |
| | light | GCCCTGGCGAGCGGGCTACACTGAGCTGTAAATCTAGTC |
| | chain | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | DNACCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |

BAP049-Clone-E HC

| | | |
|---|---|---|
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQA |
| | | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 507 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 508 | Heavy | EVQLVQSGAEVKKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 509 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |

BAP049-Clone-E LC

| | | |
|---|---|---|
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 520 | VL | EIVLTQSPATLSLSPGERATLSCKSSQSLLbSGNQKNFLTWY |
| | | QQKPGQAPRLLIYWASTRESGVPSRFSGSGSGTDFTFTISSLE |
| | | AEDAATYYCQNDYSYPYTFGQGTKVEIK |
| SEQ ID NO: 521 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTCAAGCCCCTAGACTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAG |
| SEQ ID NO: 522 | Light | EIVLTQSPATLSLSPGERATLSCKSSQSLLDSGNQKNFLTWY |
| | chain | QQKPGQAPRLLIYWASTRESGVPSRFSGSGSGTDFTFTISSLE |
| | | AEDAATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 523 | DNA | GAGATCGTCCTGACTCAGTCACCCGCTACCCTGAGCCTGA |
| | light | GCCCTGGCGAGCGGGCTACACTGAGCTGTAAATCTAGTC |
| | chain | AGTCACTGCTGGATAGCGGTAATCAGAAGAACTTCCTGA |
| | | CCTGGTATCAGCAGAAGCCCGGTCAAGCCCCTAGACTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | CCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |

BAP049-Clone-B HC

| | | |
|---|---|---|
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGAG |
| (Kabat) | | AAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |

BAP049-Clone-B LC

| | | |
|---|---|---|
| SEQ ID NO: 529 | LCDR1 | AAATCTAGTCAGTCACTGCTGGATAGCGGTAATCAGAAG |
| (Kabat) | | AACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

BAP049-Clone-E HC

| | | |
|---|---|---|
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGAG |
| (Kabat) | | AAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |

BAP049-Clone-E LC

| | | |
|---|---|---|
| SEQ ID NO: 529 | LCDR1 | AAATCTAGTCAGTCACTGCTGGATAGCGGTAATCAGAAG |
| (Kabat) | | AACTTCCTGACC |
| SEQ ID NO:530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

Other Exemplary PD-1 Inhibitors

In one embodiment, the anti-PD-1 antibody molecule is Nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 4.

In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 4.

In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 4.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

TABLE 4
Amino acid sequences of other exemplary anti-PD-1 antibody molecules
Nivolumab

| | | |
|---|---|---|
| SEQ ID NO: | Heavy | QVQLVESGGGVVQPGRSLRLDCKASGITFSNSGMHWVRQAPGKGLE |
| 535 | chain | WVAVIWYDGSKRYYADSVKGRFTISRDNSKNTLFLQMNSLRAEDTA |
| | | VYYCATNDDYWGQGTLVTVSSASTKGPSVFPLAPCSRSTSESTAALG |
| | | CLVKDYFPEPVTVSWNSGALTSGVHTFPAVLQSSGLYSLSSVVTVPS |
| | | SSLGTKTYTCNVDHKPSNTKVDKRVESKYGPPCPPCPAPEFLGGPSV |
| | | FLFPPKPKDTLMISRTPEVTCVVVDVSQEDPEVQFNWYVDGVEVHN |
| | | AKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAV |
| | | EWESNGQPENNYKTTPPVLDSDGSFFLYSRLTVDKSRWQEGNVFSC |
| | | SVMHEALHNHYTQKSLSLSLGK |
| SEQ ID NO: | Light | EIVLTQSPATLSLSPGERATLSCRASQSVSSYLAWYQQKPGQAPRLLI |
| 536 | chain | YDASNRATGIPARFSGSGSGTDFTLTISSLEPEDFAVYYCQQSSNWPR |
| | | TFGQGTKVEIKRTVAAPSVFIFPPSDEQLKSGTASVVCLLNNFYPREA |
| | | KVQWKVDNALQSGNSQESVTEQDSKDSTYSLSSTLTLSKADYEKHK |
| | | VYACEVTHQGLSSPVTKSFNRGEC |

Pembrolizumab

| | | |
|---|---|---|
| SEQ ID NO: | Heavy | QVQLVQSGVEVKKPGASVKVSCKASGYTFTNYYMYWVRQAPGQG |
| 537 | chain | LEWMGGINPSNGGTNFNEKFKNRVTLTTDSSTTTAYMELKSLQFDD |
| | | TAVYYCARRDYRFDMGFDYWGQGTTVTVSSASTKGPSVFPLAPCSR |
| | | STSESTAALGCLVKDYFPEPVTVSWNSGALTSGVHTFPAVLQSSGLY |
| | | SLSSVVTVPSSSLGTKTYTCNVDHKPSNTKVDKRVESKYGPPCPPCP |
| | | APEFLGGPSVFLFPPKPKDTLMISRTPEVTCVVVDVSQEDPEVQFNW |
| | | YVDGVEVHNAKTKPREEQFNSTYRVVSVLTVLHQDWLNGKEYKCK |
| | | VSNKGLPSSIEKTISKAKGQPREPQVYTLPPSQEEMTKNQVSLTCLVK |
| | | GFYPSDIAVEWESNGQPENNYKTTPPVLDSDGSFFLYSRLTVDKSRW |
| | | QEGNVFSCSVMHEALHNHYTQKSLSLSLGK |
| SEQ ID NO: | Light | EIVLTQSPATLSLSPGERATLSCRASKGVSTSGYSYLHWYQQKPGQA |
| 538 | chain | PRLLIYLASYLESGVPARFSGSGSGTDFTLTISSLEPEDFAVYYCQHSR |
| | | DLPLTFGGGTKVEIKRTVAAPSVFIFPPSDEQLKSGTASVVCLLNNFY |
| | | PREAKVQWKVDNALQSGNSQESVTEQDSKDSTYSLSSTLTLSKADY |
| | | EKHKVYACEVTHQGLSSPVTKSFNRGEC |

Pidilizumab

| | | |
|---|---|---|
| SEQ ID NO: | Heavy | QVQLVQSGSELKKPGASVKISCKASGYTFTNYGMNWVRQAPGQGL |
| 539 | chain | QWMGWINTDSGESTYAEEFKGRFVFSLDTSVNTAYLQITSLTAEDTG |
| | | MYFCVRVGYDALDYWGQGTLVTVSSASTKGPSVFPLAPSSKSTSGG |
| | | TAALGCLVKDYFPEPVTVSWNSGALTSGVHTFPAVLQSSGLYSLSSV |
| | | VTVPSSSLGTQTYICNVNHKPSNTKVDKRVEPKSCDKTHTCPPCPAP |
| | | ELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQ |
| | | QGNVFSCSVMHEALHNHYTQKSLSLSPGK |
| SEQ ID NO: | Light | EIVLTQSPSSLSASVGDRVTITCSARSSVSYMHWFQQKPGKAPKLWI |
| 540 | chain | YRTSNLASGVPSRFSGSGSGTSYCLTINSLQPEDFATYYCQQRSSFPL |
| | | TFGGGTKLEIKRTVAAPSVFIFPPSDEQLKSGTASVVCLLNNFYPREA |
| | | KVQWKVDNALQSGNSQESVTEQDSKDSTYSLSSTLTLSKADYEKHK |
| | | VYACEVTHQGLSSPVTKSFNRGEC |

### IL-1β Inhibitors

The Interleukin-1 (IL-1) family of cytokines is a group of secreted pleotropic cytokines with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). The IL-1 family comprises, inter alia, IL-1 beta (IL-1β), and IL-lalpha (IL-1a).

In some embodiments, a combination described herein includes an interleukin-1 beta (IL-1β) inhibitor. In some embodiments, the IL-1β inhibitor is chosen from canakinumab, gevokizumab, Anakinra, or Rilonacept. In some embodiments, the IL-1β inhibitor is canakinumab.

In some embodiments, the IL-1β inhibitor is administered at a dose between about 100 mg to about 600 mg, e.g., about 100 mg to about 500 mg, about 100 mg to about 400 mg, about 100 mg to about 300 mg, about 100 mg to about 200 mg, about 200 mg to about 600 mg, about 200 mg to about 500 mg, about 200 mg to about 400 mg, about 200 mg to about 300 mg, about 300 mg to about 600 mg, about 300 mg to about 500 mg, about 300 mg to about 400 mg, about 400 mg to about 600 mg, about 400 mg to about 500 mg, or about 500 mg to about 600 mg. In some embodiments, the IL-10 inhibitor is administered at a dose of about 100 mg, about 125 mg, about 150 mg, about 175 mg, 200 mg, about 225 mg, about 250 mg, about 275 mg, or about 300 mg. In some embodiments, the IL-10 inhibitor is administered once every four weeks. In some embodiments, the IL-1β inhibitor is administered once every eight weeks. In some embodiments, the IL-1β inhibitor (e.g., canakinumab) is administered at a dose of 250 mg once every eight weeks. In some embodiments, the IL-1β inhibitor (e.g., canakinumab) is administered at a dose of 250 mg once every four weeks. In some embodiments, the IL-1β inhibitor is administered subcutaneously. In some embodiments, the IL-1β inhibitor is administered intravenously.

In some embodiments, the combination described herein to treat myelofibrosis (e.g., a primary myelofibrosis (PMF), post-essential thrombocythemia myelofibrosis (PET-MF), post-polycythemia vera myelofibrosis (PPV-MF)), comprises a TIM3 inhibitor described herein (e.g., MBG453), administered intravenously at a dose of 600 mg over 30 minutes on day 1 of each 21 day cycle; a TGF-β inhibitor described herein (e.g., NIS793), administered intravenously at a dose of 2100 mg over 30 minutes on day 1 of each 21 day cycle; and a IL-1β inhibitor described herein (e.g., canakinumab), administered intravenously at a dose of 200 mg over 30 minutes on day 1 of each 21 day cycle. In other embodiments, the combination described herein to treat myelofibrosis (e.g., a primary myelofibrosis (PMF), post-essential thrombocythemia myelofibrosis (PET-MF), post-polycythemia vera myelofibrosis (PPV-MF)), comprises a TIM3 inhibitor described herein (e.g., MBG453), administered intravenously at a dose of 800 mg over 30 minutes on day 1 of each 28 day cycle; a TGF-β inhibitor described herein (e.g., NIS793), administered intravenously at a dose of 1400 mg over 30 minutes on day 1 and day 15 of each 28 day cycle; and a IL-1β inhibitor described herein (e.g., canakinumab), administered intravenously at a dose of 250 mg on day 1 of each 28 day cycle. In some embodiments, the TIM-3 inhibitor (e.g., MBG453), the TGF-β inhibitor (e.g., NIS793), and the IL-1β inhibitor (e.g., canakinumab) are administered on the same day. In some embodiments, the TGF-β inhibitor (e.g., NIS793) is administered after administration of the TIM3 inhibitor (e.g., MBG453) has completed. In some embodiments, the TGF-β inhibitor (e.g., NIS793) is administered about 30 minutes to about four hours (e.g., about one hour) after administration of the anti-TIM-3 antibody (e.g., MBG453) has completed. In some embodiments, the IL-1β inhibitor (e.g., canakinumab), is administered after administration of the TGF-β inhibitor (e.g., NIS793) has completed. In some embodiments, the IL-1β inhibitor (e.g., canakinumab) is administered about 30 minutes to about four hours (e.g., about one hour) after administration of the TGF-β inhibitor (e.g., NIS793) has completed.

In some embodiments, the combination described herein to treat myelofibrosis (e.g., a primary myelofibrosis (PMF), post-essential thrombocythemia myelofibrosis (PET-MF), post-polycythemia vera myelofibrosis (PPV-MF)), comprises a TIM3 inhibitor described herein (e.g., MBG453), administered intravenously at a dose of 600 mg over 30 minutes on day 8 and day 29 of a 42 day cycle; a TGF-β inhibitor described herein (e.g., NIS793), administered intravenously at a dose of 2100 mg over 30 minutes on day 8 and day 29 of a 42 day cycle; an IL-1β inhibitor (e.g., canakinumab) administered intravenously at a dose of 200 mg over 30 minutes on day 8 and day 29 of a 42 day cycle; and a hypomethylating agent described herein (e.g., decitabine), administered intravenously at a dose of at least 5 mg/m² over one hour on days 1, 2, and 3 of a 42 day cycle, or on days 1, 2, 3, 4, and 5 of a 42 day cycle. In other embodiments, the combination described herein to treat myelofibrosis (e.g., a primary myelofibrosis (PMF), post-essential thrombocythemia myelofibrosis (PET-MF), post-polycythemia vera myelofibrosis (PPV-MF)), comprises a TIM3 inhibitor described herein (e.g., MBG453), administered intravenously at a dose of 800 mg over 30 minutes on day 8 of each 28 day cycle; a TGF-β inhibitor described herein (e.g., NIS793), administered intravenously at a dose of 1400 mg over 30 minutes on day 8 and day 22 of each 28 day cycle; an IL-1β inhibitor (e.g., canakinumab) administered intravenously at a dose of 250 mg over 30 minutes on day 8 of each 28 day cycle; and a hypomethylating agent described herein (e.g., decitabine), administered intravenously at a dose of at least 5 mg/m² over one hour on days 1, 2, and 3 of a 42 day cycle, or on days 1, 2, 3, 4, and 5 of a 42 day cycle. In some embodiments, the hypomethylating agent (e.g., decitabine) will be administered first, followed by the TIM-3 inhibitor (e.g., MBG453), and the TGF-β inhibitor (e.g., NIS793) and the IL-1β inhibitor (e.g., canakinumab). In some embodiments, the TIM-3 inhibitor (e.g., MBG453), the TGF-β inhibitor (e.g., NIS793), and the IL-1β inhibitor (e.g., canakinumab) are administered on the same day. In some embodiments, the TGF-β inhibitor (e.g., NIS793) is administered after administration of the TIM-3 inhibitor (e.g., MBG453) has completed. In some embodiments, the TGF-β inhibitor (e.g., NIS793) is administered about 30 minutes to about four hours (e.g., about one hour) after administration of the TIM-3 inhibitor (e.g., MBG453) has completed. In some embodiments, the IL-1β inhibitor (e.g., canakinumab), is administered after administration of the TGF-β inhibitor (e.g., NIS793) has completed. In some embodiments, the IL-1β inhibitor (e.g., canakinumab) is administered about 30 minutes to about four hours (e.g., about one hour) after administration of the TGF-β inhibitor (e.g., NIS793) has completed.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 2100 mg over 30 minutes once every six weeks; and an IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 2100 mg over 30 minutes once every six weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 1400 mg over 30 minutes once every three weeks; and an IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every eight weeks; a TGF-β inhibitor described herein (e.g., NIS793), administered intravenously at a dose of 1400 mg over 30 minutes once every three weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every eight weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every eight weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every four weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every three weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every four weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every six weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every four weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every three weeks; a TGF-β inhibitor described herein (e.g., NIS793), administered intravenously at a dose of 2100 mg over 30 minutes once every six weeks; and a IL-1β inhibitor described herein (e.g., canakinumab), administered subcutaneously at a dose of 250 mg once every four weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every six weeks; a TGF-β inhibitor described herein (e.g., NIS793), administered intravenously at a dose of 2100 mg over 30 minutes once every six weeks; and a IL-1β inhibitor described herein (e.g., canakinumab), administered subcutaneously at a dose of 250 mg once every four weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every three weeks; a TGF-β inhibitor described herein (e.g., NIS793), administered intravenously at a dose of 1400 mg over 30 minutes once every three weeks; and a IL-1β inhibitor described herein (e.g., canakinumab), administered subcutaneously at a dose of 250 mg once every four weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every six weeks; a TGF-β inhibitor described herein (e.g., NIS793), administered intravenously at a dose of 1400 mg over 30 minutes once every three weeks; and a IL-1β inhibitor described herein (e.g., canakinumab), administered subcutaneously at a dose of 250 mg once every four weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every three weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every four weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every six weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every four weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every three weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every four weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every six weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every three weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every four weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every three weeks; a TGF-β inhibitor described herein (e.g., NIS793), administered intravenously at a dose of 2100 mg over 30 minutes once every six weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every four weeks; a TGF-β inhibitor described herein (e.g., NIS793), administered intravenously at a dose of 2100 mg over 30 minutes once every six weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every three weeks; a TGF-β inhibitor described herein (e.g., NIS793), administered intravenously at a dose of 1400 mg over 30 minutes once every three weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every four weeks; a TGF-β inhibitor described herein (e.g., NIS793), administered intravenously at a dose of 1400 mg over 30 minutes once every three weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every three weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every four weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every three weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every four weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every four weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 2100 mg over 30 minutes once every six weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 2100 mg over 30 minutes once every six weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 1400 mg over 30 minutes once every three weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every eight weeks; a TGF-β inhibitor described herein (e.g., NIS793), administered intravenously at a dose of 1400 mg over 30 minutes once every three weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every eight weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every four weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 800 mg over 30 minutes once every eight weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every eight weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every three weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every eight weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every six weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every eight weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every three weeks; a TGF-β inhibitor described herein (e.g., NIS793), administered intravenously at a dose of 2100 mg over 30 minutes once every six weeks; and a IL-1β inhibitor described herein (e.g., canakinumab), administered subcutaneously at a dose of 250 mg once every eight weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every six weeks; a TGF-β inhibitor described herein (e.g., NIS793), administered intravenously at a dose of 2100 mg over 30 minutes once every six weeks; and a IL-1β inhibitor described herein (e.g., canakinumab), administered subcutaneously at a dose of 250 mg once every eight weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every three weeks; a TGF-β inhibitor described herein (e.g., NIS793), administered intravenously at a dose of 1400 mg over 30 minutes once every three weeks; and a IL-1β inhibitor described herein (e.g., canakinumab), administered subcutaneously at a dose of 250 mg once every eight weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every six weeks; a TGF-β inhibitor described herein (e.g., NIS793), administered intravenously at a dose of 1400 mg over 30 minutes once every three weeks; and a IL-1β inhibitor described herein (e.g., canakinumab), administered subcutaneously at a dose of 250 mg once every eight weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every three weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every eight weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every six weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every eight weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every three weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every eight weeks.

In some embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 600 mg over 30 minutes once every six weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every three weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every four weeks; a TGF-β inhibitor described herein (e.g., NIS793), administered intravenously at a dose of 2100 mg over 30 minutes once every three weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every three weeks; a TGF-β inhibitor described herein (e.g., NIS793), administered intravenously at a dose of 2100 mg over 30 minutes once every six weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every four weeks; a TGF-β inhibitor described herein (e.g., NIS793), administered intravenously at a dose of 2100 mg over 30 minutes once every six weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every three weeks; a TGF-β inhibitor described herein (e.g., NIS793), administered intravenously at a dose of 1400 mg over 30 minutes once every three weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every four weeks; a TGF-β inhibitor described herein (e.g., NIS793), administered intravenously at a dose of 1400 mg over 30 minutes once every three weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every three weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every four weeks; a TGF-β inhibitor described herein (e.g., NIS793), administered intravenously at a dose of 1400 mg over 30 minutes once every six weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every three weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every eight weeks.

In other embodiments, the combination described herein to treat a myelodysplastic syndrome (e.g., a lower risk MDS (e.g., a very low risk MDS, a low risk MDS, or an intermediate MDS) or a higher risk MDS (e.g., a high risk MDS or a very high risk MDS)), comprises a TIM3 inhibitor described herein (e.g., MBG453), administered intravenously at a dose of 400 mg over 30 minutes once every four weeks; a TGF-β inhibitor described herein (e.g., NIS793), administered intravenously at a dose of 700 mg over 30 minutes once every three weeks; and a IL-1β inhibitor described herein (e.g., canakinumab), administered subcutaneously at a dose of 250 mg once every eight weeks.

In some embodiments, the TIM-3 inhibitor (e.g., MBG453), the TGF-β inhibitor (e.g., NIS793), and the IL-1β inhibitor (e.g., canakinumab) are administered on the same day. In some embodiments, the TGF-β inhibitor (e.g., NIS793) is administered after administration of the TIM3 inhibitor (e.g., MBG453) has completed. In some embodiments, the TGF-β inhibitor (e.g., NIS793) is administered about 30 minutes to about four hours (e.g., about one hour) after administration of the anti-TIM-3 antibody (e.g., MBG453) has completed. In some embodiments, the IL-1β inhibitor (e.g., canakinumab), is administered after administration of the TGF-β inhibitor (e.g., NIS793) has completed. In some embodiments, the IL-1β inhibitor (e.g., canakinumab) is administered about 30 minutes to about four hours (e.g., about one hour) after administration of the TGF-β inhibitor (e.g., NIS793) has completed.

Exemplary IL-1β Inhibitors

In some embodiments, the IL-1β inhibitor is canakinumab. Canakinumab is also known as ACZ885 or ILARIS®. Canakinumab is a human monoclonal IgG1/κ antibody that neutralizes the bioactivity of human IL-1β.

Canakinumab is disclosed, e.g., in WO 2002/16436, U.S. Pat. No. 7,446,175, and EP 1313769. The heavy chain variable region of canakinumab has the amino acid sequence of:

(SEQ ID NO: 834)
MEFGLSWVFLVALLRGVQCQVQLVESGGGVVQPGRSLRLSCAASGFTFSV
YGMNWVRQAPGKGLEWVAIIWYDGDNQYYADSVKGRFTISRDNSKNTLYL
QMNGLRAEDTAVYYCARDLRTGPFDYWGQGTLVTVSS (disclosed
as SEQ ID NO: 1 in U.S. Pat. No. 7,446,175).

The light chain variable region of canakinumab has the amino acid sequence of:

(SEQ ID NO: 835)
MLPSQLIGFLLLWVPASRGEIVLTQSPDFQSVTPKEKVTITCRASQSIGS
SLHWYQQKPDQSPKLLIKYASQSFSGVPSRFSGSGSGTDFTLTINSLEAE
DAAAYYCHQSSSLPFTFGPGTKVDIK (disclosed as SEQ ID
NO: 2 in U.S. Pat. No. 7,446,175).

In some embodiment, the IL-1β binding antibody is canakinumab, wherein canakinumab is administered to a patient with cancer, e.g., cancer that has at least a partial inflammatory basis, in the range of about 100 mg to about 400 mg, about 200 mg per treatment. In one embodiment the patient receives each treatment about every 2 weeks, about every 3 weeks, about every 4 weeks (about monthly), about every 6 weeks, about bimonthly (about every 2 months), about every 9 weeks, or about quarterly (about every 3 months). In one embodiment the patient receives canakinumab about monthly or about every three weeks. In one embodiment the dose of canakinumab for patient is about 200 mg every 3 weeks. In some embodiments the dose of canakinumab is about 200 mg monthly. When safety concerns raise, the dose can be down-titrated by increasing the dosing interval, for example by doubling or tripling the dosing interval. For example, the about 200 mg about monthly or about every 3 weeks regimen can be changed to about every 2 months or about every 6 weeks respectively or about every 3 months or about every 9 weeks, respectively. In an alternative embodiment the patient receives canakinumab at a dose of about 200 mg about every two months or about every 6 weeks in the down-titration phase or in the maintenance phase, independent from any safety issue or throughout the treatment phase. In an alternative embodiment, the patient receives canakinumab at a dose of about 200 mg about every 3 months or about every 9 weeks in the down-titration phase or in the maintenance phase independent from any safety issue or throughout the treatment phase. In an alternative embodiment, the patient receives canakinumab at a dose of about 150 mg, about 250 mg, or about 300 mg. In an alternative embodiment the patient receives canakinumab at a dose of about 150 mg about every 4 weeks. In an alternative embodiment the patient receives canakinumab at a dose of about 250 mg about every 4 weeks. In an alternative embodiment the patient receives canakinumab at a dose of about 300 mg about every 4 weeks. In one embodiment, the patient receives canakinumab at a dose of about 200 mg every 3 weeks, or at a dose of about 250 mg every 4 weeks.

Other Exemplary IL-1β Inhibitors

In some embodiments, a combination described herein includes an interleukin-1 beta (IL-1β) inhibitor, e.g., an anti-IL-1β antibody or a fragment thereof

As used herein, IL-1β inhibitors include but are not limited to, canakinumab or a functional fragment thereof, gevokizumab or a functional fragment thereof, Anakinra, diacerein, Rilonacept, IL-1 Affibody (SOBI 006, Z-FC (Swedish Orphan Biovitrum/Affibody)) and Lutikizumab (ABT-981) (Abbott), CDP-484 (Celltech), LY-2189102 (Lilly).

In some embodiments, the anti-IL-1β antibody is canakinumab. Canakinumab (ACZ885 or ILARIS®) is a high-affinity, fully human monoclonal antibody of the IgG1/k to interleukin-1β, developed for the treatment of IL-1β driven inflammatory diseases. It is designed to bind to human IL-1β and thus blocks the interaction of this cytokine with its receptors.

In other embodiments, the anti-IL-1β antibody is gevokizumab. Gevokizumab (XOMA-052) is a high-affinity, humanized monoclonal antibody of the IgG2 isotype to interleukin-1β, developed for the treatment of IL-1β driven inflammatory diseases. Gevokizumab modulates IL-1β binding to its signaling receptor.

In some embodiments, the anti-IL-1β antibody is LY-2189102, which is a humanized interleukin-1 beta (IL-1β) monoclonal antibody.

In some embodiments, the anti-IL-1β antibody or a functional fragment thereof is CDP-484 (Celltech), which is an antibody fragment blocking IL-1β.

In some embodiments, the anti-IL-1β antibody or a functional fragment thereof is IL-1 Affibody (SOBI 006, Z-FC (Swedish Orphan Biovitrum/Affibody)).

In some embodiments, the IL-1β binding antibody is gevokizumab. Gevokizumab is also known as Xoma 052. Gevokizumab (international nonproprietary name (INN) number 9310) is disclosed in WO2007/002261, which is hereby incorporated by reference in its entirety. Gevokizumab is a humanized monoclonal anti-human IL-1β antibody of the IgG2 isotype, being developed for the treatment of IL-1β driven inflammatory diseases. The full heavy chain sequence of gevokizumab is:

(SEQ ID NO: 836)
QVQLQESGPGLVKPSQTLSLTCSFSGFSLSTSGMGVGWIRQPSGKGLEWL
AHIWWDGDESYNPSLKSRLTISKDTSKNQVSLKITSVTAADTAVYFCARN
RYDPPWFVDWGQGTLVTVSSASTKGPSVFPLAPCSRSTSESTAALGCLVK
DYFPEPVTVSWNSGALTSGVHTFPAVLQSSGLYSLSSVVTVTSSNFGTQT
YTCNVDHKPSNTKVDKTVERKCCVECPPCPAPPVAGPSVFFPPKPKDTLM
ISRTPEVTCVVVDVSHEDPEVQFNWYVDGMEVHNAKTKPREEQFNSTFRV
VSVLTVVHQDWLNGKEYKCKVSNKGLPAPIEKTISKTKGQPREPQVYTLP
PSREEMTKNQVSLTCLVKGFYPSDIAVEWESNGQPENNYKTTPPMLDSDG
SFFLYSKLTVDKSRWQQGNVFSCSVMHEALHNHYTQKSLSLSPG.

The full light chain sequence of gevokizumab is:

(SEQ ID NO: 837)
DIQMTQSTSSLSASVGDRVTITCRASQDISNYLSWYQQKPGKAVKLLIYY
TSKLHSGVPSRFSGSGSGTDYTLTISSLQQEDFATYFCLQGKMLPWTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In one embodiment, patient receives gevokizumab about 30 mg to about 120 mg per treatment every 3 weeks or monthly, in the range of about 20 mg to about 240 mg per treatment, in the range of about 20 mg to about 180 mg, in the range of about 30 mg to about 120 mg, about 30 mg to about 60 mg, or about 60 mg to about 120 mg per treatment. In one embodiment, patient receives about 30 mg to about 120 mg per treatment. In one embodiment patient receives about 30 mg to about 60 mg per treatment. In one embodiment patient receives about 30 mg, about 60 mg, about 90 mg, about 120 mg, or about 180 mg per treatment. In one embodiment the patient receives each treatment about every 2 weeks, about every 3 weeks, about monthly (about every 4 weeks), about every 6 weeks, about bimonthly (about every 2 months), about every 9 weeks or about quarterly (about every 3 months). In one embodiment the patient receives each treatment about every 3 weeks. In one embodiment the patient receives each treatment about every 4 weeks. When safety concerns raise, the dose can be down-titrated by increasing the dosing interval, for example by doubling or tripling the dosing interval. For example the about 60 mg about monthly or about every 3 weeks regimen can be doubled to about every 2 months or about every 6 weeks respectively or tripled to about every 3 months or about every 9 weeks respectively. In an alternative embodiment, the patient receives gevokizumab at a dose of about 30 mg to about 120 mg about every 2 months or about every 6 weeks in the down-titration phase or in the maintenance phase independent from any safety issue or throughout the treatment phase. In an alternative embodiment the patient receives gevokizumab at a dose of about 30 mg to about 120 mg about every 3 months or about every 9 weeks in the down-titration phase or in the maintenance phase independent from any safety issue or throughout the treatment phase.

### Further Combinations

The combinations described herein can further comprises one or more other therapeutic agents, procedures or modalities.

In one embodiment, the methods described herein include administering to the subject a combination comprising a TIM-3 inhibitor described herein and a TGF-β inhibitor described herein (optionally further comprising a hypomethylating agent, optionally further comprising a PD-1 inhibitor or IL-1β inhibitor as described herein), in combination with a therapeutic agent, procedure, or modality, in an amount effective to treat or prevent a disorder described herein. In certain embodiments, the combination is administered or used in accordance with a dosage regimen described herein. In other embodiments, the combination is administered or used as a composition or formulation described herein.

The TIM-3 inhibitor, TGF-β inhibitor, PD-1 inhibitor, hypomethylating agent, IL-1β inhibitor, and the therapeutic agent, procedure, or modality can be administered or used simultaneously or sequentially in any order. Any combination and sequence of the TIM-3 inhibitor, TGF-β inhibitor, PD-1 inhibitor, hypomethylating agent, IL-1β inhibitor and the therapeutic agent, procedure, or modality (e.g., as described herein) can be used. The TIM-3 inhibitor, TGF-β inhibitor, PD-1 inhibitor, hypomethylating agent, IL-1β inhibitor, and/or the therapeutic agent, procedure or modality can be administered or used during periods of active disorder, or during a period of remission or less active disease. The TIM-3 inhibitor, TGF-β inhibitor, PD-1 inhibitor, IL-1β inhibitor, or hypomethylating agent can be administered before, concurrently with, or after the treatment with the therapeutic agent, procedure or modality.

In certain embodiments, the combination described herein can be administered with one or more of other antibody molecules, chemotherapy, other anti-cancer therapy (e.g., targeted anti-cancer therapies, gene therapy, viral therapy, RNA therapy bone marrow transplantation, nanotherapy, or oncolytic drugs), cytotoxic agents, immune-based therapies (e.g., cytokines or cell-based immune therapies), surgical procedures (e.g., lumpectomy or mastectomy) or radiation procedures, or a combination of any of the foregoing. The additional therapy may be in the form of adjuvant or neoadjuvant therapy. In some embodiments, the additional therapy is an enzymatic inhibitor (e.g., a small molecule enzymatic inhibitor) or a metastatic inhibitor. Exemplary cytotoxic agents that can be administered in combination with include antimicrotubule agents, topoisomerase inhibitors, antimetabolites, mitotic inhibitors, alkylating agents, anthracyclines, vinca alkaloids, intercalating agents, agents capable of interfering with a signal transduction pathway, agents that promote apoptosis, proteasome inhibitors, and radiation (e.g., local or whole-body irradiation (e.g., gamma irradiation). In other embodiments, the additional therapy is surgery or radiation, or a combination thereof. In other embodiments, the additional therapy is a therapy targeting one or more of PI3K/AKT/mTOR pathway, an HSP90 inhibitor, or a tubulin inhibitor.

Alternatively, or in combination with the aforesaid combinations, the combination described herein can be administered or used with, one or more of an inhibitor of CD47, CD70, NEDD8, CDK9, MDM2, FLT3, or KIT. In some embodiments, the TIM-3 inhibitor is administered with an inhibitor of CD47, CD70, NEDD8, CDK9, MDM2, FLT3, or KIT. In some embodiments, the TIM-3 inhibitor is administered with a TGF-β inhibitor, e.g., a TGF-β described herein, further in combination with an inhibitor of CD47, CD70, NEDD8, CDK9, MDM2, FLT3, or KIT. In some embodiments, the TIM-3 inhibitor is administered with a TGF-β inhibitor, e.g., a TGF-β described herein, and a hypomethylating agent, e.g., a hypomethylating agent described herein, further in combination with an inhibitor of CD47, CD70, NEDD8, CDK9, MDM2, FLT3, or KIT. In some embodiments, the TIM-3 inhibitor is administered with a TGF-β inhibitor, e.g., a TGF-β described herein, and a PD-1 inhibitor, e.g., a PD-1 inhibitor described herein, further in combination with an inhibitor of CD47, CD70, NEDD8, CDK9, MDM2, FLT3, or KIT. In some embodiments, the TIM-3 inhibitor is administered with a TGF-β inhibitor, e.g., a TGF-β described herein, and an IL-1β inhibitor, e.g., an IL-1β inhibitor described herein, further in combination with an inhibitor of CD47, CD70, NEDD8, CDK9, MDM2, FLT3, or KIT.

Alternatively, or in combination with the aforesaid combinations, the combination described herein can be administered or used with an activator of p53. In some embodiments, the TIM-3 inhibitor is administered with an activator of p53. In some embodiments, the TIM-3 inhibitor is administered with a TGF-β inhibitor, e.g., a TGF-β described herein, further in combination with an activator of p53. In some embodiments, the TIM-3 inhibitor is administered with a TGF-β inhibitor, e.g., a TGF-β described herein, and a hypomethylating agent, e.g., a hypomethylating agent described herein, further in combination with an activator of p53. In some embodiments, the TIM-3 inhibitor is administered with a TGF-β inhibitor, e.g., a TGF-β described herein, and a PD-1 inhibitor, e.g., a PD-1 inhibitor described herein, further in combination with an activator of p53. In some embodiments, the TIM-3 inhibitor is administered with a TGF-β inhibitor, e.g., a TGF-β described herein, and an IL-1β inhibitor, e.g., an IL-1β inhibitor described herein, further in combination with an activator of p53.

Alternatively, or in combination with the aforesaid combinations, the combination described herein can be administered or used with, one or more of an inhibitor of CD47, CD70, NEDD8, CDK9, MDM2, FLT3, or KIT. In some embodiments, the TIM-3 inhibitor is administered with an inhibitor of CD47, CD70, NEDD8, CDK9, MDM2, FLT3, or KIT. In some embodiments, the TIM-3 inhibitor is administered with a TGF-β inhibitor, e.g., a TGF-β described herein, further in combination with an inhibitor of CD47, CD70, NEDD8, CDK9, MDM2, FLT3, or KIT. In some embodiments, the TIM-3 inhibitor is administered with a TGF-β inhibitor, e.g., a TGF-β described herein, and an IL-1β inhibitor, e.g., an IL-1β inhibitor described herein, further in combination with an inhibitor of CD47, CD70, NEDD8, CDK9, MDM2, FLT3, or KIT.

Alternatively, or in combination with the aforesaid combinations, the combination described herein can be administered or used with an activator of p53. In some embodiments, the TIM-3 inhibitor is administered with an activator of p53. In some embodiments, the TIM-3 inhibitor is administered with a TGF-β inhibitor, e.g., a TGF-β described herein, further in combination with an activator of p53. In some embodiments, the TIM-3 inhibitor is administered with a TGF-β inhibitor, e.g., a TGF-β described herein, and an IL-1β inhibitor, e.g., an IL-1β inhibitor described herein, further in combination with an activator of p53.

Alternatively, or in combination with the aforesaid combinations, the combination described herein can be administered or used with, one or more of: an immunomodulator (e.g., an activator of a costimulatory molecule or an inhibitor of an inhibitory molecule, e.g., an immune checkpoint molecule); a vaccine, e.g., a therapeutic cancer vaccine; or other forms of cellular immunotherapy.

In certain embodiments, the combination described herein is administered or used in with a modulator of a costimulatory molecule or an inhibitory molecule, e.g., a co-inhibitory ligand or receptor.

In one embodiment, the compounds and combinations described herein are administered or used with a modulator, e.g., agonist, of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

In another embodiment, the compounds and combinations described herein are administered or used in combination with a GITR agonist, e.g., an anti-GITR antibody molecule.

In one embodiment, the combination described herein is administered or used in combination with an inhibitor of an inhibitory (or immune checkpoint) molecule chosen from PD-1, PD-L1, PD-L2, CTLA-4, TIM-3, LAG-3, CEACAM (e.g., CEACAM-1, CEACAM-3, and/or CEACAM-5), VISTA, BTLA, TIGIT, LAIR1, CD160, 2B4 and/or TGF beta. In one embodiment, the inhibitor is a soluble ligand (e.g., a CTLA-4-Ig), or an antibody or antibody fragment that binds to PD-1, LAG-3, PD-L1, PD-L2, or CTLA-4.

In another embodiment, the compounds and combinations described herein are administered or used in combination with a PD-1 inhibitor, e.g., an anti-PD-1 antibody molecule. In another embodiment, the combination described herein is administered or used in combination with a LAG-3 inhibitor, e.g., an anti-LAG-3 antibody molecule. In another embodiment, the combination described herein is administered or used in combination with a PD-L1 inhibitor, e.g., an anti-PD-L1 antibody molecule.

In another embodiment, the compounds and combinations described herein are administered or used in combination with a PD-1 inhibitor (e.g., an anti-PD-1 antibody molecule) and a LAG-3 inhibitor (e.g., an anti-LAG-3 antibody molecule). In another embodiment, the combination described herein is administered or used in combination with a PD-1 inhibitor (e.g., an anti-PD-1 antibody molecule) and a PD-L1 inhibitor (e.g., an anti-PD-L1 antibody molecule). In another embodiment, the combination described herein is administered or used in combination with a LAG-3 inhibitor (e.g., an anti-LAG-3 antibody molecule) and a PD-L1 inhibitor (e.g., an anti-PD-L1 antibody molecule).

In another embodiment, the compounds and combinations described herein are administered or used in combination with a CEACAM inhibitor (e.g., CEACAM-1, CEACAM-3, and/or CEACAM-5 inhibitor), e.g., an anti-CEACAM antibody molecule. In another embodiment, the combination described herein is administered or used in combination with a CEACAM-1 inhibitor, e.g., an anti-CEACAM-1 antibody molecule. In another embodiment, the combination described herein is administered or used in combination with a CEACAM-3 inhibitor, e.g., an anti-CEACAM-3 antibody molecule. In another embodiment, combination described herein is administered or used in combination with a CEACAM-5 inhibitor, e.g., an anti-CEACAM-5 antibody molecule.

The combination of antibody molecules disclosed herein can be administered separately, e.g., as separate antibody molecules, or linked, e.g., as a bispecific or trispecific antibody molecule. In one embodiment, a bispecific antibody that includes an anti-TIM-3 antibody molecule and an anti-PD-1, anti-CEACAM (e.g., anti-CEACAM-1, CEACAM-3, and/or anti-CEACAM-5), anti-PD-L1, or anti-LAG-3 antibody molecule, is administered. In certain embodiments, the combination of antibodies disclosed herein is used to treat a cancer, e.g., a cancer as described herein (e.g., a solid tumor or a hematologic malignancy).

### CD47 Inhibitor

In certain embodiments, the anti-TIM3 antibody described herein, optionally in combination with a hypomethylating agent described herein, or optionally in combination with a TGF-β inhibitor described herein, or optionally in combination with a hypomethylating agent and a TGF-β inhibitor as described herein, is further administered in combination with a CD47 inhibitor. In some embodiments, the CD47 inhibitor is magrolimab. In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary CD47 Inhibitor

In some embodiments, the CD47 inhibitor is an anti-CD47 antibody molecule. In some embodiments, the anti-CD47 antibody comprises magrolimab. Magrolimab is also known as ONO-7913, 5F9, or Hu5F9-G4. Magrolimab selectively binds to CD47 expressed on tumor cells and blocks the interaction of CD47 with its ligand signal regulatory protein alpha (SIRPa), a protein expressed on phagocytic cells. This typically prevents CD47/SIRPa-mediated signaling, allows the activation of macrophages, through the induction of pro-phagocytic signaling mediated by calreticulin, which is specifically expressed on the surface of tumor cells, and results in specific tumor cell phagocytosis. In addition, blocking CD47 signaling generally activates an anti-tumor T-lymphocyte immune response and T-mediated cell killing. Magrolimab is disclosed, e.g., in Sallaman et al. Blood 2019 134(Supplement_1):569, which is incorporated by reference in its entirety.

In some embodiments, magrolimab is administered intravenously. In some embodiments, magrolimab is administered on days 1, 4, 8, 11, 15, and 22 of cycle 1 (e.g., a 28 day cycle), days 1, 8, 15, and 22 of cycle 2 (e.g., a 28 day cycle), and days 1 and 15 of cycle 3 (e.g., a 28 day cycle) and subsequent cycles. In some embodiments, magrolimab is administered at least twice weekly, each week of, e.g., a 28 day cycle. In some embodiments, magrolimab is administered in a dose-escalation regimen. In some embodiments, magrolimab is administered at 1-30 mg/kg, e.g., 1-30 mg/kg per week.

Other CD47 Inhibitors

In some embodiments, the CD47 inhibitor is an inhibitor B6H12.2, CC-90002, C47B157, C47B161, C47B222, SRF231, ALX148, W6/32, 4N1K, 4N1, TTI-621, TTI-622, PKHB1, SEN177, MiR-708, or MiR-155. In some embodiments, the CD47 inhibitor is a bispecific antibody.

In some embodiments, the CD47 inhibitor is B6H12.2. B6H12.2 is disclosed, e.g., in Eladl et al. Journal of Hematology & Oncology 2020 13(96) https://doi.org/10.1186/s13045-020-00930-1. B6H12.2 is a humanized anti-CD74-IgG4 antibody that binds to CD47 expressed on tumor cells and blocks the interaction of CD47 with its ligand signal regulatory protein alpha (SIRPa).

In some embodiments, the CD47 inhibitor is CC-90002. CC-90002 is disclosed, e.g., in Eladl et al. Journal of Hematology & Oncology 2020 13(96) https://doi.org/10.1186/s13045-020-00930-1. CC-90002 is a monoclonal antibody targeting the human cell surface antigen CD47, with potential phagocytosis-inducing and antineoplastic activities. Upon administration, anti-CD47 monoclonal antibody CC-90002 selectively binds to CD47 expressed on tumor cells and blocks the interaction of CD47 with signal regulatory protein alpha (SIRPa), a protein expressed on phagocytic cells. This prevents CD47/SIRPa-mediated signaling and abrogates the CD47/SIRPa-mediated inhibition of phagocytosis. This induces pro-phagocytic signaling mediated by the binding of calreticulin (CRT), which is specifically expressed on the surface of tumor cells, to low-density lipoprotein (LDL) receptor-related protein (LRP), expressed on macrophages. This results in macrophage activation and the specific phagocytosis of tumor cells. In addition, blocking CD47 signaling activates both an anti-tumor T-lymphocyte immune response and T cell-mediated killing of CD47-expressing tumor cells. In some embodiments, CC-90002 is administered intravenously. In some embodiments, CC-90002 is administered intravenously on a 28-day cycle.

In some embodiments, the CD47 inhibitor is C47B157, C47B161, or C47B222. C47B157, C47B161, and C47B222 are disclosed, e.g., in Eladl et al. Journal of Hematology & Oncology 2020 13(96) https://doi.org/10.1186/s13045-020-00930-1. C47B157, C47B161, and C47B222 are humanized anti-CD74-IgG1 antibodies that bind to CD47 expressed on tumor cells and blocks the interaction of CD47 with its ligand signal regulatory protein alpha (SIRPa).

In some embodiments, the CD47 inhibitor is SRF231. SRF231 is disclosed, e.g., in Eladl et al. Journal of Hematology & Oncology 2020 13(96) https://doi.org/10.1186/s13045-020-00930-1. SRF231 is a human monoclonal antibody targeting the human cell surface antigen CD47, with potential phagocytosis-inducing and antineoplastic activities. Upon administration, anti-CD47 monoclonal antibody SRF231 selectively binds to CD47 on tumor cells and blocks the interaction of CD47 with signal regulatory protein alpha (SIRPalpha), an inhibitory protein expressed on macrophages. This prevents CD47/SIRPalpha-mediated signaling and abrogates the CD47/SIRPa-mediated inhibition of phagocytosis. This induces pro-phagocytic signaling mediated by the binding of calreticulin (CRT), which is specifically expressed on the surface of tumor cells, to low-density lipoprotein (LDL) receptor-related protein (LRP), expressed on macrophages. This results in macrophage activation and the specific phagocytosis of tumor cells. In addition, blocking CD47 signaling activates both an anti-tumor T-lymphocyte immune response and T-cell-mediated killing of CD47-expressing tumor cells.

In some embodiments, the CD47 inhibitor is ALX148. ALX148 is disclosed, e.g., in Eladl et al. Journal of Hematology & Oncology 2020 13(96) https://doi.org/10.1186/s13045-020-00930-1. ALX148 is a CD47 antagonist. It is a variant of signal regulatory protein alpha (SIRPa) that antagonizes the human cell surface antigen CD47, with potential phagocytosis-inducing, immunostimulating and antineoplastic activities. Upon administration, ALX148 binds to CD47 expressed on tumor cells and prevents the interaction of CD47 with its ligand SIRPa, a protein expressed on phagocytic cells. This prevents CD47/SIRPa-mediated signaling and abrogates the CD47/SIRPa-mediated inhibition of phagocytosis. This induces pro-phagocytic signaling mediated by the binding of the pro-phagocytic signaling protein calreticulin (CRT), which is specifically expressed on the surface of tumor cells, to low-density lipoprotein (LDL) receptor-related protein (LRP), expressed on macrophages. This results in macrophage activation and the specific phagocytosis of tumor cells. In addition, blocking CD47 signaling activates both an anti-tumor cytotoxic T-lymphocyte (CTL) immune response and T-cell-mediated killing of CD47-expressing tumor cells. In some embodiments, ALX148 is administered intravenously. In some embodiments, ALX148 is administered at least once a week. In some embodiments, ALX148 is administered at least twice a week.

In some embodiments, the CD47 inhibitor is W6/32. W6/32 is disclosed, e.g., in Eladl et al. Journal of Hematology & Oncology 2020 13(96) https://doi.org/10.1186/s13045-020-00930-1, which is incorporated by reference in its entirety. W6/32 is an anti-CD47 antibody that targets CD47-MHC-1.

In some embodiments, the CD47 inhibitor is 4N1K or 4N1. 4N1K and 4N1 are disclosed, e.g., in Eladl et al. Journal of Hematology & Oncology 2020 13(96) https://doi.org/10.1186/s13045-020-00930-1, which is incorporated by reference in its entirety. 4NIK and 4N1 are CD47-SIRPα Peptide agonists.

In some embodiments, the CD47 inhibitor is TTI-621. TTI-621 is disclosed, e.g., in Eladl et al. Journal of Hematology & Oncology 2020 13(96) https://doi.org/10.1186/s13045-020-00930-1, which is incorporated by reference in its entirety. TTI-621 is also known as SIRPα-IgG1 Fc. TTI-621 is a soluble recombinant antibody-like fusion protein composed of the N-terminal CD47 binding domain of human signal-regulatory protein alpha (SIRPa) linked to the Fc domain of human immunoglobulin G1 (IgG1), with potential immune checkpoint inhibitory and antineoplastic activities. Upon administration, the SIRPa-Fc fusion protein TTI-621 selectively targets and binds to CD47 expressed on tumor cells and blocks the interaction of CD47 with endogenous SIRPa, a cell surface protein expressed on macrophages. This prevents CD47/SIRPa-mediated signaling and abrogates the CD47/SIRPa-mediated inhibition of macrophage activation and phagocytosis of cancer cells. This induces pro-phagocytic signaling mediated by the binding of calreticulin (CRT), which is specifically expressed on the surface of tumor cells, to low-density lipoprotein (LDL) receptor-related protein-1 (LRP-1), expressed on macrophages, and results in macrophage activation and the specific phagocytosis of tumor cells. In some embodiments, TTI-621 is administered intratumorally.

In some embodiments, the CD47 inhibitor is TTI-622. TTI-622 is disclosed, e.g., in Eladl et al. Journal of Hematology & Oncology 2020 13(96) https://doi.org/10.1186/s13045-020-00930-1, which is incorporated by reference in its entirety. TTI-622 is also known as SIRPα-IgG1 Fc. TTI-622 is a soluble recombinant antibody-like fusion protein composed of the N-terminal CD47 binding domain of human signal-regulatory protein alpha (SIRPa; CD172a) linked to an Fc domain derived from human immunoglobulin G subtype 4 (IgG4), with potential immune checkpoint inhibitory, phagocytosis-inducing and antineoplastic activities. Upon administration, the SIRPa-IgG4-Fc fusion protein TTI-622 selectively targets and binds to CD47 expressed on tumor cells and blocks the interaction of CD47 with endogenous SIRPa, a cell surface protein expressed on macrophages. This prevents CD47/SIRPa-mediated signaling and abrogates the CD47/SIRPa-mediated inhibition of macrophage activation. This induces pro-phagocytic signaling resulting from the binding of calreticulin (CRT), which is specifically expressed on the surface of tumor cells, to low-density lipoprotein (LDL) receptor-related protein-1 (LRP-1) expressed on macrophages, and results in macrophage activation and the specific phagocytosis of tumor cells.

In some embodiments, the CD47 inhibitor is PKHB1. PKHB1 is disclosed, e.g., in Eladl et al. Journal of Hematology & Oncology 2020 13(96) https://doi.org/10.1186/s13045-020-00930-1, which is incorporated by reference in its entirety. PKHB1 is a CD47 peptide agonist that binds CD47 and blocks the interaction with SIRPα.

In some embodiments, the CD47 inhibitor is SEN177. SEN177 is disclosed, e.g., in Eladl et al. Journal of Hematology & Oncology 2020 13(96) https://doi.org/10.1186/s13045-020-00930-1, which is incorporated by reference in its entirety. SEN177 is an antibody that targets QPCTL in CD47.

In some embodiments, the CD47 inhibitor is MiR-708. MiR-708 is disclosed, e.g., in Eladl et al. Journal of Hematology & Oncology 2020 13(96) https://doi.org/10.1186/s13045-020-00930-1, which is incorporated by reference in its entirety. MiR-708 is a miRNA that targets CD47 and blocks the interaction with SIRPα.

In some embodiments, the CD47 inhibitor is MiR-155. MiR-155 is disclosed, e.g., in Eladl et al. Journal of Hematology & Oncology 2020 13(96) https://doi.org/10.1186/s13045-020-00930-1, which is incorporated by reference in its entirety. MiR-155 is a miRNA that targets CD47 and blocks the interaction with SIRPα.

In some embodiments, the CD47 inhibitor is an anti-CD74, anti-PD-L1 bispecific antibody or an anti-CD47, anti-CD20 bispecific antibody, as disclosed in Eladl et al. Journal of Hematology & Oncology 2020 13(96) https://doi.org/10.1186/s13045-020-00930-1, which is incorporated by reference in its entirety.

In some embodiments, the CD74 inhibitor is LicMAB as disclosed in, e.g., Ponce et al. Oncotarget 2017 8(7):11284-11301, which is incorporated by reference in its entirety.

### CD70 Inhibitor

In certain embodiments, the anti-TIM3 antibody described herein, optionally in combination with a hypomethylating agent described herein, or optionally in combination with a TGF-β inhibitor described herein, or optionally in combination with a hypomethylating agent and a TGF-β inhibitor as described herein, is further administered in combination with a CD70 inhibitor. In some embodiments, the CD70 inhibitor is cusatuzumab. In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary CD70 Inhibitor

In some embodiments, the CD70 inhibitor is an anti-CD70 antibody molecule. In some embodiments, the anti-CD70 antibody comprises cusatuzumab. Cusatuzumab is also known as ARGX-110 or JNJ-74494550. Cusatuzumab selectively binds to, and neutralizes the activity of CD70, which may also induce an antibody-dependent cellular cytotoxicity (ADCC) response against CD70-expressing tumor cells. Cusatuzumab is disclosed, e.g., in Riether et al. Nature Medicine 2020 26:1459-1467, which is incorporated by reference in its entirety.

In some embodiments, cusatuzumab is administered intravenously. In some embodiments, cusatuzumab is administered subcutaneously. In some embodiments, cusatuzumab is administered at 1-20 mg/kg, e.g., 1 mg/kg, 3 mg/kg, 10 mg/kg, or 20 mg/kg. In some embodiments, cusatuzumab is administered once every two weeks. In some embodiments, cusatuzumab is administered at 10 mg/kg once every two weeks. In some embodiments, cusatuzumab is administered at 20 mg/kg once every two weeks. In some embodiments, cusatuzumab is administered on day 3 and day 17 of, e.g., a 28 day cycle.

### p53 Activator

In certain embodiments, the anti-TIM3 antibody described herein, optionally in combination with a hypomethylating agent described herein, or optionally in combination with a TGF-β inhibitor described herein, or optionally in combination with a hypomethylating agent and a TGF-β inhibitor as described herein, is further administered in combination with a p53 activator. In some embodiments, the p53 activator is APR-246. In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary p53 Activator

In some embodiments, the p53 activator is APR-246. APR-246 is a methylated derivative and structural analog of PRIMA-1 (p53 re-activation and induction of massive apoptosis). APR-246 is also known as Eprenetapopt, PRIMA-1MET. APR-246 covalently modifies the core domain of mutated forms of cellular tumor p53 through the alkylation of thiol groups. These modifications restore both the wild-type conformation and function to mutant p53, which reconstitutes endogenous p53 activity, leading to cell cycle arrest and apoptosis in tumor cells. APR-246 is disclosed, e.g., in Zhang et al. Cell Death and Disease 2018 9(439), which is incorporated by reference in its entirety.

In some embodiments, APR-246 is administered on days 1-4 of, e.g., a 28-day cycle, e.g., for 12 cycles. In some embodiments, APR-246 is administered at 4-5 g, e.g., 4.5 g, each day.

### NEDD8 Inhibitor

In certain embodiments, the anti-TIM3 antibody described herein, optionally in combination with a hypomethylating agent described herein, or optionally in combination with a TGF-β inhibitor described herein, or optionally in combination with a hypomethylating agent and a TGF-β inhibitor as described herein, is further administered in combination with a NEDD8 inhibitor. In some embodiments, the NEDD8 inhibitor is an inhibitor of NEDD8 activating enzyme (NAE). In some embodiments, the NEDD8 inhibitor is pevonedistat. In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary NEDD8 Inhibitor

In some embodiments, the NEDD8 inhibitor is a small molecule inhibitor. In some embodiments, the NEDD8 inhibitor is pevonedistat. Pevonedistat is also known as TAK-924, NAE inhibitor MLN4924, Nedd8-activating enzyme inhibitor MLN4924, MLN4924, or ((1S,2S,4R)-4-(4-((1S)-2,3-Dihydro-1H-inden-1-ylamino)-7H-pyrrolo(2,3-d)pyrimidin-7-yl)-2-hydroxycyclopentyl)methyl sulphamate. Pevonedistat binds to and inhibits NAE, which may result in the inhibition of tumor cell proliferation and survival. NAE activates Nedd8 (Neural precursor cell expressed, developmentally down-regulated 8), a ubiquitin-like (UBL) protein that modifies cellular targets in a pathway that is parallel to but distinct from the ubiquitin-proteasome pathway (UPP). Pevonedistat is disclosed, e.g., in Swords et al. Blood (2018) 131(13)1415-1424, which is incorporated by reference in its entirety.

In some embodiments, pevonedistat is administered intravenously. In some embodiments, pevonedistat is administered at 10-50 mg/m², e.g., 10 mg/m², 20 mg/m², 25 mg/m², 30 mg/m², or 50 mg/m². In some embodiments, pevonedistat is administered on days 1, 3, and 5 of, e.g., a 28-day cycle, for, e.g., up to 16 cycles. In some embodiments, pevonedistat is administered using fixed dosing. In some embodiments, pevonedistat is administered in a ramp-up dosing schedule. In some embodiments, pevonedistat is administered at 25 mg/m² on day 1 and 50 mg/m² on day 8 of, e.g., each 28 day cycle.

### CDK9 Inhibitors

In certain embodiments, the anti-TIM3 antibody described herein, optionally in combination with a hypomethylating agent described herein, or optionally in combination with a TGF-β inhibitor described herein, or optionally in combination with a hypomethylating agent and a TGF-β inhibitor as described herein, is further administered in combination with a cyclin dependent kinase inhibitor. In some embodiments, the combination described herein is further administered in combination with a CDK9 inhibitor. In some embodiments, the CDK9 inhibitor is alvocidib or alvocidib prodrug TP-1287. In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary CDK9 Inhibitor

In some embodiments, the CDK9 inhibitor is Alvocidib. Alvocidib is also known as flavopiridol, FLAVO, HMR 1275, L-868275, or (−)-2-(2-chlorophenyl)-5,7-dihydroxy-8-[(3R,4S)-3-hydroxy-1-methyl-4-piperidinyl]-4H-1-benzopyran-4-one hydrochloride. Alvocidib is a synthetic N-methylpiperidinyl chlorophenyl flavone compound. As an inhibitor of cyclin-dependent kinase, alvocidib induces cell cycle arrest by preventing phosphorylation of cyclin-dependent kinases (CDKs) and by down-regulating cyclin D1 and D3 expression, resulting in G1 cell cycle arrest and apoptosis. This agent is also a competitive inhibitor of adenosine triphosphate activity. Alvocidib is disclosed, e.g., in Gupta et al. Cancer Sensistizing Agents for Chemotherapy 2019: pp. 125-149, which is incorporated by reference in its entirety.

In some embodiments, alvocidib is administered intravenously. In some embodiments, alvocidib is administered on days 1, 2, and/or 3 of, e.g., a 28 day cycle. In some embodiments, alvocidib is administered using fixed dosing. In some embodiments, alvocidib is administered in a ramp-up dosing schedule. In some embodiments, alvocidib is administered for 4-weeks, followed by a 2 week rest period, for, e.g., up to a maximum of 6 cycles (e.g., a 28 day cycle). In some embodiments, alvocidib is administered at 30-50 mg/m², e.g., 30 mg/m² or 50 mg/m². In some embodiments, alvocidib is administered at 30 mg/m² as a 30-minute intravenous (IV) infusion followed by 30 mg/m² as a 4-hour continuous infusion. In some embodiments, alvocidib is administered at 30 mg/m2 over 30 minutes followed by 50 mg/m2 over 4 hours. In some embodiments, alvocidib is administered at a first dose of 30 mg/m² as a 30-minute intravenous (IV) infusion followed by 30 mg/m² as a 4-hour continuous infusion, and one or more subsequent doses of 30 mg/m2 over 30 minutes followed by 50 mg/m2 over 4 hours.

Other CDK9 Inhibitor

In some embodiments, the CDK9 inhibitor is TP-1287. TP-1287 is also known as alvocidib phosphate TP-1287 or alvocidib phosphate. TP-1287 is an orally bioavailable, highly soluble phosphate prodrug of alvocidib, a potent inhibitor of cyclin-dependent kinase-9 (CDK9), with potential antineoplastic activity. Upon administration of the phosphate prodrug TP-1287, the prodrug is enzymatically cleaved at the tumor site and the active moiety alvocidib is released. Alvocidib targets and binds to CDK9, thereby reducing the expression of CDK9 target genes such as the anti-apoptotic protein MCL-1, and inducing G1 cell cycle arrest and apoptosis in CDK9-overexpressing cancer cells. TP-1287 is disclosed, e.g., in Kim et al. Cancer Research (2017) Abstract 5133; Proceedings: AACR Annual Meeting 2017, which is incorporated by reference in its entirety. In some embodiments, TP-1287 is administered orally.

### MDM2 Inhibitors

In certain embodiments, the anti-TIM3 antibody described herein, optionally in combination with a hypomethylating agent described herein, or optionally in combination with a TGF-β inhibitor described herein, or optionally in combination with a hypomethylating agent and a TGF-β inhibitor as described herein, is further administered in combination with an MDM2 inhibitor. In some embodiments, the MDM2 inhibitor is idasanutlin, KRT-232, milademetan, or APG-115. In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary MDM2 Inhibitors

In some embodiments, the MDM2 inhibitor is a small molecule inhibitor. In some embodiments, the MDM2 inhibitor is idasanutlin. Idasanutlin is also known as RG7388 or RO 5503781. Idasanutlin is an orally available, small molecule, antagonist of MDM2 (mouse double minute 2; Mdm2 p53 binding protein homolog), with potential antineoplastic activity. Idasanutlin binds to MDM2 blocking the interaction between the MDM2 protein and the transcriptional activation domain of the tumor suppressor protein p53. By preventing the MDM2-p53 interaction, p53 is not enzymatically degraded and the transcriptional activity of p53 is restored, which may lead to p53-mediated induction of tumor cell apoptosis. Idasanutlin is disclosed, e.g., in Mascarenhas et al. Blood (2019) 134(6):525-533, which is incorporated by reference in its entirety. In some embodiments, idasanutlin is administered orally. In some embodiments, idasanutlin is administered on days 1-5 of, e.g., a 28 day cycle. In some embodiments, idasanutlin is administered at 400-500 mg, e.g., 300 mg. In some embodiment, idasanutlin is administered once or twice daily. In some embodiments, idasanutlin is administered at 300 mg twice daily in cycle 1 (e.g., a 28 day cycle) or once daily in cycles 2 and/or 3 (e.g., a 28 day cycle) for, e.g. 5 days every treatment cycle (e.g., a 28 day cycle).

In some embodiments, the MDM2 inhibitor is KRT-232. KRT-232 is also known as (3R,5R,6S)-5-(3-Chlorophenyl)-6-(4-chlorophenyl)-3-methyl-1-((1S)-2-methyl-1-(((1-methylethyl)sulfonyl)methyl)propyl)-2-oxo-3-piperidineacetic Acid, or AMG-232. KRT-232 is an orally available inhibitor of MDM2 (murine double minute 2), with potential antineoplastic activity. Upon oral administration, MDM2 inhibitor KRT-232 binds to the MDM2 protein and prevents its binding to the transcriptional activation domain of the tumor suppressor protein p53. By preventing this MDM2-p53 interaction, the transcriptional activity of p53 is restored. KRT-232 is disclosed, e.g., in Garcia-Delgado et al. Blood (2019) 134(Supplement_1): 2945, which is incorporated by reference in its entirety. In some embodiments, KRT-232 is administered orally. In some embodiments, KRT-232 is administered once daily. In some embodiments, KRT-232 is administered on days 1-7 of a cycle, e.g., a 28 day cycle. In some embodiments, KRT-232 is administered on days 4-10 and 18-24 of, e.g., a 28 day cycle, for up to, e.g., 4 cycles.

In some embodiments, the MDM2 inhibitor is milademetan. Milademetan is also known as HDM2 inhibitor DS-3032b or DS-3032b. Milademetan is an orally available MDM2 (murine double minute 2) antagonist with potential antineoplastic activity. Upon oral administration, milademetan tosylate binds to, and prevents the binding of MDM2 protein to the transcriptional activation domain of the tumor suppressor protein p53. By preventing this MDM2-p53 interaction, the proteosome-mediated enzymatic degradation of p53 is inhibited and the transcriptional activity of p53 is restored. This results in the restoration of p53 signaling and leads to the p53-mediated induction of tumor cell apoptosis. Milademetan is disclosed, e.g., in DiNardo et al. Blood (2019) 134(Supplement_1):3932, which is incorporated by reference in its entirety. In some embodiments, milademetan is administered orally. In some embodiments, milademetan is administered at 5-200 mg, e.g., 5 mg, 20 mg, 30 mg, 80 mg, 100 mg, 90 mg, and/or 200 mg. In some embodiments, milademetan is administered in a single capsule or multiple capsules. In some embodiments, milademetan is administered at a fixed dose. In some embodiments, milademetan is administered in a dose escalation regimen. In some embodiments, milademetan is administered in further combination with quizartinib (an inhibitor of FLT3). In some embodiments, milademetan is administered at 5-200 mg (e.g., 5 mg, 20 mg, 80 mg, or 200 mg), and quizartinib is administered at 20-30 mg (e.g., 20 mg or 30 mg).

In some embodiments, the MDM2 inhibitor is APG-115. APG-115 is an orally available inhibitor of human homologminute 2 (HDM2; mouse double minute 2 homolog; MDM2), with potential antineoplastic activity. Upon oral administration, the p53-HDM2 protein-protein interaction inhibitor APG-115 binds to HDM2, preventing the binding of the HDM2 protein to the transcriptional activation domain of the tumor suppressor protein p53. By preventing this HDM2-p53 interaction, the proteasome-mediated enzymatic degradation of p53 is inhibited and the transcriptional activity of p53 is restored. This may result in the restoration of p53 signaling and lead to the p53-mediated induction of tumor cell apoptosis. APG-115 is disclosed, e.g., in Fang et al. Journal for ImmunoTherapy of Cancer (2019) 7(327), which is incorporated by reference in its entirety. In some embodiments, APG-115 is administered orally. In some embodiments, APG-115 is administered at 100-250 mg, e.g., 100 mg, 150 mg, 200 mg, and/or 250 mg. In some embodiments, APG-115 is administered on days 1-5 of, e.g., a 28 day cycle. In some embodiments, APG-115 is administered on days 1-7 of, e.g., a 28 day cycle. In some embodiments, APG-115 is administered at flat dose. In some embodiments, APG-115 is administered on a dose escalation schedule. In some embodiments, APG-115 is administered at 100 mg per day on day 1-5 of a 28 day cycle. In some embodiments, APG-115 is administered at 150 mg per day on day 1-5 of a 28 day cycle. In some embodiments, APG-115 is administered at 200 mg per day on day 1-5 of a 28 day cycle. In some embodiments, APG-115 is administered at 250 mg per day on day 1-5 of a 28 day cycle.

### FLT3 Inhibitors

In certain embodiments, the anti-TIM3 antibody described herein, optionally in combination with a hypomethylating agent described herein, or optionally in combination with a TGF-β inhibitor described herein, or optionally in combination with a hypomethylating agent and a TGF-β inhibitor as described herein, is further administered in combination with an FTL3 inhibitor. In some embodiments, the FLT3 inhibitor is gilteritinib, quizartinib, or crenolanib. In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary FLT3 Inhibitors

In some embodiments, the FLT3 inhibitor is gilteritinib. Gilteritinib is also known as ASP2215. Gilteritinib is an orally bioavailable inhibitor of the receptor tyrosine kinases (RTKs) FMS-related tyrosine kinase 3 (FLT3, STK1, or FLK2), AXL (UFO or JTK11) and anaplastic lymphoma kinase (ALK or CD246), with potential antineoplastic activity. Gilteritinib binds to and inhibits both the wild-type and mutated forms of FLT3, AXL and ALK. This may result in an inhibition of FLT3, AXL, and ALK-mediated signal transduction pathways and reduction of tumor cell proliferation in cancer cell types that overexpress these RTKs. Gilteritinib is disclosed, e.g, in Perl et al. N Engl J Med (2019) 381:1728-1740, which is incorporated by reference in its entirety. In some embodiments, gilteritinib is administered orally.

In some embodiments, the FLT3 inhibitor is quizartinib. Quizartinib is also known as AC220 or 1-(5-tert-butyl-1,2-oxazol-3-yl)-3-[4-[6-(2-morpholin-4-ylethoxy)imidazo[2,1-b][1,3]benzothiazol-2-yl]phenyl]urea. Quizartinib is disclosed, e.g., in Cortes et al. The Lancet (2019) 20(7):984-997. In some embodiments, quizartinib is administered orally. In some embodiments, quizartinib is administered at 20-60 mg, e.g., 20 mg, 30 mg, 40 mg, and/or 60 mg. In some embodiments, quizartinib is administered once a day. In some embodiments, quizartinib is administered at a flat dose. In some embodiments, quizartinib is administered at 20 mg daily. In some embodiments, quizartinib is administered at 30 mg once daily. In some embodiments, quizartinib is administered at 40 mg once daily. In some embodiments, quizartinib is administered in a dose escalation regimen. In some embodiments, quizartinib is administered at 30 mg daily for days 1-14 of, e.g., a 28 day cycle, and is administered at 60 mg daily for days 15-28, of, e.g., a 28 day cycle. In some embodiments, quizartinib is administered at 20 mg daily for days 1-14 of, e.g., a 28 day cycle, and is administered at 30 mg daily for days 15-28, of, e.g., a 28 day cycle.

In some embodiments, the FLT3 inhibitor is crenolanib. Crenolanib is an orally bioavailable small molecule, targeting the platelet-derived growth factor receptor (PDGFR), with potential antineoplastic activity. Crenolanib binds to and inhibits PDGFR, which may result in the inhibition of PDGFR-related signal transduction pathways, and, so, the inhibition of tumor angiogenesis and tumor cell proliferation. Crenolanib is also known as CP-868596. Crenolanib is disclosed, e.g., in Zimmerman et al. Blood (2013) 122(22):3607-3615, which is incorporated by reference in its entirety. In some embodiments, crenolanib is administered orally. In some embodiments, crenolanib is administered daily. In some embodiments, crenolanib is administered at 100-200 mg, e.g., 100 mg or 200 mg. In some embodiments, crenolanib is administered once a day, twice a day, or three times a day. In some embodiments, crenolanib is administered at 200 mg daily in three equal doses, e.g., every 8 hours.

### KIT Inhibitors

In certain embodiments, the anti-TIM3 antibody described herein, optionally in combination with a hypomethylating agent described herein, or optionally in combination with a TGF-β inhibitor described herein, or optionally in combination with a hypomethylating agent and a TGF-β inhibitor as described herein, is further administered in combination with a KIT inhibitor. In some embodiments, the KIT inhibitor is ripretinib or avapritinib. In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary KIT Inhibitors

In some embodiments, the KIT inhibitor is ripretinib. Ripretinib is an orally bioavailable switch pocket control inhibitor of wild-type and mutated forms of the tumor-associated antigens (TAA) mast/stem cell factor receptor (SCFR) KIT and platelet-derived growth factor receptor alpha (PDGFR-alpha; PDGFRa), with potential antineoplastic activity. Upon oral administration, ripretinib targets and binds to both wild-type and mutant forms of KIT and PDGFRa specifically at their switch pocket binding sites, thereby preventing the switch from inactive to active conformations of these kinases and inactivating their wild-type and mutant forms. This abrogates KIT/PDGFRa-mediated tumor cell signaling and prevents proliferation in KIT/PDGFRa-driven cancers. DCC-2618 also inhibits several other kinases, including vascular endothelial growth factor receptor type 2 (VEGFR2; KDR), angiopoietin-1 receptor (TIE2; TEK), PDGFR-beta and macrophage colony-stimulating factor 1 receptor (FMS; CSF1R), thereby further inhibiting tumor cell growth. Ripretinib is also known as DCC2618, QINLOCK™ (Deciphera), or 1-N′-[2,5-difluoro-4-[2-(1-methylpyrazol-4-yl)pyridin-4-yl]oxyphenyl]-1-N′-phenylcyclopropane-1,1-dicarboxamide. In some embodiments, ripretinib is administered orally. In some embodiments, ripretinib is administered at 100-200 mg, e.g., 150 mg. In some embodiments, ripretinib is administered in three 50 mg tablets. In some embodiments, ripretinib is administered at 150 mg once daily. In some embodiments, ripretinib is administered in three 50 mg tablets taken together once daily.

In some embodiments, the KIT inhibitor is avapritinib. Avapritinib is also known as BLU-285 or AYVAKIT™ (Blueprint Medicines). Avapritinib is an orally bioavailable inhibitor of specific mutated forms of platelet-derived growth factor receptor alpha (PDGFR alpha; PDGFRa) and mast/stem cell factor receptor c-Kit (SCFR), with potential antineoplastic activity. Upon oral administration, avapritinib specifically binds to and inhibits specific mutant forms of PDGFRa and c-Kit, including the PDGFRa D842V mutant and various KIT exon 17 mutants. This results in the inhibition of PDGFRa- and c-Kit-mediated signal transduction pathways and the inhibition of proliferation in tumor cells that express these PDGFRa and c-Kit mutants. In some embodiments, avapritinib is administered orally. In some embodiments, avapritinib is administered daily. In some embodiments, avapritinib is administered at 100-300 mg, e.g., 100 mg, 200 mg, 300 mg. In some embodiments, avapritinib is administered once a day. In some embodiments, avapritinib is administered at 300 mg once a day. In some embodiments, avapritinib is administered at 200 mg once a day. In some embodiments, avapritinib is administered at 100 mg once a day. In some embodiments, avapritinib is administered continuously in, e.g., 28 day cycles.

### PD-L1 Inhibitors

In certain embodiments, the composition and/or combinations described herein is further administered in combination with a PD-L1 inhibitor. In some embodiments, the PD-L1 inhibitor is chosen from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb). In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary PD-L1 Inhibitors

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety. The antibody molecules described herein can be made by vectors, host cells, and methods described in US 2016/0108123, which is incorporated by reference in its entirety.

Other Exemplary PD-L1 Inhibitors

In one embodiment, the anti-PD-L1 antibody molecule is Atezolizumab (Genentech/Roche), also known as MPDL3280A, RG7446, RO5541267, YW243.55.570, or TECENTRIQ™. Atezolizumab and other anti-PD-L1 antibodies are disclosed in U.S. Pat. No. 8,217,149, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizumab.

In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab.

In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

### LAG-3 Inhibitors

In certain embodiments, the compositions and combinations described herein are further administered in combination with a LAG-3 inhibitor. In some embodiments, the LAG-3 inhibitor is chosen from LAG525 (Novartis), BMS-986016 (Bristol-Myers Squibb), or TSR-033 (Tesaro). In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety. The antibody molecules described herein can be made by vectors, host cells, and methods described in US 2015/0259420, which is incorporated by reference in its entirety, and described in US 2015/0259420, which is also incorporated by reference in its entirety.

Other Exemplary LAG-3 Inhibitors

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

### GITR Agonists

In certain embodiments, the compositions and combinations described herein are administered in combination with a GITR agonist. In some embodiments, the GITR agonist is GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx). In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary GITR Agonists

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety. The antibody molecules described herein can be made by vectors, host cells, and methods described in WO 2016/057846, which is incorporated by reference in its entirety. The antibody molecules described herein can be made by vectors, host cells, and methods described in WO 2016/057846, incorporated by reference in its entirety.

Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76(14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

### IL15/IL-15Ra Complexes

In certain embodiments, the compositions and/or combinations described herein are further administered in combination with an IL-15/IL-15Ra complex. In some embodiments, the IL-15/IL-15Ra complex is chosen from NIZ985 (Novartis), ATL-803 (Altor) or CYP0150 (Cytune). In some embodiments, these combinations are used to treat the cancer indications disclosed herein, including the hematologic indications disclosed herein, including a myeloproliferative neoplasm, e.g., myelofibrosis (MF). In some embodiments, these combinations are used to treat the cancer indications disclosed herein, including the hematologic indications disclosed herein, including an MDS (e.g., a lower risk MDS).

Exemplary IL-15/IL-15Ra Complexes

In one embodiment, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence described in WO 2014/066527, incorporated herein by reference in its entirety, and the soluble form of human IL-15Ra comprises an amino acid sequence, as described in WO 2014/066527, incorporated by reference in its entirety. The molecules described herein can be made by vectors, host cells, and methods described in WO 2007/084342, incorporated by reference in its entirety.

Other Exemplary IL-15/IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is disclosed in WO 2008/143794, incorporated by reference in its entirety.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is disclosed in WO 2007/04606 and WO 2012/175222, incorporated by reference in their entirety.

### Pharmaceutical Compositions, Formulations, and Kits

In another aspect, the disclosure provides compositions, e.g., pharmaceutically acceptable compositions, which include a combination described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions described herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (e.g., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

A combination or a composition described herein can be formulated into a formulation (e.g., a dose formulation or dosage form) suitable for administration (e.g., intravenous administration) to a subject as described herein. The formulation described herein can be a liquid formulation, a lyophilized formulation, or a reconstituted formulation.

In certain embodiments, the formulation is a liquid formulation. In some embodiments, the formulation (e.g., liquid formulation) comprises a TIM-3 inhibitor (e.g., an anti-TIM-3 antibody molecule described herein) and a buffering agent. In some embodiments, the formulation (e.g., liquid formulation) comprises a TGF-β inhibitor (e.g. an anti-TGF-β antibody molecule described herein) and a buffering agent. In some embodiments, the formulation (e.g., liquid formulation) comprises a PD-1 inhibitor (e.g. an anti-PD-1 antibody molecule described herein) and a buffering agent. In some embodiments, the formulation (e.g., liquid formulation) comprises an IL-1β inhibitor (e.g. an anti-IL-1β antibody molecule described herein) and a buffering agent.

In some embodiments, the formulation (e.g., liquid formulation) comprises an anti-TIM-3, anti-TGF-β, anti-P-D1, or anti-IL-1β antibody molecule as disclosed herein present at a concentration of 25 mg/mL to 250 mg/mL, e.g., 50 mg/mL to 200 mg/mL, 60 mg/mL to 180 mg/mL, 70 mg/mL to 150 mg/mL, 80 mg/mL to 120 mg/mL, 90 mg/mL to 110 mg/mL, 50 mg/mL to 150 mg/mL, 50 mg/mL to 100 mg/mL, 150 mg/mL to 200 mg/mL, or 100 mg/mL to 200 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL. In certain embodiments, the antibody molecule is present at a concentration of 80 mg/mL to 120 mg/mL, e.g., 100 mg/mL.

In some embodiments, the formulation (e.g., liquid formulation) comprises a buffering agent comprising histidine (e.g., a histidine buffer). In certain embodiments, the buffering agent (e.g., histidine buffer) is present at a concentration of 1 mM to 100 mM, e.g., 2 mM to 50 mM, 5 mM to 40 mM, 10 mM to 30 mM, 15 to 25 mM, 5 mM to 40 mM, 5 mM to 30 mM, 5 mM to 20 mM, 5 mM to 10 mM, 40 mM to 50 mM, 30 mM to 50 mM, 20 mM to 50 mM, 10 mM to 50 mM, or 5 mM to 50 mM, e.g., 2 mM, 5 mM, 10 mM, 15 mM, 20 mM, 25 mM, 30 mM, 35 mM, 40 mM, 45 mM, or 50 mM. In some embodiments, the buffering agent (e.g., histidine buffer) is present at a concentration of 15 mM to 25 mM, e.g., 20 mM. In other embodiments, the buffering agent (e.g., a histidine buffer) has a pH of 4 to 7, e.g., 5 to 6, e.g., 5, 5.5, or 6. In some embodiments, the buffering agent (e.g., histidine buffer) has a pH of 5 to 6, e.g., 5.5. In certain embodiments, the buffering agent comprises a histidine buffer at a concentration of 15 mM to 25 mM (e.g., 20 mM) and has a pH of 5 to 6 (e.g., 5.5). In certain embodiments, the buffering agent comprises histidine and histidine-HCl.

In some embodiments, the formulation (e.g., liquid formulation) comprises an antibody molecule as disclosed herein present at a concentration of 80 to 120 mg/mL, e.g., 100 mg/mL; and a buffering agent that comprises a histidine buffer at a concentration of 15 mM to 25 mM (e.g., 20 mM) and has a pH of 5 to 6 (e.g., 5.5).

In some embodiments, the formulation (e.g., liquid formulation) further comprises a carbohydrate. In certain embodiments, the carbohydrate is sucrose. In some embodiments, the carbohydrate (e.g., sucrose) is present at a concentration of 50 mM to 500 mM, e.g., 100 mM to 400 mM, 150 mM to 300 mM, 180 mM to 250 mM, 200 mM to 240 mM, 210 mM to 230 mM, 100 mM to 300 mM, 100 mM to 250 mM, 100 mM to 200 mM, 100 mM to 150 mM, 300 mM to 400 mM, 200 mM to 400 mM, or 100 mM to 400 mM, e.g., 100 mM, 150 mM, 180 mM, 200 mM, 220 mM, 250 mM, 300 mM, 350 mM, or 400 mM. In some embodiments, the formulation comprises a carbohydrate or sucrose present at a concentration of 200 mM to 250 mM, e.g., 220 mM.

In some embodiments, the formulation (e.g., liquid formulation) comprises an antibody molecule as disclosed herein present at a concentration of 80 to 120 mg/mL, e.g., 100 mg/mL; a buffering agent that comprises a histidine buffer at a concentration of 15 mM to 25 mM (e.g., 20 mM) and has a pH of 5 to 6 (e.g., 5.5); and a carbohydrate or sucrose present at a concentration of 200 mM to 250 mM, e.g., 220 mM.

In some embodiments, the formulation (e.g., liquid formulation) further comprises a surfactant. In certain embodiments, the surfactant is polysorbate 20. In some embodiments, the surfactant or polysorbate 20) is present at a concentration of 0.005% to 0.1% (w/w), e.g., 0.01% to 0.08%, 0.02% to 0.06%, 0.03% to 0.05%, 0.01% to 0.06%, 0.01% to 0.05%, 0.01% to 0.03%, 0.06% 20 to 0.08%, 0.04% to 0.08%, or 0.02% to 0.08% (w/w), e.g., 0.01%, 0.02%, 0.03%, 0.04%, 0.05%, 0.06%, 0.07%, 0.08%, 0.09%, or 0.1% (w/w). In some embodiments, the formulation comprises a surfactant or polysorbate 20 present at a concentration of 0.03% to 0.05%, e.g., 0.04% (w/w).

In some embodiments, the formulation (e.g., liquid formulation) comprises an antibody molecule as disclosed herein present at a concentration of 80 to 120 mg/mL, e.g., 100 mg/mL; a buffering agent that comprises a histidine buffer at a concentration of 15 mM to 25 mM (e.g., 20 mM) and has a pH of 5 to 6 (e.g., 5.5); a carbohydrate or sucrose present at a concentration of 200 mM to 250 mM, e.g., 220 mM; and a surfactant or polysorbate 20 present at a concentration of 0.03% to 0.05%, e.g., 0.04% (w/w).

In some embodiments, the formulation (e.g., liquid formulation) comprises an antibody molecule as disclosed herein present at a concentration of 100 mg/mL; a buffering agent that comprises a histidine buffer (e.g., histidine/histidine-HCL) at a concentration of 20 mM) and has a pH of 5.5; a carbohydrate or sucrose present at a concentration of 220 mM; and a surfactant or polysorbate 20 present at a concentration of 0.04% (w/w).

In some embodiments, the liquid formulation is prepared by diluting a formulation comprising an antibody molecule described herein. For example, a drug substance formulation can be diluted with a solution comprising one or more excipients (e.g., concentrated excipients). In some embodiments, the solution comprises one, two, or all of histidine, sucrose, or polysorbate 20. In certain embodiments, the solution comprises the same excipient(s) as the drug substance formulation. Exemplary excipients include, but are not limited to, an amino acid (e.g., histidine), a carbohydrate (e.g., sucrose), or a surfactant (e.g., polysorbate 20). In certain embodiments, the liquid formulation is not a reconstituted lyophilized formulation. In other embodiments, the liquid formulation is a reconstituted lyophilized formulation. In some embodiments, the formulation is stored as a liquid. In other embodiments, the formulation is prepared as a liquid and then is dried, e.g., by lyophilization or spray-drying, prior to storage.

In certain embodiments, 0.5 mL to 10 mL (e.g., 0.5 mL to 8 mL, 1 mL to 6 mL, or 2 mL to 5 mL, e.g., 1 mL, 1.2 mL, 1.5 mL, 2 mL, 3 mL, 4 mL, 4.5 mL, or 5 mL) of the liquid formulation is filled per container (e.g., vial). In other embodiments, the liquid formulation is filled into a container (e.g., vial) such that an extractable volume of at least 1 mL (e.g., at least 1.2 mL, at least 1.5 mL, at least 2 mL, at least 3 mL, at least 4 mL, or at least 5 mL) of the liquid formulation can be withdrawn per container (e.g., vial). In certain embodiments, the liquid formulation is extracted from the container (e.g., vial) without diluting at a clinical site. In certain embodiments, the liquid formulation is diluted from a drug substance formulation and extracted from the container (e.g., vial) at a clinical site. In certain embodiments, the formulation (e.g., liquid formulation) is injected to an infusion bag, e.g., within 1 hour (e.g., within 45 minutes, 30 minutes, or 15 minutes) before the infusion starts to the patient.

A formulation described herein can be stored in a container. The container used for any of the formulations described herein can include, e.g., a vial, and optionally, a stopper, a cap, or both. In certain embodiments, the vial is a glass vial, e.g., a 6R white glass vial. In other embodiments, the stopper is a rubber stopper, e.g., a grey rubber stopper. In other embodiments, the cap is a flip-off cap, e.g., an aluminum flip-off cap. In some embodiments, the container comprises a 6R white glass vial, a grey rubber stopper, and an aluminum flip-off cap. In some embodiments, the container (e.g., vial) is for a single-use container. In certain embodiments, 25 mg/mL to 250 mg/mL, e.g., 50 mg/mL to 200 mg/mL, 60 mg/mL to 180 mg/mL, 70 mg/mL to 150 mg/mL, 80 mg/mL to 120 mg/mL, 90 mg/mL to 110 mg/mL, 50 mg/mL to 150 mg/mL, 50 mg/mL to 100 mg/mL, 150 mg/mL to 200 mg/mL, or 100 mg/mL to 200 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL, of the antibody molecule as described herein, is present in the container (e.g., vial).

In some embodiments, the formulation is a lyophilized formulation. In certain embodiments, the lyophilized formulation is lyophilized or dried from a liquid formulation comprising an antibody molecule described herein. For example, 1 to 5 mL, e.g., 1 to 2 mL, of a liquid formulation can be filled per container (e.g., vial) and lyophilized.

In some embodiments, the formulation is a reconstituted formulation. In certain embodiments, the reconstituted formulation is reconstituted from a lyophilized formulation comprising an antibody molecule described herein. For example, a reconstituted formulation can be prepared by dissolving a lyophilized formulation in a diluent such that the protein is dispersed in the reconstituted formulation. In some embodiments, the lyophilized formulation is reconstituted with 1 mL to 5 mL, e.g., 1 mL to 2 mL, e.g., 1.2 mL, of water or buffer for injection. In certain embodiments, the lyophilized formulation is reconstituted with 1 mL to 2 mL of water for injection, e.g., at a clinical site.

In some embodiments, the reconstituted formulation comprises an antibody molecule (e.g., an anti-TIM-3, anti-TGF-β, anti-PD-1, or anti-IL-1β antibody molecule as disclosed herein) and a buffering agent.

In some embodiments, the reconstituted formulation comprises an comprises an anti-TIM-3, anti-TGF-β, anti-P-D1, or anti-IL-1β antibody molecule as disclosed herein present at a concentration of 25 mg/mL to 250 mg/mL, e.g., 50 mg/mL to 200 mg/mL, 60 mg/mL to 180 mg/mL, 70 mg/mL to 150 mg/mL, 80 mg/mL to 120 mg/mL, 90 mg/mL to 110 mg/mL, 50 mg/mL to 150 mg/mL, 50 mg/mL to 100 mg/mL, 150 mg/mL to 200 mg/mL, or 100 mg/mL to 200 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL. In certain embodiments, the antibody molecule is present at a concentration of 80 mg/mL to 120 mg/mL, e.g., 100 mg/mL.

In some embodiments, the reconstituted formulation comprises a buffering agent comprising histidine (e.g., a histidine buffer). In certain embodiments, the buffering agent (e.g., histidine buffer) is present at a concentration of 1 mM to 100 mM, e.g., 2 mM to 50 mM, 5 mM to 40 mM, 10 mM to 30 mM, 15 to 25 mM, 5 mM to 40 mM, 5 mM to 30 mM, 5 mM to 20 mM, 5 mM to 10 mM, 40 mM to 50 mM, 30 mM to 50 mM, 20 mM to 50 mM, 10 mM to 50 mM, or 5 mM to 50 mM, e.g., 2 mM, 5 mM, 10 mM, 15 mM, 20 mM, 25 mM, 30 mM, 35 mM, 40 mM, 45 mM, or 50 mM. In some embodiments, the buffering agent (e.g., histidine buffer) is present at a concentration of 15 mM to 25 mM, e.g., 20 mM. In other embodiments, the buffering agent (e.g., a histidine buffer) has a pH of 4 to 7, e.g., 5 to 6, e.g., 5, 5.5, or 6. In some embodiments, the buffering agent (e.g., histidine buffer) has a pH of 5 to 6, e.g., 5.5. In certain embodiments, the buffering agent comprises a histidine buffer at a concentration of 15 mM to 25 mM (e.g., 20 mM) and has a pH of 5 to 6 (e.g., 5.5). In certain embodiments, the buffering agent comprises histidine and histidine-HCl.

In some embodiments, the reconstituted formulation comprises an antibody molecule as disclosed herein present at a concentration of 80 to 120 mg/mL, e.g., 100 mg/mL; and a buffering agent that comprises a histidine buffer at a concentration of 15 mM to 25 mM (e.g., 20 mM) and has a pH of 5 to 6 (e.g., 5.5).

In some embodiments, the reconstituted formulation further comprises a carbohydrate. In certain embodiments, the carbohydrate is sucrose. In some embodiments, the carbohydrate (e.g., sucrose) is present at a concentration of 50 mM to 500 mM, e.g., 100 mM to 400 mM, 150 mM to 300 mM, 180 mM to 250 mM, 200 mM to 240 mM, 210 mM to 230 mM, 100 mM to 300 mM, 100 mM to 250 mM, 100 mM to 200 mM, 100 mM to 150 mM, 300 mM to 400 mM, 200 mM to 400 mM, or 100 mM to 400 mM, e.g., 100 mM, 150 mM, 180 mM, 200 mM, 220 mM, 250 mM, 300 mM, 350 mM, or 400 mM. In some embodiments, the formulation comprises a carbohydrate or sucrose present at a concentration of 200 mM to 250 mM, e.g., 220 mM.

In some embodiments, the reconstituted formulation comprises an antibody molecule disclosed herein present at a concentration of 80 to 120 mg/mL, e.g., 100 mg/mL; a buffering agent that comprises a histidine buffer at a concentration of 15 mM to 25 mM (e.g., 20 mM) and has a pH of 5 to 6 (e.g., 5.5); and a carbohydrate or sucrose present at a concentration of 200 mM to 250 mM, e.g., 220 mM.

In some embodiments, the reconstituted formulation further comprises a surfactant. In certain embodiments, the surfactant is polysorbate 20. In some embodiments, the surfactant or polysorbate 20) is present at a concentration of 0.005% to 0.1% (w/w), e.g., 0.01% to 0.08%, 0.02% to 0.06%, 0.03% to 0.05%, 0.01% to 0.06%, 0.01% to 0.05%, 0.01% to 0.03%, 0.06% to 0.08%, 0.04% to 0.08%, or 0.02% to 0.08% (w/w), e.g., 0.01%, 0.02%, 0.03%, 0.04%, 0.05%, 0.06%, 0.07%, 0.08%, 0.09%, or 0.1% (w/w). In some embodiments, the formulation comprises a surfactant or polysorbate 20 present at a concentration of 0.03% to 0.05%, e.g., 0.04% (w/w).

In some embodiments, the reconstituted formulation comprises an antibody molecule as disclosed herein present at a concentration of 80 to 120 mg/mL, e.g., 100 mg/mL; a buffering agent that comprises a histidine buffer at a concentration of 15 mM to 25 mM (e.g., 20 mM) and has a pH of 5 to 6 (e.g., 5.5); a carbohydrate or sucrose present at a concentration of 200 mM to 250 mM, e.g., 220 mM; and a surfactant or polysorbate 20 present at a concentration of 0.03% to 0.05%, e.g., 0.04% (w/w).

In some embodiments, the reconstituted formulation comprises an antibody molecule as disclosed herein present at a concentration of 100 mg/mL; a buffering agent that comprises a histidine buffer (e.g., histidine/histidine-HCL) at a concentration of 20 mM) and has a pH of 5.5; a carbohydrate or sucrose present at a concentration of 220 mM; and a surfactant or polysorbate 20 present at a concentration of 0.04% (w/w).

In some embodiments, the formulation is reconstituted such that an extractable volume of at least 1 mL (e.g., at least 1.2 mL, 1.5 mL, 2 mL, 2.5 mL, or 3 mL) of the reconstituted formulation can be withdrawn from the container (e.g., vial) containing the reconstituted formulation. In certain embodiments, the formulation is reconstituted and/or extracted from the container (e.g., vial) at a clinical site. In certain embodiments, the formulation (e.g., reconstituted formulation) is injected to an infusion bag, e.g., within 1 hour (e.g., within 45 minutes, 30 minutes, or 15 minutes) before the infusion starts to the patient.

Other exemplary buffering agents that can be used in the formulation described herein include, but are not limited to, an arginine buffer, a citrate buffer, or a phosphate buffer. Other exemplary carbohydrates that can be used in the formulation described herein include, but are not limited to, trehalose, mannitol, sorbitol, or a combination thereof. The formulation described herein may also contain a tonicity agent, e.g., sodium chloride, and/or a stabilizing agent, e.g., an amino acid (e.g., glycine, arginine, methionine, or a combination thereof).

The antibody molecules can be administered by a variety of methods known in the art, although for many therapeutic applications, the preferred route/mode of administration is intravenous injection or infusion. For example, the antibody molecules can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody molecule can be orally administered, for example, with an inert diluent or an assimilable edible carrier. The compound (and other ingredients, if desired) may also be enclosed in a hard or soft-shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

The antibody molecule can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the antibody is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In some embodiments, the anti-TIM3 antibody is administered in combination with a TGF-β inhibitor, e.g. an anti-TGF-β antibody as described herein. In certain embodiments, the TGF-β inhibitor is administered intravenously. An exemplary, non-limiting ranges for a therapeutically or prophylactically effective amount of the TGF-β inhibitor are about 1000 mg to about 2500, typically about 1300 mg to about 2200 mg. In certain embodiments, the TGF-β inhibitor is administered by injection (e.g., subcutaneously or intravenously) at a dose (e.g., a flat dose) of about 1300 mg to about 1500 mg (e.g., about 1400 mg) or about 2000 mg to about 2200 mg (e.g. about 2100 mg). The dosing schedule (e.g., flat dosing schedule) can vary from e.g., once a week to once every 2, 3, 4, 5, or 6 weeks. In one embodiment, the TGF-β inhibitor is administered at a dose from about 1300 mg to about 1500 mg (e.g., about 1400 mg) once every two weeks or once every three weeks. In one embodiment, the TGF-β inhibitor is administered at a dose from about 2000 mg to about 2200 mg (e.g., about 2100 mg) once every two weeks or once every three weeks.

In some embodiments, the anti-TIM3 antibody molecule and anti-TGF-β antibody molecule are administered in combination with a PD-1 inhibitor described herein (e.g., an anti-PD-1 antibody). In certain embodiments, the anti-PD-1 antibody is administered intravenously. An exemplary, non-limiting range for a therapeutically or prophylactically effective amount of an anti-PD-1 antibody is about 100 mg to about 600 mg, typically 200 mg to about 500 mg. In certain embodiments, the anti-PD-1 antibody is administered by injection (e.g., subcutaneously or intravenously) at a dose (e.g., a flat dose) of about 300 mg to about 500 mg (e.g., about 400 mg), or about 200 mg to about 400 mg (e.g., about 300 mg). The dosing schedule (e.g., flat dosing schedule) can vary from e.g., once a week to once every 2, 3, 4, 5, or 6 weeks. In one embodiment, the anti-PD-1 antibody is administered at a dose from about 300 mg to about 500 mg (e.g., about 400 mg) once every three weeks or once every four weeks. In one embodiment, the anti-PD-1 antibody is administered at a dose from about 200 mg to about 400 mg (e.g., about 300 mg) once every three weeks or once every four weeks.

In some embodiments, the anti-TIM3 antibody molecule and anti-TGF-β antibody molecule are administered in combination with a hypomethylating agent described herein. An exemplary, non-limiting range for a therapeutically or prophylactically effective amount of a hypomethylating agent is 2 mg/m² to about 50 mg/m², typically 2 mg/m² to 25 mg/m². In certain embodiments, the hypomethylating agent is administered by injection (e.g., subcutaneously or intravenously) at a dose of about 2 mg/m² to about 4 mg/m² (about 2.5 mg/m²), about 4 mg/m² to about 6 mg/m² (about 5 mg/m²), about 6 mg/m² to about 8 mg/m² (about 7.5 mg/m²), about 8 mg/m² to about 12 mg/m² (about 10 mg/m²), about 12 mg/m² to about 18 mg/m² (about 15 mg/m²), or about 18 mg/m² to about 25 mg/m² (about 20 mg/m²). In some embodiments, the dosing schedule (e.g., flat dosing schedule) can vary during a 42-day cycle, from e.g., once a day for days 1-3). In some embodiments, the dosing schedule (e.g., flat dosing schedule) can vary during a 42-day cycle, from e.g., once a day for days 1-5. In some embodiments, the dosing schedule (e.g., flat dosing schedule) can vary during a 28-day cycle, from e.g., once a day for days 1-3. In some embodiments, the dosing schedule (e.g., flat dosing schedule) can vary during a 28-day cycle, from e.g., once a day for days 1-5. In some embodiments, the dosing schedule (e.g., flat dosing schedule) can vary during a 42-day cycle, from e.g., once every 8 hours for days 1-3. In some embodiments, the dosing schedule (e.g., a ramp-up dosing schedule) can vary during a 42-day cycle, from once a day for days 1-3. In some embodiments, the dosing schedule (e.g., a ramp-up dosing schedule) can vary during a 42-day cycle, from once a day for days 1-5. For example, the doses for Cycle 1 Day 1, Day 2, and Day 3 and beyond are about 5 mg/m², about 10 mg/m², and about 20 mg/m², respectively.

In some embodiments, the anti-TIM3 antibody molecule and anti-TGF-β antibody molecule are administered in combination with an anti-IL-1β antibody molecule as described herein. In certain embodiments, the anti-IL-1β antibody molecule is administered intravenously. In certain embodiments, the anti-IL-1β antibody molecule is administered subcutaneously. An exemplary, non-limiting range for a therapeutically or prophylactically effective amount of an anti-IL-1β antibody molecule is 200 mg once every three weeks or 250 mg once every four weeks.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the modified antibody or antibody fragment is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the disclosure is a kit comprising a combination, composition, or formulation described herein. The kit can include one or more other elements including: instructions for use (e.g., in accordance a dosage regimen described herein); other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

### Use of the Combinations

The combinations described herein can be used to modify an immune response in a subject. In some embodiments, the immune response is enhanced, stimulated or up-regulated. In certain embodiments, the immune response is inhibited, reduced, or down-regulated. For example, the combinations can be administered to cells in culture, e.g. in vitro or ex vivo, or in a subject, e.g., in vivo, to treat, prevent, and/or diagnose a variety of disorders, such as cancers and immune disorders. In some embodiments, the combination results in a synergistic effect. In other embodiments, the combination results in an additive effect. The combinations described herein can be used for treating a disorder described herein (e.g., a cancer described herein) in a subject in accordance with a method described herein. The combination described herein can also be used in the manufacture of medicament for treating a disorder described herein (e.g., a cancer described herein) in a subject in accordance with a method described herein.

As used herein, the term “subject” is intended to include human and non-human animals. In some embodiments, the subject is a human subject. The term “non-human animals” includes mammals and non-mammals, such as non-human primates. In some embodiments, the subject is a human. In some embodiments, the subject is a human patient in need of enhancement of an immune response. The combinations described herein are suitable for treating human patients having a disorder that can be treated by modulating (e.g., augmenting or inhibiting) an immune response. In certain embodiments, the patient has or is at risk of having a disorder described herein, e.g., a cancer described herein. In some embodiments, the subject is need of treatment of a disorder described herein (e.g., a cancer described herein), e.g., using a combination described herein.

In some embodiments, the combination is used to treat a myelofibrosis (e.g., primary myelofibrosis (PMF), post-essential thrombocythemia myelofibrosis (PET-MF), post-polycythemia vera myelofibrosis (PPV-MF)), leukemia (e.g., an acute myeloid leukemia (AML), e.g., a relapsed or refractory AML or a de novo AML; or a chronic lymphocytic leukemia (CLL)), a lymphoma (e.g., T-cell lymphoma, B-cell lymphoma, a non-Hogdkin lymphoma, or a small lymphocytic lymphoma (SLL)), a myeloma (e.g., multiple myeloma), a lung cancer (e.g., a non-small cell lung cancer (NSCLC) (e.g., a NSCLC with squamous and/or non-squamous histology, or a NSCLC adenocarcinoma), or a small cell lung cancer (SCLC)), a skin cancer (e.g., a Merkel cell carcinoma or a melanoma (e.g., an advanced melanoma)), an ovarian cancer, a mesothelioma, a bladder cancer, a soft tissue sarcoma (e.g., a hemangiopericytoma (HPC)), a bone cancer (a bone sarcoma), a kidney cancer (e.g., a renal cancer (e.g., a renal cell carcinoma)), a liver cancer (e.g., a hepatocellular carcinoma), a cholangiocarcinoma, a sarcoma, a myelodysplastic syndrome (MDS) (e.g., a lower risk MDS (e.g., a very low risk MDS, a low risk MDS, or an intermediate risk MDS) or a higher risk MDS (e.g., a high risk MDS or a very high risk MDS)), a prostate cancer, a breast cancer (e.g., a breast cancer that does not express one, two or all of estrogen receptor, progesterone receptor, or Her2/neu, e.g., a triple negative breast cancer), a colorectal cancer, a nasopharyngeal cancer, a duodenal cancer, an endometrial cancer, a pancreatic cancer, a head and neck cancer (e.g., head and neck squamous cell carcinoma (HNSCC), an anal cancer, a gastro-esophageal cancer, a thyroid cancer (e.g., anaplastic thyroid carcinoma), a cervical cancer, or a neuroendocrine tumor (NET) (e.g., an atypical pulmonary carcinoid tumor).

In some embodiments, the cancer is a hematological cancer, e.g., myeloproliferative neoplasm, a leukemia, a lymphoma, or a myeloma. For example, an combination described herein can be used to treat cancers and malignancies including, but not limited to, e.g., a myelofibrosis, a primary myelofibrosis (PMF), post-essential thrombocythemia myelofibrosis (PET-MF), post-polycythemia vera myelofibrosis (PPV-MF), essential thrombocythemia, polycythemia vera, an acute leukemia, e.g., B-cell acute lymphoid leukemia (BALL), T-cell acute lymphoid leukemia (TALL), acute myeloid leukemia (AML), acute lymphoid leukemia (ALL); a chronic leukemia, e.g., chronic myelogenous leukemia (CML), chronic lymphocytic leukemia (CLL); an additional hematologic cancer or hematologic condition, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome (e.g., a lower risk MDS (e.g., a very low risk MDS, a low risk MDS, or an intermediate risk MDS) or a higher risk MDS (e.g., a high risk MDS or a very high risk MDS)), non-Hodgkin's lymphoma, plasmablastic lymphoma, plasmacytoid dendritic cell neoplasm, Waldenström macroglobulinemia, and “preleukemia” which are a diverse collection of hematological conditions united by ineffective production (or dysplasia) of myeloid blood cells, and the like.

In some embodiments, the combination is used to treat a myeloproliferative neoplasm, e.g., a myelofibrosis, e.g., primary myelofibrosis (PMF), post-essential thrombocythemia myelofibrosis (PET-MF), post-polycythemia vera myelofibrosis (PPV-MF). In some embodiments, the combination is used to treat primary myelofibrosis. In some embodiments, the subject has been treated with a janus kinase inhibitor (JAK inhibitor) with selectivity for subtypes JAK1 and JAK2, e.g., ruxolitinib. In some embodiments, the subject has not been treated with a janus kinase inhibitor (JAK inhibitor) with selectivity for subtypes JAK1 and JAK2, e.g., ruxolitinib.

In some embodiments, the combination is used to treat a leukemia, e.g., an acute myeloid leukemia (AML) or a chronic lymphocytic leukemia (CLL). In some embodiments, the combination is used to treat a lymphoma, e.g., a small lymphocytic lymphoma (SLL). In some embodiments, the combination is used to treat a myeloma, e.g., a multiple myeloma (MM). In certain embodiments, the patient is not suitable for a standard therapeutic regimen with established benefit in patients with a hematological cancer described herein. In some embodiments, the subject is unfit for a chemotherapy. In some embodiments, the chemotherapy is an intensive induction chemotherapy. For example, the combinations described herein can be used for the treatment of adult patients with chronic lymphocytic leukemia (CLL) or small lymphocytic lymphoma (SLL). As another example, the combinations described herein can be used for the treatment of newly-diagnosed acute myeloid leukemia (AML) in adults who are age 75 years or older, or who have comorbidities that preclude use of intensive induction chemotherapy.

In certain embodiments, the subject has been identified as having TIM-3 expression in tumor infiltrating lymphocytes. In other embodiments, the subject does not have detectable level of TIM-3 expression in tumor infiltrating lymphocytes.

### Methods of Treating Cancer

In one aspect, the disclosure relates to treatment of a subject in vivo using a combination described herein, or a composition or formulation comprising a combination described herein, such that growth of cancerous tumors is inhibited or reduced.

In certain embodiments, the combination comprises a TIM-3 inhibitor, a TGF-β inhibitor, optionally a hypomethylating agent, and optionally a PD-1 inhibitor, or an IL-1β inhibitor. In certain embodiments, the combination comprises a TIM-3 inhibitor, a TGF-β inhibitor, and optionally an IL-1β inhibitor. In some embodiments, the TIM-3 inhibitor, the TGF-β inhibitor, and optionally the PD-1 inhibitor, hypomethylating agent, or IL-1β inhibitor is administered or used in accordance with a dosage regiment disclosed herein. In certain embodiments, the combination is administered in an amount effective to treat a cancer or a symptom thereof.

The combinations, compositions, or formulations described herein can be used to inhibit the growth of cancerous tumors. Alternatively, the combinations, compositions, or formulations described herein can be used in combination with one or more of: a standard of care treatment for cancer, another antibody or antigen-binding fragment thereof, an immunomodulator (e.g., an activator of a costimulatory molecule or an inhibitor of an inhibitory molecule); a vaccine, e.g., a therapeutic cancer vaccine; or other forms of cellular immunotherapy, as described herein.

Accordingly, in one embodiment, the disclosure provides a method of inhibiting growth of tumor cells in a subject, comprising administering to the subject a therapeutically effective amount of a combination described herein, e.g., in accordance with a dosage regimen described herein. In an embodiment, the combination is administered in the form of a composition or formulation described herein.

In one embodiment, the combination is suitable for the treatment of cancer in vivo. To achieve antigen-specific enhancement of immunity, the combination can be administered together with an antigen of interest. When a combination described herein is administered the combination can be administered in either order or simultaneously.

In another aspect, a method of treating a subject, e.g., reducing or ameliorating, a hyperproliferative condition or disorder (e.g., a cancer), e.g., solid tumor, a hematological cancer, soft tissue tumor, or a metastatic lesion, in a subject is provided. The method includes administering to the subject a combination described herein, or a composition or formulation comprising a combination described herein, in accordance with a dosage regimen disclosed herein.

As used herein, the term “cancer” is meant to include all types of cancerous growths or oncogenic processes, metastatic tissues or malignantly transformed cells, tissues, or organs, irrespective of histopathological type or stage of invasiveness. Examples of cancerous disorders include, but are not limited to, hematological cancers, solid tumors, soft tissue tumors, and metastatic lesions.

In certain embodiments, the cancer is a hematological cancer. Examples of hematological cancers include, but are not limited to, myelofibrosis, primary myelofibrosis (PMF), post-essential thrombocythemia myelofibrosis (PET-MF), post-polycythemia vera myelofibrosis (PPV-MF), polycythemia vera (PV), essential thrombocythemia, myelodysplastic syndrome (MDS), lower risk myelodysplastic syndrome (MDS), higher risk myelodysplastic syndrome, acute myeloid leukemia, chronic lymphocytic leukemia, small lymphocytic lymphoma, multiple myeloma, acute lymphocytic leukemia, non-Hodgkin's lymphoma, Hodgkin's lymphoma, mantle cell lymphoma, follicular lymphoma, Waldenstrom's macroglobulinemia, B-cell lymphoma and diffuse large B-cell lymphoma, precursor B-lymphoblastic leukemia/lymphoma, B-cell chronic lymphocytic leukemia/small lymphocytic lymphoma, B-cell prolymphocytic leukemia, lymphoplasmacytic lymphoma, splenic marginal zone B-cell lymphoma (with or without villous lymphocytes), hairy cell leukemia, plasma cell myeloma/plasmacytoma, extranodal marginal zone B-cell lymphoma of the MALT type, nodal marginal zone B-cell lymphoma (with or without monocytoid B cells), Burkitt's lymphoma, precursor T-lymphoblastic lymphoma/leukemia, T-cell prolymphocytic leukemia, T-cell granular lymphocytic leukemia, aggressive NK cell leukemia, adult T-cell lymphoma/leukemia (HTLV 1-positive), nasal-type extranodal NK/T-cell lymphoma, enteropathy-type T-cell lymphoma, hepatosplenic γ-δ T-cell lymphoma, subcutaneous panniculitis-like T-cell lymphoma, mycosis fungoides/Sézary syndrome, anaplastic large cell lymphoma (T/null cell, primary cutaneous type), anaplastic large cell lymphoma (T-/null-cell, primary systemic type), peripheral T-cell lymphoma not otherwise characterized, angioimmunoblastic T-cell lymphoma, polycythemia vera (PV), myelodysplastic syndrome (MDS), indolent Non-Hodgkin's Lymphoma (iNHL), and aggressive Non-Hodgkin's Lymphoma (aNHL).

In some embodiments, the hematological cancer is a myelofibrosis (e.g., a primary myelofibrosis (PMF), post-essential thrombocythemia myelofibrosis (PET-MF), post-polycythemia vera myelofibrosis (PPV-MF)). In some embodiments, the myelofibrosis is a primary myelofibrosis (PMF).

Examples of solid tumors include, but are not limited to, malignancies, e.g., sarcomas, and carcinomas (including adenocarcinomas and squamous cell carcinomas), of the various organ systems, such as those affecting liver, lung, breast, lymphoid, gastrointestinal (e.g., colon), anal, genitals and genitourinary tract (e.g., renal, urothelial, bladder), prostate, CNS (e.g., brain, neural or glial cells), head and neck, skin, pancreas, and pharynx. Adenocarcinomas include malignancies such as most colon cancers, rectal cancer, renal cancer (e.g., renal-cell carcinoma (e.g., clear cell or non-clear cell renal cell carcinoma), liver cancer, lung cancer (e.g., non-small cell carcinoma of the lung (e.g., squamous or non-squamous non-small cell lung cancer)), cancer of the small intestine, and cancer of the esophagus. Squamous cell carcinomas include malignancies, e.g., in the lung, esophagus, skin, head and neck region, oral cavity, anus, and cervix. In one embodiment, the cancer is a melanoma, e.g., an advanced stage melanoma. The cancer may be at an early, intermediate, late stage or metastatic cancer. Metastatic lesions of the aforementioned cancers can also be treated or prevented using the combinations described herein.

In certain embodiments, the cancer is a solid tumor. In some embodiments, the cancer is an ovarian cancer. In other embodiments, the cancer is a lung cancer, e.g., a small cell lung cancer (SCLC) or a non-small cell lung cancer (NSCLC). In other embodiments, the cancer is a mesothelioma. In other embodiments, the cancer is a skin cancer, e.g., a Merkel cell carcinoma or a melanoma. In other embodiments, the cancer is a kidney cancer, e.g., a renal cell carcinoma (RCC). In other embodiments, the cancer is a bladder cancer. In other embodiments, the cancer is a soft tissue sarcoma, e.g., a hemangiopericytoma (HPC). In other embodiments, the cancer is a bone cancer, e.g., a bone sarcoma. In other embodiments, the cancer is a colorectal cancer. In other embodiments, the cancer is a pancreatic cancer. In other embodiments, the cancer is a nasopharyngeal cancer. In other embodiments, the cancer is a breast cancer. In other embodiments, the cancer is a duodenal cancer. In other embodiments, the cancer is an endometrial cancer. In other embodiments, the cancer is an adenocarcinoma, e.g., an unknown adenocarcinoma. In other embodiments, the cancer is a liver cancer, e.g., a hepatocellular carcinoma. In other embodiments, the cancer is a cholangiocarcinoma. In other embodiments, the cancer is a sarcoma.

In certain embodiments, the cancer is a myelodysplastic syndrome e.g., a lower risk MDS (e.g., a very low risk MDS, a low risk MDS, or an intermediate risk MDS) or a higher risk MDS (e.g., a high risk MDS or a very high risk MDS)). In certain embodiments, the cancer is a lower risk myelodysplastic syndrome (MDS) (e.g., a very low risk MDS, a low risk MDS, or an intermediate risk MDS). In certain embodiments, the cancer is a higher risk myelodysplastic syndrome (MDS) (e.g., a high risk MDS or a very high risk MDS).

In another embodiment, the cancer is a carcinoma (e.g., advanced or metastatic carcinoma), melanoma or a lung carcinoma, e.g., a non-small cell lung carcinoma. In one embodiment, the cancer is a lung cancer, e.g., a non-small cell lung cancer or small cell lung cancer. In some embodiments, the non-small cell lung cancer is a stage I (e.g., stage Ia or Ib), stage II (e.g., stage IIa or IIb), stage III (e.g., stage IIIa or IIIb), or stage IV, non-small cell lung cancer. In one embodiment, the cancer is a melanoma, e.g., an advanced melanoma. In one embodiment, the cancer is an advanced or unresectable melanoma that does not respond to other therapies. In other embodiments, the cancer is a melanoma with a BRAF mutation (e.g., a BRAF V600 mutation). In another embodiment, the cancer is a hepatocarcinoma, e.g., an advanced hepatocarcinoma, with or without a viral infection, e.g., a chronic viral hepatitis. In another embodiment, the cancer is a prostate cancer, e.g., an advanced prostate cancer. In yet another embodiment, the cancer is a myeloma, e.g., multiple myeloma. In yet another embodiment, the cancer is a renal cancer, e.g., a renal cell carcinoma (RCC) (e.g., a metastatic RCC, a non-clear cell renal cell carcinoma (nccRCC), or clear cell renal cell carcinoma (CCRCC)).

In some embodiments, the cancer is an MSI-high cancer. In some embodiments, the cancer is a metastatic cancer. In other embodiments, the cancer is an advanced cancer. In other embodiments, the cancer is a relapsed or refractory cancer.

Exemplary cancers whose growth can be inhibited using the combinations, compositions, or formulations, as disclosed herein, include cancers typically responsive to immunotherapy. Additionally, refractory or recurrent malignancies can be treated using the combinations described herein.

Examples of other cancers that can be treated include, but are not limited to, basal cell carcinoma, biliary tract cancer; bladder cancer; bone cancer; brain and CNS cancer; primary CNS lymphoma; neoplasm of the central nervous system (CNS); breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia (including acute myeloid leukemia, chronic myeloid leukemia, acute lymphoblastic leukemia, chronic lymphocytic leukemia, chronic or acute leukemia); liver cancer; lung cancer (e.g., small cell and non-small cell); lymphoma including Hodgkin's and non-Hodgkin's lymphoma; lymphocytic lymphoma; melanoma, e.g., cutaneous or intraocular malignant melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system, hepatocarcinoma, cancer of the anal region, carcinoma of the fallopian tubes, carcinoma of the vagina, carcinoma of the vulva, cancer of the small intestine, cancer of the endocrine system, cancer of the parathyroid gland, cancer of the adrenal gland, sarcoma of soft tissue, cancer of the urethra, cancer of the penis, solid tumors of childhood, spinal axis tumor, brain stem glioma, pituitary adenoma, Kaposi's sarcoma, epidermoid cancer, squamous cell cancer, T-cell lymphoma, environmentally induced cancers including those induced by asbestos, as well as other carcinomas and sarcomas, and combinations of said cancers.

As used herein, the term “subject” is intended to include human and non-human animals. In some embodiments, the subject is a human subject, e.g., a human patient having a disorder or condition characterized by abnormal TIM-3 functioning. Generally, the subject has at least some TIM-3 protein, including the TIM-3 epitope that is bound by the antibody molecule, e.g., a high enough level of the protein and epitope to support antibody binding to TIM-3. The term “non-human animals” includes mammals and non-mammals, such as non-human primates. In some embodiments, the subject is a human. In some embodiments, the subject is a human patient in need of enhancement of an immune response. The methods and compositions described herein are suitable for treating human patients having a disorder that can be treated by modulating (e.g., augmenting or inhibiting) an immune response.

Methods and compositions disclosed herein are useful for treating metastatic lesions associated with the aforementioned cancers.

In some embodiments, the method further comprises determining whether a tumor sample is positive for one or more of PD-L1, CD8, and IFN-γ, and if the tumor sample is positive for one or more, e.g., two, or all three, of the markers, then administering to the patient a therapeutically effective amount of an anti-TIM-3 antibody molecule, optionally in combination with one or more other immunomodulators or anti-cancer agents, as described herein.

In some embodiments, the combination described herein is used to treat a cancer that expresses TIM-3. TIM-3-expressing cancers include, but are not limited to, cervical cancer (Cao et al., PLoS One. 2013; 8(1): e53834), lung cancer (Zhuang et al., Am J Clin Pathol. 2012; 137(6):978-985) (e.g., non-small cell lung cancer), acute myeloid leukemia (Kikushige et al., Cell Stem Cell. 2010 Dec. 3; 7(6):708-17), diffuse large B cell lymphoma, melanoma (Fourcade et al., JEM, 2010; 207 (10): 2175), renal cancer (e.g., renal cell carcinoma (RCC), e.g., kidney clear cell carcinoma, kidney papillary cell carcinoma, or metastatic renal cell carcinoma), squamous cell carcinoma, esophageal squamous cell carcinoma, nasopharyngeal carcinoma, colorectal cancer, breast cancer (e.g., a breast cancer that does not express one, two or all of estrogen receptor, progesterone receptor, or Her2/neu, e.g., a triple negative breast cancer), mesothelioma, hepatocellular carcinoma, and ovarian cancer. The TIM-3-expressing cancer may be a metastatic cancer.

In other embodiments, the combination described herein is used to treat a cancer that is characterized by macrophage activity or high expression of macrophage cell markers. In an embodiment, the combination is used to treat a cancer that is characterized by high expression of one or more of the following macrophage cell markers: LILRB4 (macrophage inhibitory receptor), CD14, CD16, CD68, MSR1, SIGLEC1, TREM2, CD163, ITGAX, ITGAM, CD11b, or CD11c. Examples of such cancers include, but are not limited to, diffuse large B-cell lymphoma, glioblastoma multiforme, kidney renal clear cell carcinoma, pancreatic adenocarcinoma, sarcoma, liver hepatocellular carcinoma, lung adenocarcinoma, kidney renal papillary cell carcinoma, skin cutaneous melanoma, brain lower grade glioma, lung squamous cell carcinoma, ovarian serious cystadenocarcinoma, head and neck squamous cell carcinoma, breast invasive carcinoma, acute myeloid leukemia, cervical squamous cell carcinoma, endocervical adenocarcinoma, uterine carcinoma, colorectal cancer, uterine corpus endometrial carcinoma, thyroid carcinoma, bladder urothelial carcinoma, adrenocortical carcinoma, kidney chromophobe, and prostate adenocarcinoma.

The combination therapies described herein can include a composition co-formulated with, and/or co-administered with, one or more therapeutic agents, e.g., one or more anti-cancer agents, cytotoxic or cytostatic agents, hormone treatment, vaccines, and/or other immunotherapies. In other embodiments, the antibody molecules are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

The combinations, compositions, and formulations described herein can be used further in combination with other agents or therapeutic modalities, e.g., a second therapeutic agent chosen from one or more of the agents listed in Table 6 of WO 2017/019897, the content of which is incorporated by reference in its entirety. In one embodiment, the methods described herein include administering to the subject an anti-TIM-3 antibody molecule as described in WO2017/019897 (optionally in combination with one or more inhibitors of PD-1, PD-L1, LAG-3, CEACAM (e.g., CEACAM-1 and/or CEACAM-5), or CTLA-4)), further include administration of a second therapeutic agent chosen from one or more of the agents listed in Table 6 of WO 2017/019897, in an amount effective to treat or prevent a disorder, e.g., a disorder as described herein, e.g., a cancer. When administered in combination, the TIM-3 inhibitor, TGF-β inhibitor, the PD-1 inhibitor, hypomethylating agent, one or more additional agents, or all, can be administered in an amount or dose that is higher, lower or the same than the amount or dosage of each agent used individually, e.g., as a monotherapy. In certain embodiments, the administered amount or dosage of the TIM-3 inhibitor, TGF-β inhibitor, PD-1 inhibitor, hypomethylating agent, one or more additional agents, or all, is lower (e.g., at least 20%, at least 30%, at least 40%, or at least 50%) than the amount or dosage of each agent used individually, e.g., as a monotherapy. In other embodiments, the amount or dosage of the TIM-3 inhibitor, TGF-β inhibitor, PD-1 inhibitor, hypomethylating agent, one or more additional agents, or all, that results in a desired effect (e.g., treatment of cancer) is lower (e.g., at least 20%, at least 30%, at least 40%, or at least 50% lower).

In other embodiments, the additional therapeutic agent is chosen from one or more of the agents listed in Table 6 of WO 2017/019897. In some embodiments, the additional therapeutic agent is chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or a 17alpha-Hydroxylase/C17-20 Lyase inhibitor); 5) an iron chelating agent; 6) an aromatase inhibitor; 7) an inhibitor of p53, e.g., an inhibitor of a p53/Mdm2 interaction; 8) an apoptosis inducer; 9) an angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) a fibroblast growth factor receptor 2 (FGFR2)/fibroblast growth factor receptor 4 (FGFR4) inhibitor; 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) a BCR-ABL kinase inhibitor; 25) an FGFR inhibitor; 26) an inhibitor of CYP11B2; 27) a HDM2 inhibitor, e.g., an inhibitor of the HDM2-p53 interaction; 28) an inhibitor of a tyrosine kinase; 29) an inhibitor of c-MET; 30) an inhibitor of JAK; 31) an inhibitor of DAC; 32) an inhibitor of 11β-hydroxylase; 33) an inhibitor of IAP; 34) an inhibitor of PIM kinase; 35) an inhibitor of Porcupine; 36) an inhibitor of BRAF, e.g., BRAF V600E or wild-type BRAF; 37) an inhibitor of HER3; 38) an inhibitor of MEK; or 39) an inhibitor of a lipid kinase, e.g., as described in Table 6 of WO 2017/019897.

### EXAMPLES

### Example 1—Pre-Clinical Activity of MBG453

MBG453 is a high-affinity, humanized anti-TIM-3 IgG4 antibody (Ab) (stabilized hinge, S228P), which blocks the binding of TIM-3 to phosphatidylserine (PtdSer). Recent results from a multi-center, open label phase Ib dose-escalation study (CPDR001X2105) in patients with high-risk MDS and no prior hypomethylating agent therapy demonstrated encouraging preliminary efficacy with an overall response rate of 58%, including 47% CR/mCR, with responders continuing on study for up to two years (Borate et al. Blood 2019, 134 (Supplement_1). Preclinical experiments were undertaken to define the mechanism of action for the observed clinical activity of the decitabine and anti-TIM-3 combination in AML and MDS.

MBG453 was determined to partially block the TIM-3/Galectin-9 interaction in a plate-based assay, also supported by a previously determined crystal structure with human TIM-3 (Sabatos-Peyton et al, AACR Annual Meeting Abstract 2016). MBG453 was determined to mediate moderate antibody-dependent cellular phagocytosis (ADCP) as measured by determining the phagocytic uptake of an engineered TIM-3-overexpressing cell line in the presence of MBG453, relative to controls. Pre-treatment of an AML cell line (Thp-1) with decitabine enhanced sensitivity to immune-mediated killing by T cells in the presence of MBG453. MBG453 did not enhance the anti-leukemic activity of decitabine in patient-derived xenograft studies in immuno-deficient hosts.

Taken together, these results support both direct anti-leukemic effects and immune-mediated modulation by MBG453. Importantly, the in vitro activity of MBG453 defines an ability to enhance T cell mediated killing of AML cells.

### Example 2—Clinical Protocol for Combination Treatment of MDS

The following example describes a proposed clinical protocol for evaluating the combination of MBG453 and NIS793 in the treatment of MDS, particularly lower risk MDS. MBG453 and NIS793 will be administered via i.v. infusion over 30 minutes. MBG453 and NIS793 will be given once every 3 weeks (Q3W). Based on emerging clinical data, an alternative dose/schedule may also be evaluated via protocol amendment.

For the purpose of scheduling procedures and evaluations, a cycle is defined as 21 days (MBG453+NIS793 arm).

During the study treatment period patients will be regularly monitored to assess the safety and efficacy of the treatment.

The planned starting doses for MBG453 and NIS793 will be at the doses selected as RD: MBG453 at 600 mg i.v. Q3W, in combination with NIS793 at 2100 mg i.v. Q3W.

### Example 3—Clinical Protocol for Combination Treatment of Myelofibrosis

The following example describes a proposal clinical protocol to evaluate combination treatments of myelofibrosis.

This is a proposed design for an open label study with multiple treatment arms. The design of this study is adaptive to allow dropping of non-tolerated or ineffective combination treatments and facilitate the introduction of new combinations.

This study is comprised of a dose evaluation/escalation part and a dose expansion part.

During the dose evaluation part, a cohort of subjects will be treated with the backbone of MBG453 (recommended dose, RD)+NIS793 (recommended dose, RD) or in combination with a third partner in order to assess the safety and tolerability of the combination at RD. Combinations of MBG453, NIS793, and a third partner (such as decitabine or spartalizumab) will be administered at their respective recommended dose.

As the study progresses and based on emerging clinical data collected from this study, Novartis, in agreement with the study investigator will decide whether or not: to proceed with any treatment arm that reaches recommended dose(s) to explore further the safety, tolerability, and anti-tumor activity in the dose expansion part; to add a third partner to comprise a triplet treatment arm in the dose evaluation/escalation part (such as Treatment Arm2 with decitabine or Treatment Arm 3 with spartalizumab); and to explore MBG453 single agent (treatment arm 4) and/or NIS793 single agent (Treatment Arm 5) in the dose expansion part in order to assess the single agent contributions to efficacy. Dose evaluation or dose escalation with the third partner may occur in parallel.

In case any given treatment combination is considered not tolerable, no RD for that combination will be defined, and enrollment in that treatment combination will be discontinued. Moreover, other investigational drugs or drug combination partners by protocol amendment, if their dosing and safety have been established in other clinical studies.

### Example 4—MBG453 Partially Blocks the Interaction Between TIM-3 and Galectin 9

Galectin-9 is a ligand of TIM-3. Asayama et al. (Oncotarget 8(51): 88904-88971 (2017) demonstrated by the TIM-3-Galectin 9 pathway is associated with the pathogenesis and disease progression of MDS. This example illustrates the ability of MBG453 to partially block the interaction between TIM-3 and Galectin 9.

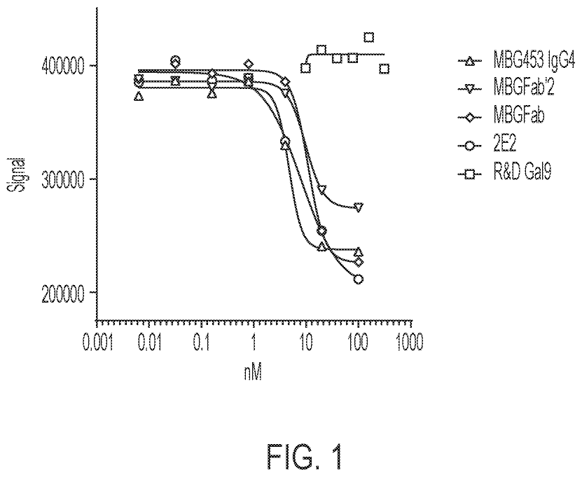

TIM-3 fusion protein (R&D Systems) was coated on a standard MesoScale 96 well plate (Meso Scale Discovery) at 2 μg/ml in PBS (Phosphate Buffered Saline) and incubated for six hours at room temperature. The plate was washed three times with PBST (PBS buffer containing 0.05% Tween-20) and blocked with PBS containing 5% Probumin (Millipore) overnight at 4° C. After incubation, the plate was washed three times with PBST and unlabeled antibody (F38-2E2 (BioLegend); MBG453; MBG453 F(ab′)2; MBG453 F(ab); or control recombinant human Galectin-9 protein) diluted in Assay Diluent (2% Probumin, 0.1% Tween-20, 0.1% Triton X-100 (Sigma) with 10% StabilGuard (SurModics)), was added in serial dilutions to the plate and incubated for one hour on an orbital shaker at room temperature. The plate was then washed three times with PBST, and Galectin-9 labeled with MSD SULFOTag (Meso Scale Discovery) as per manufacturer's instructions, diluted in Assay Diluent to 100 nM, was added to the plate for one hour at room temperature on an orbital shaker. The plate was again washed three times with PBST, and Read Buffer T (1×) was added to the plate. The plate was read on MA600 Imager, and competition was assessed as a measure of the ability of the antibody to block Gal9-SULFOTag signal to TIM-3 receptor. As shown in FIG. 1, MBG453 IgG4, MBG453 F(ab′)2, MBG453 F(ab), and 2E2 partially blocked the interaction between TIM-3 and Galectin-9, whereas control Galectin-9 protein did not.

### Example 5—MBG453 Mediates Antibody-Dependent Cellular Phagocytosis (ADCP) Through Engagement of FcγR1

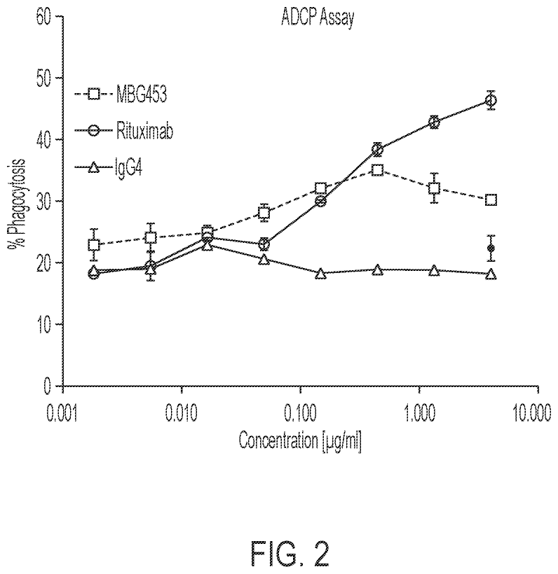

THP-1 effector cells (a human monocytic AML cell line) were differentiated into phagocytes by stimulation with 20 ng/ml phorbol 12-myristate 13-acetate (PMA) for two to three days at 37° C., 5% CO2. PMA-stimulated THP-1 cells were washed in FACS Buffer (PBS with 2 mM EDTA) in the flask and then detached by treatment with Accutase (Innovative Cell Technologies). The target TIM-3-overexpressing Raji cells were labelled with 5.5 μM CellTrace CFSE (ThermoFisherScientific) as per manufacturer's instructions. THP-1 cells and TIM-3-overexpressing CFSE+ Raji cells were co-cultured at an effector to target (E:T) ratio of 1:5 with dilutions of MBG453, MabThera anti-CD20 (Roche) positive control, or negative control antibody (hIgG4 antibody with target not expressed by the Raji TIM-3+ cells) in a 96 well plate (spun at 100×g for 1 minute at room temperature at assay start). Co-cultures were incubated for 30-45 minutes at 37° C., 5% CO2. Phagocytosis was then stopped with a 4% Formaldehyde fixation (diluted from 16% stock, ThermoFisherScientific), and cells were stained with an APC-conjugated anti-CD11c antibody (BD Bioscience). ADCP was measured by a flow cytometry based assay on a BD FACS Canto II. Phagocytosis was evaluated as a percentage of the THP-1 cells double positive for CFSE (representing the phagocytosed Raji cell targets) and CD11c from the THP-1 (effector) population. As shown in FIG. 2, MBG453 (squares) enhanced THP-1 cell phagocytosis of TIM-3+ Raji cells in a dose-dependent manner, which then plateaued relative to the anti-CD20 positive control (open circles). Negative control IgG4 antibody is shown in triangles.

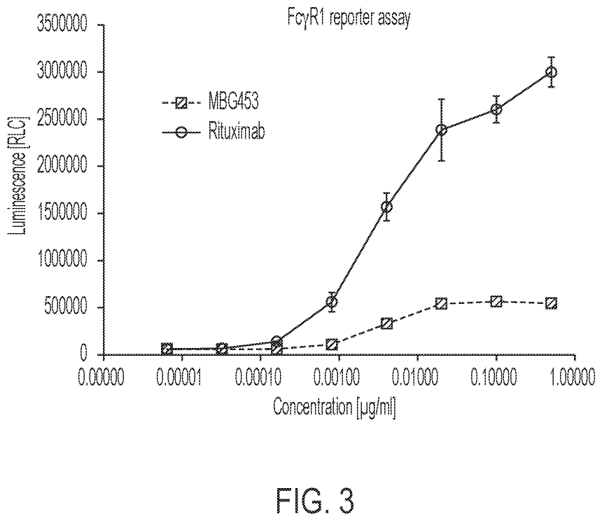

The TIM-3-expressing Raji cells were used as target cells in a co-culture assay with engineered effector Jurkat cells stably transfected to overexpress FcγRIa (CD64) and a luciferase reporter gene under the control of an NFAT (nuclear factor of activated T cells) response element (NFAT-RE; Promega). The target TIM-3+ Raji cells were co-incubated with the Jurkat-FcγRIa reporter cells in an E:T ratio of 6:1 and graded concentrations (500 ng/ml to 6 μg/ml) of MBG453 or the anti-CD20 MabThera reference control (Roche) in a 96 well plate. The plate was then centrifuged at 300×g for 5 minutes at room temperature at the assay start and incubated for 6 hours in a 37° C., 5% CO₂ humidified incubator. The activation of the NFAT dependent reporter gene expression induced by the binding to FcγRIa was quantified by luciferase activity after cell lysis and the addition of a substrate solution (Bio-GLO). As shown in FIG. 3, MBG453 showed a modest dose-response engagement of the FcγRIa reporter cell line as measured by luciferase activity. In a separate assay, MBG453 did not engage FcγRIIa (CD32a).

### Example 6—MBG453 Enhances Immune-Mediated Killing of Decitabine Pre-Treated AML Cells

THP-1 cells were plated in complete RPMI-1640 (Gibco) media (supplemented with 2 mM glutamine, 100 U/ml Pen-Strep, 10 mM HEPES, 1 mM NaPyr, and 10% fetal bovine serum (FBS)). Decitabine (250 or 500 nM; supplemented to media daily for five days) or DMSO control were added for a 5-day incubation at 37° C., 5% CO₂. Two days after plating THP-1 cells, healthy human donor peripheral blood mononuclear cells (PBMCs; Medcor) were isolated from whole blood by centrifugation of sodium citrate CPT tubes at 1,800×g for 20 minutes. At the completion of the spin, the tube was inverted 10 times to mix the plasma and PBMC layers. Cells were washed in 2× volume of PBS/MACS Buffer (Miltenyi) and centrifuged at 250×g for 5 minutes. Supernatant was aspirated, and 1 mL of PBS/MACS Buffer was added following by pipetting to wash the cell pellet. 19 mL of PBS/MACS Buffer were added to wash, followed by a repeat of the centrifugation. Supernatant was aspirated, and the cell pellet was resuspended in 1 mL of complete media, followed by pipetting to a single cell suspension, and the volume was brought up to 10 mL with complete RPMI. 100 ng/mL anti-CD3 (eBioscience) was added to the media for a 48-hour stimulation at 37° C., 5% CO₂. After 5 days culture with decitabine or DMSO, THP-1 cells were harvested and labeled with CellTracker™ Deep Red Dye (ThermoFisher) following manufacturer's instructions.

Labeled THP-1 cells (decitabine pre-treated or DMSO control-treated) were co-cultured with stimulated PBMCs at effector:target (E:T) ratios of 1:1, 1:2, and 1:3 (optimized for each donor, with the target cell number constant at 10,000 cells/well (Costar 96 well flat bottom plate). Wells were treated with either hIgG4 isotype control or MBG453 at 1 μg/mL. The plate was placed in an Incucyte S3, and image phase and red fluorescent channels were captured every 4 hours for 5 days. At the completion of the assay, the target cell number (red events) was normalized to the first imaging time point using the Incucyte image analysis software.

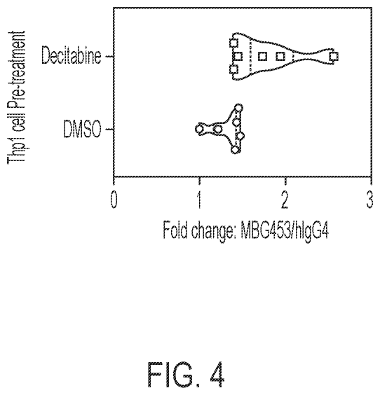

As shown in FIG. 4, co-culture of THP-1 cells with anti-CD3 activated PBMCs led to killing of the THP-1 cells, enhanced in the presence of MBG453 (bars in bottom violin plot, each dot represents a single healthy PBMC donor) relative to hIgG4 isotype control at the terminal timepoint of the assay. This killing was further enhanced by pre-treatment of the THP-1 cells with decitabine (bars in top violin plot, each dot represents a single healthy PBMC donor). Taken together, these data indicate that MBG453 blockade of TIM-3 enhanced immune-mediated killing of THP-1 AML cells, with pre-treatment with decitabine further enhancing this activity.

### Example 7—Investigation of MBG453 and Decitabine-Mediated Killing of Patient-Derived Xenografts in an Immuno-Deficient Host

The activity of MBG453 with and without decitabine was evaluated in two AML patient-derived xenograft (PDX) models, HAMLX21432 and HAMLX5343. Decitabine (TCI America) was formulated in dextrose 5% in water (D5W) freshly prior to each dose and administered daily for 5 days. It was administered at 10 mL/kg intraperitoneal (i.p.), for a final dose volume of 1 mg/kg. MBG453 was formulated to a final concentration of 1 mg/mL in PBS. It was administered weekly at a volume of 10 mL/kg, i.p., for a final dose of 10 mg/kg, with treatment initiating on dosing day 6, 24 hours after the final dose of decitabine. The combination of MBG453 and decitabine was well-tolerated as measured both by body weight change monitoring and visual inspection of health status in both models.

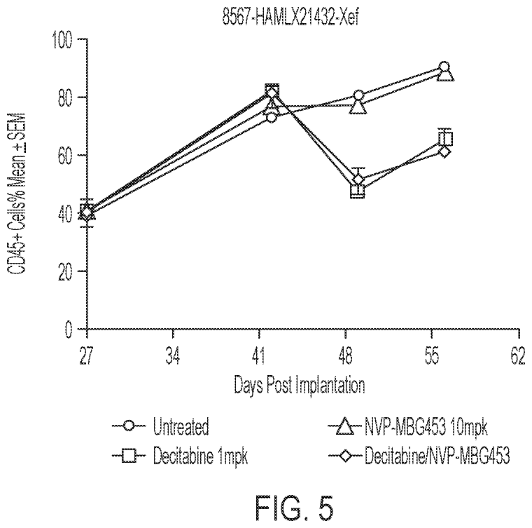

For one study, mice were injected with 2×10⁶ cells intravenously (i.v.) that were isolated from an in vivo passage 5 of the AML PDX #21432 model harboring an IDH1R132H mutation. Animals were randomized into treatment groups once they reached a leukemic burden on average of 39%. Treatments were initiated on the day of randomization and continued for 21 days. Animals remained on study until each reached individual endpoints, defined by circulating leukemic burden of greater than 90% human CD45+ cells, body weight loss >20%, signs of hind limb paralysis, or poor body condition. HAML21432 implanted mice treated with decitabine alone demonstrated moderate anti-tumor activity that peaked at approximately day 49 post-implant or day 14 post-treatment start (At this time point, decitabine-treated groups were on average at 51% and 47% hCD45+ cells, single agent and combination with MBG453, respectively (FIG. 5). At the same time point, the untreated and MBG453-treated groups were at a leukemic burden of 81% and 77%, respectively. By day 56 post-implantation, however, the decitabine-treated groups increased in leukemic burden to 66% and 61% hCD45+ cells in circulation. No combination activity was observed when decitabine was combined with MBG453 in this model (FIG. 5). Untreated and MBG453 single agent treated groups both reached the time to end point cut off of 90% leukemic burden by day 56.

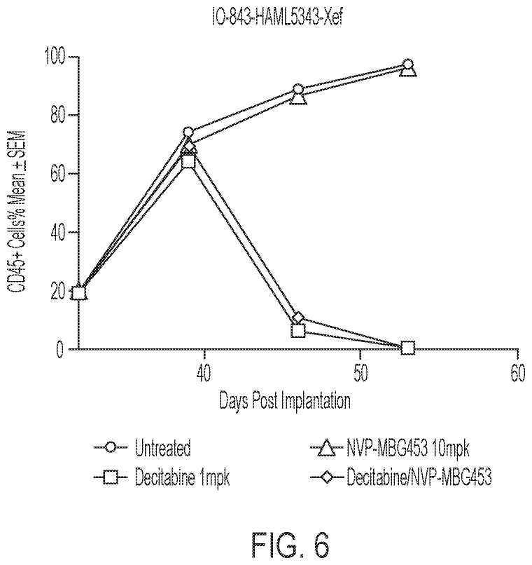

For another study, mice were injected with 2×10⁶ cells i.v. that were isolated from an in vivo passage 4 of the AML PDX #5343 model harboring mutations KRASG12D, WT1 and PTPN11. Animals were randomized into treatment groups once they reached a leukemic burden on average of 20%. Treatments were initiated on the day of randomization and continued for 3 weeks. Animals remained on study until each reached individual endpoints, defined by circulating leukemic burden of greater than 90% human CD45+ cells, body weight loss >20%, signs of hind limb paralysis or poor body condition. HAML5343 implanted mice treated with decitabine alone showed significant anti-tumor activity with a peak of approximately day 53 post-implant or day 21 post-treatment start. At this time point, decitabine-treated groups were on average at 1% and 1.3% hCD45+ cells, single agent and combination with MBG453, respectively (FIG. 6). At the same time point, the untreated group had a leukemic burden of 91%. The MBG453-treated group only had one remaining animal by day 53. No combination activity was observed when decitabine was combined with MBG453 in this model (FIG. 6). The significant reduction in tumor burden was comparable in decitabine single agent and decitabine/MBG453 combination groups in this model.

The Nod scid gamma (NSG; NOD.Cg-prkdc<scid>Il2rg<tm1wj1>/SzJ, Jackson) model used for the AML PDX implantation lacks immune cells, likely such as TIM-3-expressing T cells, NK cells, and myeloid cells, indicating certain immune cell functions may be required for MBG453 to enhance the activity of decitabine in the mouse model.

### Example 8—MBG453 Enhances Killing of Thp-1 AML Cells that are Engineered to Overexpress TIM-3

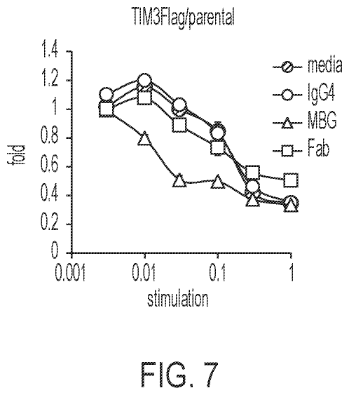

THP-1 cells express TIM-3 mRNA but low to no TIM-3 protein on the cell surface. THP-1 cells were engineered to stably overexpress TIM-3 with a Flag-tag encoded by a lentiviral vector, whereas parental THP-1 cells do not express TIM-3 protein on the surface. TIM-3 Flag-tagged THP-1 cells were labeled with 2 μM CFSE (Thermo Fisher Scientific), and THP-1 parental cells were labeled with 2 μM CTV (Thermo Fisher Scientific), according to manufacturer instructions. Co-culture assays were performed in 96-well round-bottom plates. THP-1 cells were mixed at a 1:1 ratio for a total of 100,000 THP-1 cells per well (50,000 THP-1 expressing TIM-3 and 50,000 THP-1 parental cells) and co-cultured for three days with 100,000 T cells purified using a human pan T cell isolation kit (Miltenyi Biotec) from healthy human donor PBMCs (Bioreclamation), in the presence of varying amounts of anti-CD3/anti-CD28 T cell activation beads (ThermoFisherScientific) and 25 μg/ml MBG453 (whole antibody), MBG453 F(ab), or hIgG4 isotype control. Cells were then detected and counted by flow cytometry. The ratio between TIM-3-expressing THP-1 cells and parental THP-1 cells (“fold” in y-axis of graph) was calculated and normalized to conditions without anti-CD3/anti-CD28 bead stimulation. The x-axis of the graph denotes the stimulation amount as number of beads per cell. Data represents one of two independent experiments. As seen in FIG. 7, MBG453 (triangles) but not MBG453 F(ab) (open squares) enhances the T cell-mediated killing of THP-1 cells that overexpress TIM-3 relative to parental control THP-1 cells indicating that the Fc-portion of MBG453 can be important for MBG453-enhanced T cell-mediated killing of THP-1 AML cells.

### Example 9—TIM-3 Overexpressing Cells Express Low Baseline Levels of IL-1β

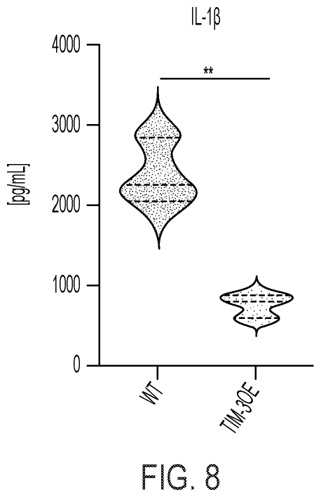

As described in Example 5, THP-1 cells were engineered to overexpress TIM-3. TIM-3-overexpressing and parental control THP-1 cells were stimulated first with 10 μM R848 (Invivogen) for 20 hours and then with 20 μM nigericin (Invivogen) for an additional 4 hours to activate the NLRP3 inflammasome for a total stimulation time of 24 hours. Secreted IL-1β in the cell culture supernatant was measured at 24 hours by a DuoSet ELISA kit for human IL-1β measurement (R&D Systems). As seen in FIG. 8, TIM-3-overexpressing THP-1 cells secreted significantly less IL-1β (unpaired t-test; **p<0.01), demonstrating a potential link between TIM-3 expression on myeloid cells and the NLRP3 inflammasome-mediated production of IL-1R.

### Example 10—Levels of IL-1β mRNA in AML/MDS Patients in the PDR001X2105 Phase I Study

### PDR001x2105 Bulk RNA-Seq Methods

RNA-Seq

Total RNA was extracted from whole blood and bone marrow samples using the Promega Maxwell 16 LEV simply RNA Blood Kit (AS1310). For whole blood samples, the extracted RNA was depleted for globin mRNA using the Invitrogen Globin-Clear Human mRNA removal kit (1980-4). Extracted RNA is enriched for mRNA using poly-T probes which bind to the mRNA's poly-A tail. The enriched mRNA is then fragmented, converted to cDNA, and then carried through the remaining steps of NGS library construction: end repair, A-tailing, indexed adaptor ligation, and PCR amplification using the TruSeq RNA v2 Library Preparation kit (Illumina #15027387 and #15025062). The resulting libraries were sequenced on the Illumina HiSeq to a target depth of 50 million reads.

Next-Generation Sequencing Data Processing

Sequence data was aligned to the hg19 reference human genome using STAR (Dobin, A., Davis, C., Schlesinger, F. et al., Bioinformatics, 2012, 29(1): 15-21). HTSeq was used to quantify the number of reads mapping to each gene (Anders, S., Pyl, P T., and Huber, W. Bioinformatics, 2014, 31(2):doi: 10.1093/bioinformatics/btu638). Gene count data were normalized with edgeR (Robinson, M., McCarthy, D., and Smyth G. Bioinformatics, 2010, 26(1):139-40) using the trimmed mean of M values (TMM) method. All downstream differential expression analyses were performed on the log₂ of the normalized gene count data, after adding 1 to all gene counts to avoid taking the log₂ of 0.

Gene Differential Expression Analysis

Differential expression analyses were performed using Limma (Ritchie M E et al., Nucleic Acids Research, 2015, 43(7):e47) comparing the specified groups. Adjusted p-values were calculated using the Benjamini-Hochberg method and are interpreted as the bounds on the FDR.

### RNA-Seq Results

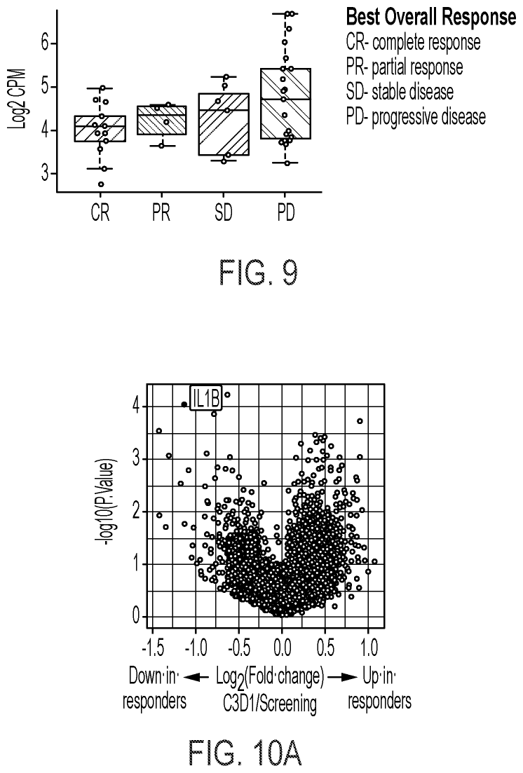

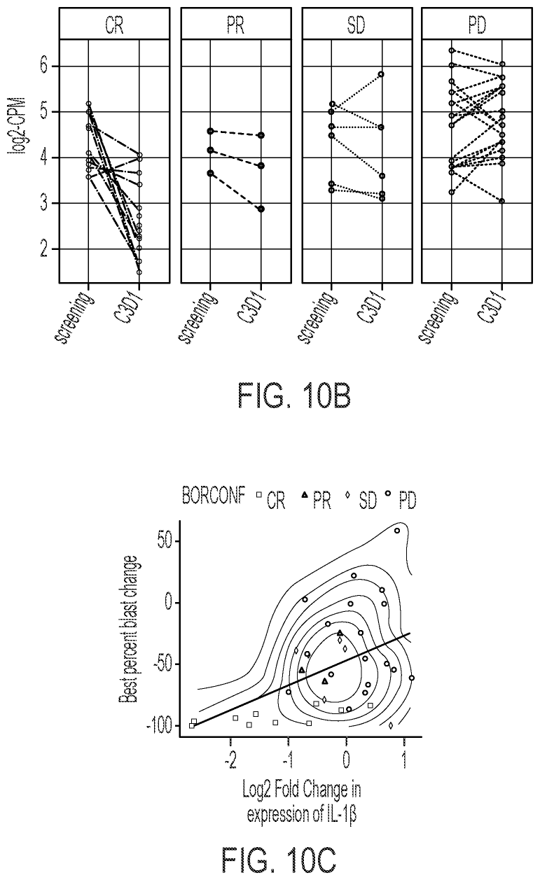

Emerging biomarker data from the PDR001X2105 Phase I study implicate IL-1β as a potential mechanism of resistance to MBG453+hypomethylating agent treatment. Transcriptome-wide analysis of AML/MDS patients treated with the Decitabine and MBG453 combination revealed that higher IL-1β mRNA expression levels were associated with lack of response. In baseline (screening day −28 to day −1) bone marrow samples, the median expression of IL-1β mRNA was higher in patients that had progressive disease (PD) compared to those who had complete response/partial response (CR/PR) in the Decitabine and MBG453 combination cohort (FIG. 9). Moreover, analysis of transcriptional changes induced upon treatment with Decitabine and MBG453 showed that IL-1β was one of the top differentially downregulated genes in the responder group (CR/PR) compared to the non-responder group (Stable Disease/Progressive Disease (SD/PD)) (FIG. 10A). While IL-1β mRNA expression was downregulated upon treatment in the responder group (CR/PR), it remained high in the non-responder group (SD/PD) at the Cycle 3 Day 1 time point (FIG. 10B). Fold changes in IL-1β mRNA expression upon treatment positively correlated with the best percent change in blasts, indicating that higher IL-1β levels on-treatment were associated with higher blast presence in patients (FIG. 10C). Together, these data show that IL-1β expression levels were higher at baseline and remained higher after treatment in AML/MDS patients that did not respond to the Decitabine+MBG453 combination. These biomarker data suggest that IL-1β may have a role in driving resistance to Decitabine+MBG453 combination in AML/MDS.

### Example 11—Dose Escalation: NIS793

Dose escalation is conducted to establish the dose of NIS793 to be used in combination with MBG453 combination arm, as well as a possible single-agent expansion. Specifically, it is the one or more doses that have the most appropriate benefit-risk as assessed by the review of safety, tolerability, pharmacokinetics (PK), any available efficacy, and pharmacodynamics (PD), taking into consideration the maximum tolerated dose (MTD).

The MTD is the highest dose estimated to have less than 25% risk of causing a dose-limiting toxicity (DLT) during the DLT evaluation period in more than 33% of treated patients. The dose(s) selected for combination and/or expansion can be any dose equal to or less than the MTD, and may be declared without identifying the MTD. MTD is not required to be identified in this study.

Each dose escalation cohort will start with 3 to 6 newly treated patients. They must have adequate exposure and follow-up to be considered evaluable for dose escalation decisions.

Dose escalation decisions will be made when all patients in a cohort have completed the DLT evaluation period or discontinued. Decisions will be made based on a synthesis of all relevant data available from all dose levels evaluated in the ongoing study, including safety information, PK, available PD and preliminary efficacy.

Any dose escalation decisions will not exceed the dose level satisfying the escalation without overdose control (EWOC) principle by the Bayesian logistic regression model (BLRM). In all cases, the dose for the next escalation cohort will not exceed a 100% increase from the previously tested safe dose. Smaller increases in dose may be recommended by the investigators and Sponsor upon consideration of all of the available clinical data.

To better understand the safety, tolerability, PK, PD, or anti-cancer activity of NIS793 before or while proceeding with further escalation, enrichment cohorts of 6 to 10 patients may be enrolled at any dose level at or below the highest dose previously tested and shown to be safe. A cohort with a sample size of 7-10 may be opened only when the probability of observing 2 or more DLTs out of 10 patients is less than 30%.

To reduce the risk of exposing patients to an overly toxic dose, when 2 patients experience a DLT in a new cohort, the BLRM will be updated with the most up-to-date new information from all cohorts, without waiting for all patients from the current cohort to complete the evaluation period.

If the 2 DLTs occur in a cohort of patients treated at a new dose level, enrollment to that cohort will stop, and the next cohort will be opened at a lower dose level that satisfies the EWOC criteria.

If the 2 DLTs occur in a cohort of patients treated at an already tested dose level, then upon re-evaluation of all relevant data, additional patients may be enrolled into the open cohorts only if the dose still meets the EWOC criteria. Alternatively, if recruitment to the same dose cannot continue, a new cohort of patients may be recruited to a lower dose that satisfies the EWOC criteria.

Besides the scenario of 2 DLTs, the current dose being tested may be de-escalated based on new safety findings, including but not limited to observing a DLT, before a cohort is completed. Subsequent to a decision to de-escalate, re-escalation may occur if data in subsequent cohorts satisfies the EWOC criteria.

### EMBODIMENTS OF THE APPLICATION

The following are embodiments disclosed in the present application. The embodiments include, but are not limited to:

1. A combination comprising a TIM-3 inhibitor and a TGF-β inhibitor for use in treating a myelofibrosis in a subject.

2. A combination comprising a TIM-3 inhibitor and a TGF-β inhibitor for use in treating a myelodysplastic syndrome in a subject.

3. A method of treating a myelodysplastic syndrome (MDS) in a subject, comprising administering to the subject a combination of a TIM-3 inhibitor and a TGF-β inhibitor.

4. A method of treating a myelofibrosis in a subject, comprising administering to the subject a combination of a TIM-3 inhibitor and a TGF-β inhibitor.

5. The combination for use of embodiment 1 or 2, or the method of embodiment 3 or 4, wherein the TIM-3 inhibitor comprises an anti-TIM-3 antibody molecule.

6. The combination for use of embodiment 1, 2, or 5, or the method of embodiment 3-5, wherein the TIM-3 inhibitor comprises MBG453, TSR-022, LY3321367, Sym023, BGB-A425, INCAGN-2390, MBS-986258, RO-7121661, BC-3402, SHR-1702, or LY-3415244.

7. The combination for use of any one of embodiments 1, 2 or 5-6, or the method of any one of embodiments 3-6, wherein the TIM-3 inhibitor comprises MBG453.

8. The combination for use of any one of embodiments 1, 2 or 5-7, or the method of any one of embodiments 3-7, wherein the TIM-3 inhibitor is administered at a dose of about 700 mg to about 900 mg.

9. The combination for use of any one of embodiments 1, 2 or 5-8, or the method of any one of embodiments 3-8, wherein the TIM-3 inhibitor is administered at a dose of about 800 mg.

10. The combination for use of any one of embodiments 1, 2 or 5-9, or the method of any one of embodiments 3-9, wherein the TIM-3 inhibitor is administered once every four weeks.

11. The combination for use of any one of embodiments 1, 2 or 5-9, or the method of any one of embodiments 3-9, wherein the TIM-3 inhibitor is administered once every eight weeks.

12. The combination for use of any one of embodiments 1, 2 or 5-7, or the method of any one of embodiments 3-7, wherein the TIM-3 inhibitor is administered at a dose of about 500 mg to about 700 mg.

13. The combination for use of any one of embodiments 1, 2, 5-7, or 12, or the method of any one of embodiments 3-7, or 12, wherein the TIM-3 inhibitor is administered at a dose of about 600 mg.

14. The combination for use of any one of embodiments 1, 2 or 5-7, or the method of any one of embodiments 3-7, wherein the TIM-3 inhibitor is administered at a dose of about 300 mg to about 500 mg.

15. The combination for use of any one of embodiments 1, 2, 5-7, or 14, or the method of any one of embodiments 3-7, or 14, wherein the TIM-3 inhibitor is administered at a dose of about 400 mg.

16. The combination for use of any one of embodiments 1, 2, 5-9 or 12-15, or the method of any one of embodiments 3-9 or 12-15, wherein the TIM-3 inhibitor is administered once every three weeks.

17. The combination for use of any one of embodiments 1, 2, 5-9 or 12-15, or the method of any one of embodiments 3-9 or 12-15, wherein the TIM-3 inhibitor is administered once every six weeks.

18. The combination for use of any one of embodiments 12-15, or the method of any one of embodiments 12-15, wherein the TIM-3 inhibitor is administered once every four weeks.

19. The combination for use of any one of embodiments 1, 2 or 5-18, or the method of any one of embodiments 3-18, wherein the TIM-3 inhibitor is administered intravenously.

20. The combination for use of any one of embodiments 1, 2 or 5-19, or the method of any one of embodiments 3-19, wherein the TIM-3 inhibitor is administered over a period of about 20 to about 40 minutes.

21. The combination for use of any one of embodiments 1, 2 or 5-20, or the method of any one of embodiments 3-20, wherein the TIM-3 inhibitor is administered over a period of about 30 minutes.

22. The combination for use of any one of embodiments 1, 2 or 5-21, or the method of any one of embodiments 3-21, wherein the TGF-β inhibitor is an anti-TGF-β antibody molecule.

23. The combination for use of any one of embodiments 1, 2 or 5-22, or the method of any one of embodiments 3-22, wherein the TGF-β inhibitor comprises NIS793, fresolimumab, PF-06952229, or AVID200.

24. The combination for use of any one of embodiments 1, 2 or 5-23, or the method of any one of embodiments 3-23, wherein the TGF-β inhibitor comprises NIS793.

25. The combination for use of any one of embodiments 1, 2 or 5-24, or the method of any one of embodiments 3-24, wherein the TGF-β inhibitor is administered at a dose of about 1300 mg to about 1500 mg.

26. The combination for use of any one of embodiments 1, 2, or 5-25, or the method of any one of embodiments 3-25, wherein the TGF-β inhibitor is administered at a dose of about 1400 mg.

27. The combination for use of any one of embodiments 1, 2, or 5-26, or the method of any one of embodiments 3-26, wherein the TGF-β inhibitor is administered once every two weeks.

28. The combination for use of any one of embodiments 1, 2, or 5-24, or the method of any one of embodiments 3-24, wherein the TGF-β inhibitor is administered at a dose of about 2000 mg to about 2200 mg.

29. The combination for use of any one of embodiments 1, 2, 5-24, or 28, or the method of any one of embodiments 3-24, or 28, wherein the TGF-β inhibitor is administered at a dose of about 2100 mg.

30. The combination for use of any one of embodiments 1, 2, or 5-24, or the method of any one of embodiments 3-24, wherein the TGF-β inhibitor is administered at a dose of about 600 mg to about 800 mg.

31. The combination for use of any one of embodiments 1, 2, 5-24, or 30, or the method of any one of embodiments 3-24, or 30, wherein the TGF-β inhibitor is administered at a dose of about 700 mg.

32. The combination for use of any one of embodiments 1, 2, 5-26, or 28-31 or the method of any one of embodiments 3-26 or 28-31, wherein the TGF-β inhibitor is administered once every three weeks.

33. The combination for use of any one of embodiments 1, 2, 5-26, or 28-29, or the method of any one of embodiments 3-26 or 28-29, wherein the TGF-β inhibitor is administered once every six weeks.

34. The combination for use of any one of embodiments 1, 2, or 5-33, or the method of any one of embodiments 3-33, wherein the TGF-β inhibitor is administered over a period of about 20 to about 40 minutes.

35. The combination for use of any one of embodiments 1, 2, or 5-34, or the method of any one of embodiments 3-34, wherein the TGF-β inhibitor is administered over a period of about 30 minutes.

36. The combination for use of any one of embodiments 1, 2, or 5-35, or the method of any one of embodiments 3-35, wherein the TGF-β inhibitor is administered on the same day as the TIM-3 inhibitor.

37. The combination for use of any one of embodiments 1, 2, or 5-36, or the method of any one of embodiments 3-36, wherein the TGF-β inhibitor is administered after administration of the TIM-3 inhibitor is completed.

38. The combination for use of any one of embodiments 1 or 5-37, or the method of any one of embodiments 4-37, wherein the combination further comprises a PD-1 inhibitor.

39. The combination for use of any one of embodiments 1 or 5-38, or the method of any one of embodiments 4-38, wherein the PD-1 inhibitor comprises spartalizumab, nivolumab, pembrolizumab, pidilizumab, MEDI0680, REGN2810, TSR-042, PF-06801591, BGB-A317, BGB-108, INCSHR1210, or AMP-224.

40. The combination for use of any one of embodiments 1 or 5-31, or the method of any one of embodiments 4-31, wherein the PD-1 inhibitor comprises spartalizumab.

41. The combination for use of any one of embodiments 1 or 5-40, or the method of any one of embodiments 4-40, wherein the PD-1 inhibitor is administered at a dose of about 300 mg to about 500 mg.

42. The combination for use of any one of embodiments 1 or 5-41, or the method of any one of embodiments 4-41, wherein the PD-1 inhibitor is administered at a dose of about 400 mg.

43. The combination for use of any one of embodiments 1 or 5-42, or the method of any one of embodiments 4-42, wherein the PD-1 inhibitor is administered once every four weeks.

44. The combination for use of any one of embodiments 1 or 5-20, or the method of any one of embodiments 4-40, wherein the PD-1 inhibitor is administered at a dose of about 200 mg to about 400 mg.

45. The combination for use of any one of embodiments 1, 5-20, or 44, or the method of any one of embodiments 4-20, or 44 wherein the PD-1 inhibitor is administered at a dose of about 300 mg.

46. The combination for use of any one of embodiments 1 or 5-45, or the method of any one of embodiments 4-45, wherein the PD-1 inhibitor is administered once every three weeks.

47. The combination for use of any one of embodiments 1 or 5-46, or the method of any one of embodiments 4-46, wherein the PD-1 inhibitor is administered intravenously.

48. The combination for use of any one of embodiments 1 or 5-47, or the method of any one of embodiments 4-47, wherein the PD-1 inhibitor is administered over a period of about 20 to about 40 minutes.

49. The combination for use of any one of embodiments 1 or 5-48, or the method of any one of embodiments 4-48, wherein the PD-1 inhibitor is administered over a period of about 30 minutes.

50. The combination for use of any one of embodiments 1, 2, or 5-37, or the method of any one of embodiments 3-37, wherein the combination further comprises an IL-1β inhibitor.

51. The combination for use of embodiment 50, or the method of embodiment 50, wherein the IL-1β inhibitor comprises canakinumab, gevokizumab, Anakinra, diacerein, Rilonacept, IL-1β Affibody (SOBI 006, Z-FC (Swedish Orphan Biovitrum/Affibody)) and Lutikizumab (ABT-981) (Abbott), CDP-484 (Celltech), or LY-2189102 (Lilly).

52. The combination for use of embodiment 50 or 51, or the method of embodiment 50 or 51, wherein the IL-1β inhibitor comprises canakinumab.

53. The combination for use of any one of embodiments 50 to 52, or the method of any one of embodiments 50-52, wherein IL-1β inhibitor is dosed at 200 mg every 3 weeks.

54. The combination for use of any one of embodiments 50 to 52, or the method of any one of embodiments 50-52, wherein the IL-1β inhibitor is dosed at 250 mg every 4 weeks.

55. The combination for use of any one of embodiments 50 to 52, or the method of any one of embodiments 50-52, wherein the IL-1β inhibitor is dosed at 250 mg every 8 weeks.

56. The combination for use of any one of embodiments 1 or 5-55, or the method of any one of embodiments 4-46, wherein the combination further comprises a hypomethylating agent.

57. The combination for use of embodiment 56, or the method of embodiment 56, wherein the hypomethylating agent comprises azacitidine, decitabine, CC-486 or ASTX727.

58. The combination for use of embodiment 56 or 57, or the method of embodiment 56 or 57, wherein the hypomethylating agent comprises decitabine.

59. The combination for use of any one of embodiments 56-58, or the method of any one of embodiments 56-58, wherein the hypomethylating agent is administered at a dose of about 2 mg/m² to about 25 mg/m².

60. The combination for use of any one of embodiments 56-59, or the method of any one of embodiments 56-59, wherein the hypomethylating agent is administered at a dose of about 2.5 mg/m², about 5 mg/m², about 10 mg/m², or about 20 mg/m².

61. The combination for use of any one of embodiments 56-60, or the method of any one of embodiments 56-60, wherein the hypomethylating agent is administered once a day.

62. The combination for use of any one of embodiments 56-61, or the method of any one of embodiments 56-61, wherein the hypomethylating agent is administered for 5 consecutive days.

63. The combination for use of any one of embodiments 56-62, or the method of any one of embodiments 56-62, wherein the hypomethylating agent is administered on days 1, 2, 3, 4, and 5 of a 42-day cycle.

64. The combination for use of any one of embodiments 56-63, or the method of any one of embodiments 56-63, wherein the hypomethylating agent is administered over a period of about 0.5 hour to about 1.5 hour.

65. The combination for use of any one of embodiments 56-63, or the method of any one of embodiments 56-63, wherein the hypomethylating agent is administered over a period of about 1 hour.

66. The combination for use of any one of embodiments 56-59, or the method of any one of embodiments 56-58, wherein the hypomethylating agent is administered at a dose of about 2 mg/m² to about 20 mg/m².

67. The combination for use of any one of embodiments 56-59 or 66, or the method of any one of embodiments 56-59 or 66, wherein the hypomethylating agent is administered at a dose of about 2.5 mg/m², about 5 mg/m², about 7.5 mg/m², about 15 mg/m², or about 20 mg/m².

68. The combination for use of any one of embodiments 56-60 or 66-67, or the method of any one of embodiments 56-60 or 66-67, wherein the hypomethylating agent is administered once daily.

69. The combination for use of any one of embodiments 56-61 or 66-68, or the method of any one of embodiments 56-61 or 66-68, wherein the hypomethylating agent is administered for 3 consecutive days.

70. The combination for use of any one of embodiments 56-61 or 66-69, or the method of any one of embodiments 56-61 or 66-69, wherein the hypomethylating agent is administered on days 1, 2, and 3 of a 42 day cycle.

71. The combination for use of any one of embodiments 56-61 or 66-69, or the method of any one of embodiments 56-61 or 66-69, wherein the hypomethylating agent is administered on days 1, 2, and 3 of a 28 day cycle.

72. The combination for use of any one of embodiments 56-61 or 66-71, or the method of any one of embodiments 56-61 or 66-71, wherein the hypomethylating agent is administered over a period of about 0.5 hour to about 1.5 hour.

73. The combination for use of any one of embodiments 56-61 or 66-72, or the method of any one of embodiments 56-61 or 66-72, wherein the hypomethylating agent is administered over a period of about 1 hour.

74. The combination for use of any one of embodiments 56-73, or the method of any one of embodiments 56-73, wherein the hypomethylating agent is administered subcutaneously, orally or intravenously.

75. The combination for use of any one of embodiments 1 or 5-74, or the method of any one of embodiments 4-74, wherein the myelofibrosis is a primary myelofibrosis (PMF), post-ET (PET-MF) myelofibrosis, or post-PV myelofibrosis (PPV-MF).

76. The combination for use of any one of embodiments 1 or 5-75, or the method of any one of embodiments 4-75, wherein the myelofibrosis is a primary myelofibrosis (PMF).

77. The combination for use of any one of embodiments 2, 5-37, or 50-55, or the method of any one of embodiments 3, 5-37, or 50-55, wherein the myelodysplastic syndrome is a lower risk myelodysplastic syndrome (MDS), e.g., a very low risk MDS, a low risk MDS, or an intermediate risk MDS, or a higher risk myelodysplastic syndrome, e.g., a high risk MDS or a very high risk MDS.

78. The combination for use of any one of embodiments 2, 5-37, 50-55, or 77, or the method of any one of embodiments 3-37, 50-55 or 77, wherein the myelodysplastic syndrome is a lower risk myelodysplastic syndrome (MDS), e.g., a very low risk MDS, a low risk MDS, or an intermediate risk MDS.

79. A combination comprising MBG453 and NIS793 for use in treating a myelofibrosis in a subject,

optionally wherein the combination further comprising decitabine;

optionally wherein the combination further comprises PDR001, and optionally wherein MGB453 is administered at a dose of 600 mg once every three weeks, NIS793 is administered at a dose of 2100 mg once every three weeks, PDR001 is administered at a dose of 300 mg once every three weeks, and decitabine is administered at a dose of about 5 mg/m² to about 20 mg/m² on days 1, 2, and 3 of a 42 day cycle.

80. A method of treating myelofibrosis in a subject, comprising administering to the subject a combination of MBG453 and NIS793,

optionally wherein the combination further comprises decitabine,

optionally wherein the combination further comprises PDR001, and

optionally wherein MGB453 is administered at a dose of 600 mg once every three weeks, NIS793 is administered at a dose of 2100 mg once every three weeks, PDR001 is administered at a dose of 300 mg once every three weeks, and decitabine is administered at a dose of about 5 mg/m² to about 20 mg/m² on days 1, 2, and 3 of a 42 day cycle.

81. A method of treating myelofibrosis in a subject, comprising administering to the subject a combination of a MBG453 and NIS793,

optionally wherein the combination further comprises decitabine,

optionally wherein the combination further comprises canakinumab; and

optionally wherein MGB453 is administered at a dose of 600 mg once every three weeks, NIS793 is administered at a dose of 2100 mg once every three weeks, canakinumab is administered at a dose of 200 mg every three weeks, and decitabine is administered at a dose of about 5 mg/m² to about 20 mg/m² on days 1, 2, and 3 of a 42 day cycle.

82. A method of treating a myelofibrosis in a subject, comprising administering to the subject a combination of a MBG453 and NIS793,

optionally wherein the combination further comprises decitabine,

optionally wherein the combination further comprises canakinumab; and

optionally wherein MGB453 is administered at a dose of 800 mg once every four weeks, NIS793 is administered at a dose of 1400 mg once every two weeks, canakinumab is administered at a dose of 250 mg once every four weeks, and decitabine is administered at a dose of about 5 mg/m² to about 20 mg/m² on days 1, 2, and 3 of a 42 day cycle.

83. A combination comprising MBG453 and NIS793 for use in treating a myelodysplastic syndrome (MDS) in a subject,

optionally wherein MGB453 is administered at a dose of 600 mg once every three weeks, and NIS793 is administered at a dose of 2100 mg once every three weeks.

84. A combination comprising MBG453 and NIS793 for use in treating a myelodysplastic syndrome (MDS) in a subject,

optionally wherein MGB453 is administered at a dose of 800 mg once every four weeks, and NIS793 is administered at a dose of 2100 mg once every three weeks.

85. A method of treating myelodysplastic syndrome (MDS) in a subject, comprising administering to the subject a combination of MBG453 and NIS793,

optionally wherein MGB453 is administered at a dose of 600 mg once every three weeks, and NIS793 is administered at a dose of 2100 mg once every three weeks.

86. A method of treating myelodysplastic syndrome (MDS) in a subject, comprising administering to the subject a combination of MBG453 and NIS793,

optionally wherein MGB453 is administered at a dose of 800 mg once every four weeks, and NIS793 is administered at a dose of 2100 mg once every three weeks.

87. A combination comprising MBG453, NIS793, and canakinumab, for use in treating a myelodysplastic syndrome (MDS) in a subject,

optionally wherein MGB453 is administered at a dose of 800 mg once every four weeks, NIS793 is administered at a dose of 2100 mg once every three weeks, and canakinumab is administered at a dose of 250 mg once every four weeks.

88. A method of treating myelodysplastic syndrome (MDS) in a subject, comprising administering to the subject a combination of MBG453, NIS793, canakinumab,

optionally wherein MGB453 is administered at a dose of 800 mg once every four weeks, NIS793 is administered at a dose of 2100 mg once every three weeks, and canakinumab is administered at a dose of 250 mg once every four weeks.

89. A combination comprising MBG453, NIS793, and canakinumab, for use in treating a myelodysplastic syndrome (MDS) in a subject,

optionally wherein MGB453 is administered at a dose of 800 mg once every four weeks, NIS793 is administered at a dose of 1400 mg once every three weeks, and canakinumab is administered at a dose of 250 mg once every four weeks.

90. A method of treating myelodysplastic syndrome (MDS) in a subject, comprising administering to the subject a combination of MBG453, NIS793, canakinumab,

optionally wherein MGB453 is administered at a dose of 800 mg once every four weeks, NIS793 is administered at a dose of 1400 mg once every three weeks, and canakinumab is administered at a dose of 250 mg once every four weeks.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A combination comprising a TIM-3 inhibitor and a TGF-β inhibitor for use in treating a myelofibrosis in a subject.

2. A combination comprising a TIM-3 inhibitor and a TGF-β inhibitor for use in treating a myelodysplastic syndrome in a subject.

3. A method of treating a myelodysplastic syndrome (MDS) in a subject, comprising administering to the subject a combination of a TIM-3 inhibitor and a TGF-β inhibitor.

4. A method of treating a myelofibrosis in a subject, comprising administering to the subject a combination of a TIM-3 inhibitor and a TGF-β inhibitor.

5. The combination for use of claim 1 or 2, or the method of claim 3 or 4, wherein the TIM-3 inhibitor comprises an anti-TIM-3 antibody molecule.

6. The combination for use of claim 1, 2, or 5, or the method of claim 3-5, wherein the TIM-3 inhibitor comprises MBG453, TSR-022, LY3321367, Sym023, BGB-A425, INCAGN-2390, MBS-986258, RO-7121661, BC-3402, SHR-1702, or LY-3415244.

7. The combination for use of any one of claims 1, 2 or 5-6, or the method of any one of claims 3-6, wherein the TIM-3 inhibitor comprises MBG453.

8. The combination for use of any one of claims 1, 2 or 5-7, or the method of any one of claims 3-7, wherein the TIM-3 inhibitor is administered at a dose of about 700 mg to about 900 mg.

9. The combination for use of any one of claims 1, 2 or 5-8, or the method of any one of claims 3-8, wherein the TIM-3 inhibitor is administered at a dose of about 800 mg.

10. The combination for use of any one of claims 1, 2 or 5-9, or the method of any one of claims 3-9, wherein the TIM-3 inhibitor is administered once every four weeks.

11. The combination for use of any one of claims 1, 2 or 5-9, or the method of any one of claims 3-9, wherein the TIM-3 inhibitor is administered once every eight weeks.

12. The combination for use of any one of claims 1, 2 or 5-7, or the method of any one of claims 3-7, wherein the TIM-3 inhibitor is administered at a dose of about 500 mg to about 700 mg.

13. The combination for use of any one of claims 1, 2, 5-7, or 12, or the method of any one of claims 3-7, or 12, wherein the TIM-3 inhibitor is administered at a dose of about 600 mg.

14. The combination for use of any one of claims 1, 2 or 5-7, or the method of any one of claims 3-7, wherein the TIM-3 inhibitor is administered at a dose of about 300 mg to about 500 mg.

15. The combination for use of any one of claims 1, 2, 5-7, or 14, or the method of any one of claims 3-7, or 14, wherein the TIM-3 inhibitor is administered at a dose of about 400 mg.

16. The combination for use of any one of claims 1, 2, 5-9 or 12-15, or the method of any one of claims 3-9 or 12-15, wherein the TIM-3 inhibitor is administered once every three weeks.

17. The combination for use of any one of claims 1, 2, 5-9 or 12-15, or the method of any one of claims 3-9 or 12-15, wherein the TIM-3 inhibitor is administered once every six weeks.

18. The combination for use of any one of claims 12-15, or the method of any one of claims 12-15, wherein the TIM-3 inhibitor is administered once every four weeks.

19. The combination for use of any one of claims 1, 2 or 5-18, or the method of any one of claims 3-18, wherein the TIM-3 inhibitor is administered intravenously.

20. The combination for use of any one of claims 1, 2 or 5-19, or the method of any one of claims 3-19, wherein the TIM-3 inhibitor is administered over a period of about 20 to about 40 minutes.

21. The combination for use of any one of claims 1, 2 or 5-20, or the method of any one of claims 3-20, wherein the TIM-3 inhibitor is administered over a period of about 30 minutes.

22. The combination for use of any one of claims 1, 2 or 5-21, or the method of any one of claims 3-21, wherein the TGF-β inhibitor is an anti-TGF-β antibody molecule.

23. The combination for use of any one of claims 1, 2 or 5-22, or the method of any one of claims 3-22, wherein the TGF-β inhibitor comprises NIS793, fresolimumab, PF-06952229, or AVID200.

24. The combination for use of any one of claims 1, 2 or 5-23, or the method of any one of claims 3-23, wherein the TGF-β inhibitor comprises NIS793.

25. The combination for use of any one of claims 1, 2 or 5-24, or the method of any one of claims 3-24, wherein the TGF-β inhibitor is administered at a dose of about 1300 mg to about 1500 mg.

26. The combination for use of any one of claims 1, 2, or 5-25, or the method of any one of claims 3-25, wherein the TGF-β inhibitor is administered at a dose of about 1400 mg.

27. The combination for use of any one of claims 1, 2, or 5-26, or the method of any one of claims 3-26, wherein the TGF-β inhibitor is administered once every two weeks.

28. The combination for use of any one of claims 1, 2, or 5-24, or the method of any one of claims 3-24, wherein the TGF-β inhibitor is administered at a dose of about 2000 mg to about 2200 mg.

29. The combination for use of any one of claims 1, 2, 5-24, or 28, or the method of any one of claims 3-24, or 28, wherein the TGF-β inhibitor is administered at a dose of about 2100 mg.

30. The combination for use of any one of claims 1, 2, or 5-24, or the method of any one of claims 3-24, wherein the TGF-β inhibitor is administered at a dose of about 600 mg to about 800 mg.

31. The combination for use of any one of claims 1, 2, 5-24, or 30, or the method of any one of claims 3-24, or 30, wherein the TGF-β inhibitor is administered at a dose of about 700 mg.

32. The combination for use of any one of claims 1, 2, 5-26, or 28-31 or the method of any one of claims 3-26 or 28-31, wherein the TGF-β inhibitor is administered once every three weeks.

33. The combination for use of any one of claims 1, 2, 5-26, or 28-29, or the method of any one of claims 3-26 or 28-29, wherein the TGF-β inhibitor is administered once every six weeks.

34. The combination for use of any one of claims 1, 2, or 5-33, or the method of any one of claims 3-33, wherein the TGF-β inhibitor is administered over a period of about 20 to about 40 minutes.

35. The combination for use of any one of claims 1, 2, or 5-34, or the method of any one of claims 3-34, wherein the TGF-β inhibitor is administered over a period of about 30 minutes.

36. The combination for use of any one of claims 1, 2, or 5-35, or the method of any one of claims 3-35, wherein the TGF-β inhibitor is administered on the same day as the TIM-3 inhibitor.

37. The combination for use of any one of claims 1, 2, or 5-36, or the method of any one of claims 3-36, wherein the TGF-β inhibitor is administered after administration of the TIM-3 inhibitor is completed.

38. The combination for use of any one of claim 1 or 5-37, or the method of any one of claims 4-37, wherein the combination further comprises a PD-1 inhibitor.

39. The combination for use of any one of claim 1 or 5-38, or the method of any one of claims 4-38, wherein the PD-1 inhibitor comprises spartalizumab, nivolumab, pembrolizumab, pidilizumab, MEDI0680, REGN2810, TSR-042, PF-06801591, BGB-A317, BGB-108, INCSHR1210, or AMP-224.

40. The combination for use of any one of claim 1 or 5-31, or the method of any one of claims 4-31, wherein the PD-1 inhibitor comprises spartalizumab.

41. The combination for use of any one of claim 1 or 5-40, or the method of any one of claims 4-40, wherein the PD-1 inhibitor is administered at a dose of about 300 mg to about 500 mg.

42. The combination for use of any one of claim 1 or 5-41, or the method of any one of claims 4-41, wherein the PD-1 inhibitor is administered at a dose of about 400 mg.

43. The combination for use of any one of claim 1 or 5-42, or the method of any one of claims 4-42, wherein the PD-1 inhibitor is administered once every four weeks.

44. The combination for use of any one of claim 1 or 5-20, or the method of any one of claims 4-40, wherein the PD-1 inhibitor is administered at a dose of about 200 mg to about 400 mg.

45. The combination for use of any one of claims 1, 5-20, or 44, or the method of any one of claims 4-20, or 44 wherein the PD-1 inhibitor is administered at a dose of about 300 mg.

46. The combination for use of any one of claim 1 or 5-45, or the method of any one of claims 4-45, wherein the PD-1 inhibitor is administered once every three weeks.

47. The combination for use of any one of claim 1 or 5-46, or the method of any one of claims 4-46, wherein the PD-1 inhibitor is administered intravenously.

48. The combination for use of any one of claim 1 or 5-47, or the method of any one of claims 4-47, wherein the PD-1 inhibitor is administered over a period of about 20 to about 40 minutes.

49. The combination for use of any one of claim 1 or 5-48, or the method of any one of claims 4-48, wherein the PD-1 inhibitor is administered over a period of about 30 minutes.

50. The combination for use of any one of claims 1, 2, or 5-37, or the method of any one of claims 3-37, wherein the combination further comprises an IL-1β inhibitor.

51. The combination for use of claim 50, or the method of claim 50, wherein the IL-1β inhibitor comprises canakinumab, gevokizumab, Anakinra, diacerein, Rilonacept, IL-1 Affibody (SOBI 006, Z-FC (Swedish Orphan Biovitrum/Affibody)) and Lutikizumab (ABT-981) (Abbott), CDP-484 (Celltech), or LY-2189102 (Lilly).

52. The combination for use of claim 50 or 51, or the method of claim 50 or 51, wherein the IL-1β inhibitor comprises canakinumab.

53. The combination for use of any one of claims 50 to 52, or the method of any one of claims 50-52, wherein IL-1β inhibitor is dosed at 200 mg every 3 weeks.

54. The combination for use of any one of claims 50 to 52, or the method of any one of claims 50-52, wherein the IL-1β inhibitor is dosed at 250 mg every 4 weeks.

55. The combination for use of any one of claims 50 to 52, or the method of any one of claims 50-52, wherein the IL-1β inhibitor is dosed at 250 mg every 8 weeks.

56. The combination for use of any one of claim 1 or 5-55, or the method of any one of claims 4-46, wherein the combination further comprises a hypomethylating agent.

57. The combination for use of claim 56, or the method of claim 56, wherein the hypomethylating agent comprises azacitidine, decitabine, CC-486 or ASTX727.

58. The combination for use of claim 56 or 57, or the method of claim 56 or 57, wherein the hypomethylating agent comprises decitabine.

59. The combination for use of any one of claims 56-58, or the method of any one of claims 56-58, wherein the hypomethylating agent is administered at a dose of about 2 mg/m² to about 25 mg/m².

60. The combination for use of any one of claims 56-59, or the method of any one of claims 56-59, wherein the hypomethylating agent is administered at a dose of about 2.5 mg/m², about 5 mg/m², about 10 mg/m², or about 20 mg/m².

61. The combination for use of any one of claims 56-60, or the method of any one of claims 56-60, wherein the hypomethylating agent is administered once a day.

62. The combination for use of any one of claims 56-61, or the method of any one of claims 56-61, wherein the hypomethylating agent is administered for 5 consecutive days.

63. The combination for use of any one of claims 56-62, or the method of any one of claims 56-62, wherein the hypomethylating agent is administered on days 1, 2, 3, 4, and 5 of a 42-day cycle.

64. The combination for use of any one of claims 56-63, or the method of any one of claims 56-63, wherein the hypomethylating agent is administered over a period of about 0.5 hour to about 1.5 hour.

65. The combination for use of any one of claims 56-63, or the method of any one of claims 56-63, wherein the hypomethylating agent is administered over a period of about 1 hour.

66. The combination for use of any one of claims 56-59, or the method of any one of claims 56-58, wherein the hypomethylating agent is administered at a dose of about 2 mg/m² to about 20 mg/m².

67. The combination for use of any one of claims 56-59 or 66, or the method of any one of claims 56-59 or 66, wherein the hypomethylating agent is administered at a dose of about 2.5 mg/m², about 5 mg/m², about 7.5 mg/m², about 15 mg/m², or about 20 mg/m².

68. The combination for use of any one of claims 56-60 or 66-67, or the method of any one of claims 56-60 or 66-67, wherein the hypomethylating agent is administered once daily.

69. The combination for use of any one of claims 56-61 or 66-68, or the method of any one of claims 56-61 or 66-68, wherein the hypomethylating agent is administered for 3 consecutive days.

70. The combination for use of any one of claims 56-61 or 66-69, or the method of any one of claims 56-61 or 66-69, wherein the hypomethylating agent is administered on days 1, 2, and 3 of a 42 days cycle.

71. The combination for use of any one of claims 56-61 or 66-69, or the method of any one of claims 56-61 or 66-69, wherein the hypomethylating agent is administered on days 1, 2, and 3 of a 28 days cycle.

72. The combination for use of any one of claims 56-61 or 66-71, or the method of any one of claims 56-61 or 66-71, wherein the hypomethylating agent is administered over a period of about 0.5 hour to about 1.5 hour.

73. The combination for use of any one of claims 56-61 or 66-72, or the method of any one of claims 56-61 or 66-72, wherein the hypomethylating agent is administered over a period of about 1 hour.

74. The combination for use of any one of claims 56-73, or the method of any one of claims 56-73, wherein the hypomethylating agent is administered subcutaneously, orally or intravenously.

75. The combination for use of any one of claim 1 or 5-74, or the method of any one of claims 4-74, wherein the myelofibrosis is a primary myelofibrosis (PMF), post-ET (PET-MF) myelofibrosis, or post-PV myelofibrosis (PPV-MF).

76. The combination for use of any one of claim 1 or 5-75, or the method of any one of claims 4-75, wherein the myelofibrosis is a primary myelofibrosis (PMF).

77. The combination for use of any one of claims 2, 5-37, or 50-55, or the method of any one of claims 3, 5-37, or 50-55, wherein the myelodysplastic syndrome is a lower risk myelodysplastic syndrome (MDS), e.g., a very low risk MDS, a low risk MDS, or an intermediate risk MDS, or a higher risk myelodysplastic syndrome, e.g., a high risk MDS or a very high risk MDS.

78. The combination for use of any one of claims 2, 5-37, 50-55, or 77, or the method of any one of claims 3-37, 50-55 or 77, wherein the myelodysplastic syndrome is a lower risk myelodysplastic syndrome (MDS), e.g., a very low risk MDS, a low risk MDS, or an intermediate risk MDS.

79. A combination comprising MBG453 and NIS793 for use in treating a myelofibrosis in a subject,
optionally wherein the combination further comprising decitabine;
optionally wherein the combination further comprises PDR001, and optionally wherein MGB453 is administered at a dose of 600 mg once every three weeks, NIS793 is administered at a dose of 2100 mg once every three weeks, PDR001 is administered at a dose of 300 mg once every three weeks, and decitabine is administered at a dose of about 5 mg/m² to about 20 mg/m² on days 1, 2, and 3 of a 42 day cycle.

80. A method of treating myelofibrosis in a subject, comprising administering to the subject a combination of MBG453 and NIS793,
optionally wherein the combination further comprises decitabine,
optionally wherein the combination further comprises PDR001, and
optionally wherein MGB453 is administered at a dose of 600 mg once every three weeks, NIS793 is administered at a dose of 2100 mg once every three weeks, PDR001 is administered at a dose of 300 mg once every three weeks, and decitabine is administered at a dose of about 5 mg/m² to about 20 mg/m² on days 1, 2, and 3 of a 42 day cycle.

81. A method of treating myelofibrosis in a subject, comprising administering to the subject a combination of a MBG453 and NIS793,
optionally wherein the combination further comprises decitabine,
optionally wherein the combination further comprises canakinumab; and
optionally wherein MGB453 is administered at a dose of 600 mg once every three weeks, NIS793 is administered at a dose of 2100 mg once every three weeks, canakinumab is administered at a dose of 200 mg every three weeks, and decitabine is administered at a dose of about 5 mg/m² to about 20 mg/m² on days 1, 2, and 3 of a 42 day cycle.

82. A method of treating a myelofibrosis in a subject, comprising administering to the subject a combination of a MBG453 and NIS793,
optionally wherein the combination further comprises decitabine,
optionally wherein the combination further comprises canakinumab; and
optionally wherein MGB453 is administered at a dose of 800 mg once every four weeks, NIS793 is administered at a dose of 1400 mg once every two weeks, canakinumab is administered at a dose of 250 mg once every four weeks, and decitabine is administered at a dose of about 5 mg/m² to about 20 mg/m² on days 1, 2, and 3 of a 42 day cycle.

83. A combination comprising MBG453 and NIS793 for use in treating a myelodysplastic syndrome (MDS) in a subject,
optionally wherein MGB453 is administered at a dose of 600 mg once every three weeks, and NIS793 is administered at a dose of 2100 mg once every three weeks.

84. A combination comprising MBG453 and NIS793 for use in treating a myelodysplastic syndrome (MDS) in a subject,
optionally wherein MGB453 is administered at a dose of 800 mg once every four weeks, and NIS793 is administered at a dose of 2100 mg once every three weeks.

85. A method of treating myelodysplastic syndrome (MDS) in a subject, comprising administering to the subject a combination of MBG453 and NIS793,
optionally wherein MGB453 is administered at a dose of 600 mg once every three weeks, and NIS793 is administered at a dose of 2100 mg once every three weeks.

86. A method of treating myelodysplastic syndrome (MDS) in a subject, comprising administering to the subject a combination of MBG453 and NIS793,
optionally wherein MGB453 is administered at a dose of 800 mg once every four weeks, and NIS793 is administered at a dose of 2100 mg once every three weeks.

87. A combination comprising MBG453, NIS793, and canakinumab, for use in treating a myelodysplastic syndrome (MDS) in a subject,
optionally wherein MGB453 is administered at a dose of 800 mg once every four weeks, NIS793 is administered at a dose of 2100 mg once every three weeks, and canakinumab is administered at a dose of 250 mg once every four weeks.

88. A method of treating myelodysplastic syndrome (MDS) in a subject, comprising administering to the subject a combination of MBG453, NIS793, canakinumab,
optionally wherein MGB453 is administered at a dose of 800 mg once every four weeks, NIS793 is administered at a dose of 2100 mg once every three weeks, and canakinumab is administered at a dose of 250 mg once every four weeks.

89. A combination comprising MBG453, NIS793, and canakinumab, for use in treating a myelodysplastic syndrome (MDS) in a subject,
optionally wherein MGB453 is administered at a dose of 800 mg once every four weeks, NIS793 is administered at a dose of 1400 mg once every three weeks, and canakinumab is administered at a dose of 250 mg once every four weeks.

90. A method of treating myelodysplastic syndrome (MDS) in a subject, comprising administering to the subject a combination of MBG453, NIS793, canakinumab,
optionally wherein MGB453 is administered at a dose of 800 mg once every four weeks, NIS793 is administered at a dose of 1400 mg once every three weeks, and canakinumab is administered at a dose of 250 mg once every four weeks.
